(19) United States
(12) Patent Application Publication
(10) Pub. No.: US 2026/0308591 A1
(43) Pub. Date: Oct. 8, 2026

(54) COMPOSITIONS AND METHODS FOR REGULATING EXPRESSION OF ATXN2

(71) Applicant: Riboway Therapeutics, Inc., Cambridge, MA (US)

(72) Inventors: Qinghong Yan, Cambridge, MA (US); Sebastien Weyn, Cambridge, MA (US)

(73) Assignee: Riboway Therapeutics, Inc., Cambridge, MA (US)

(21) Appl. No.: 19/485,399

(22) PCT Filed: May 17, 2024
(86) PCT No.: PCT/US2024/030096

§ 371 Date: Nov. 18, 2025

Related U.S. Application Data
(60) Provisional application No. 63/503,163, filed on May 19, 2023.

(51) Int. Cl. C12N 15/113; A61K 31/712; A61K 31/7125

(52) U.S. Cl. CPC C12N 15/113; A61K 31/712; A61K 31/7125; C12N 2310/11; C12N 2310/315; C12N 2310/321; C12N 2320/33

(57) ABSTRACT

The present disclosure provides an antisense oligonucleotide that hybridizes to a target region in an ATXN2 RNA transcript, wherein hybridization of the antisense oligonucleotide to the target region changes (e.g., decreases) the expression of a functional protein encoded by the ATXN2 RNA transcript in a cell. The present disclosure further provides antisense oligonucleotides, pharmaceutical compositions, and methods for the treatment, prevention, or amelioration of diseases, disorders, and conditions associated with ATXN2 in a subject in need thereof.

### CROSS-REFERENCE TO RELATED APPLICATION

This application claims the benefit of priority to U.S. Provisional Application No. 63/503,163, filed May 19, 2023, the entire contents of which is incorporated herein by reference for all purposes.

### SEQUENCE LISTING

The present application is being filed along with a Sequence Listing in electronic format. The Sequence Listing is provided as a file entitled 044369-00007_ST26.xml, created May 17, 2024, which is 300 KB in size. The information in the electronic format of the sequence listing is incorporated herein by reference in its entirety.

### BACKGROUND

Newly synthesized eukaryotic mRNA molecules, known as primary transcripts or pre-mRNA, are processed before translation. A 5′ methylated cap and an approximately 200-250 base poly(A) tail at the 3′ end of the transcript are added during pre-mRNA processing. The processing of mRNA from pre-mRNA also frequently involves splicing of the pre-mRNA, which occurs during maturation of 90-95% of mammalian mRNAs. Introns (or intervening sequences) are regions of a pre-mRNA (or its encoding DNA) that are not present in the mature mRNA's coding sequence. Exons are primary transcript regions that remain in mature mRNA. The mature mRNA sequence is formed by splicing together the exons. Splice junctions are also known as splice sites, with the 5′ side being referred to as the “5′ splice site” or “splice donor site” and the 3′ side as the “3′ splice site” or “splice acceptor site.” The 3′ end of an upstream exon is joined to the 5′ end of a downstream exon during splicing. Consequently, the unspliced pre-mRNA possesses an exon/intron junction at the 5′ end of an intron and an intron/exon junction at the 3′ end of an intron. In mature mRNA, the exons are contiguous following the removal of the intron at what is sometimes referred to as the exon/exon junction or boundary. Alternative splicing, which is defined as the splicing together of various combinations of exons, frequently results in the production of multiple mRNA transcripts from a single gene.

Antisense technology is an efficient means for modulating the expression of one or more specific gene products, including alternative splice products, and is uniquely useful in a number of therapeutic, diagnostic, and research applications. The principle behind antisense technology is that an antisense oligonucleotide, which hybridizes to a target nucleic acid, modulates gene expression activities such as transcription, splicing or translation through one of a number of RNA regulatory mechanisms, such as the nonsense-mediated decay (“NMD”) pathway. NMD is an evolutionarily conserved RNA surveillance system initially thought to selectively mitigate deleterious effects of premature stop codons (e.g., resulting from a point mutation).

Ataxin-2 (“ATXN2”) is an RNA-binding protein encoded by a gene located on human chromosome 12q24.12, which encodes several mRNA transcript variants (e.g., NCBI Accession Nos. NM_002973.4, NM_001310121.1, NM_001310123.1, and NM_001372574.1) and multiple protein isoforms (e.g., NCBI Accession Nos. NP_002964.4, NP_001297050.1, NP_001297052.1, and NP_001359503.1). ATXN2 is a regulator of stress granule assembly that has been implicated in the pathogenesis of neurodegenerative diseases. Polyglutamine (“CAG”) expansions of >34 repeats within ATXN2 protein isoforms are causative of the disease spinocerebellar ataxia type 2 (“SCA2”). Intermediate expansions that do not reach the threshold for SCA2 are now recognized as a risk factor for amyotrophic lateral sclerosis (“ALS”), and ATXN2 has been shown to be a modifier of TDP43 toxicity in yeast, flies, and mouse models of ALS. Despite efforts from researchers and medical professionals worldwide who have been trying to address ATXN2-associated genetic disorders, there still remains a critical need for safe and effective treatments for such conditions.

### BRIEF SUMMARY

The present disclosure provides antisense oligonucleotide (“ASO”) constructs that may be used to modulate the expression of ATXN2 (e.g., using the NMD pathway), as well as compositions, and methods, related thereto.

In a first general aspect, the disclosure provides an antisense oligonucleotide that hybridizes to a target region in an ATXN2 RNA transcript, wherein hybridization of the antisense oligonucleotide to the target region changes (e.g., decreases) the expression of a functional protein encoded by the ATXN2 RNA transcript in a cell.

In some aspects, the ATXN2 RNA transcript is a pre-mRNA molecule.

In some aspects, the antisense oligonucleotide has a length of 10, 11, 12, 13, 14, 15, 16, 17, 18, 19, 20, 21, 22, 23, 24, 25, 26, 27, 28, 29, or 30 nucleotides, or a length within a range with endpoints defined by any pair of the foregoing length values. For example, in some aspects, the antisense oligonucleotide is 15 to 25 nucleotides in length, or 18 to 20 nucleotides in length.

In some aspects, the target region is 10, 11, 12, 13, 14, 15, 16, 17, 18, 19, 20, 21, 22, 23, 24, 25, 26, 27, 28, 29, or 30 nucleotides in length. In some aspects, the length of the antisense oligonucleotide is equal to the length of the target region; in others, the length of the antisense oligonucleotide is longer than that of the target region.

In some aspects, the antisense oligonucleotide comprises a nucleotide sequence of any one of SEQ ID NOs: 10-137. In some aspects, the antisense oligonucleotide comprises a nucleotide sequence having 1, 2, 3, 4, 5, 6, 7, 8, 9, or 10 nucleotide substitutions as compared to any one of SEQ ID NOs: 10-137. In some aspects, the antisense oligonucleotide comprises a nucleotide sequence having at least 85, 86, 87, 88, 89, 90, 91, 92, 93, 94, 95, 96, 97, 98, or 99% sequence identity as compared to any one of SEQ ID NOs: 10-137.

In some aspects, the antisense oligonucleotide comprises a nucleotide sequence identical to a contiguous 18-22 mer portion of any one of SEQ ID NOs: 10-12. In some aspects, the antisense oligonucleotide comprises a 10, 11, 12, 13, 14, 15, 16, 17, 18, 19, 20, 21, 22, 23, 24, 25, 26, 27, 28, 29, or 30 nucleotide sequence, having 1, 2, 3, 4, 5, 6, 7, 8, 9, or 10 nucleotide substitutions as compared to any one of SEQ ID NOs: 10-12, or an aligned portion thereof. In some aspects, the antisense oligonucleotide comprises a nucleotide sequence identical to any contiguous 18-22 mer portion of SEQ ID NOs: 10-12, with 1, 2, 3, 4 or 5 nucleotide substitutions.

In some aspects, the antisense oligonucleotide comprises a nucleotide sequence identical to a contiguous 10, 11, 12, 13, 14, 15, 16, 17, 18, or 19, 20 mer portion of any one of SEQ ID NOs: 13-137. In some aspects, the antisense oligonucleotide comprises a 10, 11, 12, 13, 14, 15, 16, 17, 18, 19, 20, 21, 22, 23, 24, 25, 26, 27, 28, 29, or 30 nucleotide sequence, having 1, 2, 3, 4, 5, 6, 7, 8, 9, or 10 nucleotide substitutions as compared to any one of SEQ ID NOs: 13-137, or an aligned portion thereof. In some aspects, the antisense oligonucleotide comprises a nucleotide sequence identical to any contiguous 15, 16, 17, 18, 19, or 20 mer portion of SEQ ID NOs: 13-137, with 1, 2, 3, 4 or 5 nucleotide substitutions. Also contemplated in the foregoing are insertions or deletions in any antisense oligonucleotide identical or substantially similar to any contiguous 15-20 mer portion of SEQ ID Nos 13-137 which insertions or deletions are of 1, 2, 3, 4 or 5 nucleotides.

In some aspects, the antisense oligonucleotide comprises one or more modified nucleotides.

In some aspects, the one or more modified nucleotides comprise a modification of a ribose group, a phosphate group, a nucleobase, or a combination thereof.

In some aspects, the modification of the ribose group comprises 2′-O-methyl, 2′-fluoro, 2′-deoxy, 2′-O-(2-methoxyethyl) (“MOE”), 2′-O-alkyl, 2′-O-alkoxy, 2′-O-alkylamino, 2′-NH2, a constrained nucleotide, or a combination thereof. In some aspects, the constrained nucleotide comprises a locked nucleic acid (LNA), an ethyl-constrained nucleotide, a 2′-(S)-constrained ethyl (“S-cEt”) nucleotide, a constrained MOE, a 2′-0,4′-C-aminomethylene bridged nucleic acid (2′,4′-BNANC), an alpha-L-locked nucleic acid, a tricyclo-DNA, or a combination thereof.

In some aspects, the modification of the phosphate group comprises a phosphorothioate, a phosphonoacetate (“PACE”), a thiophosphonoacetate (“thioPACE”), an amide, a triazole, a phosphonate, a phosphotriester modification, or a combination thereof.

In some aspects, the modification of the nucleobase group comprises 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, one or more halogenated aromatic groups, or a combination thereof.

In a second general aspect, the disclosure provides a pharmaceutical composition, comprising at least one antisense oligonucleotide selected from the antisense oligonucleotides of any one of claims 1-16, and at least one pharmaceutically acceptable carrier, diluent or buffer.

In some aspects, the at least one antisense oligonucleotide comprises a plurality of sequentially different antisense oligonucleotides (e.g., the pharmaceutical composition may comprise a mixture of any of the ASO sequences disclosed herein). In some aspects, the pharmaceutical composition may comprise, 2, 3, 4, 5, 6 or more distinct ATXN2 ASO sequences.

In some aspects, the at least one antisense oligonucleotide is present in the composition at 0.001 to 100 mg/ml. In some aspects, the at least one antisense oligonucleotide is present in a unit dose amount. In some aspects, the unit dose comprises 0.001 to 100 mg.

In a third general aspect, the disclosure provides a method of treating a disease or disorder in a subject in need thereof, comprising: a) administering an effective amount of any of the antisense oligonucleotides (or pharmaceutical compositions) disclosed herein, to the subject; and b) treating the disease or disorder. In some aspects, the disease or disorder is a neurodegenerative disease (e.g., Spinocerebellar ataxia, ALS, parkinsonism, Frontotemporal dementia, Alzheimer's disease) or a proteinopathy disease such as TDP43 proteinopathy.

In some aspects, treating the disease or disorder comprises preventing, reducing, slowing, or eliminating one or more symptoms of the disease or disorder. In some aspects, treating the disease or disorder comprises improving motor function in the subject by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100%, or by an amount within a range defined by endpoints selected from any of the foregoing values. In some aspects, treating the disease or disorder comprises improving motor rotarod performance in the subject by at least 10, 15, or 20%. In some aspects, the improvement may be determined using the ALS Functional Rating Scale Revised (“ALSFRS-R”) or any other metric known in the art.

In some aspects, the effective amount is an amount sufficient to reduce expression of a protein encoded by ATXN2 in at least one cell or tissue of the subject. In some aspects, the cell is a nerve cell (e.g., a Purkinje cell). In some aspects, an effective amount comprises an amount sufficient to result in a concentration of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nM in one or more cells of the subject. In some aspects, methods according to the disclosure may comprise administering a pharmaceutically acceptable dosage form comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg (or an amount within a range defined by any pair of the foregoing values) of one or more of the antisense oligonucleotides described herein. For example, a liquid formulation suitable for administration may comprise 5-20 mg of an antisense oligonucleotide described herein in 1, 2, 3, 4, 5, 6, 7, 8, 9, 10, 11, 12, 13, 14, 15, 16, 17, 18, 19, or 20 mL (or a volume within a range defined by any pair of the foregoing values) of solution. In some aspects, administering comprises administering the antisense oligonucleotide or the pharmaceutical composition at least, at most, or exactly 1, 2, 3, 4, or 5 times per day. In some aspects, the antisense oligonucleotide or the pharmaceutical composition is administered parenterally.

In a fourth general aspect, the disclosure provides a cell comprising any of the antisense oligonucleotides disclosed herein, or a vector configured to express any of the antisense oligonucleotides disclosed herein. In some aspects, the disclosure provides lipid nanoparticles or extracellular vesicles comprising one or more of the antisense oligonucleotides disclosed herein.

In a fifth general aspect, the disclosure provides a method of reducing the amount or activity of a target mRNA encoding an ATXN2 isoform in a cell, comprising contacting the cell with an antisense oligonucleotide configured to hybridize with the target mRNA; and causing the target mRNA transcript to be degraded (e.g., by NMD). In some aspects, the antisense oligonucleotide is configured to hybridize with a plurality of contiguous nucleotides within exon 8, exon 9, or the intronic region between exon 8 and exon 9, of ATXN2.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### DESCRIPTION OF THE FIGURES

The drawings set forth herein illustrate and describe exemplary aspects of the disclosure and are not meant to limit the scope of the invention as defined by the claims.

FIG. 1 describes the genomic location of the target alternative splicing events in the ATXN2 gene as visualized by the UCSC genome browser (genome.ucsc.edu) with the location of the putative premature termination codon (“PTC”) indicated for each modulated transcript. This includes three 5′ splice site variants of exon 8 (47nt inclusion, 25nt skipping, and 70nt skipping) and the complete skipping of either of the two variants of exon 18 (61nt and 67nt in length). Between exon 18 and exon 20 are two alternatively spliced exons of lengths 45nt and 33nt; the PTC will occur in the first included exon of those three (45nt, 33nt, or exon 20) if exon 18 is skipped. Transcript tracks annotated by GENCODE V41 are shown (Frankish A et al., Nucleic Acids Res 2021; PMID 33270111).

FIG. 2 describes a mechanism by which the present ASOs regulate gene expression. The normal processing of ATXN2 RNA is shown on the top right; and the desired effect on modulated ATXN2 RNA processing is shown on the bottom right, using the 47nt inclusion of exon 8 as an illustrative example; the other events follow a similar scheme. The ASOs described herein are designed to trigger either of the following to and subject the ATXN2 mRNA molecule to nonsense-mediated decay: 1) alternative 5′ splicing of exon 8 (47nt inclusion, 70nt skipping, or 25nt skipping); 2) skipping of alternative exon 18 (which has two variants, 61nt and 67nt). Consequently, the expression of the ATXN2 protein will be reduced.

FIGS. 3-4 characterize ATXN2 mRNA isoform expression at the exon 8 target region (depicted in FIG. 1) in U2OS cells treated by control or experimental ASOs. In FIG. 3, RT-PCR products of the exon 8 region are visualized in an acrylamide gel. The bands correspond to the various isoforms as indicated. Upon treatment by some of the ASOs described herein, the canonical form was reduced (resulting in a weaker band) and the alternative forms were increased (resulting in a stronger band), suggesting effective modulation of exon 8 splicing by such ASOs. FIG. 4 shows the quantification of the expression ratio of each isoform (% 47nt inclusion, % 70nt skipping, % 25nt skipping) determined by densitometry analysis of the acrylamide gel image.

FIG. 5 is a set of agarose gel pictures showing the characterization of the 47nt alternative splicing in the exon 8 target region (depicted in FIG. 1) in cells treated by control or experimental ASOs. The upper PCR band correlates to the 47nt inclusion isoform and the lower PCR band correlates to the canonical isoform. ASOs were tested in A549 or T98G cells, as labeled.

FIGS. 6-7 characterize the 47nt alternative splicing in A549 cells treated with selected ASOs. FIG. 6 is an acrylamide gel image depicting the isoform expression and FIG. 7 shows the quantification of isoform ratio (% 47nt inclusion) by densitometry analysis of the acrylamide gel image.

FIGS. 8-9 characterize the alternative splicing of the exon 18 region in U2OS cells following transfection with ASOs. In FIG. 8, RT-PCR products of the exon 18 region are visualized in an acrylamide gel. There are 4 alternative splicing events around exon 18:1 & 2) exon18 (61 or 67nt exon), which is the targeted skipping event; 3) a 45nt exon (hg38 chr12:111,482,787-111,482,831) which contains a stop codon; 4) a 33nt exon (hg38 chr12:111,479,070-111,479,102). These events can occur in multiple combinations. Five bands are observed and labeled (some are visible only with increased contrast). FIG. 9 shows the quantification of the bands determined by densitometry analysis. Exon 18 (61/67 nt), the 33nt exon, and the 33+45nt exon combination were abundant enough for quantification.

FIG. 10 depicts the dose-response effects of ASO treatments on the 47nt alternative splicing. T98G cells were treated by different doses (10 nM, 25 nM, 50 nM and 100 nM, in duplicates) of the control and three selected ASOs disclosed herein. RT-PCR products are visualized on the agarose gels.

FIGS. 11-15 characterize the quantification of isoform ratio (% 47nt inclusion) using digital PCR (dPCR) upon treatment with selected ASOs. Two probes with different fluorescence were used to detect the isoform expression: one detecting the total isoform (fluorescence signal in y-axis), the other detecting the canonical isoform (fluorescence signal in x-axis). 2-D plots of the two fluorescence channels are shown in FIGS. 11-12. Two representative ASO-treated samples are shown. The circled region indicates signal from the isoform containing the 47nt inclusion. Quantification of the dPCR (% 47nt inclusion) is shown in FIGS. 13-15, where three different cell types (HEK293, A549 and T98G) were treated by selected ASOs.

FIG. 16 depicts the dose-response effects of ASO treatments on the 47nt inclusion event. dPCR quantification of the 47nt inclusion event is shown for cells treated with increasing doses of indicated ASOs (from 1 nM to 100 nM).

FIGS. 17-19 characterize ATXN2 RNA expression in ASO-treated cells by RNA-seq. T98G cells were used in FIGS. 17 and A549 cells were used in FIGS. 18-19. Reads obtained during sequencing are displayed in the wiggle format at their mapped genomic coordinates. Junction reads that span exons are counted and displayed as an exon-exon junction beneath the reads track along with the corresponding read counts if the number of supporting reads is >4.

FIG. 17 shows the results from the following three samples: 1) untreated cells (second track); 2) cells treated with A8 by transfection (third track); and 3) cells treated with A9 by transfection (fourth track). The quantification of the 47nt inclusion as calculated by the junction reads supporting that event is indicated on each track. FIG. 18 shows the results from the following three samples: 1) cells with sham treatment (second track), 2) cells treated with C1 by transfection (third track); 3) cells treated with A6 by transfection (fourth track); and 4) cells treated with A22 by transfection (fifth track). FIG. 19 is a graph quantifying FIG. 18 by showing the abundance of ATXN2 RNA and the relative amount of each variant of exon 8 as determined by junction reads ending at the 5′ end of exon 9.

FIG. 20 is a qPCR quantification of ATXN2 mRNA expression in HepG2 cells using TaqMan assay Hs01002848_ml (“2848”) upon treatment with control or experimental ASOs.

FIG. 21 is a qPCR quantification of ATXN2 mRNA expression in HEK293 cells using TaqMan assay Hs01002848_ml (“2848”) upon treatment with control or experimental ASOs.

FIG. 22 is a qPCR quantification of ATXN2 mRNA expression in U2OS cells using TaqMan assay Hs01002848_m1 (“2848”) upon treatment with control or experimental ASOs. A125 to A149 are ASOs designed to target the exon 18 region.

FIGS. 23-24 depict dose-response effects of ASO treatments (A9 and A28) on ATXN2 mRNA expression in U2OS cells using TaqMan assay Hs01002848_ml (“2848”). The calculated IC₅₀ values are shown.

FIGS. 25-30 characterize ATXN2 protein expression in ASO-treated cells by ProteinSimple Jess Western Blots. FIGS. 25, 27, 29 are images of the SimpleWestern Jess run with ATXN2 protein being detected by the ATXN2 antibody (BD #611378). Total protein stain in FIGS. 25 and 27 and Vinculin (detected by ThermoFisher #MA5-11690 antibody) in FIGS. 27 and 29 are used as references. The samples are from different cell types: FIGS. 25-26 show the results from T98G cells, FIGS. 27-28 show the results from U2OS cells, and FIGS. 29-30 show the results from HEK293 cells. The quantification of ATXN2 protein levels were determined by analyzing the gels with the ProteinSimple Compass software (FIGS. 26, 28, and 30).

FIGS. 31-32 characterize changes in ATXN2 protein expression in the cerebellum when 100 μg ASOs are administered by ICV injection in Q22 mice. The WT mice samples are used as control. In the quantification (FIG. 32), human protein expression is estimated based on the WT expression and percentages of human ATXN2 remaining (relative to PBS) are indicated.

FIGS. 33-34 characterize changes in ATXN2 protein expression in the spinal cord when 100 μg ASOs are administered by ICV injection in Q22 mice. The WT mice samples are used as control. In the quantification (FIG. 34), human protein expression is estimated based on the WT expression and percentages of human ATXN2 remaining (relative to PBS) are indicated.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (“NCBI”) and other data referred to throughout in the disclosure herein are hereby expressly incorporated by reference for the portions of the document discussed herein, as well as in their entirety.

The present disclosure provides antisense oligonucleotides, pharmaceutical compositions, and methods for the treatment, prevention, or amelioration of diseases, disorders, and conditions associated with ATXN2 in a subject in need thereof. ATXN2-related diseases, disorders, and conditions include, without limitation, neurodegenerative diseases (e.g., spinocerebellar ataxia, ALS, parkinsonism, frontotemporal dementia, Alzheimer's disease) or proteinopathy diseases such as TDP43 proteinopathy.

### Definitions

Unless otherwise indicated, the following terms have the following meanings:

The term “antisense” refers generally to any approach reliant upon agents, e.g., single-stranded oligonucleotides, that are sufficiently complementary to a target sequence to associate with the target sequence in a sequence-specific manner (e.g., hybridize to the target sequence). Exemplary uses of antisense in the instant application involve use of an oligoribonucleotide agent that hybridizes to a target pre-mRNA molecule and blocks an activity/effect (e.g., splicing pattern and/or non-productive splice sites) of the targeted pre-mRNA sequence. Antisense approaches commonly are used to target DNA or RNA for transcriptional inhibition, translational inhibition, degradation, etc. Antisense is a technology that can be initiated by the hand of man, for example, to modulate splicing and/or silence the expression of target genes.

As used herein, the term “antisense oligonucleotide” refers to a nucleic acid (e.g., an RNA or analog thereof), having sufficient sequence complementarity to a target RNA (i.e., the RNA for which splice site selection is modulated) to block a region of a target RNA (e.g., pre-mRNA) in an effective manner. In exemplary aspects, such blocking of splice sites in ATXN2 pre-mRNA serves to modulate splicing, either by masking a binding site for a native protein that would otherwise modulate splicing and/or by altering the structure of the targeted RNA. In some aspects, the target RNA is a target pre-mRNA (e.g., ATXN2 pre-mRNA).

An antisense oligonucleotide having a “sequence sufficiently complementary to a target RNA sequence to modulate splicing of the target RNA” means that the antisense oligonucleotide has a sequence sufficient to trigger the masking of a binding site for a native protein that would otherwise modulate splicing and/or alters the three-dimensional structure of the targeted RNA to likewise modulate splicing. Likewise, an oligonucleotide reagent having a “sequence sufficiently complementary to a target RNA sequence to modulate splicing of the target RNA” means that the oligonucleotide reagent has a sequence sufficient to trigger the masking of a binding site for a native protein that would otherwise modulate splicing and/or alters the three-dimensional structure of the targeted RNAs used herein.

The term “target gene” or “target RNA transcript” is a gene or transcript (e.g., a pre-mRNA) whose expression is to be substantially modulated. This modulation can be achieved by steric blocking of splicing regulatory elements.

The term “antisense activity” means any detectable or measurable activity attributable to the hybridization of an antisense oligonucleotide to its target nucleic acid. In some embodiments, antisense activity is a decrease in the amount or expression of a target nucleic acid or protein encoded by such target nucleic acid.

The term “target-recognition sequence” refers to the portion of an antisense oligonucleotide that recognizes a target nucleic acid. The target-recognition sequence has a nucleobase sequence that permits hybridization to a corresponding region or segment of a target nucleic acid.

As used herein, the term “sufficiently complementary” means that antisense oligonucleotide has a sequence (e.g., an antisense oligonucleotide having a target-recognition sequence), which is sufficient to bind the desired target transcript (e.g., an ATXN2 transcript), and to trigger changes in RNA processing, e.g., inhibition or induction of non-productive splicing of the target transcript (e.g., steric inhibition of splicing machinery of the target pre-mRNA). For example, a target-recognition sequence with at least 90% complementarity to a target nucleic acid sequence (e.g., a portion of an ATXN2 transcript) can be sufficiently complementary to trigger modulation of the ATXN2 transcript. The term “perfectly complementary” refers to, e.g., a target-recognition sequence with 100% complementarity to a target nucleic acid sequence. Complementary nucleic acid molecules hybridize to each other. The term “hybridization” means the annealing of complementary nucleic acid molecules to create a stable double-stranded nucleotide. In certain embodiments, complementary nucleic acid molecules include an antisense oligonucleotide and a target nucleic acid.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). An RNA nucleotide refers to a single ribonucleotide. The term “DNA” or “DNA molecule” or “deoxyribonucleic acid molecule” refers to a polymer of deoxyribonucleotides. A DNA nucleotide refers to a single deoxyribonucleotide. As used herein, the term “DNA-like” refers to a conformation of, e.g. a modified nucleoside or nucleotide, which is similar to the conformation of a corresponding unmodified DNA unit. For example, a DNA-like nucleotide may refer to a conformation of a modified deoxyribonucleotide similar to a corresponding unmodified deoxyribonucleotide. Examples of DNA-like nucleotides include, without limitation, e.g., 2′-deoxyribonucleotides, 2′-deoxy-2′-substituted arabinonucleotides (e.g., 2′-deoxy-2′-fluoroarabinonucleotides, also known in the art as 2′F-ANA or FANA), and corresponding phosphorothioate analogs. As used herein, the term “RNA-like” refers to a conformation of, e.g. a modified nucleoside or nucleotide which is similar to the conformation of a corresponding unmodified RNA unit. RNA-like conformations may adopt an A-form helix while DNA-like conformations adopt a B-form helix. Examples RNA-like nucleotides include, without limitation, e.g., 2′-substituted-RNA nucleotides (e.g., 2′-fluoro-RNA nucleotides also known in the art as 2′F-RNA), locked nucleic acid (LNA) nucleotides (also known in the art as bridged nucleic acids or bicyclic nucleotides), 2′-fluoro-4′-thioarabinonucleotide (also known in the art as 4'S-FANA nucleotides), 2′-O-alkyl-RNA, and corresponding phosphorothioate analogs. In some aspects, the antisense oligonucletoides described herein may comprise one or more DNA, RNA, DNA-like nucleotides and/or RNA-like nucleotides.

The term “nucleotide analog” or “altered nucleotide” or “modified nucleotide” refers to a non-standard nucleotide, including non-naturally occurring ribonucleotides or deoxyribonucleotides. Exemplary modified nucleotides are modified at any position so as to alter certain chemical properties of the nucleotide yet retain the ability of the modified nucleotide to perform its intended function. Examples of positions of the nucleotide which may be derivatized include the 5 position, e.g., 5-(2-amino) propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Modified nucleotides also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotides such as those described in Herdewijn, Antisense Nucleic Acid Drug Dev., 2000 Aug. 10 (4): 297-310.

Modified nucleotides may also comprise modifications to the sugar portion of the nucleotides. For example, the 2′ OH-group may be replaced by a group selected from H, OR, R, F, Cl, Br, I, SH, SR, NH₂, NHR, NR₂, COOR, or OR, wherein R is substituted or unsubstituted with C₁-C₆alkyl, alkenyl, alkynyl, aryl, etc. As another example, the ribose sugar may be replaced with a bicyclic or tricylic moiety, such as in Locked Nucleic Acid, constrained ethyl, tricycloDNA, or other bridged or bicyclic modifications. Other possible modifications include those described in U.S. Pat. Nos. 5,858,988, and 6,291,438.

The phosphate group of the nucleotide can also be modified, e.g., by substituting one or more of the oxygens of the phosphate group with sulfur (e.g., phosphorothioates), or by making other substitutions which allow the nucleotide to perform its intended function such as described in Eckstein, Antisense Nucleic Acid Drug Dev. 2000 Apr. 10 (2): 117-21, Rusckowski et al. Antisense Nucleic Acid Drug Dev. 2000 Oct. 10 (5): 333-45, Stein, Antisense Nucleic Acid Drug Dev. 2001 Oct. 11 (5): 317-25, Vorobjev et al. Antisense Nucleic Acid Drug Dev. 2001 Apr. 11 (2): 77-85, and U.S. Pat. No. 5,684,143. Some of the above-referenced modifications (e.g., phosphate group modifications) can decrease the rate of hydrolysis of, for example, polynucleotides comprising said analogs in vivo or in vitro.

As used herein, the terms “unmodified nucleotide” or “non-modified nucleotide” refers to a nucleotide composed of naturally occurring nucleobases, sugar moieties, and internucleoside linkages. In some embodiments, a non-modified nucleotide is an RNA nucleotide (e.g., a β-D-ribonucleoside) or a DNA nucleotide (e.g., a β-D-deoxyribonucleoside).

The term “oligonucleotide” refers to a short polymer of nucleotides and/or modified nucleotides. As discussed above, the oligonucleotides may be linked with linkages which result in a lower rate of hydrolysis as compared to an oligonucleotide linked with phosphodiester linkages. For example, the nucleotides of the oligonucleotide may comprise triazole, amide, peptide, carbamate, methylenediol, ethylene diol, oxymethylthio, oxyethylthio, oxycarbonyloxy, phosphorodiamidate, phosphoroamidate, phosphonate, and/or phosphorothioate linkages. Alterations or modifications of the oligonucleotide can further include addition of non-nucleotide material, such as to the end(s) of the oligonucleotide or internally (at one or more nucleotides of the oligonucleotide).

As used herein, “administer” or “administration” refers to the act of injecting or otherwise physically delivering a substance as it exists outside the body (e.g., an antisense oligonucleotide described herein) into a patient. The antisense oligonucleotides described herein may be administered to the central nervous system of a patient. The central nervous system includes the brain and spinal cord. Administration methods to the central nervous system include, but are not limited to, intravenous, intramuscular, intraperitoneal, intranasal, subcutaneous, intrathecal, intracerebroventricular or intrastriatal infusion or delivery and/or any other method of physical delivery described herein or known in the art.

“Effective amount” means the amount of active pharmaceutical agent (e.g., an antisense oligonucleotide of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

As used herein, the terms “subject” includes mammals, such as non-primates (e.g., cows, pigs, horses, cats, dogs, rabbits, rats, etc.) and primates (e.g., monkey and human). Mammals include, without limitation, humans, non-human primates, wild animals, feral animals, farm animals, and pets. In some aspects, the subject is a mammal, such as a human, having an ATXN2-related disorder (e.g., ALS).

### ATXN2 Antisense Oligonucleotides

In some aspects, an antisense oligonucleotide that targets an ATXN2 transcript is from 10 to 30 nucleotides in length. In other embodiments, the antisense oligonucleotide that targets an ATXN2 transcript is from 15 to 25, or 18 to 20, nucleotides in length. For example, the antisense oligonucleotides may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or a range defined by any pair of the foregoing values.

In some aspects, the antisense oligonucleotides of the disclosure comprise a nucleotide sequence selected from the group consisting of SEQ ID NOs: 10-137, or a 2, 3, 4, 5, 6, 7, 8, 9, 10, 11, 12, 13, 14, 15, 16, 17, 18, or 19-mer fragment thereof (or a fragment with a size within a range defined by any pair of the foregoing values). In some aspects, the antisense oligonucleotide comprises a nucleotide sequence having 1, 2, 3, 4, 5, 6, 7, 8, 9, or 10 nucleotide substitutions as compared to any one of SEQ ID NOs: 10-137. In some aspects, the antisense oligonucleotide comprises a nucleotide sequence having at least 85, 86, 87, 88, 89, 90, 91, 92, 93, 94, 95, 96, 97, 98, or 99% sequence identity as compared to any one of SEQ ID NOs: 10-137. In some aspects, the antisense oligonucleotide comprises a nucleotide sequence identical to a contiguous 16-22 mer portion of any one of SEQ ID NOs: 10-12. In some aspects, the antisense oligonucleotide comprises a 10, 11, 12, 13, 14, 15, 16, 17, 18, 19, 20, 21, 22, 23, 24, or 25 nucleotide sequence, having 1, 2, 3, 4, 5, 6, 7, 8, 9, or 10 nucleotide substitutions as compared to any one of SEQ ID NOs: 10-12, or an aligned portion thereof. In some aspects, the antisense oligonucleotide comprises a nucleotide sequence identical to any contiguous 16-22 mer portion of SEQ ID NOs: 10-12, with 1, 2, 3, 4 or 5 nucleotide substitutions.

In some aspects, a composition according to the disclosure (e.g., a pharmaceutical composition) may comprise a combination of two or more antisense oligonucleotides described herein. For example, antisense oligonucleotides that hybridize to two or more target regions in an ATXN2 RNA transcript may be more effective at reducing expression of an ATXN2 protein isoform in a treated subject, cell, or tissue. The combination may be administered to a subject in vivo or cells ex vivo or in vitro as separate antisense oligonucleotides (i.e., two or more antisense oligonucleotides in a mixture), or the combination may be administered by linking the two or more antisense oligonucleotides. In some aspects, a combination therapy may comprise separate administration of two or more antisense oligonucleotides (e.g., sequential delivery).

The present disclosure also provides branched antisense oligonucleotides comprising two or more target-recognition sequences that target a portion of an ATXN2 RNA transcript. A branched antisense oligonucleotide of the present disclosure may be, e.g., a branched antisense oligonucleotide compound. As used herein, the term “branched antisense oligonucleotide” or “branched antisense oligonucleotide” or “multimeric oligonucleotide compound” refers to two or more antisense oligonucleotides that are connected together. In certain embodiments, the two or more antisense oligonucleotides are linked together through a linker.

In some aspects, a branched oligonucleotide compound comprises two or more target-recognition sequences, wherein the target-recognition sequences are connected to one another by one or more moieties selected from a linker, a spacer, and a branching point.

The present disclosure provides an antisense oligonucleotide comprising a target-recognition sequence that targets a portion of a ATXN2 nucleic acid (e.g., an ATXN2 transcript). In certain embodiments, an antisense oligonucleotide has a nucleobase sequence that, when written in the 5′ to 3′ direction, comprises the reverse complement of a portion of an ATXN2 nucleic acid.

In some aspects, a target region is a structurally defined region of a ATXN2 nucleic acid. For example, a target region may encompass a 3′ untranslated region (UTR), a 5′ untranslated region (UTR), an exon, an intron, an exon/intron junction, an exon/exon junction, a coding region, a translation initiation region, translation termination region, circular RNA, anti-sense (such as microRNA and long non-coding RNA) binding region, promoter sequence, enhancer elements, or other defined nucleic acid region, for example, an open reading frame, or the junction between an open reading frame and an untranslated region and any combinations thereof. The structurally defined regions for ATXN2 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region. Suitable target segments may be found within a 5′ UTR, a coding region, a 3′ UTR, an intron, an exon, and/or an exon/intron junction.

An antisense oligonucleotide and a target nucleic acid (e.g., an ATXN2 transcript or portion thereof) are complementary to each other when a sufficient number of nucleobases of the antisense oligonucleotide can form hydrogen bonds with the corresponding nucleobases of the target nucleic acid, such that a desired effect will occur (e.g., antisense-based promotion of a non-productive target nucleic acid, such as an ATXN2 non-productive transcript or portion thereof). Non-complementary nucleobases between an antisense oligonucleotide and an ATXN2 nucleic acid may be tolerated provided that the antisense oligonucleotide remains able to specifically hybridize to a target nucleic acid. Moreover, an antisense oligonucleotide may hybridize over one or more segments of an ATXN2 nucleic acid such that intervening or adjacent segments are not involved in the hybridization event (e.g., a loop structure, mismatch or hairpin structure).

In some aspects, the antisense oligonucleotides provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to an ATXN2 nucleic acid, a target region, target segment, or specified portion thereof. Percent complementarity of an antisense oligonucleotide with a target nucleic acid can be determined using routine methods. For example, an antisense oligonucleotide in which 18 of 20 nucleobases of the antisense oligonucleotide are complementary to a target region (e.g., an equal length portion of a ATXN2 transcript), and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligonucleotide which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present disclosure. Percent complementarity of an antisense oligonucleotide with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense oligonucleotides encompass substitutions or changes to internucleoside linkages, sugar moieties, or nucleobases. Modified antisense oligonucleotides are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense oligonucleotides that have such chemically modified nucleosides.

The naturally occurring internucleoside linkage of RNA and DNA is a 3′ to 5′ phosphodiester linkage. Antisense oligonucleotides having one or more modified, i.e., non-naturally occurring, internucleoside linkages are often selected over antisense oligonucleotides having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense oligonucleotides targeted to an ATXN2 nucleic acid comprise one or more modified internucleoside linkages. In certain embodiments, the modified internucleoside linkages are phosphorothioate linkages. In certain embodiments, each internucleoside linkage of an antisense oligonucleotide is a phosphorothioate internucleoside linkage.

Antisense oligonucleotides of the present disclosure may optionally contain one or more nucleosides wherein the sugar group has been modified. Such sugar modified nucleosides may impart enhanced nuclease stability, increased binding affinity or some other beneficial biological property to the antisense oligonucleotides. In certain embodiments, nucleosides comprise a chemically modified ribofuranose ring moieties. Examples of chemically modified ribofuranose rings include without limitation, addition of substituent groups (including 5′ and 2′ substituent groups, bridging of ring atoms to form bicyclic nucleic acids (BNA), replacement of the ribosyl ring oxygen atom with S, NI, or C (R¹) (R²) (R═H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see U.S. Patent Application Pub. No. 2005/0130923, published on Jun. 16, 2005) or alternatively 5′-substitution of a BNA (see PCT International Application WO 2007/134181 Published on Nov. 22, 2007) wherein LNA is substituted with for example a 5′-methyl or a 5′-vinyl group).

Examples of nucleosides having modified sugar moieties include without limitation nucleosides comprising 5′-vinyl, 5′-methyl(R or S), 4′-S, 2′-F (i.e., 2′-fluoro), 2′-OCH₃ (i.e., 2′-O-methyl) and 2′-O(CH₂)₂OCH₃(i.e., 2′-O-methoxyethyl) substituent groups. The substituent at the 2′ position can also be selected from allyl, amino, azido, thio, O-allyl, O—C1-C10 alkyl, OCF₃, O(CH₂)₃SCH₃, O(CH₂)₂—O—N(Rₘ)(Rₐ), and O—CH₂—C(═O)—N(Rₘ)(Rₐ), where each Rₘ and Rₙ is, independently, H or substituted or unsubstituted C1-C10 alkyl, 2′-modified nucleotides are useful in the present invention, for example, 2′-O-methyl RNA, 2′-O-methoxyethyl RNA, 2′-fluoro RNA and others envisioned by one of ordinary skill in the art.

Examples of bicyclic nucleic acids (BNAs) include without limitation nucleosides comprising a bridge between the 4′ and the 2′ ribosyl ring atoms. A BNA comprising a bridge between the 4′ and 2′ ribosyl ring atoms can be referred to as a locked nucleic acid (LNA), and is often referred to as inaccessible RNA. As used herein, the term “locked nucleotide” or “locked nucleic acid (LNA)” comprises nucleotides in which the 2′ deoxy ribose sugar moiety is modified by introduction of a structure containing a heteroatom bridging from the 2′ to the 4′ carbon atoms. The term “non-locked nucleotide” comprises nucleotides that do not contain a bridging structure in the ribose sugar moiety. Thus, the term comprises DNA and RNA nucleotide monomers (phosphorylated adenosine, guanosine, uridine, cytidine, deoxyadenosine, deoxyguanosine, deoxythymidine, deoxycytidine) and derivatives thereof as well as other nucleotides having a 2′-deoxy-erythro-pentofuranosyl sugar moiety or a ribo-pentofuranosyl moiety. In some 26annito, antisense oligonucleotides provided herein include one or more BNA nucleosides wherein the bridge comprises one of the formulas: 4′-(CH₂)—O-2′ (LNA); 4′-(CH₂)—S-2′; 4′-(CH₂)—O-2′ (LNA); 4′-(CH₂) 2-O-2′ (ENA); 4′-C(CH₃) 2-O-2′ (see PCT/US2008/068922); 4′-CH(CH₃)O-2′ and 4′-CH(CH,OCH₃)—O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see published U.S. Patent Application US2004-0171570, published Sep. 2, 2004); 4′-CH₂—NI—O-2′ (see U.S. Pat. No. 7,427,672, issued on Sep. 23, 2008); 4′-CH₂—C(CH₃)-2′ and 4′-CH₂—C(═CH₂)-2′ (see PCT/US2008/066154); and wherein R is, independently, H, C1-C₁₂ alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In some aspects, antisense oligonucleotides provided herein include one or more 2′, 4′-constrained nucleotides. For example, antisense oligonucleotides provided by the present disclosure include those having one or more constrained ethyl (cEt) or constrained methoxyethyl (cMOE) nucleotides. In some aspects, antisense oligonucleotides provided herein comprise one or more constrained ethyl (cEt) nucleotides. The terms “constrained ethyl” and “ethyl-constrained” are used interchangeably.

In some aspects, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring. Many other bicyclo and tricyclo sugar surrogate ring systems are also known in the art that can be used to modify nucleosides for incorporation into antisense oligonucleotides (see for example review article: Leumann, J. C, Bioorganic & Medicinal Chemistry, 2002, 10, 841-854; Ito, K. R.; Obika, S., Recent Advances in Medicinal Chemistry of Antisense Oligonucleotides. In Comprehensive Medicinal Chemistry, 3^{rd} edition, Elsevier: 2017). Such ring systems can undergo various additional substitutions to enhance activity.

Methods for the preparations of modified sugars are well known to those skilled in the art. In nucleotides having modified sugar moieties, the nucleobase moieties (natural, modified or a combination thereof) are maintained for hybridization with an appropriate nucleic acid target.

In some aspects, antisense oligonucleotides targeted to an ATXN2 nucleic acid comprise one or more kinds of modified nucleotides. In one embodiment, antisense oligonucleotides targeted to an ATXN2 nucleic acid comprise 2′-modified nucleotides. In one embodiment, antisense oligonucleotides targeted to an ATXN2 nucleic acid comprise a 2′-O-methyl RNA, a 2′-O-methoxyethyl RNA, or a 2′-fluoro RNA. In some aspects, antisense oligonucleotides targeted to an ATXN2 nucleic acid comprise tricyclo-DNA. Tricyclo-DNA belongs to a class of constrained DNA analogs that display improved hybridizing capacities to complementary RNA, see, e.g., Ittig et al., Nucleic Acids Res. 32:346-353 (2004); Ittig et al., Prague, Academy of Sciences of the Czech Republic. 7:21-26 (Coll. Symp. Series, Hocec, M., 2005); Ivanova et al., Oligonucleotides 17:54-65 (2007); Renneberg et al., Nucleic Acids Res. 30:2751-2757 (2002); Renneberg et al., Chembiochem. 5:1114-1118 (2004); and Renneberg et al., JACS. 124:5993-6002 (2002). In some aspects, antisense oligonucleotides targeted to an ATXN2 nucleic acid comprise a locked nucleotide, an ethyl-constrained nucleotide, or an alpha-L-locked nucleic acid. Various alpha-L-locked nucleic acids are known by those of ordinary skill in the art, and are described in, e.g., Sorensen et al., J. Am. Chem. Soc. (2002) 124 (10): 2164-2176.

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense oligonucleotides. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense oligonucleotide for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense oligonucleotides include 5-substituted pyrimidines, 6-azapyrimidines and N-2, N-6 and 0-6 substituted purines, including 2 aminopropyladenine, 5-propynyluracil and 5-propynylcytosine.

In some aspects, antisense oligonucleotides targeted to an ATXN2 nucleic acid comprise one or more modified nucleotides having modified sugar moieties. In some embodiments, the modified nucleotide is a locked nucleotide. In certain embodiments, the locked nucleotides are arranged in a gapmer motif, e.g., a 3-9-3 gapmer format wherein 9 non-locked nucleotides are flanked by 3 locked nucleotides on each side. In some aspects, antisense oligonucleotides targeted to an ATXN2 nucleic acid comprise one or more modified nucleotides. In some aspects, the modified nucleotide is 5-methylcytosine. In some aspects, each cytosine is a 5-methylcytosine.

In some aspects, the antisense oligonucleotides of the disclosure comprise a 2′-O-(2-methoxyethyl) modification at one or more nucleotides. In certain embodiments, the antisense oligonucleotides of the disclosure comprise a 2′-O-(2-methoxyethyl) modification at 10% of the nucleotides, at 20% of the nucleotides, at 30% of the nucleotides, at 40% of the nucleotides, at 50% of the nucleotides, at 60% of the nucleotides, at 70% of the nucleotides, at 80% of the nucleotides, or at 90% of the nucleotides. In certain embodiments, the antisense oligonucleotides of the disclosure comprise a 2′-O-(2-methoxyethyl) modification at every nucleotide (100% 2′-O-(2-methoxyethyl) modification).

In some aspects, the antisense oligonucleotides of the disclosure comprise one or more phosphorothioate internucleoside linkages. In certain embodiments, the antisense oligonucleotides of the disclosure comprise one or more phosphorothioate internucleoside linkages and one or more phosphodiester linkages. In certain embodiments, the antisense oligonucleotides of the disclosure comprise phosphorothioate at every internucleoside linkage.

In some aspects, an antisense oligonucleotide of the present disclosure comprises a conjugate. In one embodiment, an antisense oligonucleotide of the present disclosure comprises an antisense oligonucleotide sequence and a conjugate, wherein the conjugate is linked to the antisense oligonucleotide sequence. In some embodiments, the conjugate is selected from any of the conjugates described herein, for example, a hydrophobic conjugate, a tissue-targeting conjugate, or a conjugate designed to optimize pharmacokinetic parameters.

In some aspects, antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense oligonucleotide to impart nuclease stability include those disclosed in WO 03/004602 published on Jan. 16, 2003.

### Pharmaceutical Compositions for Modulating Expression of ATXN2 Isoforms

Provided herein are pharmaceutical compositions and formulations that comprise one or more of the antisense oligonucleotides described herein. For example, the antisense oligonucleotides described herein can be admixed, encapsulated, conjugated or otherwise associated with other molecules, molecule structures or mixtures of compounds. A pharmaceutical composition of the invention is formulated to be compatible with its intended route of administration. Examples of routes of administration include intravenous administration, intraperitoneal administration, intramuscular administration, intranasal administration, subcutaneous administration, intrathecal administration, intraventricular administration or intrastriatal administration. In some embodiments, the administration may employ an implanted device such as an Ommaya reservoir or implanted intrathecal catheter. Solutions or suspensions used for administration can include the following components: a sterile diluent such as water for injection, saline solution, lactated Ringers solution, Elliotts B solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, carbonates or phosphates and agents for the adjustment of tonicity such as sodium chloride or dextrose. pH can be adjusted with acids or bases, such as hydrochloric acid or sodium hydroxide. The pharmaceutical compositions can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain embodiments, isotonic agents may be included, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, certain methods of preparation are vacuum drying and freeze-drying which yields a powder of the active ingredient plus any additional desired ingredient from a previously sterile-filtered solution thereof.

The pharmaceutical compositions and formulations provided herein can, in some embodiments, be conveniently presented in unit dosage form and can be prepared according to techniques well known in the pharmaceutical industry. Such techniques can include bringing into association the active ingredients with the pharmaceutical carrier(s) or excipient(s). In general, the formulations can be prepared by uniformly and intimately bringing into association the active ingredients with liquid carriers, finely divided solid carriers, or both, and then, if necessary, shaping the product (e.g., into a specific particle size for delivery). In one embodiment, the pharmaceutical formulations are prepared for intrathecal, intraventricular or intrastriatal administration in an appropriate solvent, e.g., water or normal saline. In some aspects, the formulation is designed to allow the gymnotic delivery of antisense oligonucleotides to one or more cells of a subject.

An antisense oligonucleotide targeting an ATXN2 transcript can also be administered by transfection or infection using methods known in the art, including but not limited to the methods described in McCaffrey et al. (2002), Nature, 418 (6893), 38-9 (hydrodynamic transfection); Xia et al. (2002), Nature Biotechnol., 20 (10), 1006-10 (viral-mediated delivery); or Putnam (1996), Am. J. Health Syst. Pharm. 53 (2), 151-160.

An antisense oligonucleotide targeting an ATXN2 transcript may also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88 (2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some aspects, the antisense oligonucleotide(s) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

An antisense oligonucleotide targeted to an ATXN2 nucleic acid can be utilized in pharmaceutical compositions by combining the antisense oligonucleotide with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense oligonucleotide targeted to an ATXN2 nucleic acid and a pharmaceutically acceptable diluent. In certain embodiments, the pharmaceutically acceptable diluent is PBS.

The pharmaceutical compositions can be included in a container, pack, or dispenser together with instructions for administration.

Pharmaceutical compositions comprising antisense oligonucleotides encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligonucleotides, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of an antisense oligonucleotide which are cleaved by endogenous nucleases within the body, to form the active antisense oligonucleotide.

### Methods of Treating ATXN2-Related Diseases and Conditions

The present disclosure provides a method of treating a subject having an ATXN2-related disease or disorder. Methods of treatment include administering to the subject in need thereof an effective amount of any of the antisense oligonucleotides described herein. In some embodiments, the antisense oligonucleotide hybridizes to a target region in an ATXN2 RNA transcript, wherein the target region comprises a sequence that is sufficiently complementary to and/or hybridizes to any contiguous 18-22 mer portion of SEQ ID NOs: 10-12, or to a sequence that has 0, 1, 2, 3, 4 or 5 nucleotide substitutions as compared to a contiguous 16-22 mer portion of SEQ ID NOs: 10-12. In some aspects, the antisense oligonucleotide comprises a nucleotide sequence identical to that of any one of SEQ ID NOs: 10-137 (or a 2, 3, 4, 5, 6, 7, 8, 9, 10, 11, 12, 13, 14, 15, 16, 17, 18, or 19-mer fragment of any one of SEQ ID NOs: 10-137). In some aspects, the antisense oligonucleotide comprises a nucleotide sequence having 1, 2, 3, 4, 5, 6, 7, 8, 9, or 10 nucleotide substitutions as compared to any one of SEQ ID NOs: 10-137 (or a 2, 3, 4, 5, 6, 7, 8, 9, 10, 11, 12, 13, 14, 15, 16, 17, 18, or 19-mer fragment of any one of SEQ ID NOs: 10-137). In some aspects, the antisense oligonucleotide comprises-nucleotide sequence having at least 80, 81, 82, 83, 84, 85, 86, 87, 88, 89, 90, 91, 92, 93, 94, 95, 96, 97, 98, 99 or 100% sequence identity as compared to any one of SEQ ID NOs: 10-137 (or a 2, 3, 4, 5, 6, 7, 8, 9, 10, 11, 12,13, 14, 15, 16, 17, 18, or 19-mer fragment of any one of SEQ ID NOs: 10-137). In some aspects, the antisense oligonucleotide comprises a 21, 22, 23, 24, 25, 26, 27, 28, 29 or 30-mer nucleotide sequence comprising a contiguous sequence identical to any one of SEQ ID NOs: 10-137 (or a 2, 3, 4, 5, 6, 7, 8, 9, 10, 11, 12, 13, 14, 15, 16, 17, 18, or 19-mer fragment of any one of SEQ ID NOs: 10-137).

An ATXN2-related disease or disorder includes, without limitation neuropathies, such as Spinocerebellar ataxia, ALS, parkinsonism, Frontotemporal dementia and Alzheimer's disease, or a proteinopathy disease such as TDP43 proteinopathy.

In some aspects, pharmaceutical compositions according to the disclosure may be administered according to a dosing regimen (e.g., dose, dose frequency, and duration) wherein the dosing regimen can be selected to achieve a desired effect. The desired effect can be, for example, reduction of the level of ATXN2 protein expression in the subject (or in one or more cells or tissues thereof) or prevention, reduction, amelioration, or slowing, of one or more symptoms or the progression of a disease or condition associated with ATXN2.

In some aspects, the variables of the dosing regimen are adjusted to achieve a desired concentration of the antisense oligonucleotide(s) in a subject being treated. For example, in certain embodiments, dose and dose frequency are adjusted to provide a cellular, tissue, or plasma concentration of an ATXN2 antisense oligonucleotide at an amount sufficient to achieve a desired effect. In some aspects, the desired cellular concentration may be 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nM, or a concentration within a range with endpoints defined by any pair of the foregoing concentration values. In some aspects, a subject may be administered a composition comprising 0.001 to 250 mg/mL of at least one antisense oligonucleotide described herein. For example, in some aspects, methods according to the disclosure may comprise administering a pharmaceutically-acceptable dosage form comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg (or an amount within a range defined by any pair of the foregoing values) of one or more of the antisense oligonucleotides described herein. For example, a liquid formulation suitable for administration may comprise 5-20 mg of an antisense oligonucleotide described herein in 1, 2, 3, 4, 5, 6, 7, 8, 9, 10, 11, 12, 13, 14, 15, 16, 17, 18, 19, 20, 21, 22, 23, 24, or 25 mL (or a volume within a range defined by any pair of the foregoing values) of solution.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In some aspects, dosage is from 0.01 μg to 100 mg per kg of body weight, or within a range of 0.001 mg-1000 mg, and may be given once or more daily, weekly, monthly, quarterly, biannually or yearly. For example, a dosage may comprise 10, 20, 30, 40, 50, 60, 70, 80 or 90 mg per kg (or an amount per kg within a range defined by any pair of the foregoing values), administered 1, 2, or 3 times per day (e.g., via a liquid formulation comprising 1, 2, 3, 4, 5, 6, 7, 8, 9, or 10 mL). Following successful treatment, it may be desirable to have the patient undergo maintenance therapy to prevent the recurrence of the disease state, wherein the antisense oligonucleotide is administered in maintenance doses, ranging from 0.01 μg to 100 mg per kg of body weight, once or more daily, weekly, monthly, quarterly, biannually or yearly.

### EXAMPLES

The present invention will be more specifically illustrated by the following Examples. However, it should be understood that the present invention is not limited by these examples in any manner.

### Example 1: Identification of the Targetable Alternative Splicing Events in Human ATXN2 RNA

### Materials and Methods

The human ATXN2 gene was visualized on the UCSC Genome Browser and the mRNA transcripts were inspected for alternative splicing events that may lead to the formation of a premature termination codon.

### Results

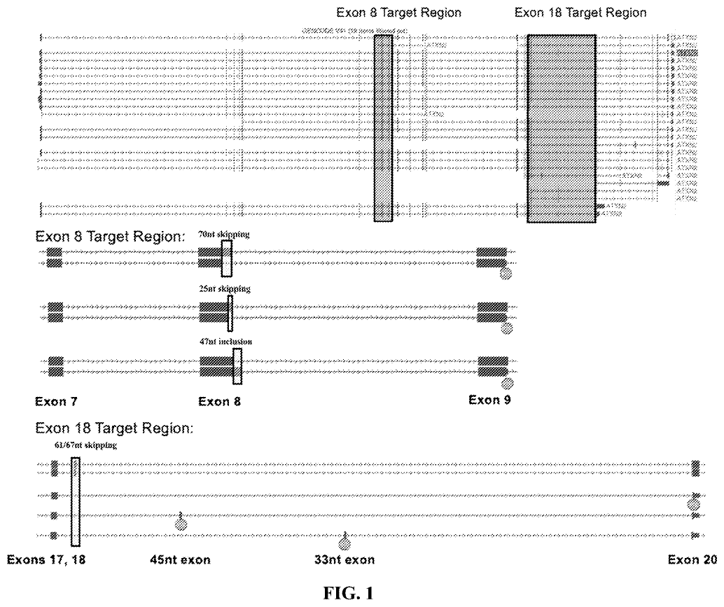

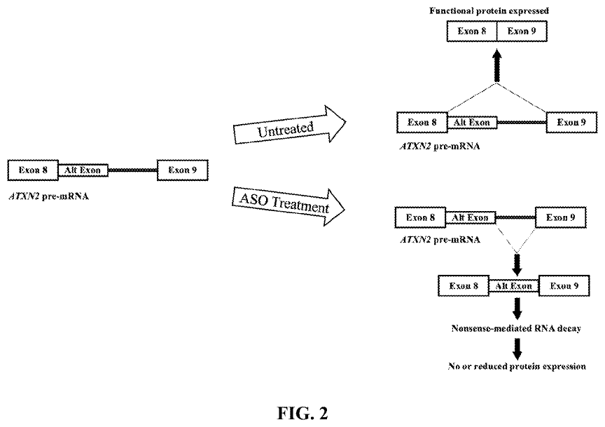

Four druggable alternative splicing events were identified (FIG. 1): three alternative 5′ splice sites were found in exon 8—resulting in a 47nt inclusion, a 70nt skipping, or a 25nt skipping event that each lead to the creation of a premature termination codon in exon 9; and alternative splicing of exon 18—skipping of a 61nt/67nt exon leading to the creation of a premature termination codon in the next included exon (either exon 19a, 19b, or 20). Various ASOs described herein are designed to increase the occurrence of this alternative splicing event and reduce ATXN2 protein levels (FIG. 2).

### Example 2: ASO-Mediated Modulation of Exon 8 and Exon 18 Splicing in Cell Lines

### Materials and Methods

ASOs were designed around the exon 8 alternative splicing events (FIG. 1) as shown in Table 1 below. ASOs C1 and C5 (SEQ ID NOs: 8 and 9, respectively) were designed as non-targeting control ASOs; C1 targets the SMN1 and SMN2 genes and C5 targets no gene.

TABLE 1
Oligos designed to modulate the splicing of
exon 8.

| SEQ ID NO | Name | Sequence |
|---|---|---|
| 13 | A1 | TTTCCAGGGCCACTCGAGCT |
| 14 | A2 | CTATCATCATTTTCCAGGGC |
| 15 | A3 | TTCCTCACTCCTATCATCAT |
| 16 | A4 | TGTATTTTTCTTCCTCACTC |
| 17 | A5 | TGAACTGCTGTGTATTTTTC |
| 18 | A6 | GGAATTTCTCTGAACTGCTG |
| 19 | A7 | CACGTTCACTGGAATTTCTC |
| 20 | A8 | CTGTGCCCCTCACGTTCACT |
| 21 | A9 | AGTGTTTATGCTGTGCCCCT |
| 22 | A10 | TTAAATACCTAGTGTTTATG |
| 23 | A11 | ATGATTTCCTTTAAATACCT |
| 24 | A12 | ATCCAAAATACTGCATCATG |
| 25 | A13 | ACCTTGAGTTGTGTATCCAA |
| 26 | A14 | CCGTCTCACACAGACCTTGA |
| 27 | A15 | AACAATACACCGTCTCACAC |
| 28 | A16 | AAGAGGAAATATAATAACAA |
| 29 | A17 | AAGCTATATTAAAGGAAGAG |
| 30 | A18 | TTGCATTCTCTACCTAAGCT |
| 31 | A19 | TAAACCAATTCTACTTGCAT |
| 32 | A20 | TCTAACAGATCTTAAACCAA |
| 33 | A21 | TCTGAACTGCTGTGTATTTT |
| 34 | A22 | CTCTGAACTGCTGTGTATTT |
| 35 | A23 | TCTCTGAACTGCTGTGTATT |
| 36 | A24 | TTCTCTGAACTGCTGTGTAT |
| 37 | A25 | TTTCTCTGAACTGCTGTGTA |
| 38 | A26 | ATTTCTCTGAACTGCTGTGT |
| 39 | A27 | AATTTCTCTGAACTGCTGTG |
| 40 | A28 | GAATTTCTCTGAACTGCTGT |
| 41 | A29 | GGAATTTCTCTGAACTGCTG |
| 42 | A30 | TGGAATTTCTCTGAACTGCT |
| 43 | A31 | CTGGAATTTCTCTGAACTGC |
| 44 | A32 | ACTGGAATTTCTCTGAACTG |
| 45 | A33 | CACTGGAATTTCTCTGAACT |
| 46 | A34 | TCACTGGAATTTCTCTGAAC |
| 47 | A35 | TTCACTGGAATTTCTCTGAA |
| 48 | A36 | GTTCACTGGAATTTCTCTGA |
| 49 | A37 | CGTTCACTGGAATTTCTCTG |
| 50 | A38 | ACGTTCACTGGAATTTCTCT |
| 51 | A39 | CTGAACTGCTGTGTATTTTT |
| 52 | A59 | GGAATTTCTCTGAACTGCT |
| 53 | A60 | GAATTTCTCTGAACTGCTG |
| 54 | A61 | AATTTCTCTGAACTGCTGT |
| 55 | A62 | ATTTCTCTGAACTGCTGTG |
| 56 | A63 | TACCTAGTGTTTATGCTGTG |
| 57 | A64 | ACCTAGTGTTTATGCTGTGC |
| 58 | A65 | CCTAGTGTTTATGCTGTGCC |
| 59 | A66 | CTAGTGTTTATGCTGTGCCC |
| 60 | A67 | TAGTGTTTATGCTGTGCCCC |
| 61 | A68 | GTGTTTATGCTGTGCCCCTC |
| 62 | A69 | TGTTTATGCTGTGCCCCTCA |
| 63 | A70 | GTTTATGCTGTGCCCCTCAC |
| 64 | A71 | TTTATGCTGTGCCCCTCACG |
| 65 | A72 | TTATGCTGTGCCCCTCACGT |
| 66 | A73 | GCATCATGATTTCCTTTAAA |
| 67 | A74 | CATCATGATTTCCTTTAAAT |
| 68 | A75 | ATCATGATTTCCTTTAAATA |
| 69 | A76 | TCATGATTTCCTTTAAATAC |
| 70 | A77 | CATGATTTCCTTTAAATACC |
| 71 | A78 | TGATTTCCTTTAAATACCTA |
| 72 | A79 | GATTTCCTTTAAATACCTAG |
| 73 | A80 | ATTTCCTTTAAATACCTAGT |
| 74 | A81 | TTTCCTTTAAATACCTAGTG |
| 75 | A82 | TTCCTTTAAATACCTAGTGT |
| 76 | A93 | TCTGAACTGCTGTGTATTT |
| 77 | A94 | CTCTGAACTGCTGTGTATT |
| 78 | A95 | TCTCTGAACTGCTGTGTAT |
| 79 | A96 | TTCTCTGAACTGCTGTGTA |
| 80 | A97 | TTTCTCTGAACTGCTGTGT |
| 81 | A98 | AATTTCTCTGAACTGCTGTGTATTT |
| 82 | A99 | AATTTCTCTGAACTGCTGTGTATT |
| 83 | A100 | AATTTCTCTGAACTGCTGTGTAT |
| 84 | A101 | AATTTCTCTGAACTGCTGTGTA |
| 85 | A102 | AATTTCTCTGAACTGCTGTGT |
| 86 | A103 | GAATTTCTCTGAACTGCTGTG |
| 87 | A104 | GGAATTTCTCTGAACTGCTGTG |
| 88 | A105 | TGGAATTTCTCTGAACTGCTGTG |
| 89 | A106 | CTGGAATTTCTCTGAACTGCTGTG |
| 90 | A107 | ACTGGAATTTCTCTGAACTGCTGTG |
| 91 | A108 | TTCTCTGAACTGCTGTGT |
| 92 | A109 | TCTCTGAACTGCTGTGTA |
| 93 | A110 | CTCTGAACTGCTGTGTAT |
| 94 | A111 | TCTGAACTGCTGTGTATT |
| 95 | A112 | CTGAACTGCTGTGTATTT |
| 96 | A113 | TCTCTGAACTGCTGTGT |
| 97 | A114 | CTCTGAACTGCTGTGTA |
| 98 | A115 | TCTGAACTGCTGTGTAT |
| 99 | A116 | CTGAACTGCTGTGTATT |
| 100 | A117 | TGAACTGCTGTGTATTT |
| 101 | A118 | CTCTGAACTGCTGTGT |
| 102 | A119 | TCTGAACTGCTGTGTA |
| 103 | A120 | CTGAACTGCTGTGTAT |
| 104 | A121 | TGAACTGCTGTGTATT |
| 105 | A122 | GAACTGCTGTGTATTT |
| 131 | A217 | TTTCCTTTAAATACCTAGTGT |
| 132 | A218 | ATTTCCTTTAAATACCTAGTGT |
| 133 | A219 | GATTTCCTTTAAATACCTAGTGT |
| 134 | A220 | TGATTTCCTTTAAATACCTAGTGT |
| 135 | A221 | ATGATTTCCTTTAAATACCTAGTGT |
| 136 | A222 | CATGATTTCCTTTAAATACCTAGTGT |
| 137 | A223 | TCATGATTTCCTTTAAATACCTAGTGT |

In addition, ASOs were designed to target the region around exon 18.

TABLE 2
Oligos designed to modulate the splicing
of exon 18.

| SEQ ID NO | Name | Sequence |
|---|---|---|
| 106 | A125 | TGAGAGAATTAAAAAAAA |
| 107 | A126 | GCTTGAGAGAATTAAAAA |
| 108 | A127 | AAGGCTTGAGAGAATTAA |
| 109 | A128 | ATAAAGGCTTGAGAGAAT |
| 110 | A129 | GGTATAAAGGCTTGAGAG |
| 111 | A130 | TTGGGTATAAAGGCTTGA |
| 112 | A131 | GTATTGGGTATAAAGGCT |
| 113 | A132 | CATAGGTATTGGGTATAA |
| 114 | A133 | GGCGTCATAGGTATTGGG |
| 115 | A134 | GCATGGGCGTCATAGGTA |
| 116 | A135 | CACTGGCATGGGCGTCAT |
| 117 | A136 | TGATTCACTGGCATGGGC |
| 118 | A137 | TGGCTTGATTCACTGGCA |
| 119 | A138 | TGTCTTGGCTTGATTCAC |
| 120 | A139 | CTATATGTCTTGGCTTGA |
| 121 | A140 | CTGCTCTATATGTCTTGG |
| 122 | A141 | TTTACCTGCTCTATATGT |
| 123 | A142 | ACCTTTACCTGCTCTATA |
| 124 | A143 | CTCACCTTTACCTGCTCT |
| 125 | A144 | ATTCTCACCTTTACCTGC |
| 126 | A145 | ATTATTCTCACCTTTACC |
| 127 | A146 | AGGATTATTCTCACCTTT |
| 128 | A147 | GGCAGGATTATTCTCACC |
| 129 | A148 | ACAGGCAGGATTATTCTC |
| 130 | A149 | AACACAGGCAGGATTATT |

The ASOs were tested in cell lines (A549, HEK293, T98G, or U2OS) by lipofectamine transfection (ThermoFisher #13778075). The cells were transfected the day following plating and total RNA was isolated using RNEasy Mini kit (Qiagen #74104) and reverse transcribed into cDNA.

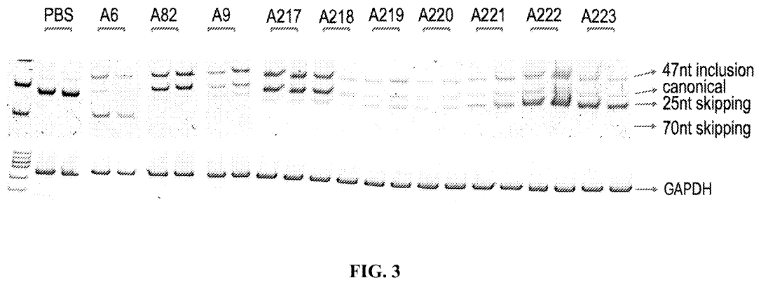

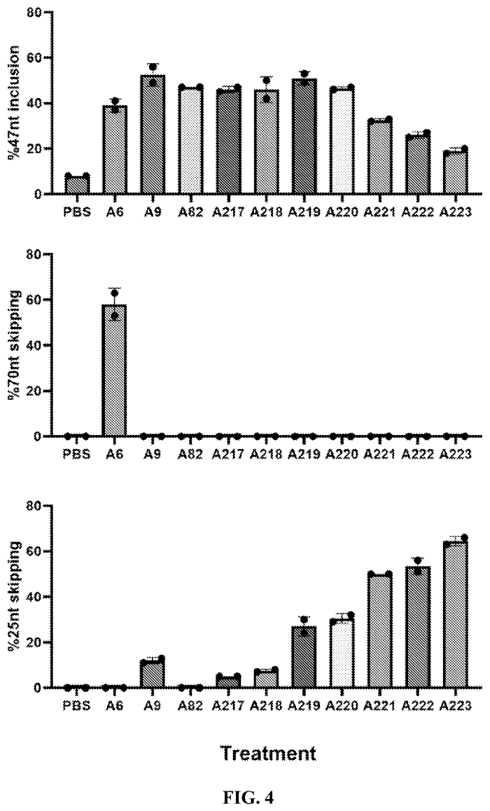

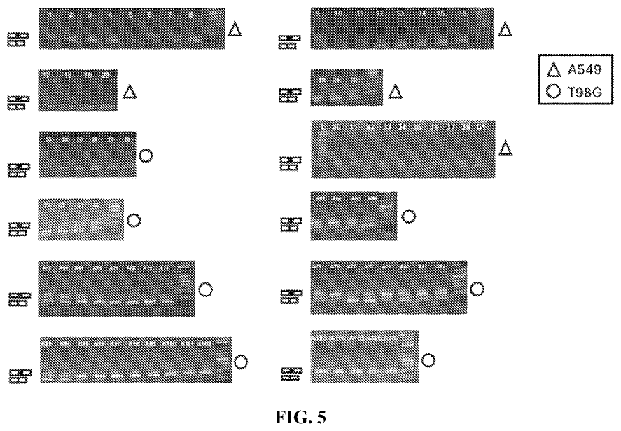

To characterize the splicing of the exon 8 region, primers were designed to amplify the regions of interest and PCR was performed. The PCR products were visualized on agarose or acrylamide gels (FIGS. 3, 5, and 6). Primer sequences for FIGS. 3-4 amplify the exon 7 to exon 10 region and were AAATTATGGTGTAGTGTCTACG (forward) and TGATGGCATGGAGCCCGAT (reverse). Primer sequences for FIGS. 5-7 amplify the exon 8 47nt inclusion region and were AATTCCAGTGAACGTGAGGG (forward) and GTGGATCTTGATGGCATGGA (reverse).

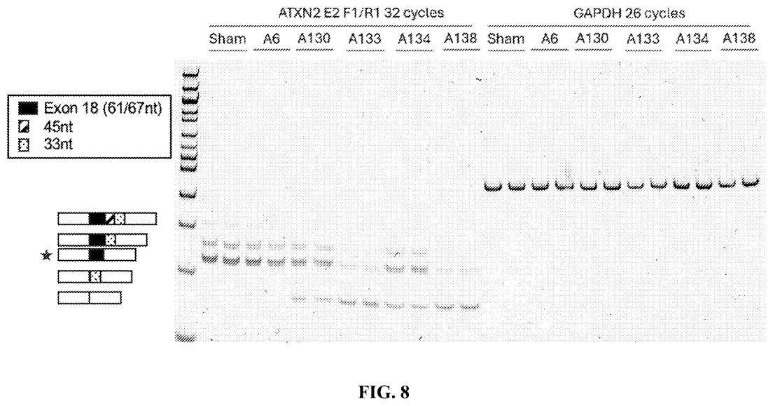

To characterize the splicing of the exon 18 region, primers were designed to amplify the region from exon 17 to exon 20 and PCR was performed. The PCR products were visualized on an acrylamide gel (FIG. 8). Primer sequences were CTACCCCAACTTCACCTCGG (forward) and CGCTGTTGGGGCATATTTGG (reverse).

To quantify the usage of alternative splicing events, the PCR product bands were quantified by densitometry analysis of the acrylamide gel images (FIGS. 4, 7, and 9).

### Results

Four different bands were observed in the first acrylamide gel image (FIG. 3), corresponding to the different splice isoforms in the exon 8 region, as depicted in FIG. 1. The bands representing alternative splice isoforms were found to be very weak or absent in control samples (C1) but become dominant in some of the ASO-treated samples. A densitometry analysis of the bands was conducted (FIG. 4) to quantify the changes in splicing. All tested ASOs promoted the use of the 47nt inclusion compared to PBS. Its inclusion was 8% in PBS and increased to a range of 18-56% inclusion upon ASO treatment. The 25nt truncation event was observed in most, but not all, of the tested ASOs. Its inclusion was 0% for PBS; 13% for A9; and ranged from 5% to 66% inclusion for A217 to A223, where inclusion increased with the length of the ASOs. The 70nt truncation event was only observed in the A6 event although A6 promoted high usage of the event ranging from 53-63%.

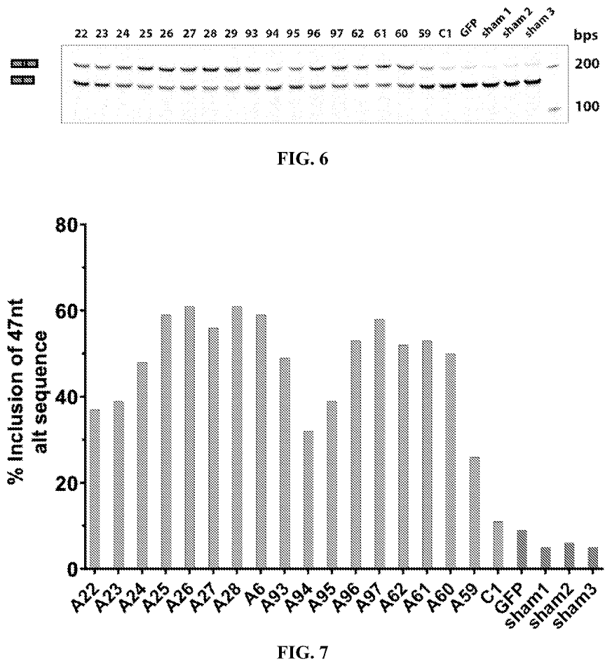

Further screenings were performed to identify optimal ASOs for splicing modulation, with results shown in FIG. 5. A subset of the results was confirmed using an acrylamide gel (FIG. 6) and quantification of the bands suggest some ASO-treated samples have as high as 60% 47nt inclusion isoform in all ATXN2 transcripts (FIG. 7). These ASOs would induce NMD-mediated decay of ATXN2 and decrease its expression. The opposite effect was also observed in some ASO-treated samples, i.e., disappearance of the 47nt inclusion band, suggesting those ASOs could reduce the NMD-mediated decay of ATXN2 and increase the level of ATXN2.

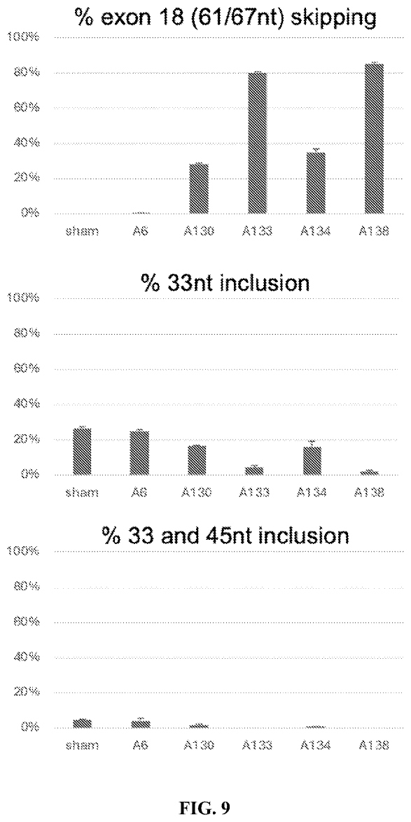

Screening of the exon 18 region identified several ASOs that effectively caused exon skipping to generate the NMD isoform (FIG. 8). Densitometry analysis indicated that skipping of exon 18 reached over 80% with the most potent ASOs (FIG. 9). An effect on the splicing of the 33nt and 45nt exons was also observed.

These assays show the ASOs are effective in modulating ATXN2 RNA splicing. It is also worth noting that they are likely to have underestimated the inclusion ratio as the inclusion isoform is subjective to the NMD decay and the ones that were already decayed in the cell cannot be measured.

### Example 3: Dose-Dependent Modulation of Exon 8 47nt Inclusion by ASOs

### Materials and Methods

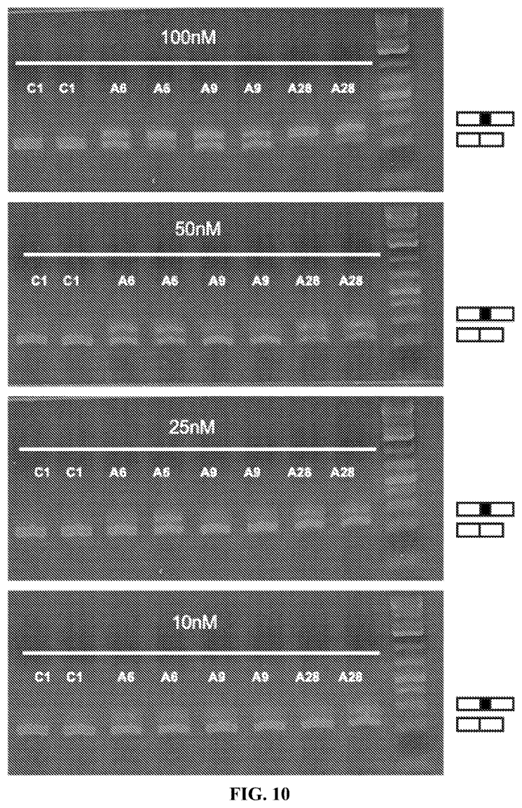

Dose-response experiments were performed to confirm that the splicing change is an on-target mechanism. ASOs 6, 9, and 28 were transfected into T98G cells at ascending doses (1, 2.5, 5, 10, 25, 50 and 100 nM ASO concentration) following the same protocols described in Example 2. Isoform expression from samples at selected doses (100 nM, 50 nM, 25 nM, and 10 nM) was visualized on agarose gels as shown in FIG. 10.

### Results

Compared to the control, all selected ASOs induced the 47nt inclusion at all the doses examined (FIG. 10). The dose-dependent changes were confirmed as inclusion was observed to increase with higher doses of the ASOs. This supports the hypothesis that the ASO directly affects the alternative splicing of ATXN2.

Dose-dependent splicing changes were observed with selected ASOs in vivo using the Q22 mouse model (transgenic mouse model containing insertions of the human ATXN2 genomic region, described in Example 8).

### Example 4: Digital PCR Confirmation of Change in 47nt Inclusion

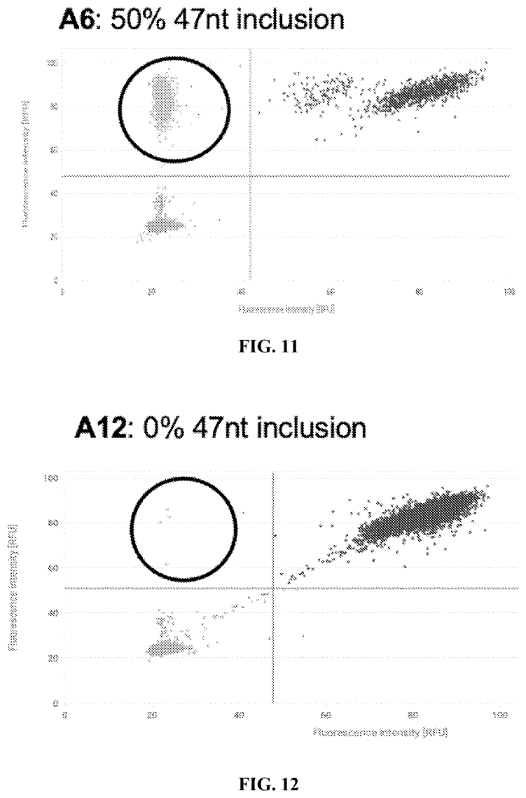

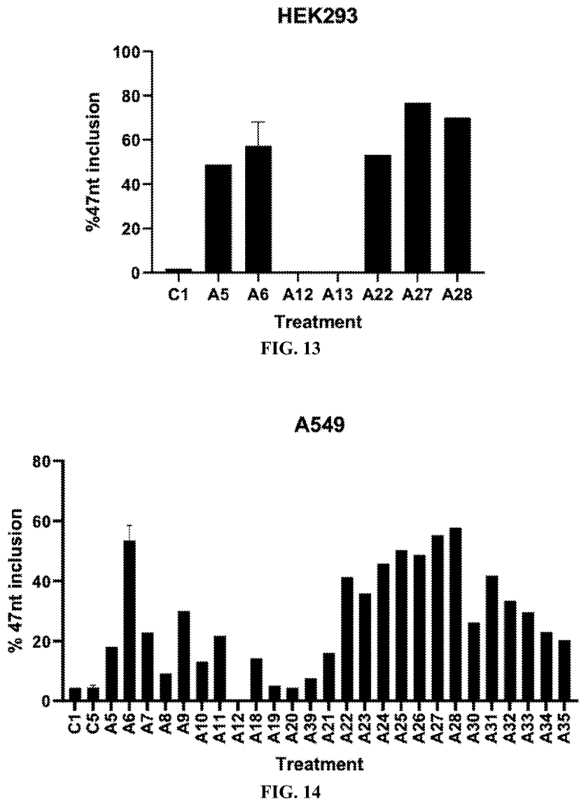

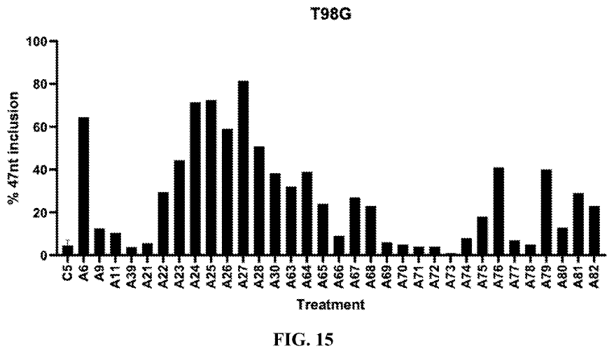

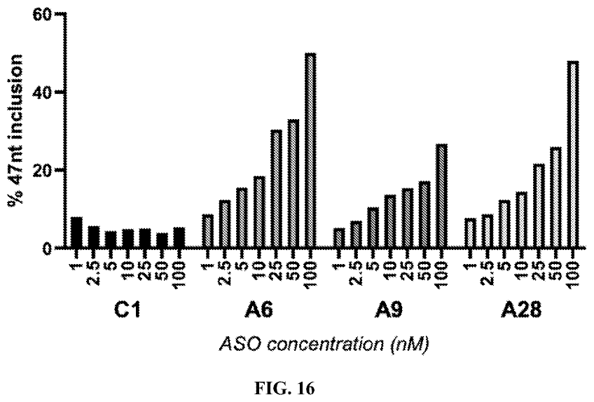

Digital PCR (dPCR) was used to accurately and directly quantify the splicing of the 47nt inclusion (FIGS. 11-16). The Qiagen Qiacuity dPCR instrument was used. The dPCR reaction consisted of the exon 8 47nt inclusion region primers described in Example 2, a HEX probe located in exon 9 to measure overall gene abundance, and a FAM probe spanning the exon 8-exon 9 junction to measure the canonical exon 8 form. Representative dPCR readouts are shown in FIGS. 11-12 and the 47nt inclusion isoform population is circled. The percentage of the total isoform population containing the 47nt inclusion is displayed as an inclusion ratio (FIG. 13-15). Cell lines measured in the screenings include HEK293, A549, and T98G. A dose-response experiment (as in Example 3) was performed in T98G cells (FIG. 16).

### Results

The dPCR quantification of splicing aligns with the results of Example 2, with some experiments showing as high as 80% inclusion in ASO-treated samples (FIGS. 13-15). In addition, the dPCR quantification of the dose-response experiment confirms the dose-responsive effect observed in the agarose gels and supports the on-target mechanism of the ASOs (FIG. 16).

### Example 5: Evaluation of ASO Modulation Using RNA-Seq

### Materials and Methods

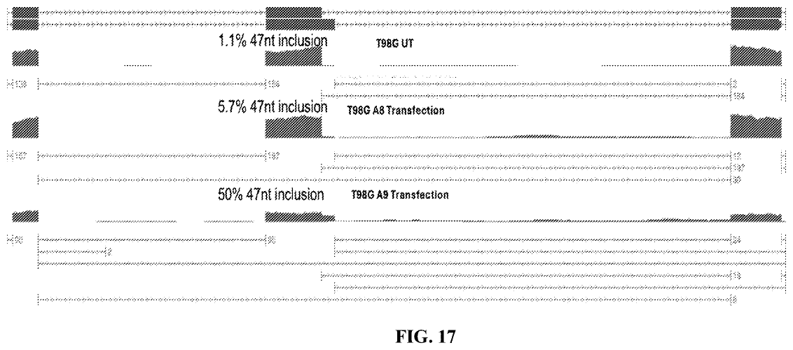

Extracted RNA was submitted for next-generation Illumina RNA sequencing. Approximately 50 million 150 bp, pair-end, stranded reads were generated per sample. Raw FASTQs were processed and inspected for quality using a variety of metrics including overall and per-base sequence quality, nucleotide enrichment, gene body coverage, and genomic distribution. For final visualization and quantification, samples were processed to remove ribosomal RNA reads, aligned to the human genome (GRCh38) using STAR 2-pass alignment, and converted into wiggle files for visualization. Junction read annotation and quantification of supporting reads was performed using a custom script. Reads were visualized in the UCSC genome browser. There are four observed junctions that end at the start of exon 9: the canonical junction (complete exon 8), 47nt inclusion junction, 70nt skipping junction, and a skip junction (exon 8 is completely skipped) and the counts of each are used to determine the splicing ratio. The proportion of each junction was calculated and multiplied by the total counts of junctions ending at exon 9 to obtain the quantification demonstrated in FIG. 17.

### Results

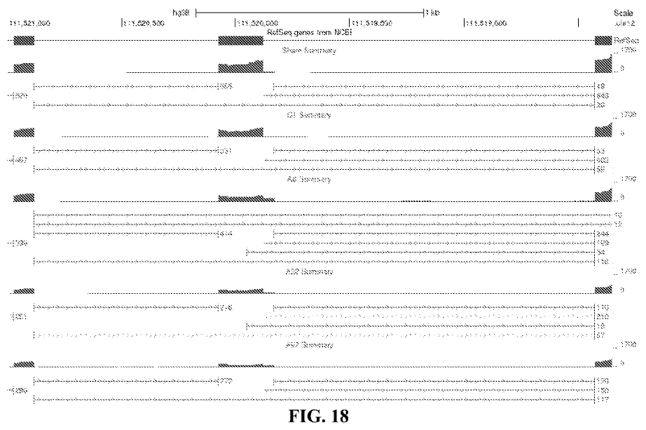

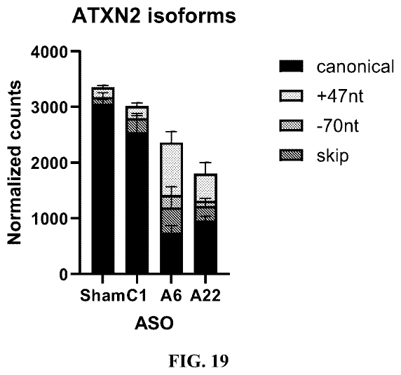

RNA-seq data revealed that T98G cells transfected with A9 show significant changes at the RNA level compared to the untreated sample (FIG. 17). An overall reduction in reads covering the ATXN2 gene was observed. Increased coverage of the 47nt inclusion is visible and quantified at 50% of total junctions ending at exon 9, indicating effective modulation by the ASO. In contrast, treatment with A8 showed little difference in either metric compared to the control sample suggesting it is not an effective ASO. In A549 cells transfected with A6 and A22, a reduction in ATXN2 mRNA is observed and the relative levels of junction reads supporting the 47nt inclusion and 70nt skipping exon junctions were greatly increased compared to the canonical junction (FIGS. 18-19). This suggests that effective ASOs such as A9, A6, and A22 modulate ATXN2 RNA as expected when transfected into cells by both promoting the use of the 47nt inclusion and/or the 70nt skipping 5′ splice sites and by reducing the overall levels of ATXN2.

### Example 6: Modulation of ATXN2 mRNA Expression by ASO Treatment

### Materials and Methods

Following transfection with ASOs, cDNA was either obtained as described above or using the Taqman 2-step Cells-to-CT kit (ThermoFisher #4399002). Quantitative-PCR (qPCR) assays were used to measure the abundance of ATXN2 with either GAPDH (ThermoFisher Hs04420632_g1) or MRPL19 (ThermoFisher Hs00608519_m1) expression as the reference. The ATXN2 mRNA expression was measured using the 2848 assay (ThermoFisher Hs01002848_m1).

A dose-response assay was performed by treating U2OS cells with increasing doses of A9 and A28 ASOs and the ATXN2 mRNA levels were measured with the 2848 assay.

### Results

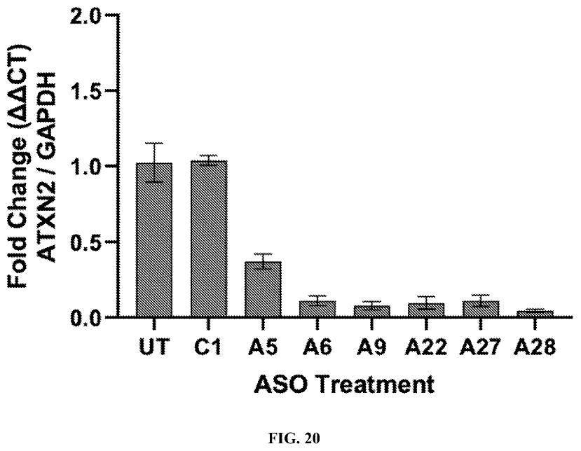

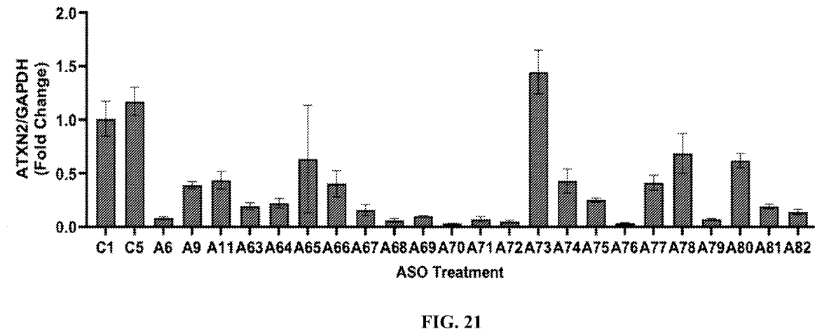

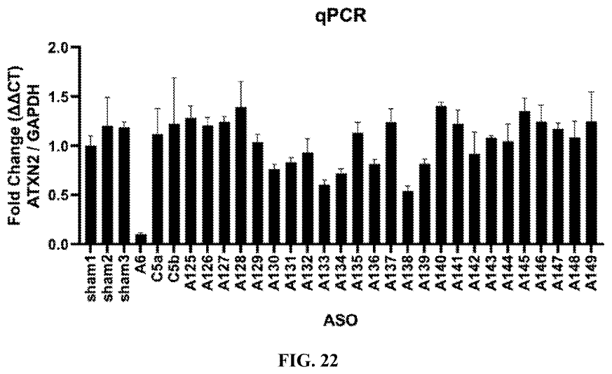

Corroborating the RNA-seq analysis, a strong reduction of ATXN2 mRNA expression was observed with the 2848 assay (FIGS. 20-22) in many of the samples treated with ASOs targeting the exon 8 region or exon 18 region. This suggests that some of the tested ASOs efficiently promoted the reduction of the ATXN2 canonical/functional isoform expression. In general, these results show that effective splicing modulation correlates with effective ATXN2 mRNA downregulation.

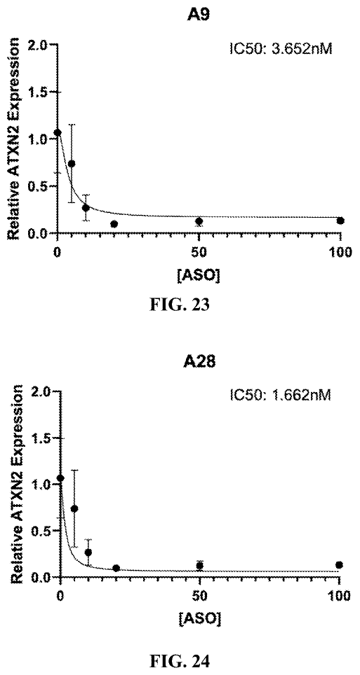

The qPCR assay performed on the U2OS cells treated with increasing levels of ASO confirmed dose-dependent reduction of ATXN2 mRNA expression by the tested ASOs (FIGS. 23-24). The IC₅₀ was calculated as 3.7 nM and 1.7 nM for A9 and A28, respectively.

### Example 7: Modulation of ATXN2 Protein Expression by ASO Treatment

### Materials and Methods

Cell lysates were collected after 2-day ASO treatment in HEK293 and T98G cells. ATXN2 was detected using the BD #611378 antibody (measured by anti-Mouse secondary antibody-HRP) and its levels were normalized either to Total Protein (Total Protein Detection Module, DMTP01, measured by Streptavidin-HRP) or Vinculin (Cell Signaling Technology Rabbit mAb, E1E9V, measured by anti-Rabbit secondary antibody-NIR). Quantification was performed using the Compass for Simple Western Jess software.

### Results

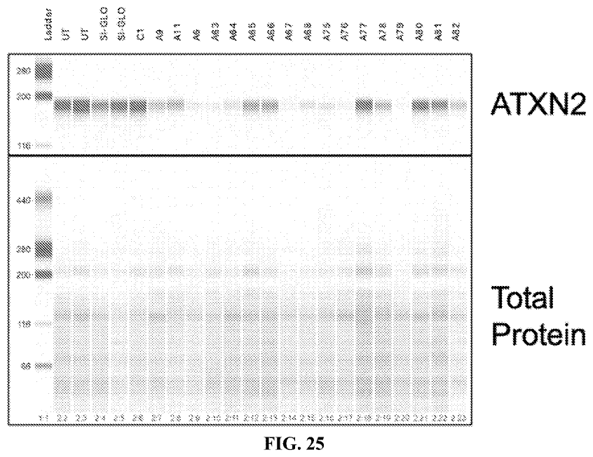

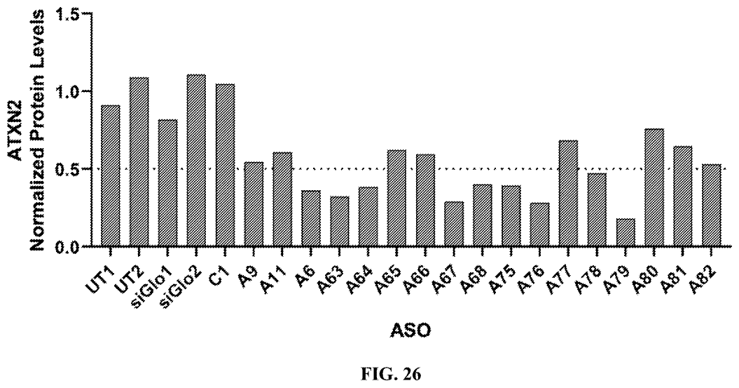

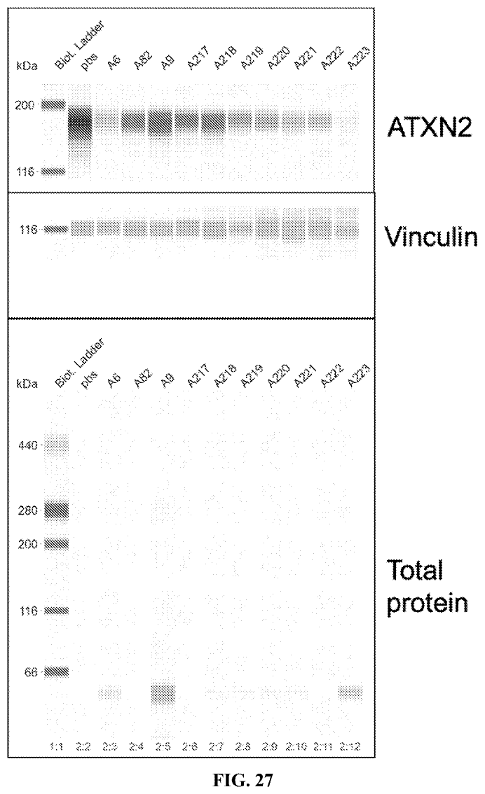

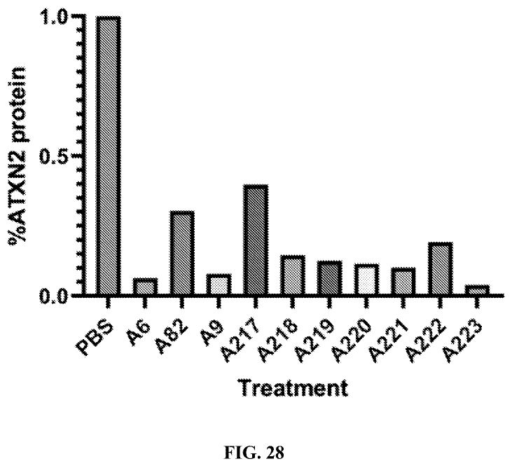

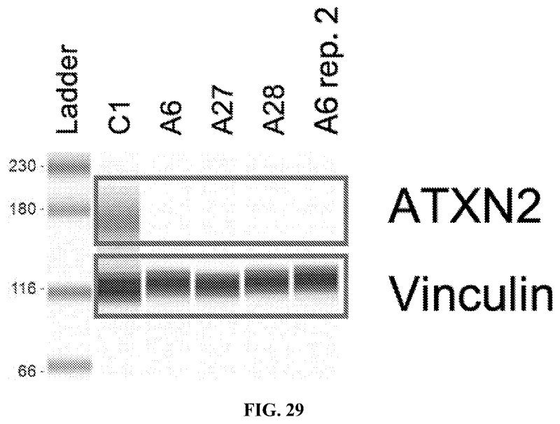

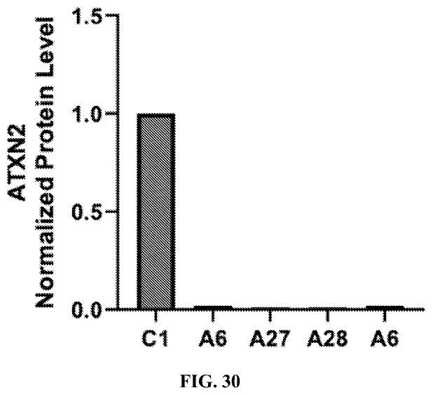

Significant reduction of ATNX2 protein levels were consistently observed for all ASOs previously noted to reduce ATXN2 mRNA expression (FIG. 25-30). This suggests that the tested ASOs, by inducing specific splicing changes which reduce canonical isoform expression and induce PTC-containing isoform expression, effectively reduce ATXN2 protein expression.

### Example 8: Effective Reduction of ATXN2 Protein Expression In Vivo Using ASOs

### Materials and Methods

The Q22 mouse model (Dansithong, PLOS Genet. 2015) includes multiple copies of the entire human ATXN2 trans-gene (16 kb at the 5′ end through 3 kb at the 3′ end, with a normal copy number (22) of the CAG repeat) on a bacterial artificial chromosome (BAC). The normal mouse Atxn2 transcript is not affected.

ASO was reconstituted in 1×PBS (Thermo Fisher Scientific, 10010023), and the concentration of ASO solution was determined by OD260 nm absorbance. For dosing solutions, reconstituted ASO was diluted to the desired concentration in PBS. PBS was used as the vehicle control. For ICV injection in adult mice, mice were anesthetized with 1.5% isoflurane Inhalation at a flow rate of 0.8 liter/min. ASO or PBS solution was injected slowly into one cerebral lateral ventricle at a rate of luL/min. Injected mice were quickly returned to the cage and observed daily for survival and signs of stress. 14 days after injection the mice were euthanized, and relevant tissues were dissected. The tissues were split into two, one for RNA extraction and the other processed to extract cell lysate for protein quantification with Simple Western Jess capillary Western Blot. RNA was reverse transcribed to cDNA, amplified, and run on acrylamide gel. Primer sequences were AAATTATGGTGTAGTGTCTACG (forward) and TGATGGCATGGAGCCCGAT (reverse). ATXN2 was detected using the BD #611378 antibody (measured by anti-Mouse secondary antibody-HRP) and its levels were normalized either to Total Protein (Total Protein Detection Module, DMTP01, measured by Streptavidin-HRP) or Vinculin (Cell Signaling Technology Rabbit mAb, E1E9V, measured by anti-Rabbit secondary antibody-NIR). Quantification was performed using the Compass for Simple Western Jess software.

### Results

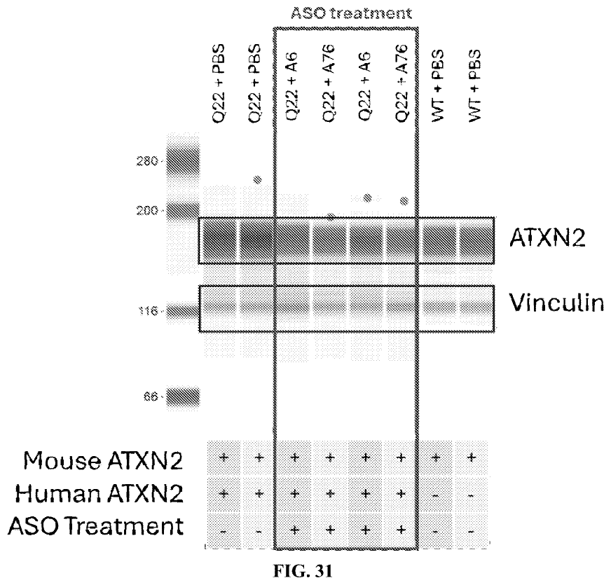

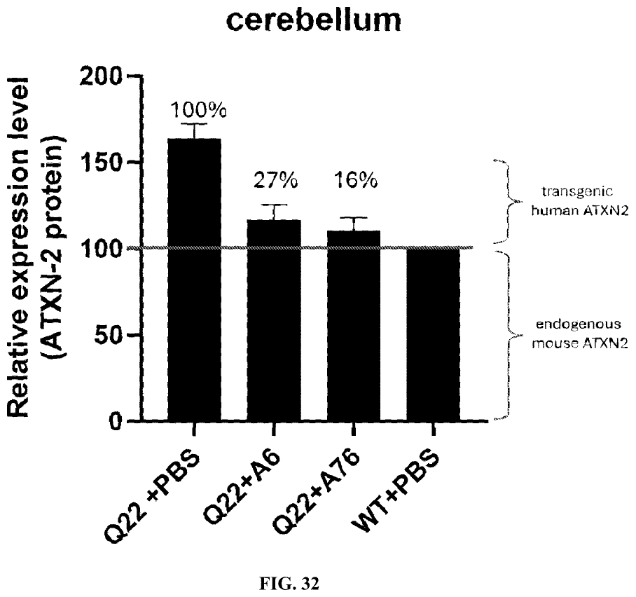

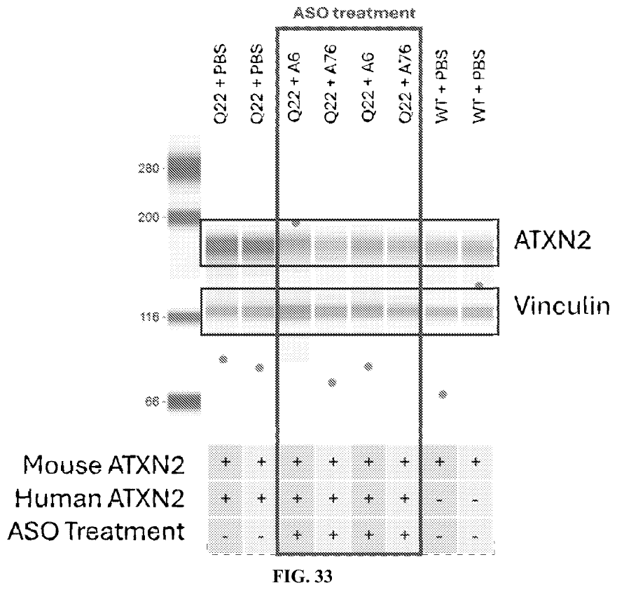

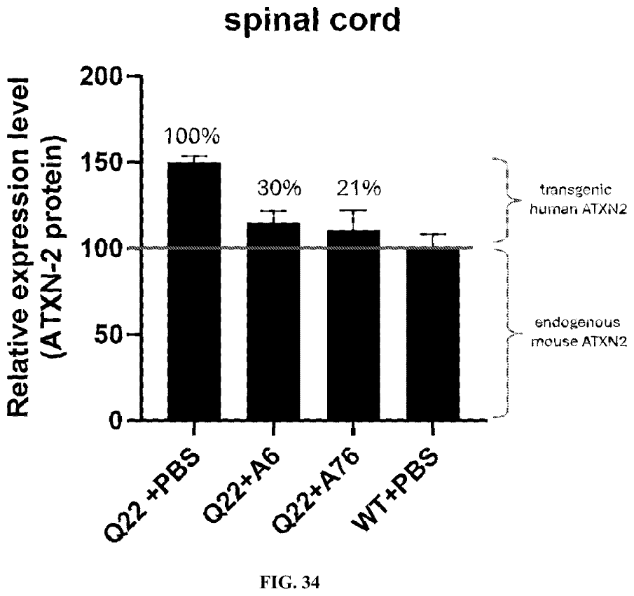

ATXN2 protein levels were higher in Q22 mice compared to WT mice, as expected because the antibody does not distinguish between human and mouse proteins and the Q22 mouse expresses human protein in addition to the endogenous mouse protein. Mice injected with ASOs showed a reduction in total ATXN2 protein expression (see FIGS. 31-34). By considering the difference between the WT ATXN2 (mouse protein only) and the Q22 ATXN2 (mouse plus human protein), the reduction of human ATXN2 can be estimated; this is possible because the splicing event is not conserved (does not exist) in mouse and the ASOs have been shown to not have any effect on the mouse Atxn2 expression (in both mouse cell lines and WT mouse, data not shown). Significant reduction of human ATXN2 levels were observed in the cerebellum (FIG. 32) and spinal cord (FIG. 34) with the ASO treatment. Dose-dependent splicing change and protein reduction was further observed with multiple ASOs tested in the mouse model.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification.

Use of the terms “may” or “can” in reference to an embodiment or aspect of an embodiment also carries with it the alternative meaning of “may not” or “cannot.” As such, if the present specification discloses that a structural feature, element, or functionality may be or can be included, then the negative limitation or exclusionary proviso is also explicitly meant, meaning that in alternative aspects the structural feature, element, or functionality may be excluded. In a similar manner, use of the term “optionally” in reference to an embodiment or aspect of an embodiment means that such embodiment or aspect of the embodiment may be included or may not be included. Whether such a negative limitation or exclusionary proviso applies will be based on whether the negative limitation or exclusionary proviso is recited in the claimed subject matter.

Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges, to the tenth of the unit of the lower limit of the range, unless the context clearly dictates otherwise.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure or any subject matter recited by the claims. No language in the present specification should be construed as indicating any non-claimed element essential to the practice of the invention defined by the claims.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. All statements as to the date or representation as to the contents of these documents is based on the information available to the applicants and does not constitute any admission as to the correctness of the dates or contents of these documents.

SEQUENCE LISTING

| SEQ | | |
|---|---|---|
| ID NO | Name | Sequence |
| 1 | ATXN2 Isoform 1 | NCBI GenBank Accession No. NG_011572.3 |
| | (genomic DNA) | Homo sapiens ataxin 2 (ATXN2), RefSeqGene (LRG_864) on |
| | | chromosome 12 |
| 2 | ATXN2 Isoform 1 | >ref\|NM_002973.4\|1-4341 Homo sapiens ataxin 2 (ATXN2), |
| | (mRNA transcript), | transcript variant 1, mRNA |
| | annotated to show | AGAGCTCGCCTCCCTCCGCCTCAGACTGTTTTGGTAGCAA |
| | Exon 8 (underlined). | CGGCAACGGCGGCGGCGCGTTTCGGCCCGGCTCCCGGCG |
| | | GCTCCTTGGTCTCGGCGGGCCTCCCCGCCCCTTCGTCGTC |
| | | CTCCTTCTCCCCCTCGCCAGCCCGGGCGCCCCTCCGGCCG |
| | | CGCCAACCCGCGCCTCCCCGCTCGGCGCCCGCGCGTCCCC |
| | | GCCGCGTTCCGGCGTCTCCTTGGCGCGCCCGGCTCCCGGC |
| | | TGTCCCCGCCCGGCGTGCGAGCCGGTGTATGGGCCCCTCA |
| | | CCATGTCGCTGAAGCCCCAGCAGCAGCAGCAGCAGCAGC |
| | | AGCAGCAGCAGCAGCAGCAACAGCAGCAGCAGCAGCAG |
| | | CAGCAGCAGCCGCCGCCCGCGGCTGCCAATGTCCGCAAG |
| | | CCCGGCGGCAGCGGCCTTCTAGCGTCGCCCGCCGCCGCG |
| | | CCTTCGCCGTCCTCGTCCTCGGTCTCCTCGTCCTCGGCCAC |
| | | GGCTCCCTCCTCGGTGGTCGCGGCGACCTCCGGCGGCGG |
| | | GAGGCCCGGCCTGGGCAGAGGTCGAAACAGTAACAAAG |
| | | GACTGCCTCAGTCTACGATTTCTTTTGATGGAATCTATGC |
| | | AAATATGAGGATGGTTCATATACTTACATCAGTTGTTGGC |
| | | TCCAAATGTGAAGTACAAGTGAAAAATGGAGGTATATAT |
| | | GAAGGAGTTTTTAAAACTTACAGTCCGAAGTGTGATTTGG |
| | | TACTTGATGCCGCACATGAGAAAAGTACAGAATCCAGTT |
| | | CGGGGCCGAAACGTGAAGAAATAATGGAGAGTATTTTGT |
| | | TCAAATGTTCAGACTTTGTTGTGGTACAGTTTAAAGATAT |
| | | GGACTCCAGTTATGCAAAAAGAGATGCTTTTACTGACTCT |
| | | GCTATCAGTGCTAAAGTGAATGGCGAACACAAAGAGAAG |
| | | GACCTGGAGCCCTGGGATGCAGGTGAACTCACAGCCAAT |
| | | GAGGAACTTGAGGCTTTGGAAAATGACGTATCTAATGGA |
| | | TGGGATCCCAATGATATGTTTCGATATAATGAAGAAAATT |
| | | ATGGTGTAGTGTCTACGTATGATAGCAGTTTATCTTCGTA |
| | | TACAGTGCCCTTAGAAAGAGATAACTCAGAAGAATTTTT |
| | | AAAACGGGAAGCAAGGGCAAACCAGTTAGCAGAAGAAA |
| | | TTGAGTCAAGTGCCCAGTACAAAGCTCGAGTGGCCCTGG |
| | | AAAATGATGATAGGAGTGAGGAAGAAAAATACACAGCA |
| | | GTTCAGAGAAATTCCAGTGAACGTGAGGGGCACAGCATA |
| | | AACACTAGGGAAAATAAATATATTCCTCCTGGACAAAGA |
| | | AATAGAGAAGTCATATCCTGGGGAAGTGGGAGACAGAAT |
| | | TCACCGCGTATGGGCCAGCCTGGATCGGGCTCCATGCCAT |
| | | CAAGATCCACTTCTCACACTTCAGATTTCAACCCGAATTC |
| | | TGGTTCAGACCAAAGAGTAGTTAATGGAGGTGTTCCCTG |
| | | GCCATCGCCTTGCCCATCTCCTTCCTCTCGCCCACCTTCTC |
| | | GCTACCAGTCAGGTCCCAACTCTCTTCCACCTCGGGCAGC |
| | | CACCCCTACACGGCCGCCCTCCAGGCCCCCCTCGCGGCCA |
| | | TCCAGACCCCCGTCTCACCCCTCTGCTCATGGTTCTCCAG |
| | | CTCCTGTCTCTACTATGCCTAAACGCATGTCTTCAGAAGG |
| | | GCCTCCAAGGATGTCCCCAAAGGCCCAGCGACATCCTCG |
| | | AAATCACAGAGTTTCTGCTGGGAGGGGTTCCATATCCAGT |
| | | GGCCTAGAATTTGTATCCCACAACCCACCCAGTGAAGCA |
| | | GCTACTCCTCCAGTAGCAAGGACCAGTCCCTCGGGGGGA |
| | | ACGTGGTCATCAGTGGTCAGTGGGGTTCCAAGATTATCCC |
| | | CTAAAACTCATAGACCCAGGTCTCCCAGACAGAACAGTA |
| | | TTGGAAATACCCCCAGTGGGCCAGTTCTTGCTTCTCCCCA |
| | | AGCTGGTATTATTCCAACTGAAGCTGTTGCCATGCCTATT |
| | | CCAGCTGCATCTCCTACGCCTGCTAGTCCTGCATCGAACA |
| | | GAGCTGTTACCCCTTCTAGTGAGGCTAAAGATTCCAGGCT |
| | | TCAAGATCAGAGGCAGAACTCTCCTGCAGGGAATAAAGA |
| | | AAATATTAAACCCAATGAAACATCACCTAGCTTCTCAAA |
| | | AGCTGAAAACAAAGGTATATCACCAGTTGTTTCTGAACA |
| | | TAGAAAACAGATTGATGATTTAAAGAAATTTAAGAATGA |
| | | TTTTAGGTTACAGCCAAGTTCTACTTCTGAATCTATGGAT |
| | | CAACTACTAAACAAAAATAGAGAGGGAGAAAAATCAAG |
| | | AGATTTGATCAAAGACAAAATTGAACCAAGTGCTAAGGA |
| | | TTCTTTCATTGAAAATAGCAGCAGCAACTGTACCAGTGGC |
| | | AGCAGCAAGCCGAATAGCCCCAGCATTTCCCCTTCAATA |
| | | CTTAGTAACACGGAGCACAAGAGGGGACCTGAGGTCACT |
| | | TCCCAAGGGGTTCAGACTTCCAGCCCAGCATGTAAACAA |
| | | GAGAAAGACGATAAGGAAGAGAAGAAAGACGCAGCTGA |
| | | GCAAGTTAGGAAATCAACATTGAATCCCAATGCAAAGGA |
| | | GTTCAACCCACGTTCCTTCTCTCAGCCAAAGCCTTCTACT |
| | | ACCCCAACTTCACCTCGGCCTCAAGCACAACCTAGCCCAT |
| | | CTATGGTGGGTCATCAACAGCCAACTCCAGTTTATACTCA |
| | | GCCTGTTTGTTTTGCACCAAATATGATGTATCCAGTCCCA |
| | | GTGAGCCCAGGCGTGCAACCTTTATACCCAATACCTATGA |
| | | CGCCCATGCCAGTGAATCAAGCCAAGACATATAGAGCAG |
| | | TACCAAATATGCCCCAACAGCGGCAAGACCAGCATCATC |
| | | AGAGTGCCATGATGCACCCAGCGTCAGCAGCGGGCCCAC |
| | | CGATTGCAGCCACCCCACCAGCTTACTCCACGCAATATGT |
| | | TGCCTACAGTCCTCAGCAGTTCCCAAATCAGCCCCTTGTT |
| | | CAGCATGTGCCACATTATCAGTCTCAGCATCCTCATGTCT |
| | | ATAGTCCTGTAATACAGGGTAATGCTAGAATGATGGCAC |
| | | CACCAACACACGCCCAGCCTGGTTTAGTATCTTCTTCAGC |
| | | AACTCAGTACGGGGCTCATGAGCAGACGCATGCGATGTA |
| | | TGCATGTCCCAAATTACCATACAACAAGGAGACAAGCCC |
| | | TTCTTTCTACTTTGCCATTTCCACGGGCTCCCTTGCTCAGC |
| | | AGTATGCGCACCCTAACGCTACCCTGCACCCACATACTCC |
| | | ACACCCTCAGCCTTCAGCTACCCCCACTGGACAGCAGCA |
| | | AAGCCAACATGGTGGAAGTCATCCTGCACCCAGTCCTGTT |
| | | CAGCACCATCAGCACCAGGCCGCCCAGGCTCTCCATCTG |
| | | GCCAGTCCACAGCAGCAGTCAGCCATTTACCACGCGGGG |
| | | CTTGCGCCAACTCCACCCTCCATGACACCTGCCTCCAACA |
| | | CGCAGTCGCCACAGAATAGTTTCCCAGCAGCACAACAGA |
| | | CTGTCTTTACGATCCATCCTTCTCACGTTCAGCCGGCGTA |
| | | TACCAACCCACCCCACATGGCCCACGTACCTCAGGCTCAT |
| | | GTACAGTCAGGAATGGTTCCTTCTCATCCAACTGCCCATG |
| | | CGCCAATGATGCTAATGACGACACAGCCACCCGGCGGTC |
| | | CCCAGGCCGCCCTCGCTCAAAGTGCACTACAGCCCATTCC |
| | | AGTCTCGACAACAGCGCATTTCCCCTATATGACGCACCCT |
| | | TCAGTACAAGCCCACCACCAACAGCAGTTGTAAGGCTGC |
| | | CCTGGAGGAACCGAAAGGCCAAATTCCCTCCTCCCTTCTA |
| | | CTGCTTCTACCAACTGGAAGCACAGAAAACTAGAATTTC |
| | | ATTTATTTTGTTTTTAAAATATATATGTTGATTTCTTGTAA |
| | | CATCCAATAGGAATGCTAACAGTTCACTTGCAGTGGAAG |
| | | ATACTTGGACCGAGTAGAGGCATTTAGGAACTTGGGGGC |
| | | TATTCCATAATTCCATATGCTGTTTCAGAGTCCCGCAGGT |
| | | ACCCCAGCTCTGCTTGCCGAAACTGGAAGTTATTTATTTT |
| | | TTAATAACCCTTGAAAGTCATGAACACATCAGCTAGCAA |
| | | AAGAAGTAACAAGAGTGATTCTTGCTGCTATTACTGCTAA |
| | | AAAAAAAAAAAAAAAAAAATCAAGACTTGGAACGCCCT |
| | | TTTACTAAACTTGACAAAGTTTCAGTAAATTCTTACCGTC |
| | | AAACTGACGGATTATTATTTATAAATCAAGTTTGATGAGG |
| | | TGATCACTGTCTACAGTGGTTCAACTTTTAAGTTAAGGGA |
| | | AAAACTTTTACTTTGTAGATAATATAAAATAAAAACTTAA |
| | | AAAAAATTTAAAAAATAAAAAAAGTTTTAAAAACTGA |
| 3 | ATXN2 Isoform 1 | >ref\|NM_002973.4\|1-4341 Homo sapiens ataxin 2 (ATXN2), |
| | (mRNA transcript), | transcript variant 1, mRNA |
| | annotated to show: Exon | AGAGCTCGCCTCCCTCCGCCTCAGACTGTTTTGGTAG |
| | 8 (underlined), the | CAACGGCAACGGCGGCGGCGCGTTTCGGCCCGGCTC |
| | 47 nt inclusion | CCGGCGGCTCCTTGGTCTCGGCGGGCCTCCCCGCCC |
| | (underlined and bold), | CTTCGTCGTCCTCCTTCTCCCCCTCGCCAGCCCGGGC |
| | and Exon 9 (bold). | GCCCCTCCGGCCGCGCCAACCCGCGCCTCCCCGCTC |
| | | GGCGCCCGCGCGTCCCCGCCGCGTTCCGGCGTCTCC |
| | | TTGGCGCGCCCGGCTCCCGGCTGTCCCCGCCCGGCG |
| | | TGCGAGCCGGTGTATGGGCCCCTCACCATGTCGCTG |
| | | AAGCCCCAGCAGCAGCAGCAGCAGCAGCAGCAGCA |
| | | GCAGCAGCAGCAACAGCAGCAGCAGCAGCAGCAGC |
| | | AGCAGCCGCCGCCCGCGGCTGCCAATGTCCGCAAGC |
| | | CCGGCGGCAGCGGCCTTCTAGCGTCGCCCGCCGCCG |
| | | CGCCTTCGCCGTCCTCGTCCTCGGTCTCCTCGTCCTC |
| | | GGCCACGGCTCCCTCCTCGGTGGTCGCGGCGACCTC |
| | | CGGCGGCGGGAGGCCCGGCCTGGGCAGAGGTCGAA |
| | | ACAGTAACAAAGGACTGCCTCAGTCTACGATTTCTT |
| | | TTGATGGAATCTATGCAAATATGAGGATGGTTCATA |
| | | TACTTACATCAGTTGTTGGCTCCAAATGTGAAGTAC |
| | | AAGTGAAAAATGGAGGTATATATGAAGGAGTTTTTA |
| | | AAACTTACAGTCCGAAGTGTGATTTGGTACTTGATG |
| | | CCGCACATGAGAAAAGTACAGAATCCAGTTCGGGGC |
| | | CGAAACGTGAAGAAATAATGGAGAGTATTTTGTTCA |
| | | AATGTTCAGACTTTGTTGTGGTACAGTTTAAAGATAT |
| | | GGACTCCAGTTATGCAAAAAGAGATGCTTTTACTGA |
| | | CTCTGCTATCAGTGCTAAAGTGAATGGCGAACACAA |
| | | AGAGAAGGACCTGGAGCCCTGGGATGCAGGTGAAC |
| | | TCACAGCCAATGAGGAACTTGAGGCTTTGGAAAATG |
| | | ACGTATCTAATGGATGGGATCCCAATGATATGTTTC |
| | | GATATAATGAAGAAAATTATGGTGTAGTGTCTACGT |
| | | ATGATAGCAGTTTATCTTCGTATACAGTGCCCTTAGA |
| | | AAGAGATAACTCAGAAGAATTTTTAAAACGGGAAG |
| | | CAAGGGCAAACCAGTTAGCAGAAGAAATTGAGTCA |
| | | AGTGCCCAGTACAAAGCTCGAGTGGCCCTGGAAAAT |
| | | GATGATAGGAGTGAGGAAGAAAAATACACAGCAGT |
| | | TCAGAGAAATTCCAGTGAACGTGAGGGGCACAGCAT |
| | | AAACACTAGGTATTTAAAGGAAATCATGATGCAGT |
| | | ATTTTGGATACACAACTCAAGGGAAAATAAATAT |
| | | ATTCCTCCTGGACAAAGAAATAGAGAAGTCATAT |
| | | CCTGGGGAAGTGGGAGACAGAATTCACCGCGTAT |
| | | GGGCCAGCCTGGATCGGGCTCCATGCCATCAAGA |
| | | TCCACTTCTCACACTTCAGATTTCAACCCGAATTC |
| | | TGGTTCAGACCAAAGAGTAGTTAATGGAGGTGTT |
| | | CCCTGGCCATCGCCTTGCCCATCTCCTTCCTCTCGCC |
| | | CACCTTCTCGCTACCAGTCAGGTCCCAACTCTCTTCC |
| | | ACCTCGGGCAGCCACCCCTACACGGCCGCCCTCCAG |
| | | GCCCCCCTCGCGGCCATCCAGACCCCCGTCTCACCC |
| | | CTCTGCTCATGGTTCTCCAGCTCCTGTCTCTACTATG |
| | | CCTAAACGCATGTCTTCAGAAGGGCCTCCAAGGATG |
| | | TCCCCAAAGGCCCAGCGACATCCTCGAAATCACAGA |
| | | GTTTCTGCTGGGAGGGGTTCCATATCCAGTGGCCTA |
| | | GAATTTGTATCCCACAACCCACCCAGTGAAGCAGCT |
| | | ACTCCTCCAGTAGCAAGGACCAGTCCCTCGGGGGGA |
| | | ACGTGGTCATCAGTGGTCAGTGGGGTTCCAAGATTA |
| | | TCCCCTAAAACTCATAGACCCAGGTCTCCCAGACAG |
| | | AACAGTATTGGAAATACCCCCAGTGGGCCAGTTCTT |
| | | GCTTCTCCCCAAGCTGGTATTATTCCAACTGAAGCTG |
| | | TTGCCATGCCTATTCCAGCTGCATCTCCTACGCCTGC |
| | | TAGTCCTGCATCGAACAGAGCTGTTACCCCTTCTAGT |
| | | GAGGCTAAAGATTCCAGGCTTCAAGATCAGAGGCAG |
| | | AACTCTCCTGCAGGGAATAAAGAAAATATTAAACCC |
| | | AATGAAACATCACCTAGCTTCTCAAAAGCTGAAAAC |
| | | AAAGGTATATCACCAGTTGTTTCTGAACATAGAAAA |
| | | CAGATTGATGATTTAAAGAAATTTAAGAATGATTTT |
| | | AGGTTACAGCCAAGTTCTACTTCTGAATCTATGGATC |
| | | AACTACTAAACAAAAATAGAGAGGGAGAAAAATCA |
| | | AGAGATTTGATCAAAGACAAAATTGAACCAAGTGCT |
| | | AAGGATTCTTTCATTGAAAATAGCAGCAGCAACTGT |
| | | ACCAGTGGCAGCAGCAAGCCGAATAGCCCCAGCATT |
| | | TCCCCTTCAATACTTAGTAACACGGAGCACAAGAGG |
| | | GGACCTGAGGTCACTTCCCAAGGGGTTCAGACTTCC |
| | | AGCCCAGCATGTAAACAAGAGAAAGACGATAAGGA |
| | | AGAGAAGAAAGACGCAGCTGAGCAAGTTAGGAAAT |
| | | CAACATTGAATCCCAATGCAAAGGAGTTCAACCCAC |
| | | GTTCCTTCTCTCAGCCAAAGCCTTCTACTACCCCAAC |
| | | TTCACCTCGGCCTCAAGCACAACCTAGCCCATCTAT |
| | | GGTGGGTCATCAACAGCCAACTCCAGTTTATACTCA |
| | | GCCTGTTTGTTTTGCACCAAATATGATGTATCCAGTC |
| | | CCAGTGAGCCCAGGCGTGCAACCTTTATACCCAATA |
| | | CCTATGACGCCCATGCCAGTGAATCAAGCCAAGACA |
| | | TATAGAGCAGTACCAAATATGCCCCAACAGCGGCAA |
| | | GACCAGCATCATCAGAGTGCCATGATGCACCCAGCG |
| | | TCAGCAGCGGGCCCACCGATTGCAGCCACCCCACCA |
| | | GCTTACTCCACGCAATATGTTGCCTACAGTCCTCAGC |
| | | AGTTCCCAAATCAGCCCCTTGTTCAGCATGTGCCAC |
| | | ATTATCAGTCTCAGCATCCTCATGTCTATAGTCCTGT |
| | | AATACAGGGTAATGCTAGAATGATGGCACCACCAAC |
| | | ACACGCCCAGCCTGGTTTAGTATCTTCTTCAGCAACT |
| | | CAGTACGGGGCTCATGAGCAGACGCATGCGATGTAT |
| | | GCATGTCCCAAATTACCATACAACAAGGAGACAAGC |
| | | CCTTCTTTCTACTTTGCCATTTCCACGGGCTCCCTTGC |
| | | TCAGCAGTATGCGCACCCTAACGCTACCCTGCACCC |
| | | ACATACTCCACACCCTCAGCCTTCAGCTACCCCCACT |
| | | GGACAGCAGCAAAGCCAACATGGTGGAAGTCATCCT |
| | | GCACCCAGTCCTGTTCAGCACCATCAGCACCAGGCC |
| | | GCCCAGGCTCTCCATCTGGCCAGTCCACAGCAGCAG |
| | | TCAGCCATTTACCACGCGGGGCTTGCGCCAACTCCA |
| | | CCCTCCATGACACCTGCCTCCAACACGCAGTCGCCA |
| | | CAGAATAGTTTCCCAGCAGCACAACAGACTGTCTTT |
| | | ACGATCCATCCTTCTCACGTTCAGCCGGCGTATACCA |
| | | ACCCACCCCACATGGCCCACGTACCTCAGGCTCATG |
| | | TACAGTCAGGAATGGTTCCTTCTCATCCAACTGCCCA |
| | | TGCGCCAATGATGCTAATGACGACACAGCCACCCGG |
| | | CGGTCCCCAGGCCGCCCTCGCTCAAAGTGCACTACA |
| | | GCCCATTCCAGTCTCGACAACAGCGCATTTCCCCTAT |
| | | ATGACGCACCCTTCAGTACAAGCCCACCACCAACAG |
| | | CAGTTGTAAGGCTGCCCTGGAGGAACCGAAAGGCCA |
| | | AATTCCCTCCTCCCTTCTACTGCTTCTACCAACTGGA |
| | | AGCACAGAAAACTAGAATTTCATTTATTTTGTTTTTA |
| | | AAATATATATGTTGATTTCTTGTAACATCCAATAGGA |
| | | ATGCTAACAGTTCACTTGCAGTGGAAGATACTTGGA |
| | | CCGAGTAGAGGCATTTAGGAACTTGGGGGCTATTCC |
| | | ATAATTCCATATGCTGTTTCAGAGTCCCGCAGGTACC |
| | | CCAGCTCTGCTTGCCGAAACTGGAAGTTATTTATTTT |
| | | TTAATAACCCTTGAAAGTCATGAACACATCAGCTAG |
| | | CAAAAGAAGTAACAAGAGTGATTCTTGCTGCTATTA |
| | | CTGCTAAAAAAAAAAAAAAAAAAAAATCAAGACTT |
| | | GGAACGCCCTTTTACTAAACTTGACAAAGTTTCAGT |
| | | AAATTCTTACCGTCAAACTGACGGATTATTATTTATA |
| | | AATCAAGTTTGATGAGGTGATCACTGTCTACAGTGG |
| | | TTCAACTTTTAAGTTAAGGGAAAAACTTTTACTTTGT |
| | | AGATAATATAAAATAAAAACTTAAAAAAAATTTAAA |
| | | AAATAAAAAAAGTTTTAAAAACTGA |
| 4 | ATXN2 Isoform 1 | >ref\|NM_002973.4\|1-4341 Homo sapiens ataxin 2 (ATXN2), |
| | (mRNA transcript), | transcript variant 1, mRNA |
| | annotated to show: Exon | AGAGCTCGCCTCCCTCCGCCTCAGACTGTTTTGGTAG |
| | 8 (underlined), and the | CAACGGCAACGGCGGCGGCGCGTTTCGGCCCGGCTC |
| | −70 nt NMD variant of | CCGGCGGCTCCTTGGTCTCGGCGGGCCTCCCCGCCC |
| | Exon 8 (underlined and | CTTCGTCGTCCTCCTTCTCCCCCTCGCCAGCCCGGGC |
| | bold). | GCCCCTCCGGCCGCGCCAACCCGCGCCTCCCCGCTC |
| | | GGCGCCCGCGCGTCCCCGCCGCGTTCCGGCGTCTCC |
| | | TTGGCGCGCCCGGCTCCCGGCTGTCCCCGCCCGGCG |
| | | TGCGAGCCGGTGTATGGGCCCCTCACCATGTCGCTG |
| | | AAGCCCCAGCAGCAGCAGCAGCAGCAGCAGCAGCA |
| | | GCAGCAGCAGCAACAGCAGCAGCAGCAGCAGCAGC |
| | | AGCAGCCGCCGCCCGCGGCTGCCAATGTCCGCAAGC |
| | | CCGGCGGCAGCGGCCTTCTAGCGTCGCCCGCCGCCG |
| | | CGCCTTCGCCGTCCTCGTCCTCGGTCTCCTCGTCCTC |
| | | GGCCACGGCTCCCTCCTCGGTGGTCGCGGCGACCTC |
| | | CGGCGGCGGGAGGCCCGGCCTGGGCAGAGGTCGAA |
| | | ACAGTAACAAAGGACTGCCTCAGTCTACGATTTCTT |
| | | TTGATGGAATCTATGCAAATATGAGGATGGTTCATA |
| | | TACTTACATCAGTTGTTGGCTCCAAATGTGAAGTAC |
| | | AAGTGAAAAATGGAGGTATATATGAAGGAGTTTTTA |
| | | AAACTTACAGTCCGAAGTGTGATTTGGTACTTGATG |
| | | CCGCACATGAGAAAAGTACAGAATCCAGTTCGGGGC |
| | | CGAAACGTGAAGAAATAATGGAGAGTATTTTGTTCA |
| | | AATGTTCAGACTTTGTTGTGGTACAGTTTAAAGATAT |
| | | GGACTCCAGTTATGCAAAAAGAGATGCTTTTACTGA |
| | | CTCTGCTATCAGTGCTAAAGTGAATGGCGAACACAA |
| | | AGAGAAGGACCTGGAGCCCTGGGATGCAGGTGAAC |
| | | TCACAGCCAATGAGGAACTTGAGGCTTTGGAAAATG |
| | | ACGTATCTAATGGATGGGATCCCAATGATATGTTTC |
| | | GATATAATGAAGAAAATTATGGTGTAGTGTCTACGT |
| | | ATGATAGCAGTTTATCTTCGTATACAGTGCCCTTAGA |
| | | AAGAGATAACTCAGAAGAATTTTTAAAACGGGAAG |
| | | CAAGGGCAAACCAGTTAGCAGAAGAAATTGAGTCA |
| | | AGTGCCCAGTACAAAGCTCGAGTGGCCCTGGAAAAT |
| | | GATGATAGGAGTGAGGAAGAAAAATACACAGCAG |
| | | TTCAGAGAAATTCCAGTGAACGTGAGGGGCACAG |
| | | CATAAACACTAGGGAAAATAAATATATTCCTCCTGG |
| | | ACAAAGAAATAGAGAAGTCATATCCTGGGGAAGTG |
| | | GGAGACAGAATTCACCGCGTATGGGCCAGCCTGGAT |
| | | CGGGCTCCATGCCATCAAGATCCACTTCTCACACTTC |
| | | AGATTTCAACCCGAATTCTGGTTCAGACCAAAGAGT |
| | | AGTTAATGGAGGTGTTCCCTGGCCATCGCCTTGCCC |
| | | ATCTCCTTCCTCTCGCCCACCTTCTCGCTACCAGTCA |
| | | GGTCCCAACTCTCTTCCACCTCGGGCAGCCACCCCTA |
| | | CACGGCCGCCCTCCAGGCCCCCCTCGCGGCCATCCA |
| | | GACCCCCGTCTCACCCCTCTGCTCATGGTTCTCCAGC |
| | | TCCTGTCTCTACTATGCCTAAACGCATGTCTTCAGAA |
| | | GGGCCTCCAAGGATGTCCCCAAAGGCCCAGCGACAT |
| | | CCTCGAAATCACAGAGTTTCTGCTGGGAGGGGTTCC |
| | | ATATCCAGTGGCCTAGAATTTGTATCCCACAACCCA |
| | | CCCAGTGAAGCAGCTACTCCTCCAGTAGCAAGGACC |
| | | AGTCCCTCGGGGGGAACGTGGTCATCAGTGGTCAGT |
| | | GGGGTTCCAAGATTATCCCCTAAAACTCATAGACCC |
| | | AGGTCTCCCAGACAGAACAGTATTGGAAATACCCCC |
| | | AGTGGGCCAGTTCTTGCTTCTCCCCAAGCTGGTATTA |
| | | TTCCAACTGAAGCTGTTGCCATGCCTATTCCAGCTGC |
| | | ATCTCCTACGCCTGCTAGTCCTGCATCGAACAGAGC |
| | | TGTTACCCCTTCTAGTGAGGCTAAAGATTCCAGGCTT |
| | | CAAGATCAGAGGCAGAACTCTCCTGCAGGGAATAA |
| | | AGAAAATATTAAACCCAATGAAACATCACCTAGCTT |
| | | CTCAAAAGCTGAAAACAAAGGTATATCACCAGTTGT |
| | | TTCTGAACATAGAAAACAGATTGATGATTTAAAGAA |
| | | ATTTAAGAATGATTTTAGGTTACAGCCAAGTTCTACT |
| | | TCTGAATCTATGGATCAACTACTAAACAAAAATAGA |
| | | GAGGGAGAAAAATCAAGAGATTTGATCAAAGACAA |
| | | AATTGAACCAAGTGCTAAGGATTCTTTCATTGAAAA |
| | | TAGCAGCAGCAACTGTACCAGTGGCAGCAGCAAGCC |
| | | GAATAGCCCCAGCATTTCCCCTTCAATACTTAGTAAC |
| | | ACGGAGCACAAGAGGGGACCTGAGGTCACTTCCCA |
| | | AGGGGTTCAGACTTCCAGCCCAGCATGTAAACAAGA |
| | | GAAAGACGATAAGGAAGAGAAGAAAGACGCAGCTG |
| | | AGCAAGTTAGGAAATCAACATTGAATCCCAATGCAA |
| | | AGGAGTTCAACCCACGTTCCTTCTCTCAGCCAAAGC |
| | | CTTCTACTACCCCAACTTCACCTCGGCCTCAAGCACA |
| | | ACCTAGCCCATCTATGGTGGGTCATCAACAGCCAAC |
| | | TCCAGTTTATACTCAGCCTGTTTGTTTTGCACCAAAT |
| | | ATGATGTATCCAGTCCCAGTGAGCCCAGGCGTGCAA |
| | | CCTTTATACCCAATACCTATGACGCCCATGCCAGTG |
| | | AATCAAGCCAAGACATATAGAGCAGTACCAAATATG |
| | | CCCCAACAGCGGCAAGACCAGCATCATCAGAGTGCC |
| | | ATGATGCACCCAGCGTCAGCAGCGGGCCCACCGATT |
| | | GCAGCCACCCCACCAGCTTACTCCACGCAATATGTT |
| | | GCCTACAGTCCTCAGCAGTTCCCAAATCAGCCCCTT |
| | | GTTCAGCATGTGCCACATTATCAGTCTCAGCATCCTC |
| | | ATGTCTATAGTCCTGTAATACAGGGTAATGCTAGAA |
| | | TGATGGCACCACCAACACACGCCCAGCCTGGTTTAG |
| | | TATCTTCTTCAGCAACTCAGTACGGGGCTCATGAGC |
| | | AGACGCATGCGATGTATGCATGTCCCAAATTACCAT |
| | | ACAACAAGGAGACAAGCCCTTCTTTCTACTTTGCCA |
| | | TTTCCACGGGCTCCCTTGCTCAGCAGTATGCGCACCC |
| | | TAACGCTACCCTGCACCCACATACTCCACACCCTCA |
| | | GCCTTCAGCTACCCCCACTGGACAGCAGCAAAGCCA |
| | | ACATGGTGGAAGTCATCCTGCACCCAGTCCTGTTCA |
| | | GCACCATCAGCACCAGGCCGCCCAGGCTCTCCATCT |
| | | GGCCAGTCCACAGCAGCAGTCAGCCATTTACCACGC |
| | | GGGGCTTGCGCCAACTCCACCCTCCATGACACCTGC |
| | | CTCCAACACGCAGTCGCCACAGAATAGTTTCCCAGC |
| | | AGCACAACAGACTGTCTTTACGATCCATCCTTCTCAC |
| | | GTTCAGCCGGCGTATACCAACCCACCCCACATGGCC |
| | | CACGTACCTCAGGCTCATGTACAGTCAGGAATGGTT |
| | | CCTTCTCATCCAACTGCCCATGCGCCAATGATGCTAA |
| | | TGACGACACAGCCACCCGGCGGTCCCCAGGCCGCCC |
| | | TCGCTCAAAGTGCACTACAGCCCATTCCAGTCTCGA |
| | | CAACAGCGCATTTCCCCTATATGACGCACCCTTCAGT |
| | | ACAAGCCCACCACCAACAGCAGTTGTAAGGCTGCCC |
| | | TGGAGGAACCGAAAGGCCAAATTCCCTCCTCCCTTC |
| | | TACTGCTTCTACCAACTGGAAGCACAGAAAACTAGA |
| | | ATTTCATTTATTTTGTTTTTAAAATATATATGTTGATT |
| | | TCTTGTAACATCCAATAGGAATGCTAACAGTTCACTT |
| | | GCAGTGGAAGATACTTGGACCGAGTAGAGGCATTTA |
| | | GGAACTTGGGGGCTATTCCATAATTCCATATGCTGTT |
| | | TCAGAGTCCCGCAGGTACCCCAGCTCTGCTTGCCGA |
| | | AACTGGAAGTTATTTATTTTTTAATAACCCTTGAAAG |
| | | TCATGAACACATCAGCTAGCAAAAGAAGTAACAAG |
| | | AGTGATTCTTGCTGCTATTACTGCTAAAAAAAAAAA |
| | | AAAAAAAAAATCAAGACTTGGAACGCCCTTTTACTA |
| | | AACTTGACAAAGTTTCAGTAAATTCTTACCGTCAAA |
| | | CTGACGGATTATTATTTATAAATCAAGTTTGATGAG |
| | | GTGATCACTGTCTACAGTGGTTCAACTTTTAAGTTAA |
| | | GGGAAAAACTTTTACTTTGTAGATAATATAAAATAA |
| | | AAACTTAAAAAAAATTTAAAAAATAAAAAAAGTTTT |
| | | AAAAACTGA |
| 5 | ATXN2 Isoform 1 | >ref\|NM_002973.4\|1-4341 Homo sapiens ataxin 2 (ATXN2), |
| | (mRNA transcript), | transcript variant 1, mRNA |
| | annotated to show: Exon | AGAGCTCGCCTCCCTCCGCCTCAGACTGTTTTGGTAG |
| | 8 (underlined), and the | CAACGGCAACGGCGGCGGCGCGTTTCGGCCCGGCTC |
| | -25 nt NMD variant of | CCGGCGGCTCCTTGGTCTCGGCGGGCCTCCCCGCCC |
| | Exon 8 (underlined and | CTTCGTCGTCCTCCTTCTCCCCCTCGCCAGCCCGGGC |
| | bold). | GCCCCTCCGGCCGCGCCAACCCGCGCCTCCCCGCTC |
| | | GGCGCCCGCGCGTCCCCGCCGCGTTCCGGCGTCTCC |
| | | TTGGCGCGCCCGGCTCCCGGCTGTCCCCGCCCGGCG |
| | | TGCGAGCCGGTGTATGGGCCCCTCACCATGTCGCTG |
| | | AAGCCCCAGCAGCAGCAGCAGCAGCAGCAGCAGCA |
| | | GCAGCAGCAGCAACAGCAGCAGCAGCAGCAGCAGC |
| | | AGCAGCCGCCGCCCGCGGCTGCCAATGTCCGCAAGC |
| | | CCGGCGGCAGCGGCCTTCTAGCGTCGCCCGCCGCCG |
| | | CGCCTTCGCCGTCCTCGTCCTCGGTCTCCTCGTCCTC |
| | | GGCCACGGCTCCCTCCTCGGTGGTCGCGGCGACCTC |
| | | CGGCGGCGGGAGGCCCGGCCTGGGCAGAGGTCGAA |
| | | ACAGTAACAAAGGACTGCCTCAGTCTACGATTTCTT |
| | | TTGATGGAATCTATGCAAATATGAGGATGGTTCATA |
| | | TACTTACATCAGTTGTTGGCTCCAAATGTGAAGTAC |
| | | AAGTGAAAAATGGAGGTATATATGAAGGAGTTTTTA |
| | | AAACTTACAGTCCGAAGTGTGATTTGGTACTTGATG |
| | | CCGCACATGAGAAAAGTACAGAATCCAGTTCGGGGC |
| | | CGAAACGTGAAGAAATAATGGAGAGTATTTTGTTCA |
| | | AATGTTCAGACTTTGTTGTGGTACAGTTTAAAGATAT |
| | | GGACTCCAGTTATGCAAAAAGAGATGCTTTTACTGA |
| | | CTCTGCTATCAGTGCTAAAGTGAATGGCGAACACAA |
| | | AGAGAAGGACCTGGAGCCCTGGGATGCAGGTGAAC |
| | | TCACAGCCAATGAGGAACTTGAGGCTTTGGAAAATG |
| | | ACGTATCTAATGGATGGGATCCCAATGATATGTTTC |
| | | GATATAATGAAGAAAATTATGGTGTAGTGTCTACGT |
| | | ATGATAGCAGTTTATCTTCGTATACAGTGCCCTTAGA |
| | | AAGAGATAACTCAGAAGAATTTTTAAAACGGGAAG |
| | | CAAGGGCAAACCAGTTAGCAGAAGAAATTGAGTCA |
| | | AGTGCCCAGTACAAAGCTCGAGTGGCCCTGGAAAAT |
| | | GATGATAGGAGTGAGGAAGAAAAATACACAGCAGT |
| | | TCAGAGAAATTCCAGTGAACGTGAGGGGCACAGCA |
| | | TAAACACTAGGGAAAATAAATATATTCCTCCTGGAC |
| | | AAAGAAATAGAGAAGTCATATCCTGGGGAAGTGGG |
| | | AGACAGAATTCACCGCGTATGGGCCAGCCTGGATCG |
| | | GGCTCCATGCCATCAAGATCCACTTCTCACACTTCAG |
| | | ATTTCAACCCGAATTCTGGTTCAGACCAAAGAGTAG |
| | | TTAATGGAGGTGTTCCCTGGCCATCGCCTTGCCCATC |
| | | TCCTTCCTCTCGCCCACCTTCTCGCTACCAGTCAGGT |
| | | CCCAACTCTCTTCCACCTCGGGCAGCCACCCCTACAC |
| | | GGCCGCCCTCCAGGCCCCCCTCGCGGCCATCCAGAC |
| | | CCCCGTCTCACCCCTCTGCTCATGGTTCTCCAGCTCC |
| | | TGTCTCTACTATGCCTAAACGCATGTCTTCAGAAGG |
| | | GCCTCCAAGGATGTCCCCAAAGGCCCAGCGACATCC |
| | | TCGAAATCACAGAGTTTCTGCTGGGAGGGGTTCCAT |
| | | ATCCAGTGGCCTAGAATTTGTATCCCACAACCCACC |
| | | CAGTGAAGCAGCTACTCCTCCAGTAGCAAGGACCAG |
| | | TCCCTCGGGGGGAACGTGGTCATCAGTGGTCAGTGG |
| | | GGTTCCAAGATTATCCCCTAAAACTCATAGACCCAG |
| | | GTCTCCCAGACAGAACAGTATTGGAAATACCCCCAG |
| | | TGGGCCAGTTCTTGCTTCTCCCCAAGCTGGTATTATT |
| | | CCAACTGAAGCTGTTGCCATGCCTATTCCAGCTGCAT |
| | | CTCCTACGCCTGCTAGTCCTGCATCGAACAGAGCTG |
| | | TTACCCCTTCTAGTGAGGCTAAAGATTCCAGGCTTCA |
| | | AGATCAGAGGCAGAACTCTCCTGCAGGGAATAAAG |
| | | AAAATATTAAACCCAATGAAACATCACCTAGCTTCT |
| | | CAAAAGCTGAAAACAAAGGTATATCACCAGTTGTTT |
| | | CTGAACATAGAAAACAGATTGATGATTTAAAGAAAT |
| | | TTAAGAATGATTTTAGGTTACAGCCAAGTTCTACTTC |
| | | TGAATCTATGGATCAACTACTAAACAAAAATAGAGA |
| | | GGGAGAAAAATCAAGAGATTTGATCAAAGACAAAA |
| | | TTGAACCAAGTGCTAAGGATTCTTTCATTGAAAATA |
| | | GCAGCAGCAACTGTACCAGTGGCAGCAGCAAGCCG |
| | | AATAGCCCCAGCATTTCCCCTTCAATACTTAGTAACA |
| | | CGGAGCACAAGAGGGGACCTGAGGTCACTTCCCAA |
| | | GGGGTTCAGACTTCCAGCCCAGCATGTAAACAAGAG |
| | | AAAGACGATAAGGAAGAGAAGAAAGACGCAGCTGA |
| | | GCAAGTTAGGAAATCAACATTGAATCCCAATGCAAA |
| | | GGAGTTCAACCCACGTTCCTTCTCTCAGCCAAAGCCT |
| | | TCTACTACCCCAACTTCACCTCGGCCTCAAGCACAA |
| | | CCTAGCCCATCTATGGTGGGTCATCAACAGCCAACT |
| | | CCAGTTTATACTCAGCCTGTTTGTTTTGCACCAAATA |
| | | TGATGTATCCAGTCCCAGTGAGCCCAGGCGTGCAAC |
| | | CTTTATACCCAATACCTATGACGCCCATGCCAGTGA |
| | | ATCAAGCCAAGACATATAGAGCAGTACCAAATATGC |
| | | CCCAACAGCGGCAAGACCAGCATCATCAGAGTGCCA |
| | | TGATGCACCCAGCGTCAGCAGCGGGCCCACCGATTG |
| | | CAGCCACCCCACCAGCTTACTCCACGCAATATGTTG |
| | | CCTACAGTCCTCAGCAGTTCCCAAATCAGCCCCTTGT |
| | | TCAGCATGTGCCACATTATCAGTCTCAGCATCCTCAT |
| | | GTCTATAGTCCTGTAATACAGGGTAATGCTAGAATG |
| | | ATGGCACCACCAACACACGCCCAGCCTGGTTTAGTA |
| | | TCTTCTTCAGCAACTCAGTACGGGGCTCATGAGCAG |
| | | ACGCATGCGATGTATGCATGTCCCAAATTACCATAC |
| | | AACAAGGAGACAAGCCCTTCTTTCTACTTTGCCATTT |
| | | CCACGGGCTCCCTTGCTCAGCAGTATGCGCACCCTA |
| | | ACGCTACCCTGCACCCACATACTCCACACCCTCAGC |
| | | CTTCAGCTACCCCCACTGGACAGCAGCAAAGCCAAC |
| | | ATGGTGGAAGTCATCCTGCACCCAGTCCTGTTCAGC |
| | | ACCATCAGCACCAGGCCGCCCAGGCTCTCCATCTGG |
| | | CCAGTCCACAGCAGCAGTCAGCCATTTACCACGCGG |
| | | GGCTTGCGCCAACTCCACCCTCCATGACACCTGCCTC |
| | | CAACACGCAGTCGCCACAGAATAGTTTCCCAGCAGC |
| | | ACAACAGACTGTCTTTACGATCCATCCTTCTCACGTT |
| | | CAGCCGGCGTATACCAACCCACCCCACATGGCCCAC |
| | | GTACCTCAGGCTCATGTACAGTCAGGAATGGTTCCT |
| | | TCTCATCCAACTGCCCATGCGCCAATGATGCTAATG |
| | | ACGACACAGCCACCCGGCGGTCCCCAGGCCGCCCTC |
| | | GCTCAAAGTGCACTACAGCCCATTCCAGTCTCGACA |
| | | ACAGCGCATTTCCCCTATATGACGCACCCTTCAGTAC |
| | | AAGCCCACCACCAACAGCAGTTGTAAGGCTGCCCTG |
| | | GAGGAACCGAAAGGCCAAATTCCCTCCTCCCTTCTA |
| | | CTGCTTCTACCAACTGGAAGCACAGAAAACTAGAAT |
| | | TTCATTTATTTTGTTTTTAAAATATATATGTTGATTTC |
| | | TTGTAACATCCAATAGGAATGCTAACAGTTCACTTG |
| | | CAGTGGAAGATACTTGGACCGAGTAGAGGCATTTAG |
| | | GAACTTGGGGGCTATTCCATAATTCCATATGCTGTTT |
| | | CAGAGTCCCGCAGGTACCCCAGCTCTGCTTGCCGAA |
| | | ACTGGAAGTTATTTATTTTTTAATAACCCTTGAAAGT |
| | | CATGAACACATCAGCTAGCAAAAGAAGTAACAAGA |
| | | GTGATTCTTGCTGCTATTACTGCTAAAAAAAAAAAA |
| | | AAAAAAAAATCAAGACTTGGAACGCCCTTTTACTAA |
| | | ACTTGACAAAGTTTCAGTAAATTCTTACCGTCAAACT |
| | | GACGGATTATTATTTATAAATCAAGTTTGATGAGGT |
| | | GATCACTGTCTACAGTGGTTCAACTTTTAAGTTAAGG |
| | | GAAAAACTTTTACTTTGTAGATAATATAAAATAAAA |
| | | ACTTAAAAAAAATTTAAAAAATAAAAAAAGTTTTAA |
| | | AAACTGA |
| 6 | ATXN2 Isoform 1 | >ref\|NM_002973.4\|1-4341 Homo sapiens ataxin 2 (ATXN2), |
| | (mRNA transcript), | transcript variant 1, mRNA |
| | annotated to show: Exon | AGAGCTCGCCTCCCTCCGCCTCAGACTGTTTTGGTAG |
| | 18 (bold), with the | CAACGGCAACGGCGGCGGCGCGTTTCGGCCCGGCTC |
| | optional +6 nt variant | CCGGCGGCTCCTTGGTCTCGGCGGGCCTCCCCGCCC |
| | (underline), which can | CTTCGTCGTCCTCCTTCTCCCCCTCGCCAGCCCGGGC |
| | be skipped to induce | GCCCCTCCGGCCGCGCCAACCCGCGCCTCCCCGCTC |
| | NMD. | GGCGCCCGCGCGTCCCCGCCGCGTTCCGGCGTCTCC |
| | | TTGGCGCGCCCGGCTCCCGGCTGTCCCCGCCCGGCG |
| | | TGCGAGCCGGTGTATGGGCCCCTCACCATGTCGCTG |
| | | AAGCCCCAGCAGCAGCAGCAGCAGCAGCAGCAGCA |
| | | GCAGCAGCAGCAACAGCAGCAGCAGCAGCAGCAGC |
| | | AGCAGCCGCCGCCCGCGGCTGCCAATGTCCGCAAGC |
| | | CCGGCGGCAGCGGCCTTCTAGCGTCGCCCGCCGCCG |
| | | CGCCTTCGCCGTCCTCGTCCTCGGTCTCCTCGTCCTC |
| | | GGCCACGGCTCCCTCCTCGGTGGTCGCGGCGACCTC |
| | | CGGCGGCGGGAGGCCCGGCCTGGGCAGAGGTCGAA |
| | | ACAGTAACAAAGGACTGCCTCAGTCTACGATTTCTT |
| | | TTGATGGAATCTATGCAAATATGAGGATGGTTCATA |
| | | TACTTACATCAGTTGTTGGCTCCAAATGTGAAGTAC |
| | | AAGTGAAAAATGGAGGTATATATGAAGGAGTTTTTA |
| | | AAACTTACAGTCCGAAGTGTGATTTGGTACTTGATG |
| | | CCGCACATGAGAAAAGTACAGAATCCAGTTCGGGGC |
| | | CGAAACGTGAAGAAATAATGGAGAGTATTTTGTTCA |
| | | AATGTTCAGACTTTGTTGTGGTACAGTTTAAAGATAT |
| | | GGACTCCAGTTATGCAAAAAGAGATGCTTTTACTGA |
| | | CTCTGCTATCAGTGCTAAAGTGAATGGCGAACACAA |
| | | AGAGAAGGACCTGGAGCCCTGGGATGCAGGTGAAC |
| | | TCACAGCCAATGAGGAACTTGAGGCTTTGGAAAATG |
| | | ACGTATCTAATGGATGGGATCCCAATGATATGTTTC |
| | | GATATAATGAAGAAAATTATGGTGTAGTGTCTACGT |
| | | ATGATAGCAGTTTATCTTCGTATACAGTGCCCTTAGA |
| | | AAGAGATAACTCAGAAGAATTTTTAAAACGGGAAG |
| | | CAAGGGCAAACCAGTTAGCAGAAGAAATTGAGTCA |
| | | AGTGCCCAGTACAAAGCTCGAGTGGCCCTGGAAAAT |
| | | GATGATAGGAGTGAGGAAGAAAAATACACAGCAGT |
| | | TCAGAGAAATTCCAGTGAACGTGAGGGGCACAGCAT |
| | | AAACACTAGGGAAAATAAATATATTCCTCCTGGACA |
| | | AAGAAATAGAGAAGTCATATCCTGGGGAAGTGGGA |
| | | GACAGAATTCACCGCGTATGGGCCAGCCTGGATCGG |
| | | GCTCCATGCCATCAAGATCCACTTCTCACACTTCAGA |
| | | TTTCAACCCGAATTCTGGTTCAGACCAAAGAGTAGT |
| | | TAATGGAGGTGTTCCCTGGCCATCGCCTTGCCCATCT |
| | | CCTTCCTCTCGCCCACCTTCTCGCTACCAGTCAGGTC |
| | | CCAACTCTCTTCCACCTCGGGCAGCCACCCCTACAC |
| | | GGCCGCCCTCCAGGCCCCCCTCGCGGCCATCCAGAC |
| | | CCCCGTCTCACCCCTCTGCTCATGGTTCTCCAGCTCC |
| | | TGTCTCTACTATGCCTAAACGCATGTCTTCAGAAGG |
| | | GCCTCCAAGGATGTCCCCAAAGGCCCAGCGACATCC |
| | | TCGAAATCACAGAGTTTCTGCTGGGAGGGGTTCCAT |
| | | ATCCAGTGGCCTAGAATTTGTATCCCACAACCCACC |
| | | CAGTGAAGCAGCTACTCCTCCAGTAGCAAGGACCAG |
| | | TCCCTCGGGGGGAACGTGGTCATCAGTGGTCAGTGG |
| | | GGTTCCAAGATTATCCCCTAAAACTCATAGACCCAG |
| | | GTCTCCCAGACAGAACAGTATTGGAAATACCCCCAG |
| | | TGGGCCAGTTCTTGCTTCTCCCCAAGCTGGTATTATT |
| | | CCAACTGAAGCTGTTGCCATGCCTATTCCAGCTGCAT |
| | | CTCCTACGCCTGCTAGTCCTGCATCGAACAGAGCTG |
| | | TTACCCCTTCTAGTGAGGCTAAAGATTCCAGGCTTCA |
| | | AGATCAGAGGCAGAACTCTCCTGCAGGGAATAAAG |
| | | AAAATATTAAACCCAATGAAACATCACCTAGCTTCT |
| | | CAAAAGCTGAAAACAAAGGTATATCACCAGTTGTTT |
| | | CTGAACATAGAAAACAGATTGATGATTTAAAGAAAT |
| | | TTAAGAATGATTTTAGGTTACAGCCAAGTTCTACTTC |
| | | TGAATCTATGGATCAACTACTAAACAAAAATAGAGA |
| | | GGGAGAAAAATCAAGAGATTTGATCAAAGACAAAA |
| | | TTGAACCAAGTGCTAAGGATTCTTTCATTGAAAATA |
| | | GCAGCAGCAACTGTACCAGTGGCAGCAGCAAGCCG |
| | | AATAGCCCCAGCATTTCCCCTTCAATACTTAGTAACA |
| | | CGGAGCACAAGAGGGGACCTGAGGTCACTTCCCAA |
| | | GGGGTTCAGACTTCCAGCCCAGCATGTAAACAAGAG |
| | | AAAGACGATAAGGAAGAGAAGAAAGACGCAGCTGA |
| | | GCAAGTTAGGAAATCAACATTGAATCCCAATGCAAA |
| | | GGAGTTCAACCCACGTTCCTTCTCTCAGCCAAAGCCT |
| | | TCTACTACCCCAACTTCACCTCGGCCTCAAGCACAA |
| | | CCTAGCCCATCTATGGTGGGTCATCAACAGCCAACT |
| | | CCAGTTTATACTCAGCCTGTTTGTTTTGCACCAAATA |
| | | TGATGTATCCAGTCCCAGTGAGCCCAGGCGTGCAAC |
| | | CTTTATACCCAATACCTATGACGCCCATGCCAGT |
| | | GAATCAAGCCAAGACATATAGAGCAGGTAAAGTA |
| | | CCAAATATGCCCCAACAGCGGCAAGACCAGCATCAT |
| | | CAGAGTGCCATGATGCACCCAGCGTCAGCAGCGGGC |
| | | CCACCGATTGCAGCCACCCCACCAGCTTACTCCACG |
| | | CAATATGTTGCCTACAGTCCTCAGCAGTTCCCAAATC |
| | | AGCCCCTTGTTCAGCATGTGCCACATTATCAGTCTCA |
| | | GCATCCTCATGTCTATAGTCCTGTAATACAGGGTAAT |
| | | GCTAGAATGATGGCACCACCAACACACGCCCAGCCT |
| | | GGTTTAGTATCTTCTTCAGCAACTCAGTACGGGGCTC |
| | | ATGAGCAGACGCATGCGATGTATGCATGTCCCAAAT |
| | | TACCATACAACAAGGAGACAAGCCCTTCTTTCTACT |
| | | TTGCCATTTCCACGGGCTCCCTTGCTCAGCAGTATGC |
| | | GCACCCTAACGCTACCCTGCACCCACATACTCCACA |
| | | CCCTCAGCCTTCAGCTACCCCCACTGGACAGCAGCA |
| | | AAGCCAACATGGTGGAAGTCATCCTGCACCCAGTCC |
| | | TGTTCAGCACCATCAGCACCAGGCCGCCCAGGCTCT |
| | | CCATCTGGCCAGTCCACAGCAGCAGTCAGCCATTTA |
| | | CCACGCGGGGCTTGCGCCAACTCCACCCTCCATGAC |
| | | ACCTGCCTCCAACACGCAGTCGCCACAGAATAGTTT |
| | | CCCAGCAGCACAACAGACTGTCTTTACGATCCATCC |
| | | TTCTCACGTTCAGCCGGCGTATACCAACCCACCCCA |
| | | CATGGCCCACGTACCTCAGGCTCATGTACAGTCAGG |
| | | AATGGTTCCTTCTCATCCAACTGCCCATGCGCCAATG |
| | | ATGCTAATGACGACACAGCCACCCGGCGGTCCCCAG |
| | | GCCGCCCTCGCTCAAAGTGCACTACAGCCCATTCCA |
| | | GTCTCGACAACAGCGCATTTCCCCTATATGACGCAC |
| | | CCTTCAGTACAAGCCCACCACCAACAGCAGTTGTAA |
| | | GGCTGCCCTGGAGGAACCGAAAGGCCAAATTCCCTC |
| | | CTCCCTTCTACTGCTTCTACCAACTGGAAGCACAGA |
| | | AAACTAGAATTTCATTTATTTTGTTTTTAAAATATAT |
| | | ATGTTGATTTCTTGTAACATCCAATAGGAATGCTAAC |
| | | AGTTCACTTGCAGTGGAAGATACTTGGACCGAGTAG |
| | | AGGCATTTAGGAACTTGGGGGCTATTCCATAATTCC |
| | | ATATGCTGTTTCAGAGTCCCGCAGGTACCCCAGCTCT |
| | | GCTTGCCGAAACTGGAAGTTATTTATTTTTTAATAAC |
| | | CCTTGAAAGTCATGAACACATCAGCTAGCAAAAGAA |
| | | GTAACAAGAGTGATTCTTGCTGCTATTACTGCTAAA |
| | | AAAAAAAAAAAAAAAAAATCAAGACTTGGAACGCC |
| | | CTTTTACTAAACTTGACAAAGTTTCAGTAAATTCTTA |
| | | CCGTCAAACTGACGGATTATTATTTATAAATCAAGTT |
| | | TGATGAGGTGATCACTGTCTACAGTGGTTCAACTTTT |
| | | AAGTTAAGGGAAAAACTTTTACTTTGTAGATAATAT |
| | | AAAATAAAAACTTAAAAAAAATTTAAAAAATAAAA |
| | | AAAGTTTTAAAAACTGA |
| 7 | ATXN2 Isoform 1 | >NP_002964.4 ataxin-2 isoform 1 [Homo sapiens] |
| | (protein) | MSLKPQQQQQQQQQQQQQQQQQQQQQQQPPPAAANVRKP |
| | | GGSGLLASPAAAPSPSSSSVSSSSATAPSSVVAATSGGGRPG |
| | | LGRGRNSNKGLPQSTISFDGIYANMRMVHILTSVVGSKCEV |
| | | QVKNGGIYEGVFKTYSPKCDLVLDAAHEKSTESSSGPKREEI |
| | | MESILFKCSDFVVVQFKDMDSSYAKRDAFTDSAISAKVNGE |
| | | HKEKDLEPWDAGELTANEELEALENDVSNGWDPNDMFRY |
| | | NEENYGVVSTYDSSLSSYTVPLERDNSEEFLKREARANQLA |
| | | EEIESSAQYKARVALENDDRSEEEKYTAVQRNSSEREGHSIN |
| | | TRENKYIPPGQRNREVISWGSGRONSPRMGQPGSGSMPSRS |
| | | TSHTSDFNPNSGSDQRVVNGGVPWPSPCPSPSSRPPSRYQSG |
| | | PNSLPPRAATPTRPPSRPPSRPSRPPSHPSAHGSPAPVSTMPK |
| | | RMSSEGPPRMSPKAQRHPRNHRVSAGRGSISSGLEFVSHNPP |
| | | SEAATPPVARTSPSGGTWSSVVSGVPRLSPKTHRPRSPRQNS |
| | | IGNTPSGPVLASPQAGIIPTEAVAMPIPAASPTPASPASNRAV |
| | | TPSSEAKDSRLQDQRONSPAGNKENIKPNETSPSFSKAENKG |
| | | ISPVVSEHRKQIDDLKKFKNDFRLQPSSTSESMDQLLNKNRE |
| | | GEKSRDLIKDKIEPSAKDSFIENSSSNCTSGSSKPNSPSISPSIL |
| | | SNTEHKRGPEVTSQGVQTSSPACKQEKDDKEEKKDAAEQV |
| | | RKSTLNPNAKEFNPRSFSQPKPSTTPTSPRPQAQPSPSMVGH |
| | | QQPTPVYTQPVCFAPNMMYPVPVSPGVQPLYPIPMTPMPVN |
| | | QAKTYRAVPNMPQQRQDQHHQSAMMHPASAAGPPIAATPP |
| | | AYSTQYVAYSPQQFPNQPLVQHVPHYQSQHPHVYSPVIQGN |
| | | ARMMAPPTHAQPGLVSSSATQYGAHEQTHAMYACPKLPY |
| | | NKETSPSFYFAISTGSLAQQYAHPNATLHPHTPHPQPSATPT |
| | | GQQQSQHGGSHPAPSPVQHHQHQAAQALHLASPQQQSAIY |
| | | HAGLAPTPPSMTPASNTQSPQNSFPAAQQTVFTIHPSHVQPA |
| | | YTNPPHMAHVPQAHVQSGMVPSHPTAHAPMMLMTTQPPG |
| | | GPQAALAQSALQPIPVSTTAHFPYMTHPSVQAHHQQQL |
| 8 | C1 | ATTCACTTTCATAATGCTGG |
| 9 | C5 | GCGACTATACGCGCAATATG |
| 10 | Potential ASO | CACGTTCACTGGAATTTCTCTGAACTGCTGTGTATTTC |
| | Sequence | |
| 11 | Potential ASO | ACTGGAATTTCTCTGAACTGCTGTGTATTTC |
| | Sequence | |
| 12 | Potential ASO | TGGAATTTCTCTGAACTGCTGTGTAT |
| | Sequence | |
| 13 | A1 | TTTCCAGGGCCACTCGAGCT |
| 14 | A2 | CTATCATCATTTTCCAGGGC |
| 15 | A3 | TTCCTCACTCCTATCATCAT |
| 16 | A4 | TGTATTTTTCTTCCTCACTC |
| 17 | A5 | TGAACTGCTGTGTATTTTTC |
| 18 | A6 | GGAATTTCTCTGAACTGCTG |
| 19 | A7 | CACGTTCACTGGAATTTCTC |
| 20 | A8 | CTGTGCCCCTCACGTTCACT |
| 21 | A9 | AGTGTTTATGCTGTGCCCCT |
| 22 | A10 | TTAAATACCTAGTGTTTATG |
| 23 | A11 | ATGATTTCCTTTAAATACCT |
| 24 | A12 | ATCCAAAATACTGCATCATG |
| 25 | A13 | ACCTTGAGTTGTGTATCCAA |
| 26 | A14 | CCGTCTCACACAGACCTTGA |
| 27 | A15 | AACAATACACCGTCTCACAC |
| 28 | A16 | AAGAGGAAATATAATAACAA |
| 29 | A17 | AAGCTATATTAAAGGAAGAG |
| 30 | A18 | TTGCATTCTCTACCTAAGCT |
| 31 | A19 | TAAACCAATTCTACTTGCAT |
| 32 | A20 | TCTAACAGATCTTAAACCAA |
| 33 | A21 | TCTGAACTGCTGTGTATTTT |
| 34 | A22 | CTCTGAACTGCTGTGTATTT |
| 35 | A23 | TCTCTGAACTGCTGTGTATT |
| 36 | A24 | TTCTCTGAACTGCTGTGTAT |
| 37 | A25 | TTTCTCTGAACTGCTGTGTA |
| 38 | A26 | ATTTCTCTGAACTGCTGTGT |
| 39 | A27 | AATTTCTCTGAACTGCTGTG |
| 40 | A28 | GAATTTCTCTGAACTGCTGT |
| 41 | A29 | GGAATTTCTCTGAACTGCTG |
| 42 | A30 | TGGAATTTCTCTGAACTGCT |
| 43 | A31 | CTGGAATTTCTCTGAACTGC |
| 44 | A32 | ACTGGAATTTCTCTGAACTG |
| 45 | A33 | CACTGGAATTTCTCTGAACT |
| 46 | A34 | TCACTGGAATTTCTCTGAAC |
| 47 | A35 | TTCACTGGAATTTCTCTGAA |
| 48 | A36 | GTTCACTGGAATTTCTCTGA |
| 49 | A37 | CGTTCACTGGAATTTCTCTG |
| 50 | A38 | ACGTTCACTGGAATTTCTCT |
| 51 | A39 | CTGAACTGCTGTGTATTTTT |
| 52 | A59 | GGAATTTCTCTGAACTGCT |
| 53 | A60 | GAATTTCTCTGAACTGCTG |
| 54 | A61 | AATTTCTCTGAACTGCTGT |
| 55 | A62 | ATTTCTCTGAACTGCTGTG |
| 56 | A63 | TACCTAGTGTTTATGCTGTG |
| 57 | A64 | ACCTAGTGTTTATGCTGTGC |
| 58 | A65 | CCTAGTGTTTATGCTGTGCC |
| 59 | A66 | CTAGTGTTTATGCTGTGCCC |
| 60 | A67 | TAGTGTTTATGCTGTGCCCC |
| 61 | A68 | GTGTTTATGCTGTGCCCCTC |
| 62 | A69 | TGTTTATGCTGTGCCCCTCA |
| 63 | A70 | GTTTATGCTGTGCCCCTCAC |
| 64 | A71 | TTTATGCTGTGCCCCTCACG |
| 65 | A72 | TTATGCTGTGCCCCTCACGT |
| 66 | A73 | GCATCATGATTTCCTTTAAA |
| 67 | A74 | CATCATGATTTCCTTTAAAT |
| 68 | A75 | ATCATGATTTCCTTTAAATA |
| 69 | A76 | TCATGATTTCCTTTAAATAC |
| 70 | A77 | CATGATTTCCTTTAAATACC |
| 71 | A78 | TGATTTCCTTTAAATACCTA |
| 72 | A79 | GATTTCCTTTAAATACCTAG |
| 73 | A80 | ATTTCCTTTAAATACCTAGT |
| 74 | A81 | TTTCCTTTAAATACCTAGTG |
| 75 | A82 | TTCCTTTAAATACCTAGTGT |
| 76 | A93 | TCTGAACTGCTGTGTATTT |
| 77 | A94 | CTCTGAACTGCTGTGTATT |
| 78 | A95 | TCTCTGAACTGCTGTGTAT |
| 79 | A96 | TTCTCTGAACTGCTGTGTA |
| 80 | A97 | TTTCTCTGAACTGCTGTGT |
| 81 | A98 | AATTTCTCTGAACTGCTGTGTATTT |
| 82 | A99 | AATTTCTCTGAACTGCTGTGTATT |
| 83 | A100 | AATTTCTCTGAACTGCTGTGTAT |
| 84 | A101 | AATTTCTCTGAACTGCTGTGTA |
| 85 | A102 | AATTTCTCTGAACTGCTGTGT |
| 86 | A103 | GAATTTCTCTGAACTGCTGTG |
| 87 | A104 | GGAATTTCTCTGAACTGCTGTG |
| 88 | A105 | TGGAATTTCTCTGAACTGCTGTG |
| 89 | A106 | CTGGAATTTCTCTGAACTGCTGTG |
| 90 | A107 | ACTGGAATTTCTCTGAACTGCTGTG |
| 91 | A108 | TTCTCTGAACTGCTGTGT |
| 92 | A109 | TCTCTGAACTGCTGTGTA |
| 93 | A110 | CTCTGAACTGCTGTGTAT |
| 94 | A111 | TCTGAACTGCTGTGTATT |
| 95 | A112 | CTGAACTGCTGTGTATTT |
| 96 | A113 | TCTCTGAACTGCTGTGT |
| 97 | A114 | CTCTGAACTGCTGTGTA |
| 98 | A115 | TCTGAACTGCTGTGTAT |
| 99 | A116 | CTGAACTGCTGTGTATT |
| 100 | A117 | TGAACTGCTGTGTATTT |
| 101 | A118 | CTCTGAACTGCTGTGT |
| 102 | A119 | TCTGAACTGCTGTGTA |
| 103 | A120 | CTGAACTGCTGTGTAT |
| 104 | A121 | TGAACTGCTGTGTATT |
| 105 | A122 | GAACTGCTGTGTATTT |
| 106 | A125 | TGAGAGAATTAAAAAAAA |
| 107 | A126 | GCTTGAGAGAATTAAAAA |
| 108 | A127 | AAGGCTTGAGAGAATTAA |
| 109 | A128 | ATAAAGGCTTGAGAGAAT |
| 110 | A129 | GGTATAAAGGCTTGAGAG |
| 111 | A130 | TTGGGTATAAAGGCTTGA |
| 112 | A131 | GTATTGGGTATAAAGGCT |
| 113 | A132 | CATAGGTATTGGGTATAA |
| 114 | A133 | GGCGTCATAGGTATTGGG |
| 115 | A134 | GCATGGGCGTCATAGGTA |
| 116 | A135 | CACTGGCATGGGCGTCAT |
| 117 | A136 | TGATTCACTGGCATGGGC |
| 118 | A137 | TGGCTTGATTCACTGGCA |
| 119 | A138 | TGTCTTGGCTTGATTCAC |
| 120 | A139 | CTATATGTCTTGGCTTGA |
| 121 | A140 | CTGCTCTATATGTCTTGG |
| 122 | A141 | TTTACCTGCTCTATATGT |
| 123 | A142 | ACCTTTACCTGCTCTATA |
| 124 | A143 | CTCACCTTTACCTGCTCT |
| 125 | A144 | ATTCTCACCTTTACCTGC |
| 126 | A145 | ATTATTCTCACCTTTACC |
| 127 | A146 | AGGATTATTCTCACCTTT |
| 128 | A147 | GGCAGGATTATTCTCACC |
| 129 | A148 | ACAGGCAGGATTATTCTC |
| 130 | A149 | AACACAGGCAGGATTATT |
| 131 | A217 | TTTCCTTTAAATACCTAGTGT |
| 132 | A218 | ATTTCCTTTAAATACCTAGTGT |
| 133 | A219 | GATTTCCTTTAAATACCTAGTGT |
| 134 | A220 | TGATTTCCTTTAAATACCTAGTGT |
| 135 | A221 | ATGATTTCCTTTAAATACCTAGTGT |
| 136 | A222 | CATGATTTCCTTTAAATACCTAGTGT |
| 137 | A223 | TCATGATTTCCTTTAAATACCTAGTGT |

We claim:

1. An antisense oligonucleotide that hybridizes to a target region in an ATXN2 RNA transcript, wherein hybridization of the antisense oligonucleotide to the target region decreases the expression of a functional protein encoded by the ATXN2 RNA transcript in a cell.

2. The antisense oligonucleotide of claim 1, wherein the ATXN2 RNA transcript is a pre-mRNA molecule.

3. The antisense oligonucleotide of claim 1 or 2, wherein the antisense oligonucleotide is 15 to 25 nucleotides in length.

4. The antisense oligonucleotide of any one of claims 1-3, wherein the antisense oligonucleotide is 18 to 20 nucleotides in length.

5. The antisense oligonucleotide of any one of claims 1-4, wherein the antisense oligonucleotide comprises a nucleotide sequence of any one of SEQ ID NOs: 10-137, or a fragment thereof.

6. The antisense oligonucleotide of any one of claims 1-4, wherein the antisense oligonucleotide comprises a nucleotide sequence having 1, 2, 3, 4, or 5 nucleotide substitutions as compared to any one of SEQ ID NOs: 10-137, or a fragment thereof.

7. The antisense oligonucleotide of any one of claims 1-4, wherein the antisense oligonucleotide comprises a nucleotide sequence having at least 85, 86, 87, 88, 89, 90, 91, 92, 93, 94, 95, 96, 97, 98, or 99% sequence identity as compared to any one of SEQ ID NOs: 10-137, or a fragment thereof.

8. The antisense oligonucleotide of any one of claims 1-4, wherein the antisense oligonucleotide comprises:
a) a nucleotide sequence identical to a contiguous 18-20 mer portion of any one of SEQ ID NOs: 10-137; or
b) a 10, 11, 12, 13, 14, 15, 16, 17, 18, 19, 20, 21, 22, 23, 24, or 25 nucleotide sequence, having 1, 2, 3, 4, 5, 6, 7, 8, 9, or 10 nucleotide substitutions as compared to any one of SEQ ID NOs: 10-137, or an aligned portion thereof.

9. The antisense oligonucleotide of any one of claims 1-4, wherein the antisense oligonucleotide is 15-30 mer in length and comprises a nucleotide sequence identical to any contiguous 16-20 mer portion of SEQ ID NOs: 10-137, with 0, 1, 2, 3, 4 or 5 nucleotide substitutions.

10. The antisense oligonucleotide of any one of claims 1-9, wherein the antisense oligonucleotide comprises one or more modified nucleotides.

11. The antisense oligonucleotide of claim 10, wherein the one or more modified nucleotides comprise a modification of a ribose group, a phosphate group, a nucleobase, or a combination thereof.

12. The antisense oligonucleotide of claim 11, wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH2, a constrained nucleotide, or a combination thereof.

13. The antisense oligonucleotide of claim 12, wherein the constrained nucleotide comprises a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNANC), an alpha-L-locked nucleic acid, a tricyclo-DNA, or a combination thereof.

14. The antisense oligonucleotide of claim 11, wherein the modification of the ribose group comprises 2′-O-(2-methoxyethyl) (MOE).

15. The antisense oligonucleotide of claim 11, wherein the modification of the phosphate group comprises a phosphorothioate, a phosphonoacetate (PACE), a thiophosphonoacetate (thioPACE), an amide, a triazole, a phosphonate, a phosphotriester modification, or a combination thereof.

16. The antisense oligonucleotide of claim 11, wherein the modification of the nucleobase group comprises 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, one or more halogenated aromatic groups, or a combination thereof.

17. A pharmaceutical composition, comprising
at least one antisense oligonucleotide selected from the antisense oligonucleotides of any one of claims 1-16, and
at least one pharmaceutically-acceptable carrier, diluent, or buffer.

18. The pharmaceutical composition of claim 17, wherein the at least one antisense oligonucleotide comprises a plurality of sequentially different antisense oligonucleotides.

19. The pharmaceutical composition of claim 17 or 18, wherein the at least one antisense oligonucleotide is present in the composition at 0.001 to 100 mg/ml.

20. The pharmaceutical composition of claim 17 or 18, wherein the at least one antisense oligonucleotide is present in a unit dose amount.

21. The pharmaceutical composition of claim 20, wherein the unit dose amount is 0.001 to 100 mg.

22. A method of treating a disease or disorder in a subject in need thereof, comprising:
a) administering an effective amount of the antisense oligonucleotide of any one of claims 1-16, or the pharmaceutical composition of any one of claims 17-21, to the subject; and
b) treating the disease or disorder.

23. The method of claim 22, wherein the disease or disorder is a neurodegenerative disease or a proteinopathy disease.

24. The method of claim 23, wherein the neurodegenerative disease comprises Spinocerebellar ataxia, ALS, parkinsonism, Frontotemporal dementia or Alzheimer's disease; and the proteinopathy disease comprises TDP43 proteinopathy.

25. The method of any one of claims 22-24, wherein treating the disease or disorder comprises preventing, reducing, slowing, or eliminating one or more symptoms of the disease or disorder.

26. The method of any one of claims 22-25, wherein treating the disease or disorder comprises improving motor function in the subject by at least 10, 15, or 20%.

27. The method of any one of claims 22-25, wherein treating the disease or disorder comprises improving motor rotarod performance in the subject by at least 10, 15, or 20%.

28. The method of any one of claims 22-27, wherein the effective amount is an amount sufficient to reduce expression of a protein encoded by ATXN2 in at least one cell or tissue of the subject.

29. The method of claim 28, wherein the cell is a nerve cell.

30. The method of claim 29, wherein the cell is Purkinje cell.

31. The method of any one of claims 22-30, wherein the administering is parenteral administration.

32. A cell, comprising:
the antisense oligonucleotide of any one of claims 1-16.

33. A vector designed to express the antisense oligonucleotide of any one of claims 1-16.

34. A method of reducing the amount or activity of a target mRNA encoding an ATXN2 isoform in a cell, comprising
contacting the cell with an antisense oligonucleotide configured to hybridize with the target mRNA; and
causing the target mRNA transcript to be degraded by nonsense-mediated decay.

35. The method of claim 34, wherein the antisense oligonucleotide is configured to hybridize with a plurality of contiguous nucleotides within exon 8, exon 9, or the intronic region between exon 8 and exon 9, of ATXN2.


SEQUENCE LISTING (137 sequences)

SEQ ID NO: 1 - DNA, 163192 bp, Homo sapiens
  source 1..163192: mol_type = genomic DNA
ccagcactttgggaggccaaggcaggcagatcacttgaggtgaggagttctggccaacacgaaaaaatgtcgtctctactaaaaatacaaaaattagctggacatggtggtgagtgcctataatcccagctacatggaaggctgagcggggaggatcacttgaacccaggaggtggaggttacagtgaactgagattgtgccactgcattccagcctgggcaagacagcaagactctgcctaaaaacaaacaaacaaatcccccaaaactaacaaaaattagctgggtgtggtggtgcacgcctgtagtccctgatactctgaaggctgagatgggaggattgctcgggaccgcctgggaggcagacgctgcagtgagccagactgtggaactgtactccagcctggttggcagagtaagaccctgtctcgaaaaaaaaaaaaaaaagtattgttttaaaattcaggtggggaggatttcacagtgtctgaacaccaggaggcaagaatcatttgggaccgccatagaggctgcctgacacaccacttcttttaaggtgcattactctaatatatatatatatatatattatttttttgagatggagtgtcactctgtcaccaggctggagtgcagtggcatgatcttgactcactgcaacctctgcctgccaggttcaagcgattctcctgcctcagcctcccgagtagttaggactacaggcgcacaccaccatgcccagctaatttttgtattttcttttttttgagacagagtctcgctctgttgcccaggctggagtgcagtggcgccatctcacctcactgcaagctccgcctcccgagttcatgccattctcctgcctcagcctcccaagtagctgggactacaggcgcccgccaccacacccacctatttttttgtatttttagtagagacggggtttcactgtgttagccaggatggccttgatctcctgacctcgtgatctgcccgccttggcctcccaaagtgctgggattacaggtgtgagccaccatgcctggctaatttttgtatttttaatagagacagggtttcaccatgttggcaaggatggtctcgatctcttgacctcaggtgatcagcccacctcagtctcccaaagtgctgggattacaggcatgagccaccgtgcccagcctactctgatattttcatataaagcttaagccttctgaagtccagagaaaaggtagtttccataaataaataaataaaataaaaataactaaataaacttaccacatcacccaacaatttcgtttgtaggaaccaacccaagataaatgaaaacatatatccacatgaagatatatgttcacaaatgttcatagcagcattattatttttctttcttcttctttgtgtgtgtgtgtgtgtgttttagcaactctattaactgaacgagacagggtcttggtctgttgcccaggctagagtgcagtggcgcattcgtagctcacagcaacctcgatctcctgggctcaagcggtccttctgcttcagcctccctagtagctgggactataggtgcgtgtcaccatgaccagctaatttttttttttttttttttttggagacagagtattgctgtgtcacccaggcttcagtgcaatggtgcaatctcggctcactgtaacctccgcctcctgggttcaagcaattctcctgcctcagcctcccaagtagctgagattacaggtgcccgccaccacgcctggctaattttttgtatttttagtagagacgggatttcaccatgtaggccaggctggtctcgaactcctgatctcaggtgatccacccacctcggcctcccaaagtgctgggattataggcatgagccaccacgcccagccaaccagctaattttttatttttaaagtttttcatagagacagggtctctctatgttgcccaggctggtttcaaactccggggtattcaagtgatcctcccatctcggtatcctaaagtgttgggattacaggagtgagccaccaatctcagccccagcagcattttcataatactcaaaaagttgacacaatctaaacatccacaattggaaagtagacacaaaatgtggtatgtccatacatggaatatgacatagcaataataaggaatgaaatactgctacatgccacaaccttagataaacctcaaaaacattatgacaaatgaaagaaggcagacacaaaaggctgcatgctgtaagattctacttatgtgaaaaatctggacaaatccatagagagaaaatgtagatgagtggttgcctggggctaaggatgggaaccaaactgcccaccatcgtgaaggatctttttggggtaatggaaatgttctgaaattagtggcaatggttgtgcaaatttactgtgtaaatttactgagaatcattaaattgtacacaaaataggtgaatttcatagtatgtaaattatatcttgataaaactattaagtttttattcaatggtagtgagttaaaacaaactttctcagctatgtcagcttcaggccttctttttttttaattttgaaaaattttttatggagaggagggtctcactatgttgcccaggctggtctcctactccttggttcaaaggatctgcctgccttggccttccaaagtgctgggattataggtgtaggccactgtatccagcctaggccttcttattcaacttccatcgacctgaaacttactattttttttttgagacggagtctcgctctgtcgcccaggctggagtgcagtggtgtgatctccgctcactgcaagcaccgcctcccaggttcacaccattctcctgcctcagcctcccaagtagctgggactacaggtgcgcatcaccacacccagctaatttttgtatttttagtagagatgggctctactaaaaattggccaggctggtctcgaactcctgacctcaggtgatccaccggccactgcctcccaaagtgctgggattacaggcgtgagccaccacactggccaaaacttgttcttaagattgtattctgggaccttgattccaatcagagaaaagtgattgtatttttttatttttattttttttagataaagtttcgctcttgttgcccaggctggagtgcagtggtgccctctttggtcactgtaacctccgcctcctgggttcaagcgattctcctgcctcagcatcctgcgtagctgagatcacagatgcccaccaccacgcccagctaattttttcgtatttttagtagcgatggggtttcaccatgttggccacgctggtcttgaactcctgacctcaggtgatccatccgcctcggcctcccagagtgctgggattacaggtgtgagccaccgcgccaggccaagtgtttgtatttctattaaagaaagaatataacgggacaccattgacgacctgctccattgcaggcctccttgctgttcctcagactcccccctcagagcctttgccctcgctgtgccctccacctggagcgtttctccccaggatcctcatgcccatgctcatttgggtccctgccccatgtcaccctctccaggagcttcccctcacagcagccctggcctgtaccacagccgggtacaggtatttttttgtttcaactggttttttagttccagtttcctttaggttactttatttatttatttatttatttattttttgagacggagtctcgctctgtcgcccaggctggagtgcatgatctcggctgactgcaacctccacctcccggattcaagcaattctcctgtatcagcctcccgagtagctgggattacaggcgcccaccaccacacccggctaatttttatatttttggtagagacggggtttcaccatgttggctaggctaggttaatttttaaagggttttgcaatggtcccttgatctactttttaccttagatgggaaataaaactgatttcctacattggcagaatacaatgatcatttttgcctggactatctaggaggttaatttcagttggactactgaaaactgctggttcaatcattctccacgtttatctaagtctttacctttatctggacagttctaggacattgaggggaattttggtgtttcttcccctattatttcctgaagtcatttcactttaaaaaacaatagattcactgctcaaaaaaaaaaaaaaaagttacctactttctacttgcttccagtttaactgcaacacattttaaaaagagtctactgtgctggctgggtaagttaaattaaaacttctaaagggtccaaggtctaaagttcgcacattgttttgaggtcggctctgtctctaccgagggagatcccattatccgtagttctaccagtcccaatcccatatatttcctttagaatctcatgaatgaggaaaaagaagttcaagtgagggaacataggttcaaatgaaggtcagatacctaaaagagttttctggtgactgtgcgcggctggggtggaaaaagtggggaaaaggtacccagatgtgggtggccccggagggttgctccactccagccccggcagggcaggacagcgcggcctgcctggtagatgccccgagccactggagcgcctactgtgtggcgggcgggggacggcaggaaaacggcaggatgctgtgtcccctgaatctggcagggttctaggtgctttacacgtagcaagacacattctccgccccaaggcactcgcagtcagtccattttctgggttgcatcaggtgggggcaaactaggtccccgcagaagtgaagatgctgaaggaatacagtaggagaagaaatgcttctctcctgtcctccacaccaggcaggccccagaggctgagaccgacacgccctccccgaagggcagacccgccttgaggaaggcggatccgggtagggaccgccgcctggccctcacccgacccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcaggtgggtgtcggcaccccagccccctccgctccgggcccggcgtcccctcccccgcggcccgcgccgccgtccccgccccgtgacccgccgggctacccggggtgggctgggggccggcagcgcgggggagactcgctcgggcctgagccccgaggctcggccggtgggcgcagccggggtcctctgggattgtcaggcctgtccagcctcccgcagcatccccgccccctcccccggcggtcaagatggagggagcgggcggcctcccctccccacgcgtgttgggaggggttctcgggtagcggcgatggtcagccccggctcccccttccgcacgatcctccgcccgcagcgtggggatgctcgggcagctcctccactcccggtttaggtgtgaacgttggaggggtctggaggctgtggtggcgttttccggaacatgtccccctccatgggggacatctctggaggggagaagttagggccgcgtcccccgtgccggttaaaggggtaggcaccgggctcctccggaatcatcagggtctgtcggggctctctccccgccccctccgagtcctgggaaagatcggaggacggggtggagacaagtgggccttggcccccgcacccctctgcgttcgtgtccgaggcggcggcgggggctcccgaactcccctgaaatcgtggggctccatgtggcctccggcagcgttccaccctcccccacctggggaagggaaggggtggggagtgcccggccccgtcccggccttcctccttcccccgccagacctctccggcgcgcgggtggtggccgatccgcattgctgttcgaggccgcagtggagaaggcgcctgtggaacatcggtgggtgagggctggacccaggctggaccctggagatccggggtggcggtgctggtggcagggggcgggcaccctgcgcacttatcccaacccccgccccaatttcggaaatgctaggagagagagattgcagcaggggacgtggtcgggttcctgaaggcagaaaggcgggtgtttactagcgtctttttccctcctaagccggggttgtagtaggggctgggggctcagtgttgtcccggctaactgggtttgactcgagggtgtgtttgtgcaggagggcctgttgggggtggcgggcggttgtcagttcgtatttcacgaactaagaaaatgcttagtgttcaaagggagaaggaaacgtcaatagactccattccattgtggccggtgtccttaacttcgggagtgccgccagagcttaccaagggcacgcaagtccatttcccttgtgcctcaagtccatccgtgttgtaggcactactgtgccttctttaggcctaggccgccggcttgacggcgggtgaccggcgtcctccttaaataggcatcttgggctttggaaggtggaataagaggatttttcattcacccgagttttctttttgaaaacacattttcagcaacccatttccaaagaatttttatttacagcagaaattccccatcaagaggaatcagctggtttttaaggaattctgctgccttcaaagggggcggaaacagtcggttatttgactttacacgccccgcccccccttccccttctctgagtctgaagcatcccaaacactacttagccaaactagttcagatgaagtgatcgtttccccaagtagggtaacttcagtttccctttttcgttggcatctagcgaaaaatgaaaaaatttaaaatacaacttttatagaaaaggatgtattctgtttttactttcttaggtattaggaagagatttggcagataattcaacatgttcaaatatataaacattaaaactaaggttattaagttgcattgactactaggcttaaaaattagattataagagaatttgctcctgagtagtttgagtgatcaaagatatttggaatgttttagtaccacaaggtcttttttctgttccttgaggctttacaacaatttaaggttaatttagatttttccttgctttaagttcttttacttgagacctaaatggcagcccttattctttctgatgaataggtgaaattttgtttactgtgttggatttgtgtaatgtgaagttttattcttgaacagatcgttaatgtacttgtagaattactttgaatttgaatcactttcctgcattccttgtaaataagtttcagcttctagaatctcctcacttaggtttgtgcgtatcaacagtgaaaataagtctctgagagcaagggtgaaaaaaaatgcagcattcggtttgacaagtttcgagatagcaaaatatgcttgaaagtctggaaattcacatctgctttaagaaacatttcataatttgactttgtgtgtgtgtgtgtgaatagtttttcatgactttcagaagtgatttattttgttctttgttatatatatttttgaaggtggctgttttaggaaagataatgtaatcacaatattagaacataattttactgtaatctaattttgtgtgtgtgtgtgtgtgtgtgtgtgtgtgtgtgtgtgtgttttggatggaatctcactctgtcgcccaggctggagtgcagtggcctgatctcagcttactgcagtctctgtctcctgggttcatttaagtgattctccagcctcagcctccccagtagctgggattacaggttcgtgctaccacacctggctaatttttttgtatttttagtgaggacgggattttgccatgttggccaggctggtctcgaactcctgacctcaagtgatccgcctgccttggcctcccaaagtgctgggattacaggcgtgagctactgcccctggccaatttttgtatttttagtagagatggggtttcaccatgttggccaggctggtctcgaactcctcctgacctcaagtgattcgccagcctcggcctcccaaagtgccaggattacaggcaggaatgagccactgcccccaaccatcagtctaattcttatttttgctttttaccttttcatttttatgtagtagaggtgattgtgtatgttattttgtagttagcttttttcccctgaacgttgtattgtaaatgtaaatttttttttttttttttgagacagagtctcggtgtttgcccagtctgaagtgcagtggtacgatctcagctcactgcagcctctgactcctgggttcaagcgattctcccacctcagcctcttgagtagctggggctacaggaatgttccaccacgcttggctaatttttgtatttttggtagagacaaggtttcaccatgttggccagtttggtctcgtactaccgacctcaggtgatgcgcccgcctcggcctcccaaagtgctgggattgcaggcgtgagccactgcgcccggctgtaaggtttttacttaaccattctattgttgggaattgggtttccacttttttgttatagatagtggtgcagtgaacatttttaaatagctttttgcttcagtgtaattatttccttagagaaagttaccaagagtggttttactagttcagagggcttcaggatttttatggctcttgctagcggtgctctattattctttagaagacttgtattacttccagtgtcaagaaggttgctcttccatggaatggtttctttgtagtttgtcaaatattgtggggaatttttaaaggaaaaattgcatttttactgtcaagtgcatatattattaagtgcttttgttagttactggattattgatatttgagtttaatttggttcctctgaggatttaataaggtaatatatgtgaagatgttttgaaacctgtaaccattattattaatgagggtacttggtttatctgtcgtgctgatagtactgagtaaagtgcaggaatgaaattcctgaggaactgttctaaagctttgttgttgttgttaacctttctttttcatctgaaagtgttttttattagctgctagcctatgaccaagttatttttggtaacttttttgtaatttcatggcactattgggaattttcgctggttgactcttcttcttctacattcccttccccattaaaaataaaaatatggatttacaattgttactctattcctaaacctaaataatatgacattagaattgcttgggatacaggattcagtctgaataaaatatttttcttttagtgattttcagcttagtatttttactgcttctttctcttgaggcattgcaacttaaaaattgtgctgtttagccaggcgcctgtaatcccagctacttgggaggctgaggcaggagcatcacttgagcccaggaggcggaggttgcagtcagctgagattgtgccactgcactccagcctgggagacagagtgagactctatctcaaaaaaaaaaaaaatgtgctgtgatttaatgtagttgttcatcatgcttccatttaaatttcagtgagactgttcatcttttgcagttaaatatcttgtagaagggcctaaaatatctacgttgaatacagctttattgaagcatctatgtacatggggtttttgggatgaatcagtgaataaagcaaacatattgtccttttggagtttacattctaatgtgactaggcagacaatgagacattaaattaccagcctatgtataatagtgtataagagctatggaattagaagaaagcagattaaaggtatagggagtgtggggaggggaatgagttacaattttaaatggattgggggaacttaattgaggagctaacatttgagcaaagatttgaaggttgggtatttagccgtttgctttttatctaggttaattagtcatgtggcttcattagtaatttataaggtttaaatggcatcatcctttgttattcttttatgtgcacattgatactaaccatctctgaagttagaccaaaaaagttaattgacattgagggtcattagaggtaaattgtagatggctattactaaccaaagagacatgttttgtttttcttttgggcttacgtattttacctaattagtttagtttttgtttcaagtatgtggagaaaataaactttttaagtttgggccaaaacttgctttggttttctttttctttttctttttttttttttaagagaaaaatgtaagcctgtagttgcttaaagattccacattctgaaacagtgaaaacatgggatcagtcatggtgttcctttttttggttaaatgtaaacttgtattttcagtgttactctaattagcaatggtttatacttctacataagggatgttaactcatattgtagctatttaatagccatatattttgacttaaaggaggatctcaaggccaggcgcggtggctcatacctgtaatcccagcactttaggaggctgaggcgggtggatcacctggggtcaggagtttgagactagtctggccaacatggtgaaacccccatctctactaaaaatacaaaaattagccgggcatggtggtgggcgcctgtaatcccagcttcttgggaggctgagggaagagaattgcttgatcccggaggttgcaatgagtgcggaggttgcagtgagctgagatcatgccattacactccagcctgggcaacagagcgagactctgtctcaaaacaaacaaacaaacaaaaaaaggaggatctcatttttttgtcctaaatagctacagccgtgttagaactgtcaccttagcaaagtattgtttttttactttgaaacgaattttaaggttttagaagattgttctctagaattacaattttctgttttgactagtgatagtattttgatgttgtgtaaatagttgagcatgaacaaaaccctatttttttttttagctatttcaagtgattgtgacaacttcaacggagatgtaaacagtttattaacagtcacacctattatcttttttttttttttttttttttgagacggagtcttgctctgtcgcccaggctggagtgcagtggcacgatctctgcttactgcaacctttgcctcccgggttcaagtgattctcctgcctcagcctcctgagtagctgggtctacatgcgcacaccaccacgcctggctaatttttgtatttttagtagagacagggtttcaccatgttggctagaatggtctcaaactcctgacctcaggtgatccacctgcctcagcctcccaaagttctgggattacaggcatgagccaccgtgcttggccgctgccgtatctttttaaatgaaagtacttgtgttttttttgtttttttccaaaggatatctgggtcatctatgatgttactgttaccatctaagggtttttttgtttgtttttgagacagagtctctgtcgcccaggctggagtgcagtggcgtgatcttggctcactgcaacctccgcctcccaggttcaagcaattctcctgccttagccctcccgaatagctgggattacaggcacccgccaccatgcctggctaagttttgcatttttagtagatatggagtttcaccatgttggccaggctgctcttgaactcctgacctcaggtgattcgcttgcctcggcctcccaaagtgctgggattacaggcgtgagccacccccgcccagcctcatgagctaaggtgttttttttttttttgagacagttttgctctttcccaggctggagtgcagtggtgcaatctcagctcactgcaacctctgtttcccgggttcaagcgattctcctgcctcagccttctgagtagctgagattacaggtgcctgctaccccactcagctaatttttgtatttttagcagagacagggtttcaccatgttggttaggctcatctcgaactcctgaccttaagcgatccacctgccttggcctcccaaagtgctgggattataggcatgagccaccgtgcgcagcctaccctgtctcttaaaaaacagtaacaacaacaacaacaacaaaaaatcctaaatcttaaaaatggaaggcaaaaactctaagctttgagagattaggggacttgcccaaagcaatatttgtaggattttattacacctctccctttatttatttttttagagtcaaggtctccctctgtcacccaggctggagtgcagcctcaatctatggggccaagcatttctcctgtcttagcctcctgagtagctggaactacaggtgtacaccagctggctaacatttaaattttttgtagagacagggtcctgccatgttgcccagattggtctcaaactcctgggctcaagtgatcctcctgcctcagcttcccaaagtgctgagattacaggtgtgagccactgcaccgagccccctccctttatttttatttttaaattttaagttctggggcccctcccttgaaataaatagaaacgtaatatatacacaagatcatgctgtgtattttaaggcaatggtcctcaacctttttaacactagggaccggttttgtggaagatggtttttccataggggcaggggatgattttgagatgaaactgttccaccggccgggcacggtggctcacgcctgtaatcccagcactttgggaggccgaggcgggcagatcacgaggtcaggagatcgagaccatcctggctaacatggtgaaacccccctctactaaaaatacaaaaaaattagctgggcgcggtggagggcgcctgtagtcacagctactccggaggccgaggcaagagaatggcatgaaacccgggaggcagagcttgcagtgagctgagatagcaccactgtacttcagcctgggggacaaagtgagactccgtctaaaaaaaaaaaaattgttccacctcagatcattatgcatttgttagattctcataaagagcatacaacctacatctcttgctatatgcagttcccagtagggtttgtgcttctataagaacctaatgctgcacctgatctaacaggtggggctcaggtgctaatgctcacacagctcctgttgtgcagtctggttcctaacaggcctgtttttttttttttaattagatggagtctcgctctgtcaccaggctggagtgcagtggcacgatctcagctcactgcaacctctgcctcccgggttcaagcgattctcctgccttagcctcccatgtagttggtactacaggcgcacactgtgatgcccagctaatttttgtatttttagtagagacggggtttcaccatgttggccaggatggtgtcgatctcctgaccttgtgatccgcccaacagcctcccaaagtgctggaattacaggcgtgagctgctgcgtccggccccctaacaggcttgttttatggaatacagtcacggacagtacttgcccttcaggatatctttttgtaaccttgattttggcttgctaaaataggaggtctattttcttttctttgtttttaatgtatgtggttctgtacttacgtggtgtgaaatctacataaatgttaaatccttggttatttatttattttgagacagagtctcactctgtcacccagtctggaaagcagtggcataatctcggctcactgtaacctccacttcccaggttccagtgattctcctgcctcagcctcctgagtagctgggattacaggcatgcaccactacacctggcaaatttttgtatttttttttagtagagatggggtttcaccatgttggccaggctcgtcttgaactcctgaccttaggtgatctgcctgtcttggcctcccaaagttttgggattacagcatgagccactgcgcctcgccttatttttttgagacaggttctagctctgtcacccaggcgggagtgcagtggtgccatcatggctcattgcaacctcgagttctcaggcccaagtgatcctcctatctcagcctcctgagtagctgggaccacaggcatgcgccactatgcccagcaaaatttttgtttcactctgttgcctagggtggggtgcagtggcaggatatcggctcagtgcaacctctgcctcttgcgttcaaatgattctcatgcctcagcctcccgagtagctgggattataggcatgcgccactacacctggctaatttttgtattattggtagagatggggttttatcatgttggccaggctggtctcgaactcccgacctcaggtgatccatataccttggcctcctgaagtgctggaattacaggcataagccactgcgcctagcttttttgtttgtttttattttgtagggacagagattttacctgttgcccaggatggccttgaattcctgacctcaaacaatttgccctccttggcctcccaaggtgctgggattacaggtgtgagccactatgcctggctggttttttaaattattattattgtttgtgtgtgtgtgttgcaggatcttaccctgtcacccaggctggaatgcagtgatgtgatctcggcttactgcaacctccgcctcctaggttcaagtgattgtcctgcctcagcctcctgagtagctgggataacagctgtgtgccaccatgcctggctaatttttgtatttttagtagagatggggtttcatcatgttggccaggctggtctcgaactcctgaccttagatgatccacccgcctcgtcctcccaaagtgctgggattacaggtgtgagccaccgtgaccagtttggtttagtttttttttttttttttttttttttttttttttgagaaatctcgctctgtcgcccaggctagagtgcggtgacacaatctcagctcactgcaagctccacctcccaggttcatgccattctcctgcctcagcctcccgagtagctgggactacatgcgcccgccaccatgcccggctaattttttttatgcattttaagtagagatggggtttcactgtgttagccaggattgtctcaatctcccgacctcttgatctgcccgcctcggcttcccaaagtgctgggattataggcatgagccaccgcgtccggcctggtttggtattttttttatgagtctgggttgtttatgaaaacttgtcacagctgttaaccttaactttttttttttcttttttttccgagacggagtctcgctctgtcacctaggctggagtgcagtggtgcgatctcggctcattgcaacctctgcctcccaggttaaagcgatttttctgcctcagcctcctgagtagctgggactgcaggcacgcaccatctcgcctggctaatttttgtattttagtagagatggggtttcaccatattggccaggctggtctggaacttctggcctcaagtgatccacctgccttggcctcccatgcctggcaaccttaactttttatttgctggtaattatttgtgtttgcattcatgtgaaaatttgaaattctcattaacatttaaagattcttacatagattgcttgtaattttaaccctgaagttgtgtcaagtgactttacaatgtcaatttgttttatttatttatttatttatttatttatttatttttgtgataggatctggctctgttgctaaggctggagtgcagtgttgcaaatacggctcactgcaacctctgtctcccgggttcaagccatcctcccacctcagcctcccaagtagttggaactactggtgcgccccacagtgcctgcctagtttttttgtattttcagtagatgtggagttttgccatgttgatcttgaactcatggcctcgagtgatccaccccacttaggcctcctaacatgctggtgttacaggtgtgagccactgtgtccagcccgaaaatgtcagtttcgtgccatgattaatagctaactacattttgggaatgtaataaaatttcattctataatgaagtctttgtaaaactcattagttgtggtatgaggcttgtcggcaatataagtgaacgtggtttatttttattaactgtatcagaactttagaatgttggtctcctgaaaccattgccttgagaggctttattgaacagtgttgccaatgatcagtttttttttaaatttccttttttttgagactgagtcttaccctgttggccaggttggagtatagtggtatggtcatggctcactgcagcctcaacatcctgggctcaagcagtcctcctacctcagtctcccgagtagctggaactacaggtgtatgccaccatgcctggcttttgtatattttgtagagacagggtttagccatgttgcccaggctggtctcaaactcttaaattcaaatgatccacccacctagttttcccaaagtgctttaattacatgtgtgaggcaccgtggctggccaggtcaaatatttttcattgacgtttttcatattgctttttaaagtcatgttaaaatattcttaataatttttctaagtggaattaatcttgattataattttagttttttataaagggcgggttttgaaacaagtactgcatttttcttttcgggtttataaacatttgctgtggactttgtgcagttaactattttcattcctgaaacacatttcgaaatcaggaattgaagactaaatgtcttttcactgaagcttgagcagattttagaaaggggagttcttttttttttttttttttttggtagaaatgggggtcttgttatgttgcccaggctggcctccaacttctgggcttaaactgtcctcctgctttagcctctggtctggagagttctttatggcctctttgagaacttttactttacacatgattctatctagctttcttttctgatgtacatattggcagcaagtagaaaagcaatgttttcagaggcagatatattaacagcaatgagaaataacagtagcgtgatagaaagttgaaagacttagctgggtgcggtggctcacgcttgtaatcccagcactttgggaggccaaggagggtggatcacttgaggtcaggagttcgagaccagtctggccaacatggtgaaaccctgtctctactgaaaaacagaaaaagggccgggcgtggtggctcacccctgtaatcccagcactttgggaggttgaggagggcggattacaaggtcaagagattgagaccattctggccaacagggtgaaaccccatctctactaaaaatacaaaaaaattaaatgggcgtggtgatgtgtgcctgtagtcccagctactcgggaggctgaggcaggagaattgcttgaacccgggaggcagaggttgcagtgagccgagatcgggccactgcactgacgacagagggagactccgtctaaaaaaaaaaaaaaaaaaaaaaaaccagacttggggctgggcgggcgcctgtaatcccagctacttgggaggctgaggcaggagaatcgcttgaacccgggaggtgaaggttgcagtgagctcagattgtgccactgtgccccagcctgggccacagagcagagtgagactctgtctcaaaaaaaaaaaaaaagtttggaagactggtggctgggcatggtggctcacacctgtaatcccaacactttgggaggctgaagcaggcagattacctgagcccaggagttcaagtccagcctgggcaacacagggaaaccccatctcaacaaaaaatattaatacaaaaaatttagccagtcatggtcgtgcacttctgtagtctcagctacttgggaggctgaggcaggtggttcacttaagtctggatgtcgaggtgagccatgattgcaccactgcactccagcctgggcgttaaaatgagaccttatctcaaaaaaacaaagcaaagagcctgggaactactaaaatgggaactactaaaaaacagacacaagagctcaacaagtataccattctgggaggtttttttttttttttttttttttttttttgagatggagttttgctcttgtcacccaggctggagtgcaatggcgccatctctgctcactgtagttccgcctcccaggttcaagcagttctcctgcctgactcctgagtagctgggagtacagatattggtcacacaccgggttaatttttgtatttttagtagagacggggtttccccattttggccaggctggtctcgaactcctgacctcaggtgatccgcctgcttcagcctcccaaagtgccgggaccacaggcgtgagccaccgcacctggctttttttttttgacatagaatcttgttctgttgcccaggctggagtgcaatggtacaatcttggcccactgcaacctctgcctcccagcttctagcgattttcctgcctctgactcctgagtagctgggattacgggtgcccgccaccacacccggataatttttgtatttttagtagagatggggttttgccatattggccaggccggtcttgaactcctgacctcagatgatccacctgcctaggcctcccaaagtgccgggattacaggcgtgagccaccactcccggcctgggagttttgactgtaagtttatagctgtatatcttaggccctaagggcattactgttttatagcacagtgtagttagttaatgtgctcataatggtgactcataacaccaggttaaatgattttttatatctcccaaagaagtatttttcaatctgcagatcatgaccccttagtagattgtgaaacacattagtggattatgacaagcatttttagaaaaatgaaaaagaataagaagtgttaggatgcattgcattattgaaataattgtttttgagatggagtttcgctcttagttgccgaggctggagtgcaatggcccgatctgcctcccgggttcaagtgattctcctacctcagcctcctgagtagctgggattacagacatgctccaccatgcctggctaattttgtatttagttttagtagagatggggtttctccatgttggtcaggctggtcttgaactcctgacctcaggtgatccacttgcctcggcctcccaaagtgctggggatacaggcatgaacccctgtgcccggcctaatttttgtatttttagtagagatggggtttcaccatgttggccaggatagtcttgatctcttgacctcgtaatctgcccacctcgactcccaaagtgctgggattacaggtgtgagccactgcacccagctgccaagaattgttttaagctttggtttgagttaatgtatatataccgcattgtaattcaaaatgtaatttttggccaactctgggcacattgcctatggactagtcctgctctgccacgagcagcaacagttcaatgaattttttttttttttttttttttttttttttttttgagacagggtctctgtcaccaaggctagaatgtagtggtgcagtctcggctcactgcaacctctgtttcctgggctcaagcgatcctcccacctcagcctcctgagtagctgggagtacaggagcacgctaccatgcctggctaatttttgtattttttgaagagatgaggttttgccatgttgttcaggctagtcttgaactctggagctcagatgatccacccaccttggtgtccagaaatgctgggattacagggatgagccaccgtgcctagccaaaaatttttttttaagtaattttttattgatatagtcaaaaaagttactgctttagagccagagaaacgcagtaaaaggattgagaaagagttttgaggttatatctaagctagggttgtcagatttggcaaatagaaatacaggacactcagttaaatttgaatttttgatgaacattgaccagttttttagtataattgtgtattaaattgcatagaaaaaagttatttatctaaagttgaaatttaactgagcatcttgtattttatctggcaactccagtctaagctggaatcatggttcactgttttttttttttttttttttttttgagtcggagtcttgctgtgttgcccaggctggagtgcaatggtgcgatcttggctcactgcaacctccacctcctgtgttcaagtgattctcctgcctcagcctcctgaatagttgggattacaggcacccaccaccatgcccagctaatttttatatttttagtagagacggggttttcgccatgttgttcaggctggtcttgaactcctgacctcaggtggtccgcccacctgggcctcccaacgtgctgggattacaggcatgatctaccgtgcctggccatggttcactcttcagtaactaaaatttaagctctatgaaagcaggaactttgttttgttcactattgattgtatccctatttcttgaatggttggcacttaactgcttggtcacatgtttgaatgggcaagttactcagccactctcaggcttagtttatttacctattaaaagagaaagaatatcttccttggctgggcgcggtggctcacgcctataatcccagcactttgggaggctgaggcgggtggatcacgaggtcaggagatcgagaccaacctgggcaacattgtgaaacctcatctctactaaaatagaaaaaattagctgggcatggtggtgcgcatctgtagtcccagctactcgagaggctgaggcaggggaatcgcttgaacccaggaggtggaggttgcagtgagccaagattgtgccactgcactccagcctgggcgacagaacgagactctgtctccaaaaaaaaaaaaaaaacaaacaaaaaaaaaaactgagatactggccgggcgcggtggctcgtgcctgtaatcccagcactttggaaggccgaggcgggtggatcacgaggtcaggagatcgagaccgtcctgcctaacatggggaaaccctgtctctactaaaaatacaaaaaattagccaggcgtggtggcgggcgcctgtaatcccagctacttgggaggctgaggcaggagaatggcgtgaacccgggaggcagagcttgcagtgagcggagatggtgccactgcactccagcctgctgggcgacagagcgagactccgtctcaaacaaacaaacaaacaaacaaaaaaactgagatactaaagtcttaatattttctgtttttatgtatttattttttgagatgggatcttgctgtattgcccaggttggagtacagtattgtgatcatggcttattgcagcctttaactcctgggttcaagtgatcctcccacctcagcctcctgagtagctgggaccacaggcacatgcaacatcacaccctgcagttctttttttttttttgagacaccgtctcgctttgtcacccaggctgcagtgcgtggtgcaatttctgctcactctaacctccacctcccgagttcaagcagttctgcctcagcctcctgagtagcttgggaccacatgtgtgtgccatcatgcctggttaattttttgtatttttagtagtgacagggtcttaccatgttgcccaggttggtctcaaactcctgagctcaagtgatctgcccgccttcgcctcccaaagtgtctgcgccctacaatttaaaaaaatttttgtagagacagtctcactgttacccgggctggttttgaacttctgccctcaagtactcctcttgccttggcctcccaaagtattgaaattaaggccatgaggcagcacacccagcctaaattcttcttatgttctgttcttggcacatagtagatgttcaacaatgtagagtcaaacgcatttggagttggaatggctctggtgttttttttttttttttaaaccagaaacacgtgcagtttattgaatgccattgtagaaaagtgtgtgaggataaacggctgatagagaacttggctctgggggcagggcgaggaatggagggtggatggagtacatgggaatcagatcacgggcagagctcctggcctagataatgcctcctgatctgttgatagacttgaaagatcaacactgggatgatgctgagcagaatggtcgtaatgatgcgcacaatcagggcccagatgttcaggcacttggcggtaaaggcataggcctgggccctgatcaggtcgccaaccatcttcttgtccctagacttcacggagtaggccaatgctatgaagcccaggcagcaggagttcatgaagtgggtgttgaacagggaccagacgacatggtcgggcacggagttctcgctgtggatggggatcacggtggacattgggggagcagggttgtggggtgcccccagcacagccacctcttgctcctccttgagcatctcatagttagggggatggccgatgttggcaggagtgaagaggtttggacattgtggttcatggtgtccagggaagaccagctgtggtcgggttgctggggtggttctcagtgggcccctccctttccctggtagtttggatttctctggctctggtggttttttagtactcattctatttacgggtgaagaaattgagaccaagagggttatttaccagagtatctcatcattggctgcataactggcattagaatctgatgtacttttatttctaatacatttctttttttttttttttttttgagatggagtctcgctctgttgccgagcctagagtgcagtggggcaatcttggctccttgcaacctccacctcctgggttcaagctattcctgtctcagcctcccaagtagctgggactacaggcacctgccaccacagccggctaggttttgtattttagtagagatggggtagcaccatgttggccaggctggtctcgaactcatgacctcaggtgatccacctgcctcggcctcccagtgctgggattataggcatgagccaccatgcctggcctttctttgtcgtttcctttctttctcttcatccctcctctccttttttcccctccccgctgcctcctcctgtcttcccttctttccttcctttctctcctttttattttttcctttctttttctttctctgtctctcccaacccttcctctctccctccctccctccccttctctctccccccctccctccccttctctctccccctccccttttgttcctaagagacagggtctccttatgttgctgaggctgaccttgaactcctgagcccagatgattctgcctccttagtagctgggactacacccacctcccgttccgttgtcatcttttttttttttttcttttttggagacagaatcttcctctgttgctcagggtggagtgtagtggcacgatcatagcttactgtaactgtgtaacctcgaattcttgggctcaagcaatcatcccatcatcccacctcagcttgctgagtacctggggctacaggtgtgtaccaccatgtccggctaattacttttcttatttttaatttttcggagataggatcttgctctgttgcccaggctggtgtcaaactcctgggctcaagtgaaactcttgccttggcctcccaaagtgttgggagggattacaggcatgagccactgcacccagcctcctctttcttcccatttaactcctaaccacaccgaactttctgtctgcagagaggagcattggtcagcagttcacaaaatggctaggtgtgatggcgtgcacccatagtcccagctacttggggagctgaggtgggaggatcgctggagcccaggagttcaaggccctgggcaacacagcaagaccttatctctggctgggcccagtggctcacgcctgtaatcccagcactttgggaggctgaggtgggtggatcacctgaggtcaggagttcgagaccagcctggccaacatggtgagaccctgtgtctactaaaagtacaaaaattagccaggcacggtggcgcgctcctgtaatcccagctactcgggggggctgagacaggagaatcacttgaacccaggaggaggaggttgcagtgaaccaagaacacgccactgcactccagcctgggtgacatagtgagactcttatctcaaaaaaaaaaaaaaaaggtcgtctgtactattgcatgttagtagtttctttctgcttattgttgagtagtagtctattgtatgcatgtaccagtttgttcatctagtggtggacattgagttagcaggttttggctattaaaaataaagctggaggccgggtgcgatgtctcacgcctgtaatcccagcattttggaaggccgaggcaggcggatcacctaaggttgggagtttgagaccagcctgaccaacatggagaaaccccatctctactaaaaatacaaaattagccaggcgtggtggcgcatgcctgtaatcctagctactcaggaggctgaggcaggagaatcgcttgaacccgggaggcagaggttgtggtgagccaagattgcaccattgcactccagcctgggcatcaagagtgaaactccgtctcaaaaaaataaataaataaagctggtatgaatatttatgtacaggttttgtgtgaacatatgattttatttctcttggttggaatgcatagaaatgagattgctgggttttgtggcaagtgtttatttttccagggtacatataatcctgtgagtgtttatttaattttaaaagtaattgctaaactgtttgctaaagtgactgctatattttctttccctagcagtgtatgaatttttttttgaggcagggtcttgctctgtcacccagggtggagtgcagtggtgcgatattgtctgactgcaacattgacctcctgggctcaagtgatcctcctgcctcagcctcctggctgggaccacaggcatgtaccaccacacctggtagtttgctttgatttttagtagagaagaggtctcactatgttgccctgggtggtgttgaactcctgggctcaagtgattcatctacctcagcctcccaaagtgctgggattatagatatgagcccctgtgcctggcctcattgtggttttaatttgcatttccctaatgcccagtgatattgagcattttttcatgtgtttatttgacattcataccatctttggtgatgagaaactatgtttatgcattgcttaatgatggggatgtgttttgagaaattttttcggtgatcttatcattgtacaaatatagagtttacttacacaagcctagatggtatacctactagacacataggctgtcgtacagagtattactcttaggctacaaatctgtatagcatgttgcggtactgaacactgttggcagatgtaacataatgttaagtatttgtgaatctaaacatatctaaacatagaaaaggtgagtaaaaatacagcgtaaaagataaaagtggtatatctgaataggtcacttaccatgaatggagcttgcaggacaggaagttgcttgggatgagtcatttatcagtggtgtgtgaatgtgcaggcctaggacattactgtatgctactgtagacaaacactgaacagttaggatacactaaattgataaatatctttcttattttgttttttgagatggagtctcgctctatcgcccaggctggagtgtagtggcgtgatgttggctcactgcagtctctgccttctgggttcaagcgattctcctgcctcaacctcctgaatagctgggattacaggtgcgtgccaccacacctggctaatttttgtatttttagtagagacgggggtttcaccatgttggccaggctggtctcgaactcctgacctcaggtgatccacccgccgtggcctcccaaagtgctgggattacagatgtgagccaccgcacctggccagagatgaggtcttgctgtattgcccagggcgttgaactcctgggctccagcaatcctcccacctcagcttcccacgtagctgggactgtgggtgcacgccatcatgcctagccgttttgtgaactgttgaccaatgctcttttctgcagacagaaagttcactgtggttaggagttaagacttttaacctctgacctcaagtgatctgcccaccttgacctcccaaagtgctgggattacaggtgtgagccatcacgcctggtcaaaaatatctttctttaagagtaaatttaccttaacttactggttgatcattgtatataggtctgttgttaattgaaacatgcgggccgggcccggtggctcatgcctgtaatcccagcactttgggaggccgaggcgggtggatcacaaggtcaggagatcgagaccatcctggctaacacggtgaaaccccgtctctactaaaaatactaaaaattaaccgggtgtggtggcgggcgcctgtaatcccagctactcgggaggctgaggcaggagaatggcgtgaacccgggaggcggagcttgcagtgagccgagatcgtgccactgcactccagcctgggcaacagagcgagactctgtctcaaaataaataaataaataaataaataattgaaacatgcggtgcatgtgtttatttgcgatctgacttgtttggaaatatttgcattatcttccttctagatttagagcatcttgacagtaggaacaagtgttttgtacaactttgtatgcttagtaagttatcaattaacttgtcgtggccaggcgcagtggctcacgactgtaatcccagcactttgggaggccgaggcgggcagatcacctgaggtcaggagttcgagaccagcgtggccaacgtggtgaaaccctgggtttgtttgtttgtttatttatttatttattttttggagacggagtctcgctctgtcgcccaggctggagtgcagtggcgtgatctcggctcactgcaacctccgactcccaggttcatgccattctcctgcctcagcctcccaagtagctgggactacaggagcccgccaccatgcctggctaatttttttatttttagtagagatggggtttcgccgtgttatctgggatggtctcgaactcctgactttgtgatccgcccgcctcggcctcccaaagttctgggattacaggcgtgagccaccacacctggcctaccctgtgtttattacaaatacacaaattggccatttgtgcgtggctcatctacagtctcagtgactcagaaggctgaggcaggagaatctcttgaacccgggaggcagaggttgcagtgagcagagatcgtgccactgtacttcagcctgggtgacagagtgagactgtgtctcaaaataataataataatttgttgaatatgtgactgttggtttaatttttatttttatgagatggagtctcactctgttgcccaggttggagtacagtggcgtgcagtggcgcaatcttagctcactgcaacctccgcctcctgtgttcaggtgattcagcctcccaagtacctgagactacagacgtgcactaccgtgcctgactaatttttgtatttttagtagaaatggggtttcaccatgttggtcagcctggtctcaaactcctattctcaagtgatccgcctacctcgaccttccaaagtggcggaattataggtgtgagccgtggtgcccggccagactattggtttggtttggtgtgatgttatgttatgttatgttatgttatgttatgttatgttatgttatgttattttaagacagagtttgtctcttgtcgcccaggctggagtgcagcggcatgatctcggcttactgcaacctccgcctcccaggttcaagtgattctcctgtcttagcctcccaagtagctgggattacaggcgcccaccaccgtgcctggctaatttttgtatttttagtagagacagggtttcaccatcttggccaggctgttctggaactcctgacctcatgatccacccgccttggcctcccaaagtgctgggattacaggcgtgagccactgcgcctggctgactattggttttattattaagcagtagtagttgaccctgtcatgtagaaagcatggcatttataggcataccacgtttaatttcctcccctttttttatttttggagtacctcctgcttgtgaggcttgggaatacagtagtgaataagccagatgaggtctctctctttttggagcttatgtggtagtatagactaggcagaaagttctcattgcccctgccaccttatggcattgaggtgtttgagatgctgatgtttacttctgtctcataaaatcttgaaaggagttcttttagatgaagaggaaaacaaaatcagaagaatgggcctgggtcatgtctgtaaacctccccacgtcatggggaggctgaaatgggaagggccaggagttcaagaccaggctgagaaacataacaagaccccatctctacaaaaaatattttttaattaatgggggatggcagcacacacctgtagtcgcagctactacgaggctgaagcgagaggattgcttgagctcaggagttaaagattgcaggagctatgatcacagcactgcgctccagcccctcttatcagcagtctggtatgttgctaagggtcttgttctttttagtgcttcagggacagccactggctatgcccagaaataagtatgtttgagaagctttctgacctcagcttgaaaaattgattagggtcataattaaaaagggagggaaacaggattgagtgaaccggacgctaccgtgagtttattctcccagggcatacataatctcatgtgattaccacatagccctgttagataatctgttatcctgtcctcattttacccatgaggaaatgaaggcccagagaggttaaatgacctattcaaattcactcagaaggtggcagagatgagttactatcattgtattttggatctctggaaagaaagaaaactagtgatggtattaaaaaatgttattaatagtttcttttaatcaaccaggaacttgagtcactagcttctctgggtgaaggactatacttcaacagtatgaaaaacggaaaagaaaatgaggaattttggctgggcacagtggctcacacctgtaattctagcactttgggaagccaagggaggagggtcgcttgagctcaggaattcaagatcagcctaggcaacatagtgaggccccatctctacaaaaataaattagctgggcatggtggtgcatgcgtatagtctcagctacttgggaggctgactcaggagggtcacttaaacccaggaattggaggttgcagtgagctatgattgcgccactgtataccatcccaggcgacagagtgagaccctatccccccaccgccaaaaaaaagaaaagaaaatgaggaatttacatttgtgacagatacggaattcagggaatttagttgttcatagtctataaatgctataagaagtctccatacctttttttttttttttttttttttttggagacagagtcttgctctgtcgcccaggctggagtgcagtggtgcgatcttggctcactacaagctctgcctctcgggttcacgccattctcctgcctccacctcccgagtagctgggactacaggtgcccgccaccacgcccggctaatttttttgtatttttggtagagatgaggtttcactgtgttagccacagatcccgacctcatgatctgtctgcctcagcctcccaaagtgctgggattgcaggcttgaatcaccgcacccggccggaagtctccatactttttaacccaatctaaaatggtaaggaaatatataagaatgtctatttattattaaattttttctatataaaacatttcagaaaataaagactagcatttctgagccaagtggtagtagtggccattttttctggaaaaaaaaaaaaaaagaaagaaaaaacacatttagctatctatgatgtgaaaagatgaacattttatttaggtaataaatgttatgtcataaaataccatttattgtgtgcctattaggtttcaggagagctgtgccaagagcattacttgtatatcttttaagccttacaacagcccagcctgtcaggctggtagtgccatatctgttttacagatgaggaagtgatggattggagaaattaaggaaattgcctttaggtcaaagagataggaagtgacaaagctgagatttttaaccttgtgagatttcaaagtctttgctttttaataactgttccattgcttctaatatagagatatgacaaaaacaagtaaaaatcagtgaagaaggctgggagcagtcgcttatgcctgtaatcccagcagtttgggaggccgaggcgtgtggatcgcctgaggtcaggaatttgagaccagcctggccaacatgacaaaactccgtctctactaaaaatacaaaaaagttagccaggcgtggtgacaagcacctgtaatcccagctactcagtaggctgaggcaaggagaatcgcttgaacctgggaggtggaggttgcagtgagctgagatcgctccattgcactccagcgtaggcaacaaagcaagactccgtctcaaaaaataaataaataaataaataaaaataataacaataatgaagaaaacaatccggtgattattgtcagcaataaaatttcttcaatcaaccatgctttagtcctggcagttctctatcagtgagtttcaatcaaaaagtttgtttataatttttttttttttaaaattttgaaatttggaaacaacatcataaatgatggttagttttctgcagctccctattttggcagatagtctgttgttactcataattaatttgaactaaaaagtagtgttgtacgatatcatgggctgtgaatgtgtttgtgacttgatctgagaacccacacaccacttaggatgcttctgtaggaaaattagagtatggaactcacttgcccacgctttccctgtctcagtccatgttggtaggctgcaaagtctggggctagaaggacactgaacaagacttcagcagtacatgttagtcttccagagggaaggaatataatagttgagagaataattcctttcctctgtgactttaggcaaattcttggctatgctgttatttatttgggccaaacaatatcaggaggttgtacattttattcttaattactgcgatacattaattttatccatgggtttaacctagcctaccttttgctgttagacttcaactctacttgtgttgggttacccctctgcttaaaaatcaccctattcccaagcctgagggagtctaccttcaaagctttctatgacctaatccaaggcctgtcaaacttcgtaaagggccagatagtaaatttgttttttttttttgagatggagttttgctcttgtcacccaggctggagtgcaatggtgccatcttggctcactgcaacctctgcctcccaggttcaagtgattctcctgcctcagcctctcaagtagctggggttataggcatgtgccaccacgctcggctaatttctttgtatttagtagagatgggggtttcaccattttggtcaggctggtctcgaactcctgacctcaggtgatccacctgctgcggcctcccaaagtgctgggattaccagtgtgaaccaccgtgcccagcccgatagtaaatattttaggctttgcagtccatatacagtcccatttttttgtgtatgtttgcacgttttctttacatattttaaaagcccctttttttttttttttgagacagagtcttgctgtgttgctcaggctggagtgcagtggtgcaatcttggctcactgcaacctctgcctcctgggttcaggcgattctcctgtctcagcttcccgagtaactgggattacaggcacatgctgccacgcccagctaatttttgtatttttagtaaagatggggtttcgccacattggccaggctggtctcctgatctcaggtgatctgcccacctctgcctcccaaagtgctgggattacaggtgtgagccaccgtgcctgacctaaaagctctttacagtgtaaaaaatattctgagctttaagccatgtgaaaataggccatgggcatttgctgacccctaatagaactccattttacctttctgatcatgtttcccattaactcttcaaaaatatgacctccatttaaatcaagatggtctccttcctcactgcttgtggaggtccagtgcccagtgtctgcctcttgcttgctcctccatcattgttctgccattcgagatcctcatacttaccctttaagatctagcccaaattttccatgaaactaattctaataattaaaaacttcctgtagaacttaactttgtctagtacaagttagctttcttattcagtagtagcttactataaattacaagaataaaaagattaccattttccctcacactgttttgtggagaatgcctaaagttactttttctttttacaggtcagtattcctatttggcatcctaatcccctttcccaaatctgaattttgggatttgaagcttgcatttgagattatgatttgtcttccttgttgtacacaggagcagggactttacaattagtattcgcatccctgctccttcatacttcgtgatgtaaggcaagttattttcacttatgcttaagtttcttcccctgtaaaaaggggatggaagaggattaaatgaattaaacatgtaacacgcttaaagcaatgcctggcaagtaataagtgctcagtaacttttagctgttcttattagcatgtttggaaaccagtagaaactacaccagcaagttaaggttgaaaagtggtattgatgggcttggggtagtacagtatgaatggctacagtttagcgtttcattaagtttgtatattcattaattcattacacatttgatgctgtcagactaggacagagacaaagatgaatgaaacattatctctgcttccaggttacccagtgtagtagagaaggcaggcatgcagatagtttaaattggtagcactgggaggggactgccatgggtgggcagtgaagaaaaagggcttcaaaataatgagagttgagatggatcttcaaggaagataagcagttttcagtaaggccatgaagagaggaggaagttccaggcgggaagagtttgtgctaaagtacagggattgctatacacatggtgtatgtagaaaaaatttggttcacagtgtgatagaagaattggagggggtcctcactgaaagtaaggaaacacatttggaagaatatgtttcagttagaaaatgaaatgagcttaaagtaaacgctaataaggtttttaaaatgtaaaatttcaacgtatttagaaagagaacagctggatgaatcttatgtacctgtcactcagctttagcagttatcagtaaatggccaacgttgtttcagctatactcccctctcctccactgatagtcttttgaaggggaatacaattgttttgtggcctccagaaagggataagtttatgagcaacgggtagatcgttgggagagacttgagtttcctgtcaggaagcattcttggtgcataagtcagaggtgatatgaatgccgtggaagggggtggcttactgtctggagaactcgagaagatgggaatgggcactgtccagtattgtggctacttccacacatggttctttaaatttaaaattatgttgattaaaatttaaatatttcagttcctcagccatactaatcgtatttcaagtgcttagctgccacatgtgcctaatggctgcaatattggacagcatgacataggacatcttcatcattgtacaaagttctcttggacagcatgggactagagccctaagatccttttctacctgagttgtttggattttttggtgtgtctaggttggatctagttgttcatggcttcatgaccaagccttttatccctttctctagagggactcaaggggtaaaggcactgaaggggtaaaacttcatatgaagagtgtggtggtggtggtggtgttttaagacagggtctcgctctgtcactcaggctggagtccagtggcatgatcctggctcactgcagcctcgacttcctgggcttaagtgatcctcccacctcagctcccaagtaactggaactgtaggcatgagccaccacacctgcctaatttttaaaactttctgtagagacgagatttcgccatgttgtccaggctggtctcaaattcctggactcaccttggcctcccagagtgctgggattgcaggtgtgggccactgcgactggccttttttttctcctttactactctagtgtatgctggaatatgaggaaataattatattagctagcagttattaaacacttaataacataccaggcactgttttaagctatgcgatctgtatggaatattacttaatttccacaaccttatgaaaagatactattttttttcttttgagaaggtactattttcatcttcatttcatagatgttgaaattgaaacacagagagctgaagtcacaggattaaggccacagagctgagaagtgatggagccggaatttgaacccaagcaattaatgctgatattagttcttgtgtgaatggtaattgttttgaaacaatgatcctagatgattatatgaccggattaatctggcagttgttctgtgtgaatttagagttgccttcccacctcagtttcctaaaaacaaaacaaaacaaaacaaaacaaaaaaaactctagcttcactgtgtttgggttgtcatggcctaccccctcttgccacctcatttgactcaactttttagggagaaaatattcaatacgtggtataggatttccctttctaataataatgtaaacaacaacaagaagtctgaaattggaagaacaaaatgactcacctaagtgagttaaccttaagaggtggaacttgatttctagattttagttaattgtctaactgatgtactaaatattagttacttaagtattaaaacgggtagacataatagttggggagctgctgtagagggggtagtttgagaaggcttctttcaggaggtgacatttaagttggtaactaacaagaaaggggcagccatgtgaatagctggagggaagagcattcttacagttttactggaagggggttagaggtatgtggtacccttatgccaaagaaaattagttacttctatacaaccagtctgattctagaaacctggatcaatgaaatattttgattatataaaaaaatctgttacccaggtcttgttgaaatagcattagaaactactgaaggacatatagaggaggagtgttgaaaaatggtgatggatgagcagaatggtgaaaaataaaaagacatgaagctctataattatattgtatggtgacagtaccaatagagattgcatgttttttctccccagtttttttttttgttttgttttttgtttttgagacagagtctcactgtgtcactcaggctggagtgcattgtcgtgatattggcttactgcaacctctgcttcctgggttcaagcgattctcctgcctcagcctcctgagtagctgggattacaggcatgtgccaccacgcccggctaatttttgtatttttatttgagaggggatttcaccatgttggcaaggctggtcttgagttcctgacctcagataatccacctgcctcagcctcccaaagtgccgggattacaggtgtgagccactgcgcccggcctcccccagttgttgaaacaataatggaaggtaattttattcttagattatttaatgtttttcagttatcaggatgtgttagattgtttgtgtatattgttttgcttgttaattaagtaacacagtgaataagacagacaaacatacgaaaatgtacatttattttatttttttgagacagtctgttgcccaggctggagtgcagtggcccaatctcggcccactgcaacctctgcctcctgagttgaagcgattctcttgtgtcagcctcatgagtagctggggccatgggtgcacgccaccatacccggctaatttttatatttttagtagagatggggtttcaccatattggccaggctggtctcgaattcctgacctcaggtgatctgcccgccttggtctcccaaagtgctgggactacaggcatgagccactgtgccaggccatttcatttttggaacgttcttttttttttttgaaatggggtctcgctctgtctcccaggctggagtgcagtggctcaatctcagcttactgcaacctctgccttccgggttcaagtgattctcctgcctcagcctcctgagtatctgggactacaggtgcatgccaccacgccaggctaatttttgtatttttagtagagacggggtttcaccatattggtgaggctggtcttgaactcctggcttcgtgatctgcccgcctcaacttcgcaaagtgctgggattacaagtgtgagccaccacgcccggcctgtttctggaatattcataatcttttgttgtcatttcaacagtgctcacagcagcttcaccaggtgtagattccatcttaagaaaccactttctttgcttatccatgagaagcaacacctcatctattcaagttttatcatgagattgcagcaattcagttacatcttctgaccccacttctaattttagttctcttgcttttttaccacatctgcagttacttgctctactgaagtcctgaacccctcaaaatcattcatgagtattagaagcaatttcctggttgggcacggtggctcatgcctgtaatcccagtactttgggaggccaaggagggcggatcacctgaagtcaggagttcaagaccagtctggcaaacgtggtgaaaccccgtttctactaaaaatacaaaaattagcggggatgtggtggcgggcgcctataatcccagctacttgggagactgaggcaggagaatcgcttgaacctgggaggtggaggttgcagtgagttgagattgtgcccttgcactccagcctgggcaacaggagcgaaactctatcttaaaaaaaaaaaaaaagaaaagcaatttcctctaaaactcctgttaatgttgatgttttaacctcctcccatgctcatggatggcattctcagtggcatctagaatggtgaatactttttagaaagttttcaatttattttgccatcagagaatggctatgaatggcagtagtagccttacagaatgtatttctttttttttttttctttttttttgagatggagttttttttgctcttgtcacccaggctggagtgcagtggcatgctatctcggctcaccgcaacctccgcctcccgggttcaagcaattctcctgcctcagcctcctgagtagctgggattacaggcatgcaccaccatgcccacctaattttgtatttttagtagaggcggggtttctccatgttggtcaggctggtcttgaactcccgatctcaggtgatctgcctgcctcggccttccgaagtgttgagattacaggcgtgagccaccgcgcccggccgtatttcttaaataaaatggcttaaacgtcaaaattatcccttgatccctgggctatggactgattcttgtgttagcagttatgaaaacatttatgtccttgtacattcccatcatagctttttgtcaatgagaagtaattttttttttttttttgagacagaatctcactctgtttcccagcgtggagtgcagtggcatgatctcagctcagtgcatcctacaactctgaggttcaagcaattctcgtgcctcagcttactgagtagctgggattacaggcgcccaccaccacgtctggctaatttttgtatttttagtagagatggggtttcacgatgttggccaggctgggctcgaactcctggcttcaagtgatccacctgccttggcctcccaaagtgctgggattgtaggtgtgagccactatgcctggcctaattggcctaatttcaatattgttatatctcagggaatagagaggcacgaggagaaagagagacaagctgactgctggttcgtggagtagtcataacacacaacatttattaagattgctgtcttatatggaccgtttgtggtgccttaaaagaaatcagggtaacatcaacgattactgattacagattactataacagatacaataataattgtaaattattatttacaattgtaaaatacaatcttttctttattatttacaattattgtaaaatacaatctgattacagattactataacgtatacaataatagtggaaaagtttgaaaatattgtgagatttatgagaatgtgacacaggcgcaaagagagcacatgttactggaaatacggcactaatggacttgcccgactcggggtttccacagacggtcagcttgtcaaaaatgcagcatctgtgaatttcaataaagcaaagcagaataaaatgaggtatgcatgtattgccatcacatgtacactagtaaaatacgttttttttttcagtaggtggatcaacctcaaattttaatataaagcattacttaaaggagaatatggggacattcatgacatttcttatatgtacataaaacttcatgaaaataatttaatgctatccagcagtttattttagaagtactggaggctaggcatggtgtcttatgcctgtaatcccagcactttgggaggctgaggtaggaggatcacttgagttcaggagctggagaccagcttgggcaatatagtgcgaccccatctctacaaaagagaaaagaagtactggagtgttgcagctcttacagaatttgtctagcaggttttccagtctttaccagaaatgcccccatgcagaagtagtaaatactgattcatgtaaaataataaacaactttatctttcagtttttaaaagacagggtcttgtaacgttgcccagactggcctttaattcctgggctcaagcgatcctctcacctgagccttttgagtagctgagactacaggctgcacctctgcacctggctctgcttgatttttaattgttgtattgctgttgcagctatgtttttttttttcttcagtgtgaggatgggcaaactttttatgtaaagtctcaggtaataagtattttaggctctagggccatatagcttctctgttgcatatcctttttttttttttccatttcccctcaaattccttttaccataagcaactcttgaggaacataaaaatcattcttagcccagaagccagaccaaaacaggttgtgggctgtagtgtcctgacccctgatttaaagattgatagctttgaaatggaaagttttaactttctttttttttctttcccttgttctgattgggctgttaattcattaggtatttactcagtgtgtatcatatgaggcatgattcctctgctaattttggtagtggtagaaagatacttttgccaagcttggttgttaggttttcatttgtccaagagttcctgaccaagtgtgaatggatgttgaaatcaaggtgtttctttggccacacaatgtgcctttgggggctatatctatgtgcttctggtaccttcttttaattttcacaaagacactgcttgccgaccacactgttttgtctaatgtggggctatgaccccctggaagaggcatcattttctgattttcacagaagcataatatggtcaggtgatggtcctgagtagtgggtatatgacagatacactagtaattataatacagatctaaactggagagttgaaaacagcatcgtatatttgattgagataatcgaaggaagacttcctgaaaagatggcatttgagtttcaaggctgagtaggattaagtattattatttaaaaaatgccttggacaatgcattaaatagagttaacaaatcacatcacttatagtctccaattaaaaacattttacttaaacataattttagacttttagaaaaattgcaaagatattataaagaattctcctatatatctcacctgtattcttcaagtaacattttaccatattcaccttaacattttctctgtattggtaattgtatatgtaagatttaatataaaataaaaattcttattaaacatatgagagacatgatgcctctttagccctaaatacttcaacttgtatgtactaataacaagggcattctatttcaaaaccacagtacagttgtcaaaataaggaaattaataattgtgtcaaactgttattctgtttatagaccttctaatgtcctttaaaacaatcaacaaatcaacatttttctggtcaagaaccagtaaatatgtatattctacatatatatatacacatatatatacacacatatattctacatatatatgtggaatatacgtatttactccctctgtccaagaaccaatccaggattgttaccttcggttatcatgtatctttggtctcctttaatccaaagcagtttctttgtcttttatgacttgacacttttgaagattacaggttattttgtagactgtccctcaactagggtttatctgaggtttccttatgattagattcagatatttatttttggcaggaatacaacagaaatgatttgtgtgtttttctcattgcatgatatcagaaagtgcattgtatatatttatcccattactggggttgttaactttgatcacttggttagagttgtgtctactaagtttcttcactataaagttatttttcacttggtcatttcatcagtatcttgtggggagttactttgtggttatataaatactctgtttctactttcccttactatatttagcttctgtggacacttttgcctgaaacagttatttactatggtgttaccaagtagtgatgcccttttcttccatcattctgtctacatttttttttttttttttttttttttgagatggagtttcgctcttattgcccaggctggagtgcggtggcctgatcttggctcactgcaacctctgcctcccgggttcaagcagttctcctgcgtcagcctcccgagtagctgggattacagacatgcgccaccactcctggctaattttgtatgttcagtagagacaggatttttccatgttggtcaggctggtctccaactcccgacctcaggtgatccacccacctcagcctcccagagtgctaggattacaggcgtgagctgccacaccaggccttctttttctcttttaagagatagagtcctgctttgtcaccaaggctggagtgcagtggcatgatgatagttcactgcagcctcaaactcctgggctcaagtgaacctcccatctgtagctgggactacaggcacctgcataacacctgactgttttttaaaactattttagagatggggtcttgcgaagttgctcaggatggtcttgaactccgggtcttaagtggtccttctgcctcagcctctggattagttggcattacaggcatgagccattgtacctggcaagtgcatattttctttttttttttttaaggtggagtctcgaggccgggcgcagtggctcacacctgtaatcccagcactttggaaggccgaggtgggtggatcaagaggtcaggagatcgagaccatcctggctaacatggtgaaaccctgtctctactaaaaatacaaaaaattaactgggcatggtggcacacgcctgtagtcccagctactcgggaggctgaggcaggagaattgcttgaacccaggaggtggaggttgcagtgagtcaagatcatgccactgcactccagcctgagcgacagaggtagactctgtctcaaaaaaaacagaaagacggagtcttgctctgtcacccaggctgcattgcagtggcatgaactccgcctcctgagttcaagcaattcttgtgcctcagcctcccaagtagctgggattacagacatgtgccaccacacgtggctaatttttatagttttagtagaggtggagtttcaccatgttggctaggctggtcttgaactcctgacttcaggtgatccacccgccttggcctcttgaagtggtgggattatgagtgtgagccactgtgcccagccaagtgagtatttgcttatgtagtattttaattttatgatttttttttctttgagacggaggtttgctcttgttgcccaagctggagtacagtggtgccatctcggctcactgcagcctccacctcctgggttcaagccgttctcctccctcagccacctcctcctgaatagttgggattataggcgcctgccaccatgcctggctaattttttgtatatctagtagtgatggagtttgagcatgttgccaggctggtcttgaacctctgacctcaggtgatccacctgccttggcctcccaaagtgctgggattaaggcatgagccaccatgcccggccagagactgttcatttattttttttttttgaggcggagtctcgctgtattgcccaggctggagtgcagtggcacaatctcggctcactgcaagctccgcctcccaagttcacaccattgtcctgccttagcctcctgagtagctgggactacaggtgcctgccaccacgcctggctaattttgtttttgtatttttagtagagatggggtttcagcccgccttggcctcctggagtgctgggattacaggcgtgagtcagggcgcctggccaatcataccttcttttactgcattaattatggttttctttcgttcttaaaacatgtttatagtgaccacttttgaaattcttattaagtcagacatctggttatacaagcaatttctattgcctacttctttttccagtgggtggggttatactttcctgtgtcttagcttgtcgtttttttttttgttgttgaaaactggacattttaagtaatgtagtaactctggatacctcattagcctatggttgggggtggtggttgttactgttatttgcttatttgtctaatgactggctgaatgattttagtgttctatccttcttccctccctgtacagtgtgacacgtctgatgctagttttcttgggatgcagccttgggtatgcctaccatcactctagaatcacagtgattttggcatggctttgtctcttttcctgactgtacccagctgttaagctacactaattactaggtgatgctgtgtagtcatttcttggtgtccttgggggattggtcccaggacccccccgttggatataaaaatttatggatgctctagtccctcataaaatggcacagtatttgcatataccggtgcacatcctcctgtatgctttgtcatttctagattacttataatacctaatatggtgtaaacactaggtaaatagttgttatatattttttatttgtcttatttttattgtatttatttttaagtgtttttaatctcgagtgattgaatctgaggatgtgaaatctgcagatatggagggcctgcattgttttccgtggagctttgggcctaaactgctccacagactgatctgatcaaatttgcgcttctttgaagggatagtttctgagatcagtgtttgaaatttgttccaatccacagaggagtcctcccagctctctttccctagttctggccaccaaactagacaactacaatttagcacttatctccaatgattctcctcctaccaagtgcctttgaaagcatcattaactctttcataccttgttgcaaatgaaatttctttgggaagagattgtgagttttttttctcctaaattatggtgcaatataagtaatataccattttaacaattttaagtgtattaagtgttttttttttttgtagtttttttttttttgttttttgagatagtcttgctctgtcgcccaggctggagtgcagtggcacgatctcggctcactggaacctccacttcccgggttcaagtgattctctggtctcagcctccccaaatatctgggattacaggtgtgcaccaccacgcctggctaatttttctatttttagtagaaacggggtttcaccatattggtcaggctggtcttgaacttctgagctcgtgatccacccacctcggcctcccaaagtgctgggattacaggccttagccaccacacctggcctatgcattgcttttatatgtattttaaaattcataagttctcctcctatgatgtttttgtcccatgtgatttatttgttaaaccgtcatctttggccgggcgtggtagctcacgcctgtaatcccagcactttgggaggctgaggtgggtggatcacaaggttaagagatcaagaccatcctggccaacatggtgaaaccccgtctctactaagaatacaaaaattatctgggcatggtgacgcgtacctgtagtcctagctacctgggaggcggaggttgcagtgagccaagatcgtgccactgcactccagcctggcgacagagtgagactctgtctcagaaaaaaaaaaaaacaaaaaactgtcattttttatgttgcatttactgcattctggatttaaactgtgaggaacctcatggtatcagttaatatattcttccatcttaatgtttctcgtaaactggtagatctgtaaacttgattaggtctatcctattgtatcacatcagaagcagaaggtgcttttttttttttttaagggaaattgtgtgaaagtagacagaatggtaaagtgaacccctgcacacctatcacccagctttaatagttatcagctcataccattcttgtttgatttacaaccccattcatttctcccttctgtattattattatttagttaattattttttgagacagggttttgctctgtcaccaatgctggagtgcagtggcataatcacagctcactgctgtcttgacctcctgggctcaagtgatcctcccacctcaccctaccaagtagcggggaccacaggcgtgtgccaccatgcctggctagttttttattttttgtagaaacagggttttgctttgttgcccagactgatctcaaactccggcactcaagtgatcctcctgcctcagcctcctaaagtgctgggattacaagcatgagctaccacattcagcatgtaaatttctttatattaatttgactggcattttaagtcacacttgaatttcatatttggcaactattaaaagcatagagtcctggatattagtgttttgttaaacctgatctatctaatcataaatatacttaggtctaaaatatgctcttggcctttgtttattgcggttcagtatttgttactatattaaatagtaaaatatttggtttgagatactaatgaaaagattaaaagtaaagcataacttgaatggatacaaaaagaaacaagaatttagacttcagtggatttcagagaatactgcttcgatatgctaacattcctgttgggtgtccaaccgtgtcatagatcagtggaaattagtggtttctgcactttactgtactgtttttttatatgataatattttcctggttgaatgattcgttcttttgagtaaactccatggtcaaacaattactttttattagtcaaagatgtaaccacataatcactaaaaagaacagtgtgacttatttaaaggggattatgtttttaagtcttttatatagctttgtagggaggccatatgagtttaaggacagttcgtggcatttgttcaaggttttgtaacttggcatctcagcagccaccaggataccagatcatcgttctaagtaagatttaggcattttagccttcatgtacagactataagtacacccccccacacccctaccaaaactgtaaattcaaatgatgtttgaaaaagcatagaatttttgttaggcgaggtagtttattccttgtgatacagttccagagaggcagcataacctaggaatgaaaaacttagacgtggaatcagatacacctggtttaaataccagctctactgctcatgaactggatgattttggtcaagatacttgactgctgaggttcagtttcctcacctgtaaagtagaggtgatagattagacatgttgcatgtgaagtacttagtatggtgtctggttttgtagtaagatctataaaagataaattattagtcatattccttagacttcaggaatttatctctgtgccatgtttgaggcaaacagttacagaattagaatgttagaaatgaaaggaatcctagatgtcatttaattcaagtccattgttttctggatgagagaagaaagtgaggaaaagtgacagagttggagaccaagctaggactggcctcagaatgttaagagtactcttctagggatcgaccagtcgtgttactagactttttggatctgaattgtgcttttccttgaatgttttgaattttggcttgagtgttgtgattattttattaaaatgagattccagtcctattgtcatgactaatgtttatgagaaatataacatttcactttaatgatgttttttaattattctaaggggcctaatctttttcagtggaataagctttaggttgtattatattctataattcacttgaaaatagaattcatctttacttgacagccaaattttgtgtactgcatcttttctgagggagagagttggcaaggaaaggcacttgttacaacgatccacacatatagacgcatattatttagaaatgaaagtgctttgaatgatttagcttattttcagtttttttttttctgcagttgtaatcatatgacctgtttttctttctttttttttttttgagacagagtcttgctctgtcaccccggctggagtacaatggggcggtctcagctcactgcaacctccacctcccaggttcaggcgattcttctgcctcagcctccctagtagctgggactacaggcgcatgccaccacacctggctaatttttttattcttagtagagatggggtttcactgtgttagccaggatggtctcgaactcctgaccttgtgatctgcccacctctgcctcccaaagtgctgggattacaggcatgagccactgcgcccggcccatatgacctgtttttcttttatagatgggggagaaatatgggaagtgacttggtgtcagtcatctgtgttggttaaatcaagaatataatccgtgttttgcttctgaatagctctttataacagtgattggttactttgggagtaaagattattatttagagacagagtcttgctttgtcgcccaggctagactgcagtggaatgatcgtagcctactgcagcctcagactcctggactctggtgatcctgcctcagcctcctgagtagctaggactagaggtgcatgccacatgcctggctataattattattaatttacgtttagcattagtttttttcttccagtaggctattttactttatttatttgattttgatgaagtttgattatttctagtttgcttccttctatgacccctacctgttgtgggtctccaggcaagcagtgcataggtagagccatccttaggtagcctttagacttaatattaggtgagctctccccacagatagcctctcctttatttgaatggaattatattttaagtttggaaatatttttcagcttatttagcctgttgaatttaataaaaataatatttaatcttttcagaggtcgaaacagtaacaaaggactgcctcagtctacggtgagtaactttaatgttacttattggggaaaattagtagctaaaacatgatctctaaccacagaccaaatgccaaggcaaaagattcccttcttttgaattttgtcatagataacttgactgtttaagtatgttattagcctatatgtgtttttttaatgactctgtataaaatgtacaattacttgttgtattagtccattcttacactgctaataaagatatacctaagactgggtaatttataaaggaaagaggtttaattgactcatgctctgcattgctggggaggcctcaggaaacttacaatcatggtggaaggggaagcaaacacatccttcttcacatagcgacaggagagagaagtgctgagcaaagcagggaaagccccttataaaaccatcagatctcctgagaactcactcactatcatgagagcagcgtaggggaaactgcccccatgattcagttatctccacctggtcttgcccttgacacacgagaattattataattaaagataagatttgggtggggacacagaaccaaaccatatcatttgtaaatagtatttttgtcacgtgtaataacaagaacaagtcgcttgttcttttctaaatgactaagtgcaaatctaagtgaaaaacctccaaaagatacgtagaacaccaagagtggagtctgcagagttctttatgctttttattttgaattaatgtgctttttttctgctgctttcatttttctcctttggctttctggtcttaaattttggaatgttatcaatgaaattgaaccggacatgaagggcagaaactataagtcccacatgatggaagaaataaatgagaagctatcacaaatttttgagactttgcctttattagattgttttacaagaatcaggaagatatacacgtatatggtagtaatatggagtagtgtggttgatcagacttaagcactgtcactgatgctgatatgctgggagaacctagtcagggttcttctatgaaggtatgacctggcttcctaccccatttatttatacttcacccttcttagggtacatttctgtgagttttaacaattgcatacaatcagtgtaactaccaccacaatcaagttaatagaacagtttcattgcccaccaaaatccctcaaatcacttttcagtgaaccctcctctctctccaaccattgatttgtcttccatccttacggtttgtgtccttcctcctctatggaagtttactcttgcttttttatgtcatgtttagtcaaaacaccattagttggtttgactgataacacttgaaaacctgaccttctgttccttctgttctctatggaagcaaaatattaaataaacaaaatcttcccttaatacatgtaagatatcataaacctaactaaacattttgcaacaaataataaacgttagctttatatgcaaatgtaaatacaggctgagcatccctaatcggaaatgctccaaaatttcatattttgaattagggatgttcaagcactaagtataatgcaaatatccccaaatccgaaaaaaatccgcagtctaaaatacttctggtcccaagcattttagatgaggaagattcagtttgtactaatttctaatagttttttttttttttaatattccagatttcttttgatggaatctatgcaaatatgaggatggttcatatacttacatcagttgttgtaagttattagattattggggataaactgccttgggggtagaataaagtaattccatgaagttaaaatgtggataaatgattgtcaaagtaacattgcttagatcatgtttagtcaggatgatttagagaaatagattagaactccttttatccagtctaatataattcattgtaaaagtacagttggtcctctgcatctgtgggttccatattcatggattcagccaaccttggatcaaaaatatttgttaaaaaggccaggcacagtgactcacgcctgtaatcccagcactttgggagtttgaggtgggcagatggcttgagctcacaagtttaagaccagcctgggcaacatggcagaactccgtctctacaaaaagtaaaaaaactagccgaacgtggtggtacgtgcctgtagtcctagtgacttgggaggctgacgtgggaggattgtttgagcctgggaggtggaggtttcactgagctgagataatgcccctgcactcagcctggtcaacagtgccagacagaccccttctcaaaaaaaaaaattttttttttttttttttttttttttttttttgagaaaaaagaggcatggttgcgtctgaaccaaagatgtacggacgtttttcttgtcattattcctaaaacaatacagtatgacaatttacatagcatttacattatattaggtattacaagtaatttagggatagtttaaagtatttgggagaatgtgcttagttatatgcaaatactattacattttatgtaagtgacttaagtattatgtaattcggtatctgaaggaggtcctggaaccagtcccctaccaataacaacagatagctgtattcttgttaaccctgctgtgtgtgtaaaataatgttagtagttgattgtcttttgtacattattttgtcacttaaaatagctggggtcagaaatgtttgacttcagtattaaaattcgtactgcaaactctgagtagagcctcctgaagaatttcaagagttcagtgtattgttaatgttttgaaatttttttattgttttgttagtgaatacctaatattgaatgaagcctgatgaggtataaaaagtaaaatgaaaacaaatatccctggtgaccgggtagtatactgtttctttgataaataaattatatgtttttagggctccaaatgtgaagtacaagtgaaaaatggaggtatatatgaaggagtttttaaaacttacagtccgaaggtaatttttactttttttctttttcttacaaagtaaaagaacattttcatagtcagtgttttacctagtttttaaagccactttgaatgattttacttctcagtttcaaatactgattattttatagactggtttgtgtaatcagagaggcttcttgatgtgtgtgcttattaaaatatttcaaccatttttaagcattgtgagctaatagagggatgtggtggtttgttttttcctcttaaaaattattattaatgtacttaagacaaaccatagaaacaaaaaacatttagatatgaggatttttaaatgatggaatggataatagatcatatgcctgggaaaaagggtatgattctcttgagattatttttgtcaaaggcatataagaactggtaccttgatgagctaaagaattcctaacaaattttattttgtaaaggtttggagtacttacttgtgtttttcattttagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagaggtgggttttgatttcctaaatatgcctcatggtttattagatttattcaagcaaagattttcacagtgatcttacaaactttttttaaagaaatatctgggctgggtatggcggctcattcctgtaatcttagcacttagggaggctgaggcgggtggatcacctgaggtcaggagttcgagaccagcctggccaacatggcgaaaccccgtctctactaaaaatacaaaaatttatttttgtgtgtggtggcgtgcgcctatagtcctagctactagggaggctgagacagaattgcttgaacccaggaggcagaggttgcagtgagctgataccgcaccactgcactccagcctgggtgacagagcaagactccgtttcaaaaaaaaaaagaaagaaaaaagaaatatctactttctagaatagcccaagtaaggtaattttttagaaaaatgagaatgttaatgcatttttgttggaaaacaattagaactttagagaaaaattaaatagagtttttgtgatctcttaaaaaattagtttgtaaagcattttctacagttttgtggtcaagaatgctactgattatattcaactgaaaatttcttgtcccatttggcctacaatgctttagtttataagtgggcatgtggcaaatctggaaagaaatcaaagtataaggctaaggaagaaaggtagagaacggttggtagaaaacaattgtctaatgaaaatgaaaaagggtgaagaagtagaacatacgtattttaaaaatattcagagtatgagacaaggttttgagaatttaaaagcgattatgtagttatattaaaaatttagtctctttttaagtgtccattgatgaacaaagtgggaattcctgttactcatttgcaaggcattattgagtgttcagtaacacgttgcaaggcacttctgggcaatcctgaacttggttctcaaattctttttttttttttttttgagacggagtcttgttctgtcccctgggtggagtgcagtggcacgatctcggctcactgcagcctctgcctcccaggttcaagcgattctcctgcctcagcctcctgagtagctgggactacaggcgtgtgccaccacaccaagctaatttttgtattttttgtagagacagggtttcaccatgttggccaggatggtctcgattgtttgacctcgtgatccgcccgcctcggcctcccaaagtgctggaattacaggcatgagccactgcacccagccggttctaaaattcttttatttatttgtatatgccaaattctgtagtgaaatacgtaattctgttgtaaattgtagttcagtacaatttgattttcactattcaaatctataccaaaagctgtttttattgttgggctgattcttctacactgttacttggaaataataatataccaggattctttctcttagacttaggagtctttctctttgcttgctttttcagaggctaacagtactgggtattctttaactgtcttgatatgctgatgaaagcacagtgttctgtttttgaatcttctcaaatgtccttgtctttgattcacaactttttgtcttaagaggccttcagcatcccatacaaggaaacaagtctttttttagctgctacctttggagttgattttgtttatgtctaggagcactaaattatttatacttatactattgaaatattcctctgttataaattcaaaaattgactttggaagataaaattttagttgaatttaatacatagcactctggaaagagtattggccacaacaaaaaaaaaggttccctactctattggataccaggtcatttaacagccatttacggtatgcattgtctttttgtttttatgatgaattgatatttcccaaatgtggaagagtgaatattactttgagatgtttgtgatagtccattccttgctcctcttcaaaattaatgtcattaaatttttattactttattagatcttcatttctcagataattttagttcattatagaaaggcaagaaaatacagatcagagtgacaactttgaaaatctcactctactcataaggggatgggtgtattttgctatatattacaaaattagttttcttgatgaggacatccactattggagtaatttcaggtatcttattttttcttttctctctctttttttttttttttttggagacggagtttcgctctgttgcccaggctggagtgcagtggcctgatctcggctcaccgcaacctctgcctcctgggttcaagcgattctcttgcctcagcctcccgagtagctggttactgaggcatgtgccaccatgcccggctaatttttgtatttttagtagagacggggtttcactatgttggccaggctggtcttgaactcctgaccttgtgatcctcctgccttggcctcccagagtgctgggattataggcgtgagccaccacgcctgggcaggtatcttatttcaaaacttacagtggtttagtgaattatacaattgcgtccagtgcgtagtatcctgaaaatagtattaagtcatgtgtttaggacatcaggtctcttaagctaagactatccaggcagaaattgccctcttctataaaagaagaaaagtattaattaggaagtactatcagtatggagaaaaccattttagaattattaattggcatggtttccttctttttttttttatttcgagatggagtctcactctatttcccaggctggagtgcagtggtgcgatctcggctcactgcaacctctgcctcctgggtttaagcgattctcctgcctcagcctcccgagtagctgggattataggcacataccaccatgccctgctaatttttttttttgtttgtattcttagtacagactgggtttcaccatgttggccaggccgcatggttttccttaataacaaaattaaggcatttattactgcatctagattttttttattttttattagagacttactcagattactcccaaagtaaaggaaggtatggtttaatcaatgcttcttaatgctgggttcacgtttagtcacctggggagtttttaaaaatgttctcacttctagggatcctggtttaattataattagcctgggtgaggctctggacagtcagggtgtgagctatgggtttcatgtgatgagatcccaggagtggctctgttctgtggccttgagaatttgtgctttctaggccaggtgcggtggctcactcctgtaatctcactttgggagaccaaggtgggcagatcatttgaggtcaggagttcgagaccagcctggccaacatgttgaaaccccgtctttactaaaaaagtaaaaaattagcgggacgtgatggcacatgtctataatcccagctacttggggagaggctgaggcagaagaatcgcttgaacccgggaggcagagattgcgagatcatgccactgcactccagcctgggcaacagaataaaaaaagaatttgtgctttattttcttgcctcacagtcccctttctgtctcagaattggcaactgcctgaaatagtctctgctgttatcatttgatagtacttttccacatcttgaatggatagatagagtgttttttataatagaagtggatgaatgattagagtatactaatatgacattgtattttcctaaaagatatgaattgatttcatttctgagcttttataattctcttctgtaatagtctgtcaaattattaaggttgataatattaactaaaatttgagtgcatattctatgtgccagactctgtgctaacagatttacctacatttgttcacataatcatcacaagttgtttctgtagtagatacagctattatccacgtcatagatgaggaaacaggcatatttaggaaacttgctaaagtgaggacacaaatctagcttttctactctaactcatgttcttaacattatactgcagtgacataaattatgtggtttggtttgttgtttatctcagttgtcataagtcgaattaatgtttgtttgtttgttttgagacagagtcttgctctgtcgcccaggctgggtacagtggcgtgatcttggcgcactgcaacctccacctcctgggttcaagcagttatcttgcttcagcctccctaataactgggattacaggcacgtaccaccacacccgggtaatttttgtatttttagtagagatggggttttaccatgttggccaggctgatttcaagctcctgaccttaggtgatccacccacctgggcctcccaaattgctgggattgtaggcatgaaccactgtgcccagccagtaagttccatggttgttaaaggatttctccacaaataaagctaaaagtaaaaaaaaaaaaaaaaaaaaaaaattctcaagcaatataagatgcagactattatgttgttcaagttttttttttttttttttaatctttggctttatttttggggaaaccttttttttcttttttgttttccttgggacggagttttgctcttgtcgcccaggctggagtgcaatggtgcaatcttcgctcactgcaacctccgccttctgggttcaagcgattctcctatctcagcctcccgagtagctgggattacaggcatgtgccaccatgcccggctaactttgtatttttagtagagactgggtttctccacgttggtcaggctggtcttgaactcctgacctcaggtgatccacctgcctaggcctcccaaagtgctgggatcacaagcgtgagccaccgcgcccagccagggaaacctttattttgaggcggagtctcgctctgtcacccaggctggagtgcagtggcgtgatctcagctcactgcaacctctgcttcctaggttcaagcaattcttctgcctaagcctcccgaggagctgggattataggcgtctgccaccatgcccagctaatttttatatttttagtagagacggggtttcaccatattggccaggctcttctcaaattcctgacctcatgatccacccaccttggcctcccaaagtgctaggattacaggcgtgagccaccacactcggctgctggggaaaccttttaacatgagtaaggtcagtgtgacttttaagttcttgatgctaacatcattgatttcaataaagtttaaaagttatattcatgcatatatgcaaatgaataaaaggctttgaaatagtgacttcttacggtacagtgaataagtttcctttggtctcttgaatgttatacatgttccagtttgatttactgagaaactgaaagtacctttacgtcatatgagctgtgagtcaccttggcacattcataattagaagagaccatcagattatcattggaaaatcagtttgtatttatcctttatttgaattccagtgcagacagatctgaggttctcttcattttgctaaaacttcttagggccttcagtcgcttttggctctgtattcgtgtatctttggaattgtcctgttatctctgcttgttttttacttgattttccatccatttccagtattcctttctcctctatttttttccttcattttctttctgctcttcctgttgcgccattattcatgttttcctctttactccaactcaactatggctttacttctgtttccttattccattgttcctcatactttttcctactgcttcattttctttgcagtattctcagcctagatgataggggtcagcaaatctgctcatcagtaaataaattttattgtagcatagctatgcccatgcgtttgtgcattgtctatggctgttttgatggctgtagccatagagttgagtagttgtagctgactgtaggacttgcaaagccagaaaatttgactgtctctttacagaaaagtttgccagctcttggcctaaatcatattttccgctgcatttagggctttttaggactgatcaaaaatacatgctatactggctttggtgaagtaacagaatgtgctctgtcctttaaacttacaactaattgcatgctttgattctaatactgtataatatcctgcgattcttattcatgaccattctaattggatttagtctgaagaattacttttgcttaacagattctttgtcacatttagtgaaaaatcataaaaggggaaggttggttaatggaaaagatctccatcaactaaccactaccttccttatctacaaatttatcttcttcctccgtgccatctttttttttttttttttcagatgatcttgctctgttgcccaggctggagtgcagtgatgcaatcacagctcactgcagcctcgacttcccaggctcaggtgatcctctcacctcaacctcctacataactgggactgtatgtgcacatcactatgcctgactaattttttatatttatattttttgtagagatggggtttccctgtattgcacaggctggtctcaaactgctgggcctaagagtcttcccaccttggcctcccaaagtcctgggattacatgagtcaccgcacccggcctcattattatttttcctctggttttagtagagaggatttttaagccaacttcaatcatgcccttgactctctcccttctacttacctccttgttctctttttctttttctttttttttagatggagtctcggtctgtcacccaggctgaagtgcagtggcgtgatttcagctcactgcagcctcagcctcctgagtagctggggctataggtgcctgccaccacgcccggctaatttttgtatttttagtagagatggggtttcaccatgttggccaggctggtctcgaactcctgacctcaagtgatcacctgcctcagcctcccaaagtgctgggattacaggcgtgagccaccacgcctggccatctttttttttctccttgctcttttataccacttctctgtttctgggctcttcaacatctgcctttctagttaatctttccctttagcatgaaaacctattcacttcctgctcatcctaaaaaggattcttttttgttttgttttgtttttgtttttgagacagagtctcgctcttgcccaggctggagtgcagtggcactatcttggctcactgcaagctccgcctcccgggttcacgccattctcctgcctcagcctcccgagtagctgggactacaggcacctgccaccacgcccagctaaatttttgtatttttagtagagatggggtttcaccgtgttagctaggatggtctcgatctcctgaccttgtgatccatctgcctcggcctcccaaagtgctgggattacaggcatgagccaccgcactgggcccaaaaggattctttttaatcctgaattcttctagccattatcctgcctaaggctacgattaacctctaactgccaggtcctttggaatctttttctgtctttattgctgcacttgaatgttggtttcaccctccttcagaatttcctcttctgtattttttatgtttattgatcattccttccctgcctcattcctgggcttcttttccttcacacaccccttagatgtgtgtccccagtgtttgtttctttgcctgctgctcttgccacatgacacacactgccagctaccacacacaagttccctcctatcatgtgtgtatcattgcccttataccatgttgtattaaaattatatgcttgtctcccctgttacagtttgagctctttgtgctccaagtaaagacagtgatactgtctttattatttattctcatggtctagtatagtgctttggcacatagtacaggctcaatataaatgtgtttgaataaatgaaattcagtgccttaatacacttttgtagaagcattattttatggaaagaatgaaaaagctgtaagtggtcttacatatatagtcatccagcagatacttagagagctctgggatgtgttccttgctgtgcttgttgctatggacagtacggagaaatacaagaatctattttgggtcccttttgagaacctagtgaaactgtgtacctagtgaaactgtataccctcaccctagaaaaatttacacacatgtagattttacatgtaattcttttaaaaattaattttttttcttttttttaaagaaacagggtcatgctctgtcactcaggctggaatgcagtggtgtgatcatggcttactgtagcctcgacctcctggctcaagcgactctcccacctcagcctcccaagtagctggggctacaggtgcacgccgctatgcccggctaatttttaaaaatattttatagacactggttctcactatgtttcccaggctggcctttacctcctgggttcaagcaatcctctaccttggccttcaaaagtgatgggattataggtgcaagccactgtgcccacgctaatgtaatttcatggtgttcacagtttcttcagggagttcatatacgccatgtactctattctaagcatttttagagttagagatagcaaagcacgtgaataaattcaagaaaaatggaatgttgtactgcatgacattgaatatcaaatggagtcagcgatgcaaataattgtctagattttacaaaaaaaattagcctggtgtgctggtgtgcgcctctaatcccagctactcgggaggctgagacaggagaatcatttgaacccagaaggtggaggttgcaatgagctgagatcgtaccactgcactccagcctgagtgacagagcgagactccatctcaaaaataaaaaataaaagaattgtgtagattttagtagttggaagaagttggagtgttaatgtgtaattagagaacagtgagaaataaaattctacagattgttttattctggtgtgctgttgtgttctcatatggttgtctttttggtcttgatagtgtatcagtaacagagtacgagtaacaaacagggatctcttctgaacggcgtgacattagaaaagctgtttacggcctcaactttgctgtggtttattaagacacagatatgtgttcattctggggccaagcagtaactggagagtggcacttattgaggccagtatggaggcagtacagagattattgagattaaaagaaagaaacaggtggaacggatctatgtaatggaaagctaaacagaatagttcgtggtacacagtagaaaagcattacatgtttattaagatatggtcatcttccatttattaaagttacatgttttataatttttagagtatatagaaattctctaccctatcatgtttgccaaagtcagaacaataacttcatttattaaatataaaaaaaataaaaacctctagcataaaatagaattttatttggacaaacgataaaaaaatactgtgtggtactagtaagagtaaggttgattcaagatacatgggagcagaatccaaagtgtagaaataggccaggtgcagtggctcatgcctgtaatttcaacacttttggaggctgaggcgggaggatgagttcaggagttcaagactcgccttggcaacttggcaaaaccccatctctacaaaaagtacaaaaattagccgggtgtggtggtgtactcctgtaaacccagctacttggtgggctgaggtgagaggttcacttgcagccagtaagtcaaggctgcagtgagctgtggttatgccacggcactccagctgggtgacaagcaagaccttgtctcaaaaacaaaccagccaggcgtggcggatcacctgaggtaaggagttggagaccagcctggccgacatggctctactaaaaatacaaaaattagctgggcgaggtgacgggcacctgtaatcccagctacttgggaggctgaggcaggagaatcgcttgaatccaggagacggagtttgcaatgagccgagatggtggtgctgcactccagcctgggtgacagagccagactctgtctcaaaaacaaaaataagcataggacatggggataaattgaagatttatgaagacacagctgaaggagacataaaagtagatttggctaaatggaaacatgccatactttgaatggaattatttaatactacaacgttgtcaattttcctcaaataaatctctaaagataatatattcagttttggccgggcacgttggctcacgcctgtaatcccagcactttggaaggctgaggtgggccgatcacttgaggacgggagtttgagaccagcctggccaacatggtgaaaccctgtctctactaaaaatacaaaaatcatctggacatggtggcaggtaccagctacttgggaagctgaggcaggagaattactcgaaccccgtaggtggaggttgcagtgagctgagattgcactccagccgggtgactccatctcaaaaaaaaaaaaatttttataatatatatatatatatccgtttttgtagaaattgacaaaatgattctaaagcttattagattatgtgtattaacagaagaactttggaaatttttttccacaagagtcataaaggaggacttgccctacaaaatatgtcagaattaaaacataacttgtcagctgggtgcggtggctcacgcctataattccagcactttgggaggctgaggcaggcagatcatgaccagcctgaccaacatggagaaaccccgtctctactaaaaatacaaaattagccggtcatggtggcgcatacctgtagtcccagctactcgggaggctgaggcaggagaatcgcttgaactcgggaggtggaggttgcagtgagccgagatcgcgccattgcactccagcctgggcaacaagagtaaaactctgtttcaaaaaaaaaaaaaaaaaaaaaagaattataactgtcacagtggctacgtatggagcatccaaaactgaatttatgtgggtattttattaatatgcaatatagcactttaattctggaggaaaggtggattattcagtaaatgattctgggacattggggacaaattagatacctacttcacactgataaataaaaccaaatagattaatgagaaaactgtgattaaacaaaacaacacccagactacactggagcaaatctgtgaatttgtttaattttgagtggagaaggactttataagcatgactaccagagcaaaaaaatcatgaagtaaaagatcgatacctttgattataaagagattaaagatttaggccgggtgtggtgctcacgcttgtaatcccagcactttgggaggccaaagcgggtggatcacttgaggtcaggagtttgagaccaacctggtcaacctggtgaaaccccatctctactaaaaatacaaaaaaattagtcaggcatggtagcacatgcctgtaatcccagctactcaggaggctaaggcaggagaattgcttgaatttgggaagtggaggttgcagtgagccgagattgtgccacatcactccagcttgggcgacagagtgactccatctcaaaaaaaaaaaaaaaaaagacttagacgtgtccaaaagtaccatacatttaaaaagacatgccacaaactgggaaaagtagaaaaatagttttaaaaatgaccagtgaatgtatgaaaaggtggccctcctcacttgtaatgatttaagaaatgcagtttatttttattttattgtatttttaaagaaattcagttttaaagcagtggaatatgattgtctatcagcttgcgctgaatggtaaatgtgagaaagattactactacttagtggtactgagggagttgcaaaacacttaacactgctagtgggatggtttaagtaaaacaagtagcattcttaaactctctattaggtaaagaataggtaagtaatgcatatgtttccaggacattttcagtaagactgtttactgatagggttgtgtaatgctaatatacttactatctagttttagtattatttttttctcttgtcttggatggtttcaatggagtcttatgcatgcagatatattaaaactagtaataaagcaagagaaggaatgtggataaattatctctaatttctattttgttctatttctatttcatactcctgggaaagaatattaagtgggcatgtgtacttgaacagttgttctgttttttattagaaaagaatccgaatctataaaatgttttacatatttgccagggaaacagaaaagatatttgtacagctgtaagaattggaattaatttcattttactgacttttccttaacctaattctgaacacttttgccataggtttgagaataagttgttataaaatgactactattcttcactaatagtattggcatttcaattcctaaattctgttttttgattcttgaacatttctgaatttactttttttgtcttagttcttctacagaatcattttcttcttttttctttttttatttttattttttatttttgagacagagtcttgctctgttgcccaggctggagtgcagtagcgcgatctcggctcactgcaagctccgcctcccgggttcatgccattttctcctgcctcagcctcccgggtagctgggactagaggtacccgccacagcgcccggctaattttttgtatttttagtagagacggggtttcaccgtgttagccaaggtggtctcaatctcctgacctcgtgatccatccgcctcggcctcccaaagtgctgggattacaggcatgagccatcgcacccggccttctttttttctttctctttaacttctgagctgaaaatagtaccttttataaagaagtgctcaaacgatgattggactgatttctccttatttctctctttctctctgtctctttcactctctttttagaatttttcttttttaagtagagacgaggtcccactatgttgcccaggctgtcttgaactcctgagcccaagcaatcctctttgcctcagcctcccaaagtgctcggattacaggcttaagctatcacaccaggcctaggctaatttcatattttgagatggcacaaatttctttcaggtagctagcttttcctcctcctccccacttaaaatagatcctgatccagaagcctaatggagaaaatgaaaacagaatgttcacccataaacagtatctttgtattggaatcttttctaaaacttcttttgatctttttaggagatagtgtgggaatcagcaatctagtattacgtacgtggaatctgtcaccttgtttttttaaatacagcaaacctcatgaagtgaatttccatattttttcttgttcttgttagttttgcaccactcaggctttgctgtagaatttgatgtatatttgattctgtagagcatgggctattgatcttcactcagctttcagaggaatctgattagtaagtttgagttttttattattttttagttgattttgaagtaaaatacagcaccattttaactgataccatttctaaacaattttcagttcaaattttaagttagctaatttagagcttaagaaaattgctttaaaaacataaaattactggctgggtacagtggctcattcctgtaatctcagcactttgggaggccaaggcagatgaattgcttgagcccagtagttcaagaccagcctgggcaatatggtggaaccccgtttctacaaaaaaaatacaaaaagtagccagacacggtggtatgtacctgtagtcccagctattcgggtggcagaggtgagaggatcatctgagcgcagggagattgaggctgcagtgagccaagtgagaccctggtttcaaaaaaaaaaaggttactaattgcagtgccttttatcttatttaatgggcttagtcaaactaagatgatgtattttatcttataaatgttttcccttgaattttaactgaagaatccaatttgtacctctcacaaacagaatgtattagtaaggaaaataaatactgctttttattacttaaataggatatatttttctcttagggattttttttctattttatctcactttatcgtagtgctagaaaatttaatcattcatttgagatagggagaaaattaggtttttttttttcttctattttgagacagggtctcattttgttgtccaggctggagtgcagtggcgccatcgtagctcaccataacctcaaactcatgggttcaggtgattcaccttagcctcctgattaagctgggactgcagatgtgtatcaccactcctggctaatttttgttgttattttttgtttgatgaggtctcattatgttgcccaggctggtctcaaactctgggcctcaaatgatcctcctgccccagcctcccaaagtgctgggattacaggcatgaacctctgctcccagcccattttttaaaatatattcacagcattgtgcaaccatcactacaatcaatttacattttcatcaccctgaaaagaaactctgaaccccttagcagttcctctctgtttgtttcaattttccccagctccaggcaactattgatttattgtcttcataggtttgcccattctggacattgcgtattaatggaatcatataatatatagcctttttttttcttttttttttttgaaacagagtctcactgtgtcgcccaggctggagcgcagtggcatgattgcagctcactgcatcctctgcctcccaggttgaagcgattctcctgcctcagcctcttgagtagctgggactataggcgcctgccaccacacctactaattttatatttttagtaaagacggggttgcaccatgttggccaggctggtctcgaattcctgacctcaagtgatctgcccacctcggactcccaaagtgctgggattgcagccatgagccaccgcatctggccatatatattatgataggcttgtttcacttagtatgtttcttccatgctgtagcatgtattagtacttctttctttttcatggccaaatattccattatacagttacacaggtacactacattttgtttattcatcagttggtggacattttcattgtttccaccttttgatttatacataatcctgctgcgaacagtgacttttaaagtttttgtgtgggccgggtgtggtggctcatgcctctgtaatcccagcactttgggaggctggggctggcagatcatttgaggccgggagttcgagaccagcctgcccaacatggtgaaaccctgtctctactaaaaatacaaaaatgagctgggtgtggtggcgtgcacctgtaatctcagctactagggaggctgaggcagagaatcacttgaagctgggaagccgaggctacagtgagccgagatcacgccactgcactccagcctgggtgacagagtgaaacttcatctcaaaaaaaaaaaaaaaaaaaaaaaactgcgtgtggacataggttttcaattctcatgggggtgtgtgtgtatgcatactcatacatacatacacatacctgcaagataattgctggctcgtatgctaaatctatgttgaaccttttacataactgttgggctgttttgttttctttttattattttttgaaaatagagttggggtctcactgttgcacaggctgatttcctgggcatagtggctgtatcattttacaatcctacatagctgtttccaacgtagctgtatcattttacaatcctactagcagtgtctgaggtttcttatgtttttcacatcctcaccagcatttgttattgtctgtctctttgattatacccatcctagtgggagagtaagaagtagtatctcactgtagattttttttttctgtttacaactttactttaaaaattatatatgcacacatggtaaaaagttcaaaacgtgtgtaccaaaagatttaacagtgaaaatagaaaataagtgtggtccttgttttcttccaccaaggcaaatattgttataatctcctaaacaacttgtcttccagatttctcattttcagtcaatcttgggcattgacataaagaaattcttagacattgcttttattagatcatctcatcccttgctcaaaatcttcagtggccactgttgtttacagaataaagttgggatgctatacagggcccttcccagtggaacttctctttttcaaccttatctctcattatttcccaatgtttttttttttttttttgagacggagtctcgctctgtcgcccaggctggagtgcagtggcgggatctcggctcactgcaagctccgcctcctgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccacctcggcctcccaaagtgctgggattacaggcgtgagccaccgcgcccggcctatttcccaatgttaatctacttattgacctactaagctggcatgttctgtgtgttagacatcaccaactttgtgccttctttttttgtttgtttttgagttggagtctcactctgttgcccaggttggagtgcagtggcgcgatcttggctcaccacaacctctgcctcccgggttccagtgattctcctgcctgagcctcccgagaagctgagacgacaggcgcgcgccaccatgccctgctaacttttgtatttttagtagagatgggtttcactgtgtttcccaggctggtctcgaactcctgaccttgtgatccacctgccttgggctcccaaattgctgggattacaggcgtgagccaccgcggccccctgtgccttcttcttttactcctggatttaatcccaacgtgaagaatctaccttactaactagagttttagatactttttcaaaaccaagcccacatctgtcctttttagagtcttctctgaccttccctgctcattgtggtttgtttttattgcctgtaacaatggctgttaaactttacattttaaattaatttatgtttgtatgtatttatttgttgagaaagggtctctctctgtcacccctactagaatgcagtggcgccatcatggcttactgcttcctgggctcaagctgttctcccatttcagcctccccatgcaccaccctacctggctaatttttttgtttgttttttttagtttagtttttgtagagacagatgtctcactgtgttgcacaggctgatcttgaactcctgggctcacttgatcctcccatctcagcctccccaagtgctgggattacaggtgtgagtcaccatgcccagactttaacattttctttttagtatagaataggtcagtttttttccctctgatgagatcccatgctgactcttagttaaaacaaggctttggttggaagaagagctagtgatgtcctagctccctacttactccactttcccttgccttctggggtgtcctgaagacatcatagggtgtcatgaagtacagttggagaaccagtggtctccatcatgtaccaaacactcatcttcacgaagcagtatgtagtgtcttttttaccggtatattttctctctcccaatgcattaaacttttctggagttcagaaaacaaatttatagaattaaggaaatgcgtcccccccaaccatggtgtctagtatatatacagtgacttacagataacaggtgttcaacatatatatattcctttgattgatttttgaaaagtttacatgtatatattttttatatacggggtctcactctatcactgaggttggagtgtggtgatgcagatcttggctcaccgcaacctcctcctcccaggctcaagtgattctcccacctcagcctcccgagtacctgggaccacaggtgcgcatcaccatgcctggctaattttttatatttttggtagagacaggattttgccgtgttgcccaggttggtttcgaactcctgagctcaggcagtccacctgccttggcttcccaagtgtgagccaccactgaaatacttatatttttaaacttaatttatttatatttattatatttttatgtttttatattttaaaaaatatttttatactcactagacccaattttatactcctaaaccagggaataactgtttttttttctcttacataggcatgataccatagacaatgattaaaattgtaattaccattcatttcttagttttgtggctgggacactgatgtcttcaaatgttagtttgcaaatacagtcagccctctctatccatgggttacacagctgtgaattcaaccaaccatggatccaaaatatatgggaaatacgctggggctgtgggtcacacctgtaattccagcacttagggaggctgaggcagatggatcacctgaggtcaggagttcaagaccagcctggccaacatggcaaaaccctagctctactataagtacaaaaaattagctggccatggtagtgcacatgtgtaatcccagctactcgagaggttgagacaagcaatttgcttgaacctgagaagtagaggtttccatgagctgagattgtgtcactgcactccagcctgcgcaacagagtgtgagaagaaaagaaaaaaaactgtctgaaaagaaaaaaaaaaattatatgggaaatcaaaagcatctatactgaacatgtacagactttttttcttgtcattattccttaagcagtaccacaactatttccgtagcatttactttgtattaggtattataggtaacctagaggtttaaagtatgcgagagtatgcaaatactacaccactttgtatcagggacttaagcatccctggattttggtatccctagggggtattagaaccaatcccccatagatgctgaaggacaactgtagtgtgtgttggaataatttattttcaaatggatcatttggagaacactattctttaggaaacatagcctcctaagttctgttccatacatccctttcacctccacggcgttgtagcatcctgctttcatgactgtgtcatcactcggaaggaactgcttctcttccagaatgcttttcaagatctactctgaccacagctataaactttacacttctattctcttcttgcccctcacagtgttctctgttcctctaagatcttaaactctgtctactcctaatccagcctgctgggtgtggctggagaaagtcccactggggggctgattagttaggaatgtagggtttccagctcttgctggagcctcagaagagttcagcagacttttttttttttttttttccttaaacctatttctgcagccttgatgaccactccttccagtccctcacctatttgctttattcatggcagaggctctttcttcctgcttgtcagtacaaagaggcaggattcttcacctggatctgtggattctcaaagaatttgtggagagaattcagggcattgatgaccttggatgaagagaaatttacatctttatttacactaaccttcaagtgaaatttagcattttttgccatttaaaaatatgggcaacaaacaactagtagtattagcagtatttatgacttaagcacctatagaactcagttaatttcatatcgcttgatgttatgggtatctcaaattattattttatgtatatatatttttgagatggagtctcgctctgtctcccaggctgagtgcagtggtgcagtctcagcccattgcaacctctgcctcctgggttcaaacgattctcctgcctcagcctcctgagtagctgggattacaggcgcacaccaccacgcctagctaatgtttgtattttcagtagagaaggggtttcaccatattggccaggctggtcaccaactcctgacctcaagtgatccgcctgccttggcttccaaagtgctgggattacaggtgtgagccaccgcacccggcctcaaattatttttagaaacagaatcttgatatggtatccgctctggccttgaacttgtgggctcaggcagtcctcccacctcagcctcctgagtagctgggattataggcatgtgccactgcaccaggcttcaaattattatgtatgttcatcacctctttaaatttataatagttattaaacctgttactggatcttaatatttaatgctttaattaagaacatgtatgttactatgccaacagatttttttagtttttgataactgcatttcattgttacttgttctcatttgatttcctgtgtattttacgaatttaagtacattctgaatacggtttcataggcttccctaaaatattgaaggggcccatggattaagaaaaaggctaagaatccctaatctagaggctccccacagtcctcttttgtcatcatacccctaccccattctagcctgaggagcgtggctccacctgtgcccttggttttgttgttccagtccatacatcctgcacccttaactgtgtttcttatccccaacttgtttctttgtgttattcttcagtattatagtctttaatataatctgtataatacatggtgtagtagtatatgctcgtagtatacaattcagttagaacagatgagtattcaatgaaaagataatctcctctctaacccccagtcccacttccctggggaagcctgtgttcttgtgtacaattcagaaaatgtttatacacatattttttatttatttattttttgagacggagtctcgctctcgccaggttggagtgcagtggcgcaatcttggctcactacaacctccgcctccctagtagttcaagcaattcaaggttcaagcaattcgcctgcctcagcctcccgagtagctgggactataggcgtgtaccaccacgcctacctaatttttgtatttttagtagagacagggtttcaccatgttggccaggatggtctcgatctcttgacctcatgatccacccgcctcagcctcccaaagtgctgggattacagatgtgagccactgtgcccagcctgttgatttaattttaaacagagtttcgctcttgttacccaggctggagtgcaatggtgcgatctcggctcaccgcagcctctgcctcccaggttcaagtgattctcctgcttcagcctcccgagcagctgggattacaggcatgcaccaccatgcacagctatatttagtagagatgggggtttctccatgttggtcaggctggtctcgaactccggacctcaggtgatccgcccgcctcggcctcccaaagtgatgggattacaggcgtcagccactgcaccccgcctatacacatttttttgttttttgtttttttgagatggagtctcgctctgttgtccaggctggagtgcagtggcgcgatctctgctcactgcaagctctgcctccctggttcacaccattctcctgcctcagcctcccgagtagctgggattacaggcgccggccactacgcccatctaactttttgtatttttagtagagatggggtttcaccgtgttaaccaggatggtcttgatctcctgacctcgtgatctgcctgactgggcctcccaaaatgctgagattacaggcgtgagccaccgctcccagctatacacgtatttttaatgccactccagtctatgttggaaccattttacttcccctttcttattttcttcttgtgttcttgaaggcctagatcagctgttgctgataggctgtcactgtcactttagaaagcccagagccttttgttccttagaactttgtttttaattgtattgtagcactcattgtattcgattctaaaagatttgcttcatttctgtaactagtctcttacacccaggagctcctagttcctacaggaaatgctgggaattgtatcagtcaaatgtgaatccccacctcgtccagacttatgagtgcattgtaggtactcagtaagtgctaaaaatgactaaatagtcccactgataccaatctatatactgatactttatatagtatatagattggtccacatataacgatgacacataatgagaaactgtcttaaaaagttgttgaaagtgccgcaggaataggaattgatcaaaacaatatgattttttaggtttatatggaactttgatgtttgagaaaaggctgatttagttgagaagaaatggttagctgaggattttgatgacttctctggaagcacatttgagggtttgtgatgttaaatctgatgttaatgattatttcatccagttttatgtcattttatagtttttatacatttaagtatatttatttctaatgtttaacactaccattttagttatttgaccattattctggccctttaaaaaatgctcagacaagtttgaatgatttttcagaggcattattggctcagaggtaaaagaggaaagattgagaagctgaatatgtactctgtttcctgggtatggggctggggatacccagaagaggttcacacgttggtcgagacatttctttatgaccaccagcaggtggcatcaccggcccaaaatgactaagtttctgcccagaatcagaagagaaggtgttgagagcccactgctgtgggggtagcatggaggtgggatacaggggctggaggtgatacaattttgtttcttcctccaacatcgcctgctagtctagaggcttttataaattgaaaaactaattctttatcatctcatctgatggtttttatgtttttccttttttctctctatacctgtagttccttcagaaacaggtaacacttttctaatagtcacgttgtattcttgcatcttgttgttacaatgcttttgtttctcaccataggggatgatggaaaattaatattctttgacttatggcattggtaaaatctgcatgcaaattcccacagttgcctgtagattagagccagttgtttttttctcaactttgcaggaatcctggttacaacattgtactatttactaccaacagtgttttttttttttaaaatccagacttgctgggcatagtggctcatgcctgtaatctcagcgacttgggaggctgaggtgggaggattgcttgagcccagggctgcagtgattgcggcactacactccagcatgagtgacaaagaccccatctctgaaaaaacaaaaacaaaaacaaattttttttaaagaaacagaaacaaaaatccaaacttgtaaccactgtaaaacaaatcagaatttacgatagtggatattattaatagtgcagaatggatacccagatcttgcttcctttctagctaatgatgcaatgttggcctgaaatgcattacttatagccagggattttctcagcatcctgatgatatagcctcatttcgtgctaactctccacttctgcacatcttcccctaagtcctttactcatctttagaaagagctacttttggtgaaattttaaaaccaaggaatatcattctttatagaatcacacttctgtgttttccccttccccatttctgtctcgaaagcgacagactgctacataacctgtgaatacttttttttaaaaaaagtttggtattgtaaacagaagatttaagattaaaatgtagcattgagaaaaatagatttattaataatgccctcttaacacaacctaaattctggtcagtggaataaagcctgggtcctaaagttttagacgcttgcttgcttttccacactggctcttacttggggatccttttagaaatttgtttagaataatactgtaaaaacatatttaagctactttgtgtgtacatttgggatcttttggtttgaagacggcttgactcaagactttctaaatattttcacacacacacacataccctgtagtgagaaaaaaatccgtttatatggttctataaaaatctctagctgcttcgagctttaatttcttgaatcaaaagagtattgtttttaatactgagcttctatctaaataaatgctttatttacttaaatgtgtgcttttcaaaaactagtatgattaagacattaacaggatcttagacgtaaaggaacagtcctgttgcttcttccagaagataatatgactcgtttggaattttcctatagtgtagttttttgtctagtgttgtgagaattaaagggatttcaggatcttaaggtaggttattatttgatgttttcttggaacattttacattcttgaaaatacacatggctaaattaatttttgccagcaatccacataactttaagataatgtagagaagaacgtgattcaggttagtatcaaataaggtcagatttctagtgccatcagtagctttcagcaaagatgaggtgttggtaagatagcattagtctcttagaatctcttagagagattttccaaaattcagccatttctagtgaatgctccattccacccccagctgagtcctgctgctctggggaactccctcagcacactcttggctcttagaattgctagcaatgggagtagtgctgctggtggagctggcagctaagcccagaggtggattaatgcttttattccctgatgtacaggtacacacactcatacctacccacacctagtttgggataagaagaggttagaattagctaggcttgaagttccatgcttaaatttgctggctcagatttcttattttggcatcactttgcccattagggagacaatgacagttatagaagcattgccaaataaaaaatccatctggaataacctcttttgtaggagtattgtgtgtttagttgttgattcgtcccttcctcctcttagtggcaacttacagtactgggaaggaacagtggctgggagcttatattcctcagcagagccagatcagcagaagtattactccttagttcgtagtaggtggtaccctatgggtccagtcatttaaatgcaagcctgtatctacagagcgtttcctagtgccatcattgcccagtgggcctttatttagctgagtctaactcccaactagagaaaatttcctgtgccagacagcagtatggtcagctaacatgtggatgctacatttgctttcataagtcagtactcttcaataacattagtagaagagaagaggacacaaagtgagagtgtgttaataggaagtccaggtatgcctgctacctgaactttctgagacaggtaatactgtagggcctgaactttgtagcagagtggttatatatgaagaagtgggttctgggaggggttaaaccacttagaatggcttcatttactaatggcaagagtttgctgggatattgaccactgtacatagacatgaatatggaaagttaaaaacaaaatccacatatatttggctgcaagtactccgaaggtatatctaattagtgcatccattaaacaaaagagatattttaggccgggcatggttgctcacacctgtaatcccagcactttgggaggccaaggtgggtggatcacctgaggtcaggagttcgagaccagcctggccaacatggtgaaaccctgtctctgctaaaaatacaaacattagctgggcgtgttggtgggcgcctgtaatcttagctacttgggaggctgaggcaggagattcccttgaacctggaaggtggatgttgcagggagccgagatggtgtcactgcactccggtctgggtgaaagagcaagctccatctcaaaaaagaaaaaaaaaaaaagagatatttttgatggattgatagaaattttctttttctttttttttttgagacagggtctcactctgtcgccaggctggagcacagtggcgtgatctccattcattgcaacctccacctcccgggttcaaacgattctccttcctcagcctcccgagtagctgggactacaggcatgtgccaccatgcccaactaatttttgtatttttagtagagagagggtttcaccatgttggccaggatggtctcgatctcttaacctcatgatccacctgcctgggcctcccaaagtgctggtattacaggcatgagccaccacatctggccagaaattttcttggtcacttctgagacatgcagagtaattacctgtaatataatttaatgaattatgtcaatatattaaaatatgcttcatgtgggctgggcatggtggctcatgcctgtaatcccagcactttgggaggccaaggtgggggtatcactaggtcaggagatcaagaccagcctggctaacacggtgaaaccccgtctactaaaaatacaaaaaattatccgggcgtggtggtacacacctgtagtcccagctactcgggagactgaggcaggagaatcgcttgaacccgggaggcagaggttgcagtgagccgagatcacgccactgcattccagcctgggcaacagaacgagactctatctcaaaaaaaaaaaaaaaatgcttcgtgtggcttaaaattatatgaaaagaaaatacctttactgatagtcatctgtgattccatttgctaaattaaacgtgaaagcatacttttactgaatactatatattccgtatcagtttagatagcagtttatcttcacatacataagttttaagtttacctttattatagtgcattggtcttttgttttcatcaacctaaattatgttcaataaatgtttctgttagattttaagttaaacaattatgtgaaattcatttttcgtaattgttttttaacatatgtctttgttggtaattcacgtgtgtgagtgtaactgattgccagattatataaactttcaaccaaaaccattctttgcagatgcttttactgactctgctatcagtgctaaagtgaatggcgaacacaaagagaaggacctggagccctgggatgcaggtgaactcacagccaatgaggaacttgaggctttggaaaatgacgtagtaagtaacatctttgtaattattgctagactctggtcagtatgacatcctgtcacttggttgtaatttaaatgtgcttttgttgttgttgttattgtagtgagtgtatttagagcagcaggtttgttgtataactagagactttctcccaagcaatatataaagaaaaatgtttgtcattttacttgtaggggttaagcaggagtactgtctgttcttgtggatgctcatgaattacttctttgtgattaaaataaataataagaagtagcttaaattaaaattagaaaccatgggaaatgccggtgtgttttgctttaacacccagccaaataaggtagcctaaggaaagtggtgtcttaattgttgacttcacctagagaagaggttgaagtaggacattttaagcctcttgtctgaagaaaaggttgtcattaagataaataattaggttacattggaattaaagcattacataaatttcttggtcttaaatttggattattctccacaaaattcttttatttctaaaacgcctcttgtcacatactagttttgtttctctctttaatgcattatctgtacttgaagtgcttagctgggtatgctggcacatgcctgcagtcccagctacttgggaggctgaagcaggaggatcacttgagcccaggagttggagtccagcctgaatgacataaggagaccccttctctaagaaataaaaataaaaacaaatacttaataaagactctgtctttaggatagagagcatagagatataaagcaaagtgtcttgccaaaaatgagtgttatggtaccaatatttgagtagaatgaagaatcttccattgagtagaaagagaatttgtaacatatctgtgtttgatgtttaaggcataacagcttaataatgacactcttcctcagacaggaagcctgaaatgtcctactttgacctaaagtctagtaataaaactggacatacacaggcaacatgtcattaattctcaaactttaacaaatcatatataacctaatataatggttctcaagtctgtacatcacgtcacctgtatgaaaaatatgaggaaacagagacttcttttacactattggtgaggtggataaattgatagagtctttctggagagaatctggcaatgctaatcaaaatttaaaatgcacatacactttgttccagcagttctatctctagtaatttatttttgccctcatatatccataagacatgcaaataattatatgtgaagattttttttttttctttttctgcagagacagggttttaccatgttgcccagggtgatctggaactcctgagctcaggtaatccacccacctcagcctcccaaagtgctgggattacaggtgtgagccatcatgcctgaccaggatttttttttttttcagcattatttcttttgttgttgttgctgttgttttgagagatggagtctcactctgtcacccagactggagtgcagtggtgcgatctcggctccctgtaacctccacctcctgggttcaagtgattctactgcctcagctttccaagcagctgggactataggcgtgcgccaccacacccagctaatttttgtatttttagtagagacggggtttcaccatatgttggccaggctggtcttgaactcctgacctcaggtgatctgcccacctcggcctcccaaagtgctgagattataggcgtgaaccaccatgcctggccatagcattatttctaatagtgaaaaattggaaacatgctaagtgtctatcaatatagcatgagttagatttatgatgtcaccattcaattgaaacactacatatctcccaaaaagaatggtgttccaatatggaaagatatctaagatttattaagagaaaaagcacattgcagaacactgggatcctatttgcttttttttttctttttttgagacagagtcttgctctgtcacactgcaacctccgcctcccgggttcaagcgattctcctgcctcagcctcctgagtagctgccaccatgcccagctaatttttgtgtttttagtagagaaggggtttcaccatgtttgtcaggctggtcttgaactcctgaactcgtgatccacctgcctcagcctcccaaagtgctgcgattactggcatgagccaccgcacctggccatgaaatttttttttttttttaaagagctgttcatattcttattgcctagaagatgtctgaaattacacccaagaaactctttttgagacggagtcttgctctgttgtccaggctggagtgcaatggcgtgatcttggctcactgaaacctctgccttccaggttcaagcgattctcctgcttcagccttctgagtagctgggactacaagcgcccgccaccacatctggctaattttttgtatttttagtagagacagggtttcaacatgttggccaggctggtcccgaactcctaatctcaggtgatccacccaccttggcctctcaaagtgctgggattacaggcatgagccactgcgcccggctgaaactctttttttttcttttaagatggagtctcgctctgtcgcccagacttgagtgcagtggtgtgatctcagctcactgcaagctctgcctcccgggttcacaccattctcctgccctagcctcccaagtagctgggactacaggctcccgccaccacacctggctaattttttgtatttttagtagagacagggtttcaccatgttagccagcatggtctcaatctcctgacttcgtgatcctcctgcctcggcctcccaaagtgctgggataccaggcatgagccaccgtgcccggccagaactcttaatagtagttatttatgcacgctgggattggaagacatttactttttactggatgtctttccgtattgtgtgcttttttttttttttttttatgtagggcatacattacttaagtaattttaaagcctccataagtaagtgtgatttcctgcccatgtgtttggcaaaaggaattgcattggtggtagacttacattatagtcttacctggagtagcacaggaggacccaaggttaataggtgaacttcgaggcaagccttagcattgaggttgccatcagcattgcttggttgatgtgttcattcttctgggatggattacaacctttactggactttatacttttcaccagtaaggctttaaaaaaggagttgaaacattagagaataattatccaggcagtaatattcactggtaaatagtcttccagcctgtggcccaattggttgattcttttacgttaaagaatgcagcctcagctgctctgcctatggagtaggattcttttatttactttcttaataaacttgcttgcccctggctcccccccaccaaaaaaagaaggcagcctcccttttgcgaatggtaatttcctatagtttcctcgtagaattgtggagttacctatgctgaggttataggttagggtattgagatccagagttgccacttctgaggtgtcacaactgctaatggtaaaaccatttctaaagcccagttcttgtgactttgtccagtgattgcctgttcaccgtttcatgctgccttcccatttgagcattcccaggaggaaggggaggttgccagggacctagtaccatagtccgaccttggaatcgttgaatatgagggaaagcgttggcttctcccttctttctcccaaacattggaagtatttttggctgttaaaaagcaccccttgttccatgtggaatcccttgtttaaaagaagtaaaatatgtacctcctgtcctccacagacctgaggaccagtgtgatctcaagaaggttacaggtaaatgtagatgtctctaactgaaaggtggcttttacaggttagagaaaagagagaaccctgatctgaaggctattttatgaagtaattaaaatgttctaaactttaaaaataactgctcaaataattgtgttgtatagttacttatcaactggaggggctgataagtatttttctaaaacatttttaaggaaattttttcctattttctaatttgctaattttgctcaagtagtttgttagatattgttaatatagatgttggttataactgaatgaaagggaacaactactttgacattttgaaaaacaagcttcattttcttctagtctaatggatgggatcccaatgatatgtttcgatataatgaagaaaattatggtgtagtgtctacgtatgatagcagtttatcttcgtatacgtaagtttgaaaagtttgtttttattttagtgcatttgtctttgattttcatcagcttaatttatgatgaataaatgtttgttagtttttaagttaaacaattacatgaaataatttttctcttattaccaactgtgataaatttccattaaaaaaagggaataaatgtagtttgcctataccctgtttttatgctctaaacaaattttggttttgtctttttttttcttttgagagggaatctcgctgtgtctccaggctggagtgcagtggtgcaatctcggctcactgcaacctctgcatcccgggttcaagcgattctcctgcctcagcctcccgagtagctgggactataggcgcgtgctaccatgcccatctaatttctgtatttttagtagagacggggtttcaccatgttggccaggatagtctcgatctcttcacctcgtgatccacctgcctcggcctcccaaagtgctgggattacaggtgtgagccactgtgcctggccggttttgtcttctaagttgttaaaaaatatctaaatttgcaagggcagagattatggtgaacagtttaaccagtttttgaaatatgttcctctggagaaaaggtaacagaaaaaaaagttagaattttgatttataaatacacagatcactataacttttagttttagttttagttttagtttctgtttttaccagtattctaaactctaaactttcttagtagttgattatgacagatacataaactgtggctttaaaggactcattttgcttttcttttcctcatgtttcagagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactaggtatttaaaggaaatcatgatgcagtattttggatacacaactcaaggtctgtgtgagacggtgtattgttattatatttcctcttcctttaatatagcttaggtagagaatgcaagtagaattggtttaagatctgttagagaaaaggttatggtgatcttggaaaatatgcttttgagagtaagctctgtggagccaagtgttggtatatcacggtgagcaatccaagatcttgaagagcttgttaaaatagttatctggtgggggacacgtgtaacaatcacagcagtacaatatgatttgcttggttaaaggcatgttcaaagtactaggaacatacagaatgaggaggagctagcataacctgtagagtcagagaaaacctcattgaggaggtgacattttgtgataagataatagggtctttgacacttagagaagagttgggagaagagtttatcacctgatgaaaagccatgtacaagcatggctatgagaaaatttggccagctcaggagagggctggttgttgcatgtgtctggaacacaggatctgtgtcaggtgcagcagtggcagttgatagtaggaactgaggtcattaaaggacttggcatgtcatgctaaagagcaccctgttggaaggagatggggtgaataaaccctggggcattgaggactggctgagacacagagaacagttagtgcactgaaatagttcaactgtgagaatttggtaaccacctagttaagggatgagcctgaggtttatttgataactaagtgacttaatggatgtactggtaagagagagaggaaacatggagcaagtttgaggggaaaaacagtgactccgtttgtgcagctaattgcatatgtgggcttgtgggtctttcatttattcataaacgtgttgagaaatacctgctacctatctagtaaagtaagagatgcatcctctcttaaaggcagtcagcttagagtctggtgatttgaattgacatgtccactgatagatgttgacactgtgagactggcggttcagtttgaggtttcatcagcattgccgatattggagccatgaaaaaccaaagaacagccagtgagagaagagatctcagagaaaataaaattgagaaagtgaaggacaaaaaatgttgtgaagatagaccaagattgatggaatcagccatagagaggtcaagtgggatgagaatgagcacgcatctgttaaactttgtgcttaggagcagaatctaagggaagggacagtccagaggttagaactcagggtaagatggaagaacaggggcatctgggagtgaggcagtttggtttagtgtagaacctttttgtaacaagcattcccttctgtctagatgacttttagatatgtttcattggcttggtaccttttagaataaaatgatttagaggatctctcattttcagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggcaagtattttgaccagacttgtcaatatcattgataaaatagttttctaaatacttaaaatacttaaaatagtttacataactgatatgaatgtgcactttaatgatttggtgagtagctttcacttcagcattacttaaaattggcttttgtggatattaaattagtaaaacattgtatatgtcattgacatatatattatttagcatgatgaaatattcatgatgtactaagataaagtgctacatttaacccaagacaatcacttggccaaaaacacttcacatataaagaaattggaaactttgggtaggttctcaattttaaaaacactggataataaaattttttagacataatttatatggaaaattctaacctatgtgcaacactgtggttaatatagatcaattttcattatttgtttctatattatgcttacttcaagaaaggatctgaggtaacttataatacaagacatgatcaagagtcatgtgaagaaagtgactagagaaatttgcttaaaaaacaacaaaaacaacccttagtctaagggtggatgttacagtttagcaacttaagtaaaagaaacctgaatctttagtaggaagacattttttactctacctctaaatctaggttgaatatatcttgtaggttgtggatcttttccataaatcagggatactgaacaacagttctatggatggtatggaaatagtaatagcaatagtatgttactaactttgtgggaaaagagtggacattcaattttagctatttaaatttggaaagttagatgaaaatagagaacactaagtttccaatttcatttgttttcattgagtcttttctccagaattcctctccaaatggacactcttgagtattttcagtacttaatattgggggtgaaatttctttgctcactgaggaaagattttagttgtttataaacagaattttaaagttaaaaaacctgaagggggctgagaaatatatgatacttaagtgtgtggaaccctatggagaggagacctggactgtttgataagattaaggtaagtgatatgtaatgttaaatactagctgtatctttacctaggcatatccatcagtataaatttatttggtgatgactgctttgtagttgcagtatttattaagcagtcgcttagataagtgtttaactgtataaattatttagaaggtctccctttttctagtttaatgaggtcaagactttttttttgaaatagcaatgaatattatcatttgatactcacaggagtcacaaactctagaagagtaatgttttatttctacttaaatgggacttgcttaataagattccaaactgagttctgggttcaagtgtaaacctgatgaaaatcatagataattgtaaggaaccagcatttctaattggatataatagctactgcttattttcgttatgcctcagagttaaaactaatacagtaaataatcttactcctgagtaggaattattgtgatttattatgtgaaattatctagtgtatgttatattcctttaaacaaccagttactgagaaacagttatagaagcaggattaataggcaaagtcttaactgtcttcttcaatagtgtgtatagatcctaattaaccctttgggaacgtgtattcatttaaacagacttaatcttaaggaggttaaagtaaaatgtgaatttatgtcagttaagttatgctaaaacttatcacaaatcaaatgactgtcctcaaagggttaaaatgtacaagaaatcatttttgtcattttactttttttctgtttacttttttccctcatttttttctttagtttttatactttccttcatatcatttgttctgtcaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaaggtacaataccacaatttgttcatgtttttgtttgtctttgtttaactcctatgtgagtttataattacaaaatagtttcctcttcattatttaataacctataatttctgtgttttaactttagtttattaaaactatttctattaaccttttgttcattagagagaaatttgataaatgtgtgaagctataaactctcttgaattgttgttaaaaagggggtttatctctgcctgataattatgcttctttacagccccagaagggtctgccccacagccttccccctccttatttgcactgtatacagtagttaaacaaatgaactttcttcagccagtcttgaacttaggttcattttacagctctttggccaaggtcctagtgaaccttcctattggccataagcagggatggtgttttctgggtcttttttgagagcgacagcccatgtagctgactttgcgtgtctgcccttagattaaagtagttgatttttagaatgccagaagaattctaaatttaactgagtaatttttttaaagttagctttgcaatcttacatagtgaaaggctgctttaatctggaagaagtccttgatctgagataaaattgataaaaacgacatatgaatttgaatatttagctatttctttcctcgtcaaaaataagaataaaatcttgtaattcttattcagtatttggcgctaaatccatcattgccacatatcaaatacagggatatgttgtagaaaggtaacattctaatttaaatgccacccatatattaaaaacctgttttctgaatcataatgtccttttgatactagttctgaatatttgtgttaaaattttaatctgatttgttcattaaaattagttaatattgcttatgttgggactaataaagttttccgcacaaaatgtgtttctcctgcttccctggagaaaactgtattggctacttttaaataaattgttaccatctaagcaggcaggtcatatgactttgactgaagcatctaaccttgaagagcaagttccactgattttcaaggtgacttctttgctcaaaagggccttaatagtggtcactaaatgcaaaattctgttgatatttttcttgtagtccatcatttgagtaagcgatgtttatttaatgagaatatattaaataaaacatgatcattaatgactgtgaacatctttattacattaagatttaaggactgctcatgtattaacttcacacagaaatatactttctgtgtcattcagagatgttgaatatttccatttgaaaattatagtgtataacattagcattcttctaaagatcatgttcgtgtttaaattcctgttggaagccaggcatggtggctaacgcctgtaatctcagcactttgggaggctgaggcaggtggatcacttgaggtcaggagtttgagaccagcctggccaacatggtgaaacctcgtctctactaaaaatacccagctacttgggaggctgaggcaggagaatcacttgaacctgggaggcagaggttgcagtgagttgagatcgtaccactgcactccagcctgggcgacagagacagactctgtcttataaaaataaaaataaaataataattctattggcaacatatattaatttgaagttctaaagagtttggcagccgggtgagagagtgaggagatttggctttgacattagggaagttttcgcttggtgttaacaccagtaggcttctctgatgagggccattctgtccactcttttacctgatagattggtctaatgcacagtagactgatttagaaagagtagtcactagtggcatggcagaatcaataatgtagaattttgacaattcatatagtgctgatttctcccccaaatgtcagttattttggtcatctattaatagactaatacaagtcatccctttaatagaattttcagctcacagcctgctaagcctaagaaactgcttacaggttactgcttactgttttaagccgagttttaaaattgatgatcatgatagaagagataaataaactaaaattttagagaaatttaagaagggtatgtacatatgttttagtggtatcggggtgtatagggattaatagtcttctgtttaaattttttttttctaattttagaagtaatgtagaaaattcgggtcagggaaaggtaaaatatatggaaagttaaaaatattttatcatgtagtcataatttctagtaacatatttctttacaaataagacatagttgaaacagattgctacagttcttttaagagttgacatcttattgttgatttcttaccaccaacttcatccctccctttctttaaaaataaagggaaataataaaatttatttataaaactttgtggcattccacaaaataattctgaaagaattagtatggccaaaaaaatatgtatggtgttttttttttttctatttttaaccaaggaaaaactgtagagtgagtgagtgtgtgtgcatgtgtgtgtgaatgggtgtatttagcagaaaagtagtactgatgaatatcatggaatttatgtgatgttcactgtttcttccttagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtgggggtaggtaacacttgggcataatgatggtactcattttgtcattacactagatataaagagggctgagctacaactctgtttgaggaagtgtaagtatgtatatgttaaaaatagtagaatcaccaggaattgggaaacccatatttttattctgggctctaccacttattcatcatatattaaagcaagtcagacactcattctgaagttgagatttcgcagtgagtaaagtgttaataattcttgcctagtctacattatgggattgtgatgagattcctataaggttcataaatacagatatattgtaaaactataaagttttgtaaagtacctctctaatatgaggcaaacacagtatgtaacactatttggagggaccgtatttccttatctttttagcagctttgtttatcagtacattctataaacatttatttttggcttacattgtagtgtgtttctatagcatctgtatatggcactaattcccaactatatttccataataaggaatatcaaatacaaataaagggtccaagttttatttgtgattagcataaggaatatgctgacagcagctataaaagtataaaaattaggctgggtgtggtggctcacgcctgtaatcccagcactttgggaggctgaggtgggcggatcacaaggtcaggagatcgagaccatcctggctaacacggtgaaaccccgtccctactaaaagtacaaaaaaaattagccgggcatggtggcgggtgcctgtagtcccagctacttgggaggctgaggcaggagaatggcatgaactcgggaagcggagcttgcagttagctgagatcacgccattgcactccagcatgggcaacagagcaagactctgtctcaaaaaaaaaaaaaaaaaaaaagtttaaaaactagacgttgacatgattttacaataaggctgactgcttttgctactttgccaatcagtccttagtgctttgttcccataactgtggtaagcaagagcttacaaagaatacttaaaacaaacaaacaaacaaacaaaaaaaacactttttctcttttaatcagtccagagaacctttaaaagaaacaagatcggccagttgctgtggctcatgcctgtaatcccagcactttgggaggctgaggtgggtggatcacttgaggtcaggagttcaagactggcctgaccaacatgatgaaaccccatctctactaaaaatacaaaattagctgagtgtggtggctatttgagaggctgaggcaggagaatcatttgaacccaggaggtgaaggttgcagtgagccaagatcacaccattgcactccagtctgggtgacaagagcgaaactctatctcaaaaaaaagaaaaaagaaacaagatcttcaagcttaaggaaacaaaaacaaaactcagctgtgttaaatctgtttttagttgctatacatttctgctcagcttcatgtgatgcacattcatgtaattgtatcctaaattcctttgtactttttattttcttccttggtcttcaattatcttaagactaccaagaaaacaaaaattttaaaaatcttcttcagccggtcaggcgcagtggctcacggctgtaatcccagcacttggggaggctgaggcgggtggatcacgaggtcaggagttcaacaccagcctggccaacatggtgaaacgtcgtctctactaaaaatacaaaaattagctgggcattgtggcgcgttcttgtaatcccagctgctcaggaggctgaggcaggagaattgcttgaaccaggacccgggaggtgtaggttgcggtgagcggagatcgcgccactgcactccagcctgggctatagagtgagactccatttcaaaaaaaaaaaaaaaaatctgcttcagctattctgttaatcttttgacattacttagatggtctggaaataaattttgagaataacatgattagaagtgagagagtataagcatagttttggagatacactcagaatagcattatagattttctctttttactaattggaaaaatggcagttgttgaataatagttttcttccgtgacccttgtgacttaaaaaaaaaaaaacactgaaatgaaataatcgaaccattttctctaaacctttgaatctgagctctgcagttaggtttataatggtatatgaaacctattagatatatacttggaagtcatatgggatacaaaccctgcttttattatcttccccttttgactaacttgggtctcaagtttccttaattactgcacagtggaccttgatgttgctataaagaatgtgtagggctgggcatggtggctcatgcctgtaatcccagcactttgggaggccaaggtaggcagatcacctgaggtcaggagtttgagaccagcctggccagcatggtgaaaccccgtctctactaaaaatacaaaaaaattagctggttgtggtggcgagtgcctttaatcccagctactccagaggctgaggcaggagaatcacttgatacatttagttaggagagaaaatcatacttatgttagtaattgctgctgttcttcatatacttgtggttttgattgccagcaaattcctaacattttggaaaagaaaacagtaatgggataaagggtaagggctagagaggacagttttatttacctagatcttcagagaagcctgaagcctcttttaggaagtaacatttgaactgagaatgtaataaatacattttccctttcttctagttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgagggtatgtaacaaagggcttctggatccataatctcagctgtgaaattgaatgttagagggtgatattatatgaaaaaattctaggttatttttattcatagacaagtatttttagtgcacatttaaaagtttatgtaaattttgatgttgtttaatactactaatttaatatagtgtctgtgttacaaaggttaacattcctgggtgtcaaatacctacataaataaaattattggtgtttcatatgacatctgcaaaggaaaaaaagcctctgtttaaatgaaagcattattttccaaaaacataggaaatcaaaattattgttcagtgttttcttgttttgcttttctaacttatctgaattttttttaaaaaattgttttctagctaaagattccaggcttcaagatcagaggcagaactctcctgcagggaataaagaaaatattaaacccaatgaaacatcacctagcttctcaaaagctgaaaacaaaggttagagtttaaagagtcattaagcttaactgtaggaataggaagaagtatgtctaatttcatgcccatacagaatatttttgttcaacatttcttcttactattgtgatagataaatgtattgcttgacaaattccaaaatccaaatttaatatttgaaattattttctgatcttatatcttattctaatttctatcatctcatactaaaaagaatgtgatgttaaagtttaaaaataaacctgtgtcttaacagttcttaattttacaggtatatcaccagttgtttctgaacatagaaaacagattgatgatttaaagaaatttaagaatgattttagggtaagtattgtactaactgatgaatttgagttttagaaaataagcattactaaagatttatctatttataaaaatgcgttatgtatacagtcagaaacatcaaaccatatatgtagaaagcagaacatttttaaagtggtctttgcctatcctttaagtgggataactaaaatcatgagatttggtaacaacaatatgtaggtatcaaatgagagtatagccctgacatttgaaaccaccatagcacagcttactatttgatggtcatttgtactttgttcagtgaagctagatattagtagagcaaggccaagtcattaataatctagtgtggcaaatggaagatgtactggactctggtgttctgaggtagttggagatttatactttgtacacaaatatattgtggtcaaaatctttctgtaacattatttctctgtcttagcacaggctttacttaacatctctccttgattgtcatttcattcttttgcatgttatttactataggtatcgaggtagattttgagaccaaccaataaatcttcttgaaacttagcttcttagaaaggaaaatctaaataccagccttttaaaaaaagtagctgaattaaaggatgagtgaaccaaaggcaaaggtagcctttcctcagcctgtgttttagctttctaaatgttaacaatagcttcattcttgacttattggtaacattcaaaatactacttattatttcatactttagcacatgtatctattcagctttaatgctattaacagttgttaacctaagttttcatttgttggcgggcacggtggctcacacctgtaatcctagcactttgggaggccgaggtgggcagatcacctaaggtcaggagttcgagaccagcctggtcaacatggtgaaaccctgtcttgaccaaaaatagaaaaattagctaggcatggtggcgcacacttgtaatcccagctacttggcaggctgaggcaggataatcgcttgaacccaggagacagaggttgcagtgagccgagatcacaccactccactccatcctgggcgacagagcaagactgcatctcaaaaaaaaaaaaaaaaaaaaaaagtttttcaatttgttaaacaatagttaacacatacaaatgatacaaagaatattgaatatgatcatgtgcccactacccagcttagtaaataaagcattctaacacagttaaactcctcttatgtatctgcccctcctcagctgcttccccctgtttccttccaaaaggaagggtttcttttctgtgcagttctttatatttatactgcatatgaatatatctgtgagcaatagatgatattttgcataatcttaaatttgctataaagtcttttttttttttttaattgatcattcttgggtgtttctcgcagagggggatttggcagggtcataggacaatagtggagggaaggtcagcagataaaaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccatttaaccctgagtgacacagcacatgtttcagagagcacagggttgggggtaaggtcatagatcaacaggatcccaaggcagaagaatctttcttagtacagaacaaaatgaaaagtctaccatgtctacttctttctccacagacgcagcaaccatccgatttctcaatcttttccccacctttcccccttttctattccacaaagccgccattgtcatcatggcccgttctcaataagctgttgggtacacctcccagacggggtggtggccgggcagaggggctcctcacttcccagaaggggcggccgggcagaggtgccccccacctcccggacggggcggctggctgggcgggggctgaccccccacctccctcccggatggggcggctggccgggcgggggctgacccccacctccctcccggacgggttggctgccgggtggagatgctcctcacttcccagacggggtggctgccaggcggaggggcttctcacttctcagacggggcggctgccgggcagaggggctcctcacttctcagacggggcggccaggcagagacgctcctcacctcccagacggggtcgcggccgggcagaggcgctcctcacatcccagacggggcagcggggcagaggcgctccccacatctcagacgacgggtggccgggcagagacgctcctcacttcctagacgggatggcggccgggaagaggtgctcctcacttcccagactgggcagccgggcagaggggctcctcacatcccagacgatgggtggccaggcagagacgctcctcacttcccagacggggtggcggccgggcagaggctgcaatctcggcactttgggaggccaaggcaggtggctgggaggtggaggttgtagcgagccgagatcacgccactgcactccagcctgggcaccattgagcactgagtgaacgagactccgtctgcaatcccggcacctcgggaggccgaggctggcagatcactcgcggttaggagctggagaccagcccggccaacacagcgaaaccccgtctccaccaaaaaaatacgaaaaccagtcaggcgtggcggcgcgggcctgcaatcacaggcactaggcaggctgaggcaggagaatcaggcagggaggttgcagtgagccgagatggcagcagtacagtctagcttcggctcggcatcagagggagaccgtggaaagagagggagagggagaccgtggggagaaggagaaggagggggagggggagggggggagagggagagggacaatgatgtcttgctgtaggtattcttccccatttgaattttttcctcagcattattttttttaacatcattcagtctcctcttatactacacttggattgaatttaatatctcatgaagaaaaaacatttctactttgaagcatgtgaattagcatgtttttataacagctttattgagatataatttacatatataaataaaccgtttaaagtgtataaatcagtggtttttaatgagatataatttacatatataaatcaaccatttaaagtgtataaatcagtggtttttaaaatattcacaatgttgtacaaccgtcttctcagttgattttaaaacatactcttcacccccaaaagaaaccccgtgcccagtttagcagtcgttccacatttgcctccagcccttctctttcccctactcccaaccctaagcaaccgttaatctactttctgtctctatggatgggcttatttggggcaaattccatttcatacaaatggaataataaaatatgtggcttttatgactggcttctttcactcagagtagtgttataaaagttcatccatgttggagcatgtttcagtacttcatttctttttgtgactgactaatattccttgatgtggataataccacattttgtttatccattaatcagtttgtagctatttgtggtgttctcactgtttgactattctgaataacactgccacaaacatgagtgtgcagtttttttctcgtcctatcttttcatttcttttgtgtacctacctaggagttgaattgctgggtcatatggcaactgtgtttaaccttttgaggaactaccaagctatttgccaagatatctacactattttacattcccaccagcagggtatgagggtttctgtttctccacatccttgctaacacttattgtcttgtcttttttgattatagtcatccttgtgggtgtgaagtgttaacctcattgtggctttaatgtgcagttctttcatggctaatgatgttgaacatcttttgtgtttattggccatttatatatcttctttggattgatgtctgttcaaatctttacccattttaaaaattgagttgtctttttattattgggttgtgggagttctttatatattgtgtgtacaagtccctgttagatacatggtttgcaaatgttttctcctgttctgttggttgtctttttactttttcatcccttgaagcacaaaaatttttaattttgatgaagtccaatttatctgattttgaagtaagcttttggtgtcgtatctaagaaaatactgtttcatcaatcattaaggtttattactcttctgggtttttttaagaattacatttagaggtgtgatccatttggagcaactttttttttcttttgacacagaatctcgctcttttgcttaggctggagggcagtggtgcaatcttggctcacagcagcctcagcctcctgggctcaaatgagtagctggtactacaggtgtgcaccaccacaccttgctattaataacttttgtatttttttgtagagacagaatttcgccatgttgcccaggctggtctcaaacacttggactcaagtgacacgcccacctcagcctcccaaagtgaaaaattgctttcaccttgcactgcggactcgccctgaattctttcttgtgcaagatccaagagccctctctgggggtctggatcgggacccctttcctataacaatattatgagaataacatttgattttttttaagtgaaacaaattgttattaaaaaattaaaaaaggtcataggagagtgacttggtgctcagcccattttgagcagttatttaatatagcataaggtggggttcaaattcattctttatattaattttttatttctaattgacacataaccatacacttataaccatttttactgtgtaagttcagattcattcttccgtatgtaggtattagttgtcccagcaccatctgttaaaaagactattcttggccaggcacagtggctctcaacgcctgtaatcccagcactttgggagtcccaagcaggcagatcacatgaggtcaggagttcgaaaccagtctgaccaaatggtgaaaccgcatgtctactaaaaatacaaaaattacctgggtgtggtggcgcacacctgtagtctagtcccactactgtagtggctgaggcaggagattcgcttgaacccaggaggtagaggttgcagtgagctgagatcatgcactccagtgtgggcgacagagtgagactccatctcaaaaaaaagactattctttcctccattgaattatcttcacatgcttgttggaagtctgttgactacaaatgtgaaagtttattactggactctgaattgtcctccactgaatctctatgtcttatccttatggcagtaccatactgtcttgattagagttactgtattttaaaaggctgtactttttcagttagcagaaaacattttagctatcagcacaactttctgtaaaccttcattaatgcttgacttaaattccaagaaggagcaacataaaaagtcttatctctttaggagttttagtcttactacttttaggtgcctgaataaccaaatgtattatttagcctcttactaataactccttgatccataggggcataccaggaagaaaagaagtggtttttaaaaaatgagagtgggccgggcacggtggctgatacctataatcctaacactttgggaggctgaggcgggtggatcacttgaggtcaggagtttgagaccagcctggataacatggcgaaaccctatctttattaaaaatatataaattagccgggcatggtggcacatgcctgtaatcccagctactcaggaggctgaggcaggagaatcacttgaatccaggaggtggaggttgcagtgatccgagattgcatcagtgggcgacagagcgagaatctgtctcaaagaaaaaaaaagagagtggaaaaaaaaaatatgtgtcccagaacttaaattttaattaaaaaaaaataaaagagtgaactttctaattgttctcttcagataatataatgttattctcttatgttttattgcgtatttcctgtgtaccagatgctgttcttcatgcttgtatgttaaatcttgtctaacatctctgtcaagcaagttctgtttgtatctgcactgtgtatattaggcagcttgggcaaagagaagttaagtaatctgcccaaactcacatggctagtaagtaagagggctgaccatctggtgtttaagcttctagcagtgctttgaatagtaactaatgcatagtgcatgctgcactgtcagtcagtgattcattagagctaacttcatgacatgctcatagccccaaactgcatttgttcacaaatatctgtagtccttcatttaggcagaaatagaaataccttgtgtgtttgttgttccttcccttttgagccatatgcagagtgctgatagctttatttgtgtaagaattgctagtaatttgatctgttttgggttaataatgtgggttttagaggtaaatggacctaggtttgaatgttggcctctatacatcatgtgcgtaacattgtggcatgctatctacttcccccaagccaaaatgggttaattttagaacctgcttcatagtgttcctgtgagagctcgatgagatattgcctataaagtgtttagcatagtgcctagcacatggtatgtattcaatacatgttcattcttactagcaaaatatagatgacccagtattgtacagagtatgtacaatggtgtcattgtaccatttcatgtggagtcacataagaatttcagttttctgctgggcatgatggctcactcctgtaatcccagcactttgggaggctgaggtggatggatcagctgaggtcaggagttccagaccagcctggccgacatgatgaaaccccatctctactaaaaatacaaaaaattagccaggcgtggtggcaggtgcctgtaatcccagctactcgcaagactgaggcaggagaaatgcttgaacccgggaggcggtggttgccatgagttaagatcgtgccgctgcactccagcctgggcaataagagcgaaactccgtctccaaaaaaaagaaaaaaaaagaacttaagttttccattagatttagtatagtgcagagaggaaatacagcagagtgctatattccatatatagcaatatagcattagaacaatatattccaatacagcagagtgctatattcagataccaactagtggacttgctatttgtaagatggcaataatagtatctacatcaaatagggctgttgtgaagactaaatgaataagtctataaatagtttagaacagtgtctggacaggtacagtggctcatgcctgaatcttagcactttgggaggctgagacaggtggatagcttgagctcaggcattaaagaccaacctgggtaacatggtaaaaccctgtttctacaaaaaaatacacacattagccaggtgtggtggcacatgctaatagtaccagctactcaggaggctgaggtgggagaatcacttgagcctgggagatggaggttgcagtgaggtgagcttgcaccactgcgctccagtctgggcaacggagtcagaccctgtttggaaaaaaaaaaaaaagtgtccaacccatagtaagaaatgcagatgtgtttgacattgtaagaaaaagcaacaccaaaagtctgatttttgccttcactcaagaactcttatgataattaaactccgaagtccttggcaatatatatagttggtctgttatgtggatcgcctctactaaagatttttgtgaacaaatgaaagtttaagtagtaagttcctacatcgtgacttaaattgccagtgtgcccacataaataccctgtcaacatttgcccttagccacttgactctttagctatattggtaatgcagtaaagcttgcgatgcgccagagttgcataatgctgtttgccatgacaccaagagccttggtaatgaaaccattgaaattggtttgcctatactgaggctgaagaggtatcttggctctctaattttaaggcaacctttttggctgtgtaggtttctctttagcttgtttctcaccacctggggctgtggcttaggtccgttgtcctaacctgtggcttaggttctgtttttgttgcttgtacttgctcccccttttttcagccattcctgttttctttcttttgtagaggatgccatcttaaatcatcttcagccagtggtagcattttattttttctggtctgcaaacttaaaaacctcatcacttattttgctaatatctttgtcttctgttctttttgatggtccttggttttgcagtctactttaaaggtttttatttttttatgggtacatagtagacgtattattcatagggtctgtgagatatttagataaaggcatataatgtgtaataatcacattagggtaaatggggtatccatcaccatcatcattcatcatttctttgtgtaatgaacgttgcaattgtactccctcagttattctaaaaagtacaacaaattaatgctgactgtagtcaccctgctttgttgtcaaatactagatcttattcattctttatttaactttttaaattttaaacttattttatttatttatttttagacggagtctcactctgtcgccaggctggagtgcggtggcgcagtctcaactcactgcaacctccgcctccagggttcaagtgattctcctgcctcagcctcctgactagctggaactacaggcacgtgccaccacgcccagctaatttttgtatttttagtagagacggggtttcactatgttggctgggatggtcttgatctcttgaccttgtgatccggctgccacagcctcccaaagtgctggggttgcaggcgtgagccaccgtgcccggcctttaaaattattttaaatcattttaatatctttttcatttctgcctccggtcctgcagagttcttattcgttctttctaaattttctttgcacccactaatcacctcatttcccttcttctccccattacccttcccaacttctggtaaccattctgctatctccatgtgttcaattgtttttatttttagtgcctgcaaacgagtaagaatatgcaaagtttatctttctgtccctggcttattttacttaacataatgtcctccagtgccatctacattgctgcaaatgacaggatctcattcttttttatggctgaatggtaatctattgtgtatatataccacattttctttctccatttgtctgtcagtggacacgtaggttgattccaaatcttggctgttgtgtatatagtgccgtagtaaacatgggagtgcagatattccttcaataaactgatttcctttctgagtatatacctagcagtgcaattgctggatcatatggtagctctatttttagttttttgaggaatttccatactgttctccatagtggttttaccaatttacatgtccaccaacagtgtgtgaaggttcccctttatccacatcgttaccagcatttgttattgcctgtcttttggataaaagccattttaactggggtgagatgatatcttgttgtagttttaatttccatttttctggtgatcagtagtattgaatacctttcatatacctgtttgccattcataaataacgatgaggtcttgctgtttggcccaggctggtctcgaactcctgggctcaagcaatcctcccaccttggcttcccaaaatgctgaaattatagttgtgagccactgcacctggccttgtatgtcttccttttttttttgttttgttttgtttttgagacagagtctcactttgttgcccaggctggagcgtagtggtgtgatcttggctcactgcgccctacacctcccggattcaagcaattctcctgcctcctgccaccatgtctgcctaatttttgtatttttagtagagacgggatttctccttgttgcccaggctggtcttgaactcctaacctcaggtgatttacctgcctcagcctcccaaagtgctaggattacaggcgtgagctgctgcgcccagcctgtatgtcgtcttttgagaaatgtctattcagatcttttgcccatttttaattgagttactaaaattttccctatggagttgcttgagtgccttttatattctggttattgatcccttgtcagatgagtagtttgcaaatattttctcccattctgtgggctgtctcttcactttgttgatggtttcctttgctgtgcagaagctttttaacttgatgtgatcccatttgtccatctttgctttggttgcctgtacttttggggtattactcaagaaatctttgcccagagtaatgtccctgggagtttaatgttttcttttagtagtttcatagtttgaggtcttagatttaaatctttagtccattttgatttgatttttttttaatatggtgggacacaggggtctggtttcattcttctgcatatggatatccagttttcccagcaccatttattgaagagactgtcctttccccagtgtatgttcatggcttctttgtggaaaatgagttcacttagacgtatggattcatttctgagttctctgttctgtttcattgatctatatctttttttatgccagtaccatgccattttggttacaataatttgaagtcagataatgattcctcccgttttgttcattttgctcagtatggcttttgctcttttgggccttttgtggttccctacaaattttagaattatttttgtctacttctgtgaggaatgtcattggtattttgatagggattgcactgaatctgtagattgctttgagtattatcaacattttagcaatattaattcttctaatccataaacatggaatctcttttcatgttttttctgtgtcatcaatttcagtgttttaaagttgtcattatagaaatcttttactcatttggttaagtttattcctaagtattttattatatttgtagctattgtaaatgggattgcgtttaaaaaatttttcagattgtttgctgttaaatataaaaatgctcctgatttttgtgtgttgatttttgtatcctgcaattttactgaatttgtttgtcagttctaataggtttttcttttttggagtctaggtttttccaaatgtaagatcatattatctgcaaacaaggataatttgacttcttccattccagtgtggatgctttttatttctttctgttgtctgattgctccaattaggacttccgagtattatgttgaataacaatggtgaaagtgggcatccttgtcttgttccagatcttagaggaaagcctttcagtttttcccttttcagtatggtactagttatgggtctgtcatatatggcttctgttttgttgaggtatattccttctatacccagttctttggggtttttttgtttgtttgtttttgagatggagtctcactctgtcacccaggctggagtgcagtggcgcaatgttggctcactgcaagctccacctcctgggttcatgccgttctcctgcctcagcctcccgagtagctgggactacaggtgtccgctaacacgcccggctaattttttgtatttttagtagagacggggtttcaccgtgttagccaggatggtctcgaactcctgacctcatgatctgcccgtctcagcctcccaaagtgctgggattacaggcgtgagccaccacgcccggccaagggttttaatcataaggggatgtggcattttatgtgatataaattatatatttatatcatgtgatatatatttatatcatacacagtataaataatatatatatatattttttagtctttgtcttttattctgttaagatgtaccatgtttattgatttgcgtatgtcgaaccatccttgcatccctgggatgaatcccacttagtcatgatgaatgatctttttaatgtgttactgaattcggtttgctagtattatattgaggatttttgcataatgttcttcagagacactggcttctagttttccctttttgatgtgtcctttggttttgtatagggtaatagtggccttgtagaatgagtttagaagtattccctcttcctgtattgtgttggaatagtttgagtaggattggtattagttcttctttaaaggtttagtagaattcagcagtgaagccatcaggtccatggcttttctttgctgggagactatttcttatagctttgatctcgttacttgttattggtctcgttacttgttattgtatttgggttttggatttctttgtggttcagtcttggtaggttgtatgtgtctaggaatttatccatttcttcaaggttttccaatgtatcagcatatagatgctcatagtagtctctaatgatcctttgaatttcggtggtaacaattataatgtctcctttttcatctctcattttattatttgggttttctcttttttttctgagtctggctaaaggtttgtcagttttgtttatctcttcaaaacaatttactgttttattgatcttttgtattttcttcatttcaattttatttatttctgctttgattttttttatttcttctactgattttaggttttgtccttgcttttctagttctttaggatgtattggcagatgaagtttttccacttttttgatgtaggcacttactgctgtaaacattcctcttattgttgcttttactgtatcctataggttttgataagctgtgtttccattttcatttgtttcaaggaattttccagttttcttcttaatttcttcatggacccactggtcattcaggagcatattgcttaattttcatgtatttgtatactttccaaagttcctcttgttatctagtgttattttattttatttttatttttgtttttttgagatggagtctcgctctgtcacccatgctggagtgtagtggcgcgatctcggcttactgcaacctctgcctccccagttcaagtgattcttctgcctcagcctcctgagtagctgggattacaggcatgtaccaccactcctggctaatttttttttgtatttttagtagagagggggtttcaccatgttggtcaagctgatctcgaactcctgacctcagatgatccacccaccttggcctcctaaagtgctggaattacaggcatgagccaccgtgcccggcctctagtgttatcttattgtgatcagagaagatagttgatatgattttaacttttttgaatttttatttatttatttgtttgtttgtttgtttgtttgtaacagagtctcactctgttacccaggctggagtacatgtcatgatcttggctcacctgcaacctccgccttcctggctcaagcaatcctcccaccttagccttccaagtagctgggactacaggcacatgccgtcacatatggctgatatttttggattttttttttttttgtagagatggggctttgcgatgtgtcccagggttgtttcgaactcctgagctcaagcaatccacctatttcggcctcccaaggtgctgggattacagacatgagccactgtgccacgtcaaatctttagacttgttttgtggcttaacatagggtctatctttgagagcaatccatatgttgaggagaagaatgtgtattctatagctgttggacacaatgttctgtaaatatgtattgggcctatttggtctatagagcaaattaggtctaatgtttctttgttgattttctgtctgaatgatctgtccattgctgagagtggggtgttgaagtttccgactgttactgaggtctgtttctcttttttgctctaataatgtttgctttatatatctggatgctccagtattggttgcatatgtatttatacttgttataacctcttgccgaattgatccctttatcattatacaataatcttctttgtctgtttttatagactttgtctcaaaatctattttatctaagcatagctactcctgttcttttctggtttccatttgcatggaatattgttttccagctcttcaattttagtctatgtgtgattttataggtaaagtgtgtttcttgtaggcaatggatctttggtttttttttttttttttttgagacagagttttgctattgttgcccaggctggagggcaatggcgctatctcagctcactgcaacctccgcctcctgagttcaagcgattctcctgcctcagcctcccaagtagctgggattacaggcgcctgccaccaagcccagctaaattttttgtattttcagtagagatggggtttcagtatgttcgtcaggctgttcttgaactcctaacctcaggtgatttgcctgccttggcctcccaaagtcctgggattacaggcgtgagccaccgcacccagccttttttttaaatccatttagccactctgtatcttttgattggagagtttagtcgatttacattcagtgttgttactgattagtgaggacttaactactaccattttgttacttattatctggttgttttgtagtcctactccctcccttcccccttcttttttacttcctcttcgctccttttttccctccctcccttccttgttttgaaagtgattttctctggtggtatgttttaatttcctgctttatattttttgtgtatctgttgtaggtgtttttgatttaagatcaccatgacagctgggtgcagtggttcacacctgtaatcccagcactttgggaggccgaggtgggtggatcaagaggtcaggagattgagaccagcctggctaacatggtgaaaccccatctctactaaaaatacaaaacttagccaggcgtggaggcacgtgcctgtaatctcagatactcaggaggctgaggcaggagaattgcttgaacccaggaggcagaggttgcagtgagtcaatattgtgccactgcaccccagcctgggcgacagagtgagactccgtctcaaaaaaaaaaaaaaaaaagagatcacataagggttgcaaataacattttataacccattattttaaaccaatgacaacttgaaactttgattgcaaaaacaagcaagcaaagagaaaactaataaaaactctacacttcatctgcccgctttttaacttttgttgtttttatttatatctttattatactatgtcttaaaaaactgtagttataagccaggcgcagtggttcacgtgtgtaatcccagcactttgggaggctgaggtgggcggatcacctaaggtcaggagttcgagaccagcctagccaatatggcaaaaccccctctctactaaaaatagaaaaattagccggacatggtggcgggtgcctgtaatcccagctactcggaggctgaggcaggagaatcacttgaacccaggaggcccaggttgcagtgagccgagagtgcgccactgcactccagtctgggcaacagagtaagactgtctcaaaaaacaatacaaaacaaaacaaaaccctggcctagtggctcacgcctaatcccagcactttggaaggcaaaggtggggcgaatcacaaggttaggagttcgagaccagcctgaccaacgtggtgaaactctgtctctactaaaaatacaaaaattagccaggcgtggtggcacgcacctgtaatcctagctactcaggaggctgaggcaggagaatcgcttgaacctgggaggcggaggttgcagttagccgagatcgcgccactgccgtccagcctgggcagcagagcaagactctgtctcacaaaaaaaaaaaaaattgtagttcttatttttgaaaggttcattttttattcttcctgctcaaaatatgagtagtagtttatacaccacaattacagtgttacaatattctgtatttttctgtgtacttgttaccagtgagtttttgcaccttcaggtgatttattattgtttgttaacatccttttcttgcagattgaagaactttttttttttttttttttttttgagacagagtcatgctctgttaccagcctggagtgcagtggtgccatcttggctcactacaacctccaactcccaggttcaagcgattcttctgcctcagcctcccaagtagctgggattacaagcatgtgccaccacgcccagctactttttgtatttttagtaaagacggggttttgccatatttgccaggctggtcttgagctcctgacctcagggtgatccgcccgccttggcatcctaaagtgctaggattataagcgtgagtcatcgtgcccaacttggttgtttattttcaaatagcctgaattcaagctcactaatgttttctgctgcttgatacatttctgctattgagagactgatgcatttttcagtttgtcaattgaatttttccactttgggatttctgcttgattctttttactaataattattgcagtctcttttttaaatttataggattctgaatttgttctctgtattatcttggatttcgttgaactttctcaaagcattcagcttgaattctgtctgaaagttcacatatctcttatcacttgggaattggtcactggtgtcctttatttttagttcatttggtgaggtcatgttttctcagatggccttgatgcttgtggatgttcatcagtgtctgggcattgaagagttgggtattctgttctttgtagtctggttttgtttgtacgcattcttttttttttttttctgtttttgagacagagtctcgctctgtcgcccaggctggagtgcagtggcacagtctttgctcaccgcaacctccgtctcccggattcaagcaattctcctgcctcagcctcctgagtagctgggattacaggtgcgtgccaccacgcctggctaatttttgtatttttagtaaatatggtgtttcaccatgttggtcaggctggtctcgaactcctaacctcgtgatctgtccgccttggcctctcagagtgttgggattacaggcgttagccactgcatccggctcccattcttcttgagaaggtttttcaagtattcaaagggaattaagtgttgtcatctaagtcttcgctcactgcagccatacatgcattagagggcaccccaagactagtaatgttgtgactctgtagaggtatcaccttggtagtcttggggaagatctgggagaattccctgtattaccaggcagtctcttgtcctcttacatttctccaaacaaatggagtctctctttgtgctgagctgcttggagtttggggaagggtgacacaagcactgccatggccaccgtcactggaactgtacttggtctcacccaaggcctgtggcagctattttctggccaccactgatgttaatttaaggcccaagggtgctttagtcagtaggtgaagaatcctgcaagaactgggtctttactttcagtgcagcaggttcccttctggcccagggtgtgtctagaaatgctgcccaggagccagggcctgggatcgggagctttaggaatctgctttattgtactggggctgagctggcacccacttgcaagataaagtcctttttactcttctctcacctcaagcaggtgggtctccccatggacaccacagctgtgaatgtgcggggtcatatctgaagctggcacaatacgacatggcaccttgttttttattcaaggcacaagggctctttagtcagctggtggtgaatcctactaggactaggtatttcccttcaaggcaatgggttcccttctggtccagaatatgtctagaaatgtcatctgggagctatggcctagaattgaggcttcagaactatgcttggtgctttattttactgtggctgaactagtatccacattgcaagacaaagtcctccctactcttccctctcctcccagagctgtgagctgtggtacctggagttgggggaaggctggcacaagcactcccttggccaccctagctggtgtctcagtgggtcacatgtaccccaagtccactgactatgagcccagcacagtaccatgacttgtccaggaattgcagtccttctggtctagactgcctttcaagtttatttaggaccccagaggactttacccacggtggtggggcttaccaaaattaagattcttttggttttttttggcagagtttcgctcttattgcccaggctggagtatagtgacgcaatctcagctcaccacaacctccgcctcccgggttcaaataattctcctacctcagcctcctgagtagctgggattaccggcatgcgctaccacctctggctaatttttttgttttttagtagagatgaggtttctccatgttggtcaggctggtcttgaactcccgacctcaggttatccgtccgcctcggcctcccaaagtgctgggattacagaccatagtgcccagcccgaaattcagattctaatcactgggatggacaattcccctctgactagggctagtctaaatactccctctgtgggtgctggctgaattctgtcctatgctgctttccactgtgacagggcagcactgagtttcaatgcaaaatcccacagtcatttctctctctctcccccgagcacacagattctttctccaccccacactgcattgtgggggaatgtcaggggtgttggaggggcagttcaagactatcttccttatcttttttggtgtctttttccttgataggatgtcaaaactgggtactgtgatcgcttacctaatttttggttcttatgaaggtgctttcttgtgtggatagttgttcaatttggtgctccttgttggggatgatcactggaaggttctgtttggccaccatgctctgtctcttctcccctgccatctcctttttttacttaggggtttagaatgtctaactgaccaatatgtacagtcaggtctcattctgaattcacctacttaatgaccttccaagctgactaggcccagcgcttagtccagcctccatgacggtccctccacatcctaattagcctccctccagttcatttcacacaaagctgctgtgttcacctttctgaactataaatctgcccagtactctaccctacttaaaattccgtatagactgcccatttgccctgagaattaaaagccaaagtcctaaacgtagctttttaaaacttttttttttttttttttaatttttagatggagtcttgctctgtcacccaggctggagtgcagtggtgtgatcttggctcactgcaacctccgcctcctgggttcaagcaattctcatatgtcagcctcccaagtagctgggatttacacgtgtgccatcacgcctggctaattttttttttttatctttagtagagacggagtttcaccatgttggccagtctggtcttaaactcctgacctcaagtgatccacctgccttggcttcccaaagtgctaggatgataggtgttagccactgcacgcagccctgaacatagcttttaagttcctttattgtcatattccttttgacgagtctatcattttctgactcacttgtacatgtgtgtctcacccttggtccagccattggtgcttttctttacttctttatttttgttattttattttattttattattattttttaaatgagacagggtatcactatgttgcccaggctggtcttgaactcctgagcttaagcagtctgcttgtctcagcctcccaaagggctggaattacagtgatgagctactgtgcccagctcattggtgctatctttttttttttttttgagacggagtctcgctctgtcacccaggctggagtgcagtggcgtgatcttggctcactgcagctccacttcccaggttcacaccattctcctacctcagcctcccgagtagcagggactataggcgcctgccaccatgcctggctaatttttgtatttttagtagagatggggtttcagcgtgtgagccaagatggtctcgatctcctgacctcgtgatccgcctgccttggcctcccaaagtgctgggattacaggcgtgagccaccgtgcccggcccccattggtgctattgttttatgtgatagagccagcttctcccttttctttggatttttaaacatactcttccttttacttagactattctccatcccaacacctttcctaaacttctttcacaccttagactagctgacactttactgagaaacctttcttttttataggttgctttttctatagactctcttagcatttactcattttattgtgaagtgtctgatcttatttaaatgacaagtataagaggatagaaactatttcatatttttctcacccagcaggcacaatttctgacatgtggtaagcactcagtaaatattgaactttagaggctaggacatttgagtgctttggtgactgtggttgtgctatataggtactctgttattgttagtttatagtaaaagcattactcttaaagtatgaaaaaagccttattcagaacatttcatgcgtatagttaatattacgtagcttgtgctcatggcaaaaatgtattactaaagttatttaagatatttaagtataattgtttcctttatttagttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagtgagtaaacctggaacttagaccatcctgttactcaattaacttttttttttttaaaggcatttaggtccttccaactgtgaagaatccatctggacttttagactactttatacattgcccttagtttacaaacagctagtccaaacaaatgacatcttaagtaaatgaggttattgcaccctgtgctactcttctgttcttccccttttttgtaccccagggctagaaaaacaaggcataaattaagaaaagtttttctgtaaatgaacaggagttgaaaaattatcaattcaggggacctatctttactggattccactcattagtcaccctcactgtgctgctaggttgaaaaactgccactgtcaaggagagaagcatgcggtgcttctacttggaattcaaaatatttttcatcagaaactgtgttttagttaatgtttagatttgttaagatagacttaattctgcacattcagtatattaattaaatggacttttaggggctaacctcagaacttaactaccattgacttaggtgtttgggtaccaaacaatccagttaaagctgaagttttggaatgcagcttattgataaattggggactgcttattcttgatttgaggcaatttttttttacagccatgactttttccaggtatgtcatgtaaaatatcttctcacataagaattactgcatgctagaatattggtatgttgactggtagctcatacctataatcccagcactctgggaggtccaagcaggtagattacttgaggttaggagttgaagaccagcctggccaacatgtgaaaccctgtctgtactaaaaatacaaaaattagccaggcatggtggtaggtgcctgtatcccagctactcgggaggctgaggcaggagaattgcttgaacccagaaggtggaggctgcagtgagccgagatcatgccactgcactccagcctgggtgacagagcgagactctgtctcaaaaataaataaataaataaataaaaggatactgttatgttaagaattgcttttaaggatatttcataagtagctactgtcttttcagctcaagtgtttgttgattggccaggcgtggtagctcatacctgtaatcccagcactttgggaggctgagtcaggcagatcacttaaggtcagcgtggccaaaatggtgaaaccccatctttactaaaaataaatattaaaaaaaattagctgggcgtggtggcagtctcctgtaatcccagctaatcaggaggctaaggcaagagaatggcttaaactcgggaggcagaggttgcagtgagccaagattgcactgctgcactccaacctgagcaacagagtgggactctgtgaaggaaaaaaaaaaagtattttttgattgcctttgagaggaacggttgtatattactcagatttttaaaaaattgttcttttatggctgtattctttaagggattaaggaatgggcaatataagtgtatatgtttcaataaaaacgattagtgatcttctagtgagaacagtttaaatctatatttagcaattttttttaaattgtcaggtatggaagattttagagcaacgtaaagtccatgtagatttcactggcctttatattttttttaggcaagttaggaaatcaacattgaatcccaatgcaaaggagttcaacccacgttccttctctcaggtaggtttattactttctttgaggttatctagtcccaaaaaaagaaaaattattagtaatagtccttcttccatacctgccatctgaattttgttttagtgtgctgaaccaaccttctttcttttttttacatggccattaatgaatactttttaaacattaaaaaaaggtctttgttttgtcatcaattagatgtgatcttgggcaaatctttgaatttctctgacccagaatttgacgatggttggctagctaggctgtcaggtttatagatacgtcctctgcacctgagggttttgcatcactggattcaaccaaccatggatcaaaaacatagttaggataatctatactgaacacatgcagacgtttccttgtcattattccaaaacaatacagtaaagcatttaccttgttttaggtattataaataatctagagatgatgtaaagtatataggaggatatgcataggttgtatgcgaatactacatgattttatgtaagggacttgagcattccaagactttggtatcttcacagggtactgtaaccaatcccccacagatactaagagatgactgtactattgttattattcgactgagatcataagaagatatatttatttttaatttttaaaaacacttccatcagtttcttaaaaatagctgccactgtttttaatattttttaattgacaaagttttaagttcctactgaaacattttttcttttattgaaatgtgaaaatttatgtgctgtgtttttgttttcaataaaagggacatagttaaagcaagtaaaattagaaagactgggaaaatccgtctttaaattgcaataatagttcatctgttaccttgagataattgaatttattgttgtttttgtagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaagtaagtcatagaatttgatgttcacttagcctccccaattgtttgtatctgacaccaagcactctttaggttttcagtgacttgagggtgtgatggttatgcatatgcatttgaaacagacaggcatgcagagattcagtgtgttgttaagtatgaggacctaaatctgagaatgttttctgtgaaaaagatggtttagatttactgtagtttggggtttgttccttttagctgtgggtatgatctaattttttaatgactaatggagaatcaggaaaccttctcatgcctagctctctagcaatataaaactaagagtgacagaataccttgttattatcataggtgcctaatgttaattttttttttaattctctcaagcctttatacccaatacctatgacgcccatgccagtgaatcaagccaagacatatagagcaggtaaaggtgagaataatcctgcctgtgtttgcttgtagtttgcatgctgcatgaattgagtaactaagtttataatgaataaatagttgtagtttagctctgactttttgatgaggctatgcattggcttttgatgaacaacattacatagatattcacatggattttatgaagaaaaacaggggagaaaaaatgcccatcagttgtgattatatagtatcctcttcaaaaagagtaattggaggcctggtgtgatggctcacacctgtaattttagcactttgggaggccaaggcaggaggattgcttgagctcaggagcccaagatcagcctggacaacagagactttgtctctactaaaattcaaaaaaattagctgggcatggtggcatatgcctgtagccccagctgtttgggggactgaggcgagaggatcacttgagcccaggaagtagaggctgcagtgagctgtgattatgccactgccctccagcctgggcgacagagtgagaccccgtctcaaacataaatactggctgggcatggtggcttatgcctgtaatcccagcactttgggaggccgaggtgggtgtatcacctgaggtcagtagtttgagaccagcctggccaacatggcgaaaccccatctctactaaaatacaaaaattagccggacatggtggcacctgccgcctgtaatcccagctactaggtggggctgaggcaggagaattgcttgaacccgggaggcaggggttgcagtgagccaagatcgtgccactgcacttcagcctgggcaacagagtgagactccatctcaaaacaaacaaacaaacaaaaaacaaacaaacaaaaaaaccagactaattggctggacacagtggctccatgcctgatatcccagctggaggatgacttgaacccatgagttcgagagcagcatgggcaatatagtgagaccctatctcaaaaaaaaaaaaaaaagttaattccaaagctttttgatctgaaatctgatttaaatctgaacttaaatttgaagaagagggtttgctagattaatttactagattgctaaccttgctttatatatacctacagttatttccccaaagccagaatttcttttgaagcagaggggcaactaacttcaaccaatgttaagatcctattagaaggatgtttcggctaggcttggtggctcacgtgtaattccagcactttgagaggctgaggtgggcagatcacatgaccgggagttttaagaccagcctgggcaacatggcaaaaacctatctctgcaaaaaaaaaatagaaatcttagccagccgtcatggtgtgctcctgtagtcctagctacttgggagactgaggtgggaggatcaattgaaaccagaaggtccaggctgcagggaactgtgactgcaccactgggctccagcttgggtgaaagagcgaaaccctgcctcaaaaagaaaaataagatggatgtttctgcattaaaattagggagttgtcgtataatgtagttgcataaactagtattctgtgcttgtgtggttaaagagccttcgtagaaaaaatcccacatttttcttaaaaggaaatcttttggccaggtgtggtggctcacatctgtaagcccaacactctgggaagccgaggtgggcagatcacttgaggtcaggagtacaaaaccatcctggccaacatggtgaaaacccgtctctactaaaaatacaaagatcagctgggcatggtggtgcgtgcctgggtgacagagcgagactccgtccaaaaaaaaaaaaaaaaaaaaagagttcttttaatgttggaaaatgctaaagggttttttttttgccaaccagttaatttagagtgattaactgctatcagttgagaaactatagaaagtagaataatttatacagaaaagacatttctcagtgcccaataattgcctttctgacataaagttttcatttttcctgaattaataagatttcctcaatgtgtttttttgggtgttttgtgtgtgtgtgtgtgtgtgtgtgtgtgtgtgtgtgtgtgtgtgtatgtgtttgatacagggtcttgctttgctgctgaggctggaacgcagtggcgctatcatggctcaatgcagccttgacctcctgggctcaagcgatcctcccttctcagtcccctggatagcgggggctacaggtgcacaccaccacacctagctaatttttgtattttttgtagagatgggttttgccatgttgcctaggctggtctcaaactcctgggctcaagcgatctgcctggctctgcttcccaaagtgcctgcgcccagccaattttctccatgtttgacctaattgtgatttcatagatgttaactaaaactcttaattttcgttttctcagtatgctattttttttttttttagccttggaacatatgaacctgttgaaagaactctgcctgaaataatgtaatcaaattatagagtttaatcttattttgagggcctttagaaattctgagaagaaagtgggtttttttttttactgccattttaatgtagtgttaaggtgttcatgtatcaccagcaggtgtagctgttttcaatgattacttaaaacaatgcaatgggaactttttgttgtcattaaaatataaaaggttactgtagtaagagcaagcatgacagtttggctatctgatgggagagtcacattctaacttcaggaggtactgtctttttaatagaaatgatatactcagagtctgggcacggtggctcacgcctgtaatccagcactttgggaggccgaggtgggcagatcacgaggtcaggagatcaagaccatcctggctaatacagtgaaaccgtgtctctactaaaaatacaaacaattagctgagcgtggtggcaggtgcctatagtcccagctactcgggaggctgaggcaggagaatggcatgaacctgggaggcagagctggcagtgagctgagatggtgccactgcactccagcctgggtgacagagcgagactccgtctcaaaaaaaaaaaaaaaaaaaaaatagtagagaaagggctttgccatgttggccgggctggtcttgaactcctggcctcaagtgatccacctccctcggcctcccaaagtgctgggattacaggtgtgagccactgctcctggcctgaatataccacttttacctatcatcagttgatgaacatttggattatttcctttttctggcaatgagtaatgcttttgtggattttcatgtacaaattttcatatgaggctgggagcagtggctcatgcctataatcccagcagtctgggaggctgaggtgggcagatgacttgaggtcaggagtttgagaccagcctggccaacatggtgaaatcccatctctactaaaaatacaaaaattacactggcatggtagcgtgcacctataatcccagctattcaggaggctgaggcaggagcatcagaatcgcttgaacctgggaggcggaggctgcagtgagctgagatcacaccactgcaccccagcctgagtgaaagagtgagtctcaaaaaataaaaaataaaattttttttcatgtggccttagattttcatttctcctaaagtagaaatgctgtgatggaactgccaaacttttccaaagcagctgcatcattttgtatttctaccagtaatgtacaagtgttccagtttctccacatcctcataaataaccgatatgtctttggtttgggttatgtccattctagtggttatgaagtgtcattgtggttttttgtttttttgtattgttttgagatcgtgcccaggctggagcacagtggcacaatctcggctcactgcagccttcgcttcctgggttcaagcaattctcctgcctcaccctcccagatagctggggctgcaggcatacgccaccacaccaggctaatttttatattttttgtagagatggagcttctccgtgcttcccaggctggtctcgaattcctgagctcaagcgatccccctgcgtcagcctccagagtagctggggttataggcgtgcaccaccgcgctcggcccatttttgtatttttagtagagatggaatttcaccatgttggccaggctggtcttgaactcctgacctcaaatgatccgcctgcctcaccttcccaaagtgctgagattttagacgcgaaccaccatgccctgactataggttatctttttacttgcttgatggtgttctttgtaacacagtttttaattttgatgaagttcaatttatctgtttgttttttcttttgttgctgttgctcctgatgtcatatcagacaaagcattgcctaactcaaggccacagagatttactcctatgaaacgcctataaaactcctatgatttttatagtttagctcttaacatttaagtctacaatctcttttgagttaatttttgtgtatgagatgagagtagtggtccaggtttttccttttgcttgtggatatccgttgtccccacctcatttgttgaaaagactattctttcctcttaaattgtttgtttgtttatttatttttgagatggagtgtcgctctgatggagtggcgctaacttagcttcactgcaacctccgcctctcagattcaagcgattcccctgcctcagcctcctgagtagctggaattacaggggtgcgccaccacacccagctaatttttgtatttttagtagagacggggttttaccgtgttggtcaggctggtctcgaactcctgatctcgtgatctgcctgtctcctggcaccctgggaggctgagaggctgaggtgggaggatcacttgagctcaggagtttgagaccagcctgtaccattatgcctggctaattttagaatttatcttaaagtataaaatgtgaatccaatttatcttgttctaaatgactatccaaaatgttttaaccagttttattagtctgtaatttacatacaagaaaatgctcatctttttatgtttacattttaatgagttttgacaaatatatttgctcatgtaactacttgcttcatcagtgaagatggaaaacattgtgcctgttcctcttctctgtccaactgtactttattaccactagctccagttaaccagtaatctgccttcttttactatagattagatttatcctctttagatttctttttctttttttttttttgattaggtttttttttttctttttttacgtaaaaaaatctttttttggagacgtctcattatattgcccaggttggtctcgaactcttgagctcacctcagcctcccagagtgctaggattacagatgtgagccacctcagccagcccctagattttttttttttttttaataaatggaatcaaacagcgtgtaacagaggtgttcaatcttttggcttccctgggtcatattggaagaagaattgtgttgggccacacataaaatacagtaacactaatgatagctgatgaacaaaacaaaaaaaaatagcaaaacttataatgttttaagaaagtttatgaatttgtgttgggccacattcaaagccgtcccaggacgcaagttggacaagcttggtatataatttcatatgtgtgtcctaaacagtgtagtaatttgaatttcatgttagtatcagcttattcctttttgtttgtttgtttgtttttgagatggagtcttgttctgtgtcccagaattggtctgcaattccactgcctcagcctcccaagtagctgggattacaggcacgtgccaccacacctggctaatttttgtctctctctcttttttttttttttttttttttttttagcagagacgggatttcaccatgttggccaggctggtctcaaactcctgaccccaaatgatccacctgccttggcctcccaaagtgctgggattacaggtgtgagtcaccgtgcccagccagcttattcctttttattgctgggtagcatttcattttatgattataccacagttaatttacccattactagtcgatgggcatttgagttattgccagcttttggctattatgaatgaagctgctgtgagcatttgtgtacaagtgtttgtgtttttatttcttttagttaaatacctagaattggaattgctgaggtatggtaagtgcatatttcatttttttaaaaaatttattttattttttatttatttattttttttgagatgaagtctcactctgttgcccaggctggagttcagtggcgtgatttcagctcatggcaacctccctgtcccgggttcaagcaattctcccgcctcagcctcccaagtagctgggattacaggcgcgcaccaccatgcctggctaatttttttgtatttttagtagagacggggtttcaccacgttggccaggctggtctcgaactcctgaccacaagtgatccacccgccccagcctcccaaagtgttgggattacagatgtgagccaccacacactgcctggtaaatacatatttcaattaataagaaactagcaatcttctaaagtgattgtgtcattttacattccaactgatcaggtacatgtgtaggttccatgtgttctgcatccttgccaacacttggtattgtgttatctttttaatttcaacaggtctaatgggtgtcttatggtatctcattgtgatcttaaatgtacatttctctgatgatgactgatccaggagcacctcatcatgtgtgtgtttgttttcagctgtcaacctttttttagtaaatggttcaaatcttttttccattttatttatttatttatttatttgatggaatctcactctattgcccaggctggaacgcagtggtgccatcttggctcactgcaacctccgcctcccaggttcaagcaattcttacgccttagcctcccaagtagctgggattacaggcatgcgccaccatgcctggctaattttgtatttttagtgtaggtggggtttcaccatgttggtcatgctggtctctaactcctgacctcaggtgatctacctgcctcggcctcccaaagtgctgagattacaggtgtgagccactgcgcctgtcctaataatttctttttgtctcaatgtttctgcctgggtgcactggctcacgcctgtaattccagcactttgggaggccaacctggatggatcatttgagccaacagtttgagaccagcctgaggaacatgacaaaaccctgtctttgcaaaaaaaaaaaagaaaaaagaaaaattagccaggcacagaagcgcattcctatggtcccagctacttggggggctgaggtgggacaatcgcttgagcgaggttgcgggggtttggagggcgatggaggggtgatcgaggttgcagtgagctgagattgcactactgcactccagcctgggcaatagagccagaccctgtctcacaaaaaaaagaaaaaaaagtcatgtttcttttcttactgtgaaaataaagttactacttttagtaaattattttaagttatttatatattctggttacaagtcctttctcagaatattgtgaatattttctcccagtctgcggttttttttgaagagccagtattgttaattttaatgaagccttatttatcaagcttttctcttaaggttcatgcttttttgtatcataataagaaatcttttacgtaccctaggttatgaatgtttttatggttaggtatatggttgatttcaggttaggttttgtgtagggtgtgatgtaaaggtctagcttcattttctccaccataaatatttactcggtttctctggcaccagcctctgttttccattggtggctttattttttttctgttcttgaaacaagagtctcgatcttgttacccaggctggagtgcagtagtgtgaccttggctcactgcaacctccacttcccagggtcaagcgattctgcctcagcctctcgagtagctaggattacaggtgcccgccactacacccagctaatttgtattttttttttttttttttttagtagagacagggtctcaccatgttggccaggctagtctcgaactcctgacctcaggtgatctgctcatctcagcctcccaaagttctgggattacaggcatgagccactgcgcccagccatagtagctttattgaattcagttgactgtattgtatgtgtgtctatttgtgaactgttttgttgtattgatctttgtatatatccttatgccaattctctctttattgctgttactttgtaaccaacctttaagttcatatgagtctcccagttttattctcgtcaaaattactcttattctgcgttctttgaatttgcaaataaattttagaatcagcttgggattgtgcactgaatctttatatcagttctgggagaaatatcttaacaatatggaatcttcattgaggtcatcatatactgctccatttatttaagtcttaagtttcaccagtgttttctagttttctttgtatcagttttgtgcctgctttcttaaatttatcccttaatatttcatctgttttgtgctgttgtgagttatattttaaaaactttcaacgtttgtttattcgtaaatagagatgcacttgatttttgaatattgaccttgtgtcttgatgtgttggtaaacccactgtttctggcagccctttaagacttaaacatacaatcatgatctaatcaccatgttggtgtttttgggttttttttttttgtcttattgtactggtgcattactgaaaaaggcatgagattttgccatgctcccatttttaggggtgagacattgtctttcactattaagcatacagttaggtgttacttcagttcctaatttgcagaggtgggtttgttttctttttaatcatgaatggttgttggattatgttcaaatacttatcatctactaagtatatcatattgaccaggaacagtggctcatacctgtaacctcagagctttgggaggccaaggcaggaggatcgcttgaggccaggagttcaagaccaacctgggtgatgtaggaaaaccccatatctacaaaacaatttaaaaatttgctgggtgtggtggcacacacctgtagtcctaactacttgagaggctgaggaaggagaattgcttgagcccagtagtttaaagcagcagtgagctgtgattgtaccactgtactccagcctgggtgacagaaggagaccctgtatttaaagtgtgtgtgtatgcgtgcgcatagatggatagataataatgtaattccattatggtcatacaaactgatatgaaatgccattttatcatataacaagtgtctttttgtggttgaatttgtttctggatttttcactctgcttcactaatctaataggactaccttctcatccactcactgccaacattgattttttttttcagattaccttgaattttctgtttatttttccatatgaactctataattaacttactactaaaaaaatcagttgcctttttaaaaccaactgatctttaaaatatatcttggctgggcccggtggcaggcacctgtaattctagctacttgggagactgaggcagaagaattgcttgaacccaggaggcggaagttgtagttgagttgagattgcgcacctgtactccagcctgggtgacagagcaagattccctcttaaaaaaaaaaaaaaaaaaaagaaacagaaaagataaatctttttacaataatttgttccaattagggtccaagtcaggcttgcaatttggatttgtttatatgttgaagtctttttttttttttaattgtttcatattgtggtaactttttttttttttttttgagatggaatcttggctctgtcacctaggctggagtacagtggcacaatctcaactcactgcaacctccccctctggggttcaagcaattctcctgcctcagcctcccaagtagcccagccttttttttttgagacagagtctcgctctgttgcccaggctggagtgcagtgatgcgatctcggctcactgcaagctccgcctcttgggttcatgccattctcctgcctcagcctcctgagtagctgggactacattcgcccgccaccacacccggctaatttttttgtatttttagtagagacagggtttcaccgtgttagccaggatggtatcgatctcctgacctcgtgatccgcccgcctcggcctcccaaagtgctgggattacaggtgtgagccactgcgcccggccttgtatttttaatagagatggggtttcaccatgttggccagcccggtcttgaactcctgacctcaaatgatccacccgcctcggcctcccaaagtgctgggattacaggtgtgagccatcgctctcagccttgcggtaactttttattacgaatgtattgagacattaataacctaggccagtcatgtttcatccctacccattgtctcttaaaagctttgagtccactggattattctgaagcaaattctagacattgcatcagtttatccaccaacattttagtgtgtatctttaagttggttttggttttgttttttgtttttgagatggggtctggctttgttgcccaggcttggagtgcagtagtgcaatcatagctcactgctgctgcgaattcctggtctcaaaggatcctccctcctcagcctctcaagtaactgtgactacaggcacatgccaccttgccagcttttcttttcttgtcttgtctttcttcttctttgtttttttgtttgttttttgtttttttttgagacagagtctcaccatctttctatcttgcccaggctagtcctaaattccagggcttaagttatctttctacctcagcctcctaaagtgctaggattacaggccagcactttaggaggtgctggatgagccatcacacccagccaagtcataggtttttttgtttgtttgttttttgagacagtgtctaactctgtcacccaagctggagtgcagtggcatgatttcagctcagtgcagtctctaccaattgggcttaggtggtcctcccacctcaacctcccaagtagctgggactaaaggtgcgcgccaccatacctggctaatttttgtattttttgtagagacagggtttcgaattcctgagctcaagcagtctgcctgccttgactcccaaggtgccaggattacaggcatgagccactgcactcagccctcacagttttaattacagtttttcccttagtttttgtcttgttcatatccagcttgtcttgtatttttttcccacgatctgaattttgctgactgtatccctgtgttgatatttaaagtagacttctgtcccctgtaatctttgtaaactgatagtaaataatgaaggcttgatcagattgggttttttttttttttccccaatgtttcacagatgtgtgtactttcagtgaggagtcatgtaatcagtctttttcctgataggagtagtcagtgagttcctagatgttttatctatccaggagataatatgtccctttagcgccttaatttttttggtgtgttttttagcagccattgatgataattgtctagcccaagatcagttatttccttaggggttgtaaaatggtgacattcttttcctttcatcccttcttcaattattgcctggaatatttctataaagaaaaactttcccatatccagctgtttggttaccctgaggtatagctttcttaggaaaagtaatttaaaatgttaatcatttccctttttaaggcagtcttcaaaataatgagttggttttctgttatcctccaaaggtaaccagtgaggtggtttttttgtcgttggttcttactatcagtataaacttctggaattttttttttttttttaattttttggagacaaggtctggctctgttacctaggctggagtgcagtgggatgatctgggcatactgcagcctcaacttcccgagctaaggcaatccccccacctcagcctcccaagtagctgggactacaggcaagcaccaccgtgcctggcttaatttttgtatattttgcagagacagggtttcaccatgttgcccaggctggtgtcgaactcctgagctcaagcagtctgcctgtgtcagcctcacaaagtggtgggactacaggcatgagccaccatggcaggccagaatcacaataaacttataaattaacttgagaagaaatgattgatgtcttcatgatgttgagtcttcctgttcaagaacaaagtataccttcaatagcatattaaagtttatccttggctggatgcagtggctgacgcctgtaatcccacctctttgggaggcagaggtgggcagatcacctgaggtctggagttcgagaccagcctggccaacatggtgaaaccccgtctctactaaaaatattttaaaaaaagtattagctgggtgtggtgtgcacctgtagtcccagctactctggaggctgaggtaggagaatcgcttgaacccaggaggcagagagtgcagtgagtcaagattgcaccactgcactccagcttgggcaaccgagcgacactctgtctcaaagaaaataaataaataaaaataaagtttatctttaaggttttgtacatttttttcagtgtatgccttaggtaggttcttttttaatgttagtgtaacccagggacttctcttccattgcatcttctaagtaattacttatgaagtaccatatatgaaggctattgctgtttatatgttagtttttaccctgctcctttactaaattccaatcctttgaggtattggataaaaatatttttagcatttttcaaataacaggcagagtcaagggcttggtttcttttcttcccctcctgtcccctaccctccccttttttgagacagggtctcacttcttcgccgaggctggagtgcagtggtgcagttacggcttaccgcggcatctgcctccctggctgaaaagttcctcccacctcagcctcctgagtagctgggaccatagatgcacagcaccgcagctggctaatatttttgtattttttgtggaggcagtgtctccccatgttgcccagggtggtcccaaactcatgagctcaagcagtccgctcgccctggcctcctaaagtgtagggattataagcgtgagccactgcgcctggcctggggatcatgttttaacatgagaattagtggagacaaacacatgatatctaaataatagcaccatagtatacttgactagctttttaattattttttaaatatacaggaaggtaataagtaacaaagtaataatagtgaatagtttaagctcagttagcataatcgggcaaactttcatttgataaaagtgataagtagttttcagtggcttttttgtttaccagaaggaggtggtttttaaatacgtgcatccaagataaaatataaaaaaatgttcaggtttgctttcctacatagataaaataatatgtaactagctctcccaaatttcagcaacagttagtgaatgtttagccacaaatttgcagttaattatataatcagttcttaggattttatgaacaagttctatattctttgtgccttatacctagttgtaagcagtcattccacaattattttcctgaagtggcttggttaatgccacaccagaaacaggtcacagacaatagtgctgtaagaaatgtgtgaggaaagaggcacatgggaagtagctagctcgtgctggaggaactggaaaaaaacctcacatgggagatgacagttgagctgaattcttaactagagttgtaacagggcgaggcccttacatgcagaccacctgtgtggattaagataagacataaagtaatcttttaaaagaactattatttagaaacctggtatatgctacatggtgctgtgttatactgggtttgagaaagaatgggaagtgttacaaggattcagtggttggaaattaaggaagatagaaagttagtgttggatctgttttggctctttggtcatgcctttgtttttctcaaaatgaatgcagtgcccgtcccagaaaataccatatgagaagcgatttcataatgctgtgagagtctgttacagggacttgatcaagtctgagggccatgagagaaagtccctctgaggaagttgctttcaagctgacacctgaaggatgaagcagaattatcccagctgggatttgggaactggtgtttgaggctgaggactagcatgcatgataggaaaataacccagagtggcagaagtgggagtggtatgagatggcatcagagacgcagattcagggtcaaatcattcagagcctcctagaccatgtgaacacatgtattatgctgtggagatactgtttaataggcagtctgcttttttttctgcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcaggtaaggctggtaaggcctaactcttaatttttgtaccatataaaaaaacttttaatatggtaaagggattttcctttataatttttgcttttgtgtgatggtagggtagatagctaaggacttggggacccttttcaatatatattcgaaggttactgatgattgtaagaggttcagaggaaacagccaagaaagatttgagagtttacagctgtttctggaaatctggaaaccatggagttaaaaatcttaactaaagtctgcttggctctatttgcagtgttaatgtgctttctttattttttgtttgaacacagcatcctcatgtctatagtcctgtaatacagggtaatgctagaatgatggcaccaccaacacacgcccagcctggtttagtatcttcttcagcaactcagtacggggctcatgagcagacgcatgcgatgtatggtaggaagcactttgtttgtctcttccagtgtgtgtgactcttcttaatttaagtttctgaaaacatactctatctaagaataacctgaccttttatgacattgagggtcaagaatctgaaggaaaagatgaacccatttctttgcctgacttgctttataacttttggcaaatagtttctacttctgtacctggtcttcagatctctttcctgctttaactaaaatgtaatgatgtatataatggcaaagcatctttgtggagaaaggtacctttctcctcttcctcatcaatattatgctttggtatatcctgcctacgacatgcaagagaattttataataataaaagcataaaggtgttctccagcatgaaaacattttgcttcactacttgatctgagggtcactggcattacatattttttttgctgtttgttataatgataatactatgtttctacatcatgctgtattttaatggttgaatattatgtcatattagatatattttagacatgagtcacactttaaatataaccaatgtgaacagaatgctgaaatgaaaatgagaagtattttatgtaaaactaagcagtatttatatgtgagaataataagcaaaaaaacccatcttcgttttgtgactaaacagagaaatttgtgtagatcaacttagcagctgtctaaagtaccaaaataatagatttttcactgttgataatttaaaataaaatgtccatttgtatatcttatgatacagaattaatggattgcttcaaatgtttttcagaatatgtttttaaatagtactgatttcattaagatgttttgttctgaatatttctgagaactaccgtagtgtcgtttagttttcctatttgcgtttttggttgtttggagtaggggataattttggtttattcatacagttgaaaagtgtactgctatgagaatgagattatggttacatgtaactacatgggcatttcatttttaaagcctctttgaactttttgaaatactaagaatataaaatttttattttttaagtttagatgtcctgaacgagtatgtttaggcaaaattgagttatttaagaatttataggctgggcgcagtggctcacgcctgtaatcccagcactttgggaggccaaggctggcggatcatgaggtcaggagatcgagaccagcctggccaacatggtgaaaccccatctctactaaaaatccaaaaaattggccgggtgtggtggcatgtgcctgtagtcccggctacttcggaggctgaggcaacagaattgcttgaacccgggaggcagaggttgcagtgagccgagatcgcgccactacactctagcctgagcgacagagtgagactccatctccaaaaaaaaaaaaaaaaaaaaagaatttacagatttctggcaaaccttcttcttgagacattactacttttcataccacctctgtcctttttgaagaataaaagttttaacattccgtaggttaatgagaataggacttgggcagcagcaatcatccttcctgtcacctgtaacccacagcttatgctttcttcctggaggttcttgtctgccacaaaggctcactgctgataggaatttgtatatgatcaaaggtgtttagttttataaaacagttaagtccagtcttaattttccacattatcactttcaattttgtattgtggattacgcattttaaataaaaaattgtgtgattgctacattttggaaaacatttttttcaagaggcccatccgtaatttaattgtaaaagatactgacaaactaacttggtttattattttggttatgaccccgtcatttgacttgtctttagttgtcttaacggggactgaatatgcgtgcaaaggcacgattgatttatcatgctggcttttatgcaacttgtatatattttaacaattttcctgtttgctaaaggcttaggttaaaagttcattatgattgtttatacatttctggtgaatacatcatgatttaacaagtggaaagaacatctctttccttccattttctggcatactccccttggaatcagatctgaaacttttaagctaaaatttccattgcatttggagagtagttatttgtgtatgcatgcttttgagacattgtagcaataatactgtaatgttgagccgaatctttctcctcattgtgttcattcactgccaacatctggcttcatcttttggatgaatgttcattggttttgaaacagcctatagggtaaatactgtgtttgaggtacagatgattttcataactacttcctagaacatgtccatttgaagagcagtggggccttagaccccaaagtccatttatgtgtgggcaaataggaaatgttgcaaacaaaacaaagcactagatctaatgtccagtgaaatctggaatgaactagtcattagagccggttctttcatgccaggaaaaagttactcagccaaatctgaactactctcctgcagtttacacaggtggtatttaattgctgtctgtatggaggcaggctaggagcaaggctgtggacttgttgtgattgtcactagttaatcaagattccctttgtggtgcttaagaccctaaaaaggacactaggagctgggcatggtggctgacacctgtaatccaagaacttggggaggctgaagtggaggatcgcttagcccaggtgttcaagaccagtctaggcaagatggcgagatcccatctctaccaaaaaaaaaaaaaaaaaaaaaaaaaaaaagcccagtcatggtggcacatgcctgtagtcccacctacacaggaagctgagatgggaggatcacttgagtccaggactttgaggctacagtgagctatcatggcaccactgtaatccagcctgggtgacagagcaagaccctgtctctatttaaaaaaaagaaaacataagaaagaattgttttgttctatgccatcataagccataatttaatctgcttaagcatgttcttcattaaatctgcagtgatttatttgaattattagactttcaaagccttattatatcaaatataaacaaaatttgaagtacattcttataaactacaacaaacttacatagaagtgttaattttatactcatcttccctgaacaatttatattttataaatatattaaatatattgtcataaattttctcaaaggaaccaaatactttgagtatgaattgtgcttttctttttaagctacatcatatctaggtttttaaaacatttaatgcaaacagaagaacatgcacccagatgttggtgacaattttatgtcaccttttctcattcattaattgttatagccatagccaaaggcattgaaaacataggaccactaatgactgcaaaatgaaatcctgattattgtttttaaatttttagtatgtttaatacacatatgctaacattactgaacagttaaatgataaaataggataattattttattctaaaaaagtattgaccttgacctctttctagctatcttagaaagggcttttgtcaaaaaccttatctctttgatgtctctttttttgagatggagtctctccctgtcgcccaggctggagtgcagtggcgtgatctcagctcactgcacgctccgcctcctgcgttcacgccattctcctacctcagcctcccgagtagctaggactacaggcgcccgccaccatgcccggctaattttttgtattttgtttagtagagatggggtttcactgtgttagccaggatggtcttgatctcctgacctcgtgatccgcctgcctcagcctcccaaagtgctgggattacaggcgtgagccactgtgcccagcctctttttttttttttattttttatttattttttattttttttttaatttttgagaaggagtctccctctgccacccaggctggagtgcagtggcgcgatctcagctccctgcaaactccgcctcctgggttcaagcagttctcctgcctcagcctcctgagtagctgggactacaggtgcccgccaccacacctggctaatttttgtgtttttagtagagacagggtttcaccatgttggtcaggctggtcttgaattcccgacctcaggtgatccacccacctcagcctcccaaagtgctgggattacaggcgtgagccactgccccggcctctttgatgtctcttaatctaacttccatcattgcctctaccccatcccttctaagaagttactttaattttttttcctctcacatctactctttttttttttttttttttttttttgaggtagtctcactctgtcacccattctgaagtgcagcggtgcgatctcagctcactgcaacatctgcctcccaggttcaagcggtttttctgcctcagcctcccgagtaggtgggactacaggtgtgcgccaccacgaccggccaatttttgtatttttagtagagacggggtttcaccgtcttggccaggctgatctcgaacttctgaccttgtgatttgtctgcctaggcctcccaaagtgctgggattacagatgtgagccaccacgcccagcctcacatctactcttctaatccatctaattttgttttatggtgatgcttttacctttcagaaacagtaataatacaacttttccgactaactagagccattaggaagaattagatccagaatccttttttgatttgtttttggtagtttaatgcagataagtaagaaaatatagttaagttaaaaaaaaaaaaaatgaaaagcatccataatccctccacctgacaactgccttttaacattttgatgtgtatccttccaggtgtatttaaatacactcaaataccctacccctttatgtagacatgttttaataagaaataatattcatgtttatattcttgctatgatcctaaatttttggatccattactagataatctttcaggataatgacatttccattagtaatgtttttgcaaaattgtgtgtctattgaattaaacttgtaaaatagttttattttggtacatgatttatatcaaggttgttcagtagaatgccatgttggtgtttttattagataatgattttattccttttacttttaagcaagtcagcatgacaacttgacacctaagtacagaagaacagtgtcttccggtttagtcctttcttttaaaattctgtagcagtgtttaaagtgcttgtcatctcttatgaaaatgaattatgcatgaatacaaaaagaaattactaatatgtcaacctttccagaaaatttggaaaatgcacacctcaaaaggctaatttacctttctatttcccaaattcagcatgtcccaaattaccatacaacaaggagacaagcccttctttctactttgccagtgagttgggttttttatactaatttttaattgtacagtaaaacactttttaaaggatacatgttaagggagtagacttgttgaacaatattttccttgtgccagtcaaattattgaaagtacttatatatataaataattcagtttttaaaatggaaatacccaatttaagaaggctggagttaatgaaaaatggagttgtttcagaaatcaatttttgcataccaagcaaatgtgactgggaaatgcctaatattttccttgttagagaaacttcctaaacagctttatacacacacacacacacacacacacacacacacacaaacacacacacccaagccacaagcttggtataaatttaaaatgtttatttatacacacacacacacacacacacacacacacacacaccccaagccacaagcttggtataaacttaaaatgtttatttatattctgataagatgaaatttatgcctaccaggatttttaattgaataggattgatgaaatactaagggaaaaacttttcagtcctgtgcatggctaaaggtttaaaatactcaggaagggccaggcacggtggctcacacctgtaatcccagtgctttgggaggctgaggcgggtggatcatctgaggtcagcagttcaagaccagcctagccaacatggtaaaactccatctctactaaaaaatacaaaaatcagccatgcatgctggcatgcgcctataatctcagctactagggaggctgagacaggagaattgcttgaacttgggaggcagaggttgcagtgagccgaagtcgtgccactccactccagcctgggtggcagagcgaaattctgtctcaaaaaataaaatattcaggaagcagacccctcaggatatcttgagcttaagcaagagatcatgacctctcaggtcattatcttggacagcacaggtcccctctccccacctggcaaaaagtacagaaatagttgctccttcatggagaaagtctgggcagagctttcttctggaaatgaacttttaaggtacatttttcctatttgtagggcaatttgtaaaaataagggccggacgtggtggctcacgcctgtaatcccagtactttgggaggccgaggtgggtggattgcttgaggccaggagttcgagaacagcctggccaacatggtgaaaccctatctctaccaaagcatggtggcacgcacctgtagtcccagctacttgggaggcggaggcacaagagttccatgaaccctggaggtggaggttgcagtgagctgagattgtaccactgcactcaggcctgggcaacagagagagactctgtctcaaaataaaaaataaaaataaggctagtcttggactttggtatttaaataggaaggagtactaatatttgtagaaatcctttagaaatttgtgccattaatattgtcaccttgtatgaaatgttgtgttctagaggatattaaggattcaaattttatgttaggcacattttgagttattttggggtgactcaatgtctgactctactaaatgccatattagcatttaaaatgcatttgaccttaaatctttgttaattatgccatgacttggtatccaaaaataagctgatacatacatacatacatatatgtgtgtgtgtgtgtgtgtgtgtgtatatatatatatatgtatgtgtgtatatataatttatttggtgctaggaaatgttaaatttaatcctttaatagatgctctttaaaaaggagtcttgctgtatgtatatactattaaaggggaaactatgtctgtgattgtagtgtgtaaaagatagtaggtgattttattatgtactcaatttgaggtctcaaatgtagttatcctcaccatcttactgtctctgttagtagtttggtgttgttttcctggtaagtagctaaggtccttaatcattaacacctaagccttaattgccttagcacaacttcccctaaaagggagtatcagtactttttaaaagaaactaacagttgggctgctaatttaatctgctgcttcatttccccctgttctaagccattttatgatggtttggtcaagttgccttttattccccttttagagttttcaactttccttcacttccctttttctgaatttaacatcagatttacaagttggaagattttgttttgttttataagttttgcaatgctggtgatctcttatgacttgtgcatccaaagtcaaaatgacaaaacctagttacaaattaaacacacagctttctgtacttaatttgcttcagtgagatcacagctgaggaaactagttctggaatgtggttagtgttattaaggatttttgactgatcatatgtttagaatcttaaatatttatgtcaaggaacactgagtgggaaacttctggactaggtctggaccaaagaagcatatgtctttgattatctttaatctaaaagattttatgaagactaaagttttataaatagaagtttaactgatgaataaatcagtattacaaataaaattaactttatttttaacctctctgggatctttagccagaatgagcatatataacaaaagcagtgaaataatatgtgtgggtcagaacccactgcccttcccactccactctccttttccctgattctcctgtgttttttccttctttaccttatcttggttcctttttttttttttttcttttgagatggagtctcactctgtcgtccaggctggagtgcagtggtgcgatctcggctcactgcaacctccgcctcctaggttcaagcaattctctgcctcagcttccagagtagctgggattacaggcgcctgctgccacacccagctaattttttttgtatttttagtagagacagggtttcaccatcttggccaggctggtcttgaactcctgacctcgtgatcacctacctcggcccctggttccttttttgtctctcttgtcttccaagctatttttttccttggcttttaaattttcttcctaccctgctttgtgtcactgtcacttaactggcctatcaaggaaccgaactgtatttttgttactagtattgatttaaagtataagtttcacatttctcccaatttattattattatttatttatttatttgtttattttattttttgagacggagtttcgctcttgttgcccaagctggagtgcaatggtgtgatgtcggttcactgcaacctccacctcccgggttcaagctattctccttccccactctccctagtagctgtgattacaggtgcctgccaccacgcccagctaatttttgtatttttagtagagacagggtttcgccgtgttggccaagctggtctcgaactcctaacctcaggtgatccgcccgcctcggcctcccaaaatgctgggattacaagcgtgagccaccgtgcccggctccatttctcccaatttcaaattcaaggaggaaaagaattcctgattaaggtacttctttcagatcttttgagctagaacaaaaaaacaaagggaaatatttctaattaactctttttaaattttgtttacaacgtatgatacatattttacacatcctttgtggtttttgttcgtcttgtttttaatcaatgccttgcaagtttaccggtatttaggtagggaaaggattttgtttttgtttttttaaacaaagcctatgtacattcactcagcttgggtatttgtgctatgcatgcaaattagctatagattagaaaaccgtattatagtctttaaatactggtaaacttaaattgcagagatgccttttaaaaatgcatagtaaaaatatttcatctttacttttctcttcaaatgattttaagatttttacatttttccagttgatgaataacttaaattatgagatttcatgggcataattattttctatatttattgttactttttaatattcttaatactttgcttagaaggtatttaaaagtgaaatttcaaactttttagtacaaaatttcttgaataaataaagttacaaaaaaaaaacaaaaacctctgagattccgtactgtatctttatgaacctccatgaacagaatttgggatttgggaattgcttttccttagacagatttagattgttacaaatgacatttttaagaggctggggtggcggtaggggttagtgctaatggtttaacagtaggggaccatggacaactgtagacatcactatccagtagaacattttgtggctgggcgcggtggctcacgcctgtagtcccagcactttgggaggccaagacaagtggatcacctgaggtcaggagttcaagaccagccagaccaacatggtgaaaccctgtctctactaaaaatacaaaaaagttagccaggcgcgcctgtagtcctagctactcaggaggctgacacaggagaatcgcttgaacccgggaggcagaggttgcggtgagctgatatcacgccactgcactccaccctgggcaacagagcgagactccgtctcaaaacaacaacaaaactgcactgtccaccgtattagctacttagctacatgtggcttttttattattcaaaaataaatttttaggccgggtgcagttgctcacacctgtaatcccaacactttgggaggccgagatggacggatcacttgaggccaggagtttgagaccagcctggccaacatggtgaaaccccgtctctactaaaaatacaaaaattagccaggtaatcccagctactcagaggctgaagcaggagtatcactttaacccaggaggcggaggctgcagtgagccgagatcgctccactgcactccagcctgggtgacagcaagactgggtctcaaaaataaacaaacatggccgggcgcagtggctcatgcctgtaatcccagcactttgggaggccgaggcggatggatcacttgaggccagtagttcgagaccagcctggccaacatggtgaaacccgtctctactaaaaatacaaaaatcagccaggcatggtgatgcttgcctatagttccagctactcggcaggctgaggcaggagaatcgcttgaacccgggaggcggaggttgcagtgagccgagatggtgcccctgcactccagcctgggcaacagagcgagactctgtcaaaaattaaacaaataaatacatttttaaaatgaacgtaagatttttacaagtacaacaaactcaggttcgaaatttacatcaaatcttttagaccaagtcagtgcctatacaacttggaggagctggaagtaaacttaatgagtatgatgatgatggagggcctgttaataagccaccaagttagaaaaaaaggactgtcttatagacttatgggactgtgaagctcaggaaggcttcatcgtttgtacatcatttgttctagctcccagaagacgttcactactcttaaaaacattcagagactatgttgccacagttttcttgttaaaatattctggcatatgttaattcctacagtctggaaaattttcccagtgtataaacaaagctgctgtatccagtctaaactggatatgaaggaatattaatgccagctgtggcattggcagtggatgcacaggtgatcctagaactggctctttgccttgccctttcccctgctaagagatagctttgcagctggagacgtaactgttagggctggagagttggtggcccttagccctacaacacctaggattatagaactgctccatgtgcctagcctaaccctctgcacaccatttacgtggaatatacccagagccgtctatgctggtgactcggcagccttgcctaccagactgctggaactagggtgcctcttcccaaagctgtgcttgcttctctcaccaatcagtcctgcatatgtctgtgtttgctaacacgttatatgaagaatgtggggaactattttggaatcatttctgtgtatgggcttattatcttgagggattttaggatttgtttctcaagagagggctgggaactataccttgctagagttgtcttgagaacgctctattctcagctcattgcctcgtggaggttagttttttatcatcggtgtgctgtccatagtcactggaagcagtgaacacatcctactctgcttctgattctcaacttactgtttttgaagcacatgaacaggccaggcacggtggctcacgtctgtaatcccagcactttgggaggctgaagtgggcggatcatttgaggtcaggagtttgagatcagcctggccagcatggcgaaaccccatctctactaaaaatacaaaaattagctgggcgtggtggcacatgcctgtaatctcagctactcgggaggctgaggcaggagaattgcttgaacctgggaggcagaggttgcagtgagcctgggcaacagagtgagtgagacttatatctcaaaaaaaaacaaaaaacaaaaaactgaaagacatgaagaaatggtttttgtaccaaggtttggcccacgctgagattcacaaagaactggctttcagttcttatctttattttgatttaaactggcccatcatgttgtcctttgaagttagtctagtaaatttctttccaaagggctggggcactcagaagggagtttacttttctatatttatttcataaagcaaagatgggagatcctccattagggcttgggaaagtaaactgagtggcagaagggctcctgtgattagctgagagagactgtggtccttcggccctgatgatagatccctggccttgccacataccatacacagtgcccgcacccccatcccccaccacacccaatatagtctgtgccctcaggacattgctccagggcagtagcatggtgaggttagcctgatgatggccttgagctaaagagtgtgcacctaaaatgcacttgtttgagtagtttctgcctatgccttcaagttgcctttttgggaaaacctagtgaccgttaagagtaaatgcaaactaatttgattttaatatcatatgtagagctgtattatatgaaccaaatgctagtctgttaagcaatagctacacttattttttcaagacaatggatggtttaaatggagtcatctatagaaattggtagtggcgtgagttatgcattgtaaccatcaagaaagttcagttgatgaagtgtagaggagcgatggaggttgtcagacatcggttgtgtacatgctcctttttctttcactttagtttccacgggctcccttgctcagcagtatgcgcaccctaacgctaccctgcacccacatactccacaccctcagccttcagctacccccactggacagcagcaaagccaacatggtggaagtcatcctgcacccagtcctgttcaggtaagggcaactcagaggtctgcatggagtggcttctttatcctagtatctgagtgctttcttcaggtgccaggtatcgcatcgtcagaacacatggcatgtccaccctcgtgaagatggatacagctgtgcccctggggtggtggttttaagaatcacatttaaaggctgggcgcagtggctcacgcctgtaatcccaccactttgggaggccgaggcgggtggatcacgaggtcaggagattgagaccatcctggcgaacactgtgaaactccgtctctaataaaaatacaaaaaaattagccgggcgtggtggtgggcgcctgtagtcccagcttctcgggaggctgaggaaggagaatggcgtgaacccgggaggcggagcttgcagtgagcagagatcgcgccactgcactccagcttggacaacagcgagactctgtctcaaaaaaataaaaaattaaaaaaaatcacatttaagatacatgttgataataaggtgattggataagctctggaaacttgcagtaatgaaaaatcaaatttaacataaagttcataaggcaaattcctatttgcttgggactttttaatttctaaggtttatgtgatgaggttattttcctatgagcttcttgaattatgtttgctaatggaggcagttaaagatgtctttgatatctatcagttccctggggcagtagtcttttttgactttagtatgtatgctcagaagtttctaactgccagactgagaatcaggcttctgtaccctagaaaggagttgtccagatgggaggcacctccagccttgctcttaccaccctgtacattctcctgtactttccagtgaccctcatcataggcccaagtgtgcaaagcttagctttgtgggtatcccttggctgcttttcattaaagaagttttcctctcaattctttcctgtcgctttgcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggtaataccagctttagccaactttctgtgaaggccaagtagaatgtgaaggttatcagtaagcagctagaggctctcccagctaggaaaccctgtgtgtcatgccatttgcctgtctccctttccctctcaaatacacgtgatctggccctaagggaatgtttgtgtggttttgtcatgggatcagtgaaggtgctgattggtcagtcctttagttttccaactgagaccttaaaaatatctttgactctggaatgcaacccagtccttctttcctttctgtgtctgctttgctatgtctatatagcctcactactatatatatgtgtacatatatattcccctacacacttaccttggaagccaggcagggatgatggccttcacagagtctcagctctccgaagtgactaccggggcctgtcaacttgattgttactcacatgagttccagacacatctctccaattgttttccctggttatccatatatctgctttgaccataagttgtactcttgagagggcttggccttggacattggtgcagtgtaactagaagctggaagcacccaggtggtcccatttttctttaagagcagccctggaagcactttggagctcacctccagtgtaagctgctacaggtgaaaggtgtgcttgccatctcagtggttgctgtctgcatcagctgctgacaaaggtccctgcactccagggcccaggggattgtcttaatgaggagaaggagctgcactgaagttgggctctaacgctggccttgaggccctccctggggctgttacgggtgaattggctgtattagatgtctctgctactttcataacagaactctctgaggcgggtctaagtgagacctgccacaatgaattccatttcctgttaaatagtgcgccagtgaggctctggcaaggtgtgggctagagatgcgactcagttggatctatctctcagaaggctaccttgtaagtagagttccacagctctgggaagtttgggcgtcctcaccctgcaaagtttaggttctgtggtgtagcgcactgcagttgatttgctttttgatagtggggagggaagccggtttggtccgtgtgggccagcgtggtttggtggagtcagcttcataagagctggggtcctgtaggtgtctaccagaggctggtggctaagtaggcatgtgaacttacatgtaagtcagggatccctaaaacctcactctgtttttgtgctgaaagggcaaaaaggttaacacagggaagctcaaatttgccatgtgcccgtttgaatatgtgagagtaaaaacggcatttcatccaaggcttatcgtagtctagaacagtgcacagtgtgggaaaaaggaaacaagggctcttcctggccctgccaaccccctgcagagctggaatccagctgtttgggctgactaaaatcacctttccaacttgacagtgagtgagaccaggttgaacttggtacagagacgctgggctggcccagatgacttcaggttactcctttccatctcactggagccattaaaaactccaactcctcctcctcctcctgctccatcagcatatctctgagagagtcacgggggcctaagagtctcttttcactgcctggtgagcagaccagaagcagagggagagaggcaaatgaacagaggtccaagtaattcacatacttgactgtgacagtctctgcttattaatgtaatctgttttcctatttgaaagggatgttatctgcaaaactacctcaggccccacatggcagcctgattctgaagcatcattgaatcttgtatgatattaagttgagaaagctgcccttggatccagtgtctaatctttgtgaagatcttaccccatacatagaatacaatgatcagaaatgtcaagggttaggacagcacagccctgacttctacccaggctcacttgttgcctgctccctgacccttgcaggatctgcccaaaggtgaagcgcgtcttcaggtcaatagataatctactagagattgtccccagagaacagaactgggccctgaggcccaccgttgccctttcctgagagtcccagcccagtgaaaggaacacagttgacatgttgttgaagccggagatgttgcctgtatgcgtaaaagagctctctgtttcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcaggtgaggcgtgtgtgtgcaggggccgccggggcaccccaaagcattctgctcgcacaggtggaatggcaggcagggccagtgcttcaagccccgcatttgagaactagcaagacccgtccaggagtgtgcacaggagggactgtgacgatcagttcagcatcagggcctgaggcttccgggagccgagtctgtgtgtgttctgatggtatacaggatttggcttgatgagaagcagcagcagcagcaacagcagcctgatgcatgcctaggactcagttggccttccttgttatgacaggctggacagggcagtgttttccttcctgagtcccaaaagtctgacatgtggggggttattaccatggcagagtttgattgtagctctggagaagatactgctgagaaagcgctgtggatggactggctttgagtgtagcgttagccccagcccctgaacaggggagagcgccctgtgattgtgctctactacttgatggctgccatggcgatacttcacagtctgacctgttattctgaaagcaatactggtgcttggctaatatttggggagggggtttgttaaggcctttttttctaccccatgaacaagtcttctgggagttttatctgaagtggttttacgtctgactggtttgtttctacccacccacccaaccctccccactttggtgcagatgggagggggaaaagcgaattcaattttgagttttgttcagctagcacgaggatagtttacaatcatgtgctgcagagacactaggctgatgtgtggtgttgccagttttctgtttcaatgttcgcttttctttttacagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgatcaagttagtgtgtgtctgtataagctacttctttgtaggatacttaatatcaaagcaggtgtgctaagggtgcattttgaatatcccggaaggtagctgtgaaatgatttttcttcttcacccttagttctggttcaaggtatctctagaaaaagacaagactgagctattctctttggtggattagagatctgcttcaggaggaggaaggttggccagagttgggcagcactgaaattccacatcctcgggctgacaccgattctgtaagcttcctttttaatatctcctgaaccaaaatgagtgtcattagctggaagttcccaattcgggcatttttctactttaccagtagggggcaggagacactcagaaaaaaattgcaataaagaaatccagagggcatgaaggctgaaaagatacaaagatgtacaaagctgcttattgacatggatggactcataagcatttgttagtattcccagattgcaacggggaggacaaagggaagagcgagtatttgggcagggcaaggattttgtagagacaccatggtcttaatagagcctttaaatattatgacagcaaatcaagattctgaaaactttttaattcacatatagcaatttgtacattatagcaaaatttgcattattcaagaataagttacttgtacagtacataaaacaatacataaaaatttgccaaataccttctgcctataatgatacaagatgaatccactttatgttatcacaatgtgctgtatattctaaccaaacacaggatgtcagatgtgtccttgttaatatactcgcaagttcctctagcttgtgggagatgttagagctaacacatttgcagtaagggacttagtcctgaatagaaagcatgaaggaatctcaggcaaccctcagggaagagtccaaggccttgactttaggttaagaaactgttatgtaaaaatagtgttctctggcccaagattttaatgattgctattcctttttcctacggtccagaaatgatcaaaggcagaagatttataccagataaagccatatggattgctggtctaaaattcaaggcaggttagttgacttaattctttggtgctggtgactgttagtttgtaaaagttcaataagtcagatgaaggaagggatggtgccgggagctgtcaagctgtactggtggggtctgtaattagagctaactggagggatcatgatgtctactgtccagtttggtgttgagccatggctctcggtagaagttgccggctggggcctggtcaggactggaaggagagtggtgggatgtgctgtgcctatggtgggctagctgcagccagtggggtgcctgccccacactgctgcccaccccttcatcagctgattctgctcccacataaagaaaggtgttggcttagtgtcacttcttcctagagccatgggagttttctgtcagcatgtttttgagctgtcctggtaacttggacgggaagcagtctggaggtgggtgccttccaaatctctgccacagaatctgaacttgcactttgcagcctgttagtcagagctgctaagtttgctctacctggtgtgcctcttggcaccacctgagagatgtatgtagagaggctgctaagaaatgagtgcccctggcatgaagacgtcatatcccagatgtttaacttctggctggagggagggaacttaatttggttcattatctactgggtacctttcctgtgcggaggggaggaccagtgactttccataataagctttctcctttaaaaaaacttcagactcttacttactcttttggttcacactaattagcattcattttccagagaaagctgcttgactttgaggaaataaagacaggtgtccttaaaaatggactgcaaatctggatcagtgtcaggtctgctttagggtccatgaaatcacccaagagtgttggtactaaaagtggtgtaggctgggcgcggtggctcgcgcctgtaatcccagcactttgggaggccaaggcgggtggatcacctgaggtcagacattcgagaccagcctggccaacatggtgaaaccctgtctctactaaaaatacaaaattagccaggcatggtggctggtgcttgtaatcccagctacttgagaggtcgaggcagaatcgcttgaaccaggaaggtggaggttgcagtgagccaagattgcaccactgcactccagcccggttgacaagagaaactccatctcaaaaaaaaaaaaaagaaaagtgcataggggtttgttcccccaccctctgaaagaccccaaggaaattctagaagcaaaaatttgacctcttggagcacagagactaagtataagtggaagaagcagacctaaccctcctcccaggcagtcagtcctatgagttttaggctgaggtttcattttagcctctgatcgatgtatctgtaaagctgatctaaactcccagtttcctagaagccactgctgtgagcctgggggttgtactgaatggctctgctggcacccaagagcagctgactccactgtcactgcccccgtttctgagcctccagacaggttctgggaggagccagccccctatgaattgtatcacttgtatagatagagatttgacttcaggacctccctggatggtggagattgtgtgtgcccatgtgtgcaaggagatagatagattctcaatccagtgaagcagtagccagctagaacagaacactgattgtgggctattaaaggagtttctcattagttatggcaccagattcccagcttccattttggctcaaaaaaggagcctaaaacccacttgtgggataatttaataagccattagatgcttggagggcaccaagccagccttgttgctgaaggatttactagaaagggcctaggagtccttcttccctttttgcttgctcttcccctagccccagttaatttaactatcactgtaaagctgctgtaaaccagaatccagttttttattcgtaaggcaccaggatagaactgttcttgccaccctcctatttcagtaattgcctgaatttccctgaaagggtgacatacttcacttcttgacatttaggaaggacagtggccactcagtggcaggtgactgtcccgaggatgcagtagacaagccagcatgctggctcagactgtaaattaggtagggtttgcatagctgtttctgactacaaaaatagcatgggtgttagttgaaacagtgcactgaaataagcagtggttaagttttgaacctgaatcctttaaagaaattagtctgaggtaaaatgttttaaatcatttttgaccttcagcttgtgcctctgacagagcttttgtcctaaagctgagagtagaagcaggattagatgtggtcatctgtggtgtggacacagcagacctgccatgtggactgacggtagatgggagaaccgcttgttagtgttctgggcttcctgatgatccccccctcctccccgtagaaagtctgtcatccgctgtgacagtgtcagtgaaaaagagaacaagtttccactgccagctctggccctcaccccaactcctttctctaagagtaacggggactgggcatagggaccccgggcaggagctagcggcatcctctaacaagaactacatctgtaaacaataaggagaaagccaggtggatagatgaaacctacaggagggcccttcaacaggcctaatagaagggtcacttccaaactggcacgagcagttaacagtgtcccttaaatctttctctggaaggaaggaaagatcaattacattcacaagttcacacttctgcctgtgctcctcaagggcagctgttgaggcctggaattcctcactggtcagagaggtgtgtactgattgtctatggcccgcaggtggctccgggaggatgagtccatttcgtagtccgagtcccgggctcgattcacaggctcatgctccaggtgctgcaggctgttggccagttcttcgggcgaaggcaccaggtggaagatctgctcggggggtgaggatcgccctgggagaccctctgggctgagcccccggggacagccagaagaactaaggtggggaaggcccaactctgaaccccagtgaggaagggactctgaatcccagtgaggaagggagaaagggaagaggaccgtgttgcaggaacctaggacaagtgaccaagagagaccacaccatcagtcttgaggcaagtgctgacaggacacagaacaagatacctgtacccctgggtctgcagcaagcctctaccctctacccagtgtcagccacccccttcagcaaagggcagtagggtcagacctggtggttgggagacgactaccacgtagctggagagccggacatcacaggcggcgccgcactcgagtgggatgggcgagcgctggaagtggtggagcatgtccacgaccgagggaaagtggaggtgctgcacacggcactggccccgctctgtcagcgacaggcgcaggtgctgtgggaggagaggatggtgggctcgggctgtccctctaggaccctgaactccagtggagaggggtggcacggtcctgcctgccccaccccaccccaccccaccccataccttggctatcccctgaaagttgaaagtgagcacgtattccccacgccgcgtctcgctctgccgcaccaggaacactccatgagcatcagggccctgcagctgaaccagctgagctgctttcactctggagatggggccgtggaaccaggggtagcaggacaggaaatggtccgtcttctggcaggccgggtccagcagccccccaggagaagcacctgttagataagatgggtgatggtggtcgcagggggctaccttatgtcctgggctgagtctgggttccagcgtacccagaagacaatggcctagctattggtttacccaccttggttaagggaatctgtgctgccccttggggagctggacgtgctaggctctagggctgagggaatatgcatctctgcttctgtgctctccagcctgctggagaagaggctgaggttaaggatctgggcaggtcaggggtggtagcagcaaaggtggtgccagggacgagggcccaggacctcaccctcggcctgtgcactccgagagctcagccatccatgaattcagctgctgctcgtctcccacctcaaagatgatgtctgtccggtccttcacctggcagggaggaagggaacagggttgggtctgctgtatgtgtatgtatttgtattgccccagggtgtgaaaagccttgtcactcaccttcagcacaaaggtgtaaaggttgtcaggcatctcaagccgtgtgcaccaccggacctcctggatgctggagcaagctgcttgtagcttgggccttgaactctacgagaccaagttggggtactcaaggcttgttcctgatgctctttcttccagccaggcctgagtttgtctatagtctccccaggagtcccccttttagagagagtatccatggtgttgctgggcaggctctgagcctgtagtgaggtccagggtgccttcccctagcctcctgaacaccattcacactgagatcccaagaagcatgtgctggcagaaggccagccctcccagcccattggacctggacttggagacattcccactctgccatgtgtctgacatccgtttctcttcccactggtgagcaaacagctccctgcccagccaggtgatagcagctgctgacggtctcatcacaccaagcccaaaggggcagagcctagcatggctagcctaagttatagccactgtaaaaccacaaaatcttggccctggaccttcccaggacacccggcacatagggctgcagggtggataggagttagccactgttcctttgccagcaggaaacctgctcccaggtctcaactgaccctgcagatgcttcctgtgctggggcactctgcagccaaggagccagctgcttccggttgggaatctgctcaggagggggtttgcccctccccttcctcttgtgataagactgaaaagatggccgaaagaagaaaaggggagaaagagatgagataaggaatggacagaagtggccccactgggccccctccattcaggtgttcctggaaaatactacaacccctaacaaactgggagcagggggatggaaagcagaggcagcccaaaggatgttctagcaactgtggcttcaggtgatctgacctccacctcagcccaaaagggcataccttggactggagctgctagtgctgttgaattgacccccaaaaccagatgcttaaccttgctttacacacagagctgccaggtacctctgtcggaaggatcatgacccagtggcctggccacctcctttatcccccttccccacctgcaggagagtcaatgaacagtgaggtaagagggcagcctggcaggagcaggatggccacctgggcccttccagcaggtctcctgggagctcccgggcctgctctggttggcggacacaacaggctgggggagagtaatgactgcaggaatgaccaacactggccacagctcactgcggcctctcactagaaagctgcatgactagaaaattccagcctgaagtgaccagcaccagccagggcattttcacctcctactcccagaggtccagcagccaggatgaggcaggcagagtgaggcctcacatggcatccacacgggcactggacacagcgcagtgtgcagggagtggggcctctgcgctcttgcccatttttacagccagccagccctgcagagccttcatttcctgtctgcaagatgggtaaggtgcgagtggccacttccttctgctacttttccagttggccctgcctggcccccacccttccccctagctgcaggccttaaggatcactgggaggatataggccaggcctggaggggaacgggcctagtctccctttcagttccgctccaggcctagtccccagtgtcagtgctgagattaaccagcagagctgccggtttcactctgactctggcccctgcaagccagctccacccccagcagctgaagaactgctgagcaggcaggcagtggtgaaaagaggattcccctcctcacccacagccagcctggctgccacctgcctgctggctgagcacaacccaggggttcttccccaactccccacagcagcccttgagagccgcccaggcctagagccacaatctcaacccaagcctgccaagtatcagagccggggtccgccacagctacctggtggtatgggtgtcctatgccagaggctccagccaggttgcagcgtgtgggcacatgggcgcaggacaacccctgcttcagtcgaaacctcagcctggacagctcaggctctggccactgcagggaaggaccaccagatgcccatcagattaagctatatggggggatgggtgagaggggagacaagctcaaacctgggggctgtgtcagcagctgtgggggctttaggttccctcctctcacatgcccgctattccacctccctcaaaagtcaagccaggccagcaacagctgctccctggacctgcttaggctctactgtggttgggtgatcaaagtggccagatcatttcaggtatttgtggtttgaacagtagactgtcacccaccacatcctgcagcgtctatttctggggagcaagtcttctccattcccagcatctaaacagatggcttctgtttgttatggccactgtggctccagtgggaataatctatttctttctttgctctggctgctaggaatctcaaagtcaaagacgtaggccagctctgacactaccccaacggtaggacttgtatttatttcaacagctaactttatcccagatgttatagttcttttatcctctagggttttttgtttttgtttttgttttttgagacagattctcactctgttgcccatgctggagtgcagtggcgcaatctcggctcactgcaacctccacctcctgagttcaagcgattctcctgcctcagcttccgcagtagctgggattacaggcacctgccaccacacccagctaatttttgtatttttagtagagctggggtttcaccatgttggttggccaggttggtctcgaacttctgacatcaggtgatccgcctgccttggcctcccaaagtgctgggattttaggcatgagccaccacgccgggccctaccctctaggttttaagattcagccctgaccctgccacttcctatacaaccctggacaaagggttcggctctccatgccttagttgtcatctgtaattcagctcattttttcacaaggtgagctaacacaagcgaaaggcatgactcgcacagctgttaacaggccggtgagtaaagcctgcttggctcccactacactactgacagcatgacagtcccctctgagtagcctagccactgggtacacccccagcttctctcagccttacagaactattgccccattttgtcacatgctcccttaccatcctgcaataaattccacgtaatataaccaacatactaccctaactaattacaagacagggacaacaaaggactgtcacttagccgaaataaaacgtgcttcagtgcaactgtagacaacacaaccaactacttggctgcttctgagtttgttgtgaaaagaaggctaagaacataagggagccaaagggcgggggagctaggaaagccagcctggttccagatccacccagggtccacatggaaatgcagttccaggctcccttcaaatggccctgtgctgtgatcagtccccctgcaaaggtctctgagccacagtcccgtcatctgagaagtgaagatgaaacaccagcctcgtggggtcacgcagctctccctgactcccgcctacctgaggatccctctttatcccacctggctgttcagcccgcctggagggaatgtgacgggctggaggcaacagacccttcctctctaggcctcacttgccctcatgctgttacctggaaactgtcttgctacctactgccccttctgtgtgctgagggacaggggacctgtcttggcacaaacatttatagggccaggatggtccttctagagtcttccgaggtactgggccacaccccaccctgccaggtcctcataaccagcttgagtggggtctgttactggctttgccctcaggcatccagcagagacaggcaagtcggggcagggcagggctggacacaccagtcaaaggccagagctctggtggcgtgaccacacctacgtggagagcaaggctgatggcatggcctcccctggtgagagagcaggaagaggcacaggtgagcctgccacagcagcccccagaagccaccatggggcttcaaggctgtgggaatcagcttgagctttttctaaggtggggaagctcttaacaggagcaggcctgctggctgtccccctcctcctgggacgctggagggcaggacacagggcagcatctgacacaccccagcagcaagggcttggcccagaccagccttggggagagaccgcccacccccgggaacccccaggccacactagctccctggtggtcttaccttccccagagcctttgagcccacattcatcggtgtacagcagcagtgggacaggcctcagtcgaccctccagcaccagccacgcctctgccacacctatgccctcaaggaggcatctggcagcctgggcctagctctgcaggtggttagaagagccctagctctaccacttacttacctttgaaaccttgggcaggacactaactggaggttcagtttgctttcagtaatagggggcccgtaagcaggtcctgttgaccttcttactggaggaggagagggagggggtctgaaaaggcgcagagcaaattccagatcctgtcctggatggctgatccctcaccccagcccagaacctgaagtggatgctgccgggatggatgaatgagcattattatggaaaatacttaatccaagcttactgtctagtatcaaaaagttaacctacttgggaagctgaggcaggagaatcacttgaacctgggagacagaggttgcagtgagctgagatcacaccactgtactctagactggtcgacagagtgagaccctgtcttaaaaaaaaaaaaattccttgccttcttccaaaaatgattttagtcaggttagagacacccagaaagagctgggagacagctgctacaacacagacaagagtccctcttgccacagatttgtctcagcttcctggcagccaatgcaaaggattctccgagagaggacagtcatttgtggaaagagaatgctgctgcttagctccattaaagactctgagaagtcctgcagcagagaaacctattaaacccatgcattacccagactttgacctatttcatagaccttattaccatcccaaggtaatatgggattgaggtgaggaaggtcctcacctcgcaaactctccttaaacgtggtgcgtctcagccattctctggggtgggcagcatcaagactgagcctagttgctaaaagccatccagtatcctgcccagaaatccctcagggctacagaaccatgcagc

SEQ ID NO: 2 - RNA, 4341 nt, Homo sapiens
  source 1..4341: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 3 - RNA, 4388 nt, Homo sapiens
  source 1..4388: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactaggtatttaaaggaaatcatgatgcagtattttggatacacaactcaagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 4 - RNA, 4341 nt, Homo sapiens; identical to SEQ ID NO: 2
  source 1..4341: mol_type = mRNA

SEQ ID NO: 5 - RNA, 4341 nt, Homo sapiens; identical to SEQ ID NO: 2
  source 1..4341: mol_type = mRNA

SEQ ID NO: 6 - RNA, 4347 nt, Homo sapiens
  source 1..4347: mol_type = mRNA
agagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcaggtaaagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactga

SEQ ID NO: 7 - protein, 1153 aa, Homo sapiens
MSLKPQQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL

SEQ ID NO: 8 - DNA, 20 bp, Homo sapiens
  source 1..20: mol_type = other DNA
attcactttcataatgctgg

SEQ ID NO: 9 - DNA, 20 bp, Homo sapiens
  source 1..20: mol_type = other DNA
gcgactatacgcgcaatatg

SEQ ID NO: 10 - DNA, 38 bp, Homo sapiens
  source 1..38: mol_type = other DNA
cacgttcactggaatttctctgaactgctgtgtatttc

SEQ ID NO: 11 - DNA, 31 bp, Homo sapiens
  source 1..31: mol_type = other DNA
actggaatttctctgaactgctgtgtatttc

SEQ ID NO: 12 - DNA, 26 bp, Homo sapiens
  source 1..26: mol_type = other DNA
tggaatttctctgaactgctgtgtat

SEQ ID NO: 13 - DNA, 20 bp, Homo sapiens
  source 1..20: mol_type = other DNA
tttccagggccactcgagct

SEQ ID NO: 14 - DNA, 20 bp, Homo sapiens
  source 1..20: mol_type = other DNA
ctatcatcattttccagggc

SEQ ID NO: 15 - DNA, 20 bp, Homo sapiens
  source 1..20: mol_type = other DNA
ttcctcactcctatcatcat

SEQ ID NO: 16 - DNA, 20 bp, Homo sapiens
  source 1..20: mol_type = other DNA
tgtatttttcttcctcactc

SEQ ID NO: 17 - DNA, 20 bp, Homo sapiens
  source 1..20: mol_type = other DNA
tgaactgctgtgtatttttc

SEQ ID NO: 18 - DNA, 20 bp, Homo sapiens
  source 1..20: mol_type = other DNA
ggaatttctctgaactgctg

SEQ ID NO: 19 - DNA, 20 bp, Homo sapiens
  source 1..20: mol_type = other DNA
cacgttcactggaatttctc

SEQ ID NO: 20 - DNA, 20 bp, Homo sapiens
  source 1..20: mol_type = other DNA
ctgtgcccctcacgttcact

SEQ ID NO: 21 - DNA, 20 bp, Homo sapiens
  source 1..20: mol_type = other DNA
agtgtttatgctgtgcccct

SEQ ID NO: 22 - DNA, 20 bp, Homo sapiens
  source 1..20: mol_type = other DNA
ttaaatacctagtgtttatg

SEQ ID NO: 23 - DNA, 20 bp, Homo sapiens
  source 1..20: mol_type = other DNA
atgatttcctttaaatacct

SEQ ID NO: 24 - DNA, 20 bp, Homo sapiens
  source 1..20: mol_type = other DNA
atccaaaatactgcatcatg

SEQ ID NO: 25 - DNA, 20 bp, Homo sapiens
  source 1..20: mol_type = other DNA
accttgagttgtgtatccaa

SEQ ID NO: 26 - DNA, 20 bp, Homo sapiens
  source 1..20: mol_type = other DNA
ccgtctcacacagaccttga

SEQ ID NO: 27 - DNA, 20 bp, Homo sapiens
  source 1..20: mol_type = other DNA
aacaatacaccgtctcacac

SEQ ID NO: 28 - DNA, 20 bp, Homo sapiens
  source 1..20: mol_type = other DNA
aagaggaaatataataacaa

SEQ ID NO: 29 - DNA, 20 bp, Homo sapiens
  source 1..20: mol_type = other DNA
aagctatattaaaggaagag

SEQ ID NO: 30 - DNA, 20 bp, Homo sapiens
  source 1..20: mol_type = other DNA
ttgcattctctacctaagct

SEQ ID NO: 31 - DNA, 20 bp, Homo sapiens
  source 1..20: mol_type = other DNA
taaaccaattctacttgcat

SEQ ID NO: 32 - DNA, 20 bp, Homo sapiens
  source 1..20: mol_type = other DNA
tctaacagatcttaaaccaa

SEQ ID NO: 33 - DNA, 20 bp, Homo sapiens
  source 1..20: mol_type = other DNA
tctgaactgctgtgtatttt

SEQ ID NO: 34 - DNA, 20 bp, Homo sapiens
  source 1..20: mol_type = other DNA
ctctgaactgctgtgtattt

SEQ ID NO: 35 - DNA, 20 bp, Homo sapiens
  source 1..20: mol_type = other DNA
tctctgaactgctgtgtatt

SEQ ID NO: 36 - DNA, 20 bp, Homo sapiens
  source 1..20: mol_type = other DNA
ttctctgaactgctgtgtat

SEQ ID NO: 37 - DNA, 20 bp, Homo sapiens
  source 1..20: mol_type = other DNA
tttctctgaactgctgtgta

SEQ ID NO: 38 - DNA, 20 bp, Homo sapiens
  source 1..20: mol_type = other DNA
atttctctgaactgctgtgt

SEQ ID NO: 39 - DNA, 20 bp, Homo sapiens
  source 1..20: mol_type = other DNA
aatttctctgaactgctgtg

SEQ ID NO: 40 - DNA, 20 bp, Homo sapiens
  source 1..20: mol_type = other DNA
gaatttctctgaactgctgt

SEQ ID NO: 41 - DNA, 20 bp, Homo sapiens
  source 1..20: mol_type = other DNA
ggaatttctctgaactgctg

SEQ ID NO: 42 - DNA, 20 bp, Homo sapiens
  source 1..20: mol_type = other DNA
tggaatttctctgaactgct

SEQ ID NO: 43 - DNA, 20 bp, Homo sapiens
  source 1..20: mol_type = other DNA
ctggaatttctctgaactgc

SEQ ID NO: 44 - DNA, 20 bp, Homo sapiens
  source 1..20: mol_type = other DNA
actggaatttctctgaactg

SEQ ID NO: 45 - DNA, 20 bp, Homo sapiens
  source 1..20: mol_type = other DNA
cactggaatttctctgaact

SEQ ID NO: 46 - DNA, 20 bp, Homo sapiens
  source 1..20: mol_type = other DNA
tcactggaatttctctgaac

SEQ ID NO: 47 - DNA, 20 bp, Homo sapiens
  source 1..20: mol_type = other DNA
ttcactggaatttctctgaa

SEQ ID NO: 48 - DNA, 20 bp, Homo sapiens
  source 1..20: mol_type = other DNA
gttcactggaatttctctga

SEQ ID NO: 49 - DNA, 20 bp, Homo sapiens
  source 1..20: mol_type = other DNA
cgttcactggaatttctctg

SEQ ID NO: 50 - DNA, 20 bp, Homo sapiens
  source 1..20: mol_type = other DNA
acgttcactggaatttctct

SEQ ID NO: 51 - DNA, 20 bp, Homo sapiens
  source 1..20: mol_type = other DNA
ctgaactgctgtgtattttt

SEQ ID NO: 52 - DNA, 19 bp, Homo sapiens
  source 1..19: mol_type = other DNA
ggaatttctctgaactgct

SEQ ID NO: 53 - DNA, 19 bp, Homo sapiens
  source 1..19: mol_type = other DNA
gaatttctctgaactgctg

SEQ ID NO: 54 - DNA, 19 bp, Homo sapiens
  source 1..19: mol_type = other DNA
aatttctctgaactgctgt

SEQ ID NO: 55 - DNA, 19 bp, Homo sapiens
  source 1..19: mol_type = other DNA
atttctctgaactgctgtg

SEQ ID NO: 56 - DNA, 20 bp, Homo sapiens
  source 1..20: mol_type = other DNA
tacctagtgtttatgctgtg

SEQ ID NO: 57 - DNA, 20 bp, Homo sapiens
  source 1..20: mol_type = other DNA
acctagtgtttatgctgtgc

SEQ ID NO: 58 - DNA, 20 bp, Homo sapiens
  source 1..20: mol_type = other DNA
cctagtgtttatgctgtgcc

SEQ ID NO: 59 - DNA, 20 bp, Homo sapiens
  source 1..20: mol_type = other DNA
ctagtgtttatgctgtgccc

SEQ ID NO: 60 - DNA, 20 bp, Homo sapiens
  source 1..20: mol_type = other DNA
tagtgtttatgctgtgcccc

SEQ ID NO: 61 - DNA, 20 bp, Homo sapiens
  source 1..20: mol_type = other DNA
gtgtttatgctgtgcccctc

SEQ ID NO: 62 - DNA, 20 bp, Homo sapiens
  source 1..20: mol_type = other DNA
tgtttatgctgtgcccctca

SEQ ID NO: 63 - DNA, 20 bp, Homo sapiens
  source 1..20: mol_type = other DNA
gtttatgctgtgcccctcac

SEQ ID NO: 64 - DNA, 20 bp, Homo sapiens
  source 1..20: mol_type = other DNA
tttatgctgtgcccctcacg

SEQ ID NO: 65 - DNA, 20 bp, Homo sapiens
  source 1..20: mol_type = other DNA
ttatgctgtgcccctcacgt

SEQ ID NO: 66 - DNA, 20 bp, Homo sapiens
  source 1..20: mol_type = other DNA
gcatcatgatttcctttaaa

SEQ ID NO: 67 - DNA, 20 bp, Homo sapiens
  source 1..20: mol_type = other DNA
catcatgatttcctttaaat

SEQ ID NO: 68 - DNA, 20 bp, Homo sapiens
  source 1..20: mol_type = other DNA
atcatgatttcctttaaata

SEQ ID NO: 69 - DNA, 20 bp, Homo sapiens
  source 1..20: mol_type = other DNA
tcatgatttcctttaaatac

SEQ ID NO: 70 - DNA, 20 bp, Homo sapiens
  source 1..20: mol_type = other DNA
catgatttcctttaaatacc

SEQ ID NO: 71 - DNA, 20 bp, Homo sapiens
  source 1..20: mol_type = other DNA
tgatttcctttaaataccta

SEQ ID NO: 72 - DNA, 20 bp, Homo sapiens
  source 1..20: mol_type = other DNA
gatttcctttaaatacctag

SEQ ID NO: 73 - DNA, 20 bp, Homo sapiens
  source 1..20: mol_type = other DNA
atttcctttaaatacctagt

SEQ ID NO: 74 - DNA, 20 bp, Homo sapiens
  source 1..20: mol_type = other DNA
tttcctttaaatacctagtg

SEQ ID NO: 75 - DNA, 20 bp, Homo sapiens
  source 1..20: mol_type = other DNA
ttcctttaaatacctagtgt

SEQ ID NO: 76 - DNA, 19 bp, Homo sapiens
  source 1..19: mol_type = other DNA
tctgaactgctgtgtattt

SEQ ID NO: 77 - DNA, 19 bp, Homo sapiens
  source 1..19: mol_type = other DNA
ctctgaactgctgtgtatt

SEQ ID NO: 78 - DNA, 19 bp, Homo sapiens
  source 1..19: mol_type = other DNA
tctctgaactgctgtgtat

SEQ ID NO: 79 - DNA, 19 bp, Homo sapiens
  source 1..19: mol_type = other DNA
ttctctgaactgctgtgta

SEQ ID NO: 80 - DNA, 19 bp, Homo sapiens
  source 1..19: mol_type = other DNA
tttctctgaactgctgtgt

SEQ ID NO: 81 - DNA, 25 bp, Homo sapiens
  source 1..25: mol_type = other DNA
aatttctctgaactgctgtgtattt

SEQ ID NO: 82 - DNA, 24 bp, Homo sapiens
  source 1..24: mol_type = other DNA
aatttctctgaactgctgtgtatt

SEQ ID NO: 83 - DNA, 23 bp, Homo sapiens
  source 1..23: mol_type = other DNA
aatttctctgaactgctgtgtat

SEQ ID NO: 84 - DNA, 22 bp, Homo sapiens
  source 1..22: mol_type = other DNA
aatttctctgaactgctgtgta

SEQ ID NO: 85 - DNA, 21 bp, Homo sapiens
  source 1..21: mol_type = other DNA
aatttctctgaactgctgtgt

SEQ ID NO: 86 - DNA, 21 bp, Homo sapiens
  source 1..21: mol_type = other DNA
gaatttctctgaactgctgtg

SEQ ID NO: 87 - DNA, 22 bp, Homo sapiens
  source 1..22: mol_type = other DNA
ggaatttctctgaactgctgtg

SEQ ID NO: 88 - DNA, 23 bp, Homo sapiens
  source 1..23: mol_type = other DNA
tggaatttctctgaactgctgtg

SEQ ID NO: 89 - DNA, 24 bp, Homo sapiens
  source 1..24: mol_type = other DNA
ctggaatttctctgaactgctgtg

SEQ ID NO: 90 - DNA, 25 bp, Homo sapiens
  source 1..25: mol_type = other DNA
actggaatttctctgaactgctgtg

SEQ ID NO: 91 - DNA, 18 bp, Homo sapiens
  source 1..18: mol_type = other DNA
ttctctgaactgctgtgt

SEQ ID NO: 92 - DNA, 18 bp, Homo sapiens
  source 1..18: mol_type = other DNA
tctctgaactgctgtgta

SEQ ID NO: 93 - DNA, 18 bp, Homo sapiens
  source 1..18: mol_type = other DNA
ctctgaactgctgtgtat

SEQ ID NO: 94 - DNA, 18 bp, Homo sapiens
  source 1..18: mol_type = other DNA
tctgaactgctgtgtatt

SEQ ID NO: 95 - DNA, 18 bp, Homo sapiens
  source 1..18: mol_type = other DNA
ctgaactgctgtgtattt

SEQ ID NO: 96 - DNA, 17 bp, Homo sapiens
  source 1..17: mol_type = other DNA
tctctgaactgctgtgt

SEQ ID NO: 97 - DNA, 17 bp, Homo sapiens
  source 1..17: mol_type = other DNA
ctctgaactgctgtgta

SEQ ID NO: 98 - DNA, 17 bp, Homo sapiens
  source 1..17: mol_type = other DNA
tctgaactgctgtgtat

SEQ ID NO: 99 - DNA, 17 bp, Homo sapiens
  source 1..17: mol_type = other DNA
ctgaactgctgtgtatt

SEQ ID NO: 100 - DNA, 17 bp, Homo sapiens
  source 1..17: mol_type = other DNA
tgaactgctgtgtattt

SEQ ID NO: 101 - DNA, 16 bp, Homo sapiens
  source 1..16: mol_type = other DNA
ctctgaactgctgtgt

SEQ ID NO: 102 - DNA, 16 bp, Homo sapiens
  source 1..16: mol_type = other DNA
tctgaactgctgtgta

SEQ ID NO: 103 - DNA, 16 bp, Homo sapiens
  source 1..16: mol_type = other DNA
ctgaactgctgtgtat

SEQ ID NO: 104 - DNA, 16 bp, Homo sapiens
  source 1..16: mol_type = other DNA
tgaactgctgtgtatt

SEQ ID NO: 105 - DNA, 16 bp, Homo sapiens
  source 1..16: mol_type = other DNA
gaactgctgtgtattt

SEQ ID NO: 106 - DNA, 18 bp, Homo sapiens
  source 1..18: mol_type = other DNA
tgagagaattaaaaaaaa

SEQ ID NO: 107 - DNA, 18 bp, Homo sapiens
  source 1..18: mol_type = other DNA
gcttgagagaattaaaaa

SEQ ID NO: 108 - DNA, 18 bp, Homo sapiens
  source 1..18: mol_type = other DNA
aaggcttgagagaattaa

SEQ ID NO: 109 - DNA, 18 bp, Homo sapiens
  source 1..18: mol_type = other DNA
ataaaggcttgagagaat

SEQ ID NO: 110 - DNA, 18 bp, Homo sapiens
  source 1..18: mol_type = other DNA
ggtataaaggcttgagag

SEQ ID NO: 111 - DNA, 18 bp, Homo sapiens
  source 1..18: mol_type = other DNA
ttgggtataaaggcttga

SEQ ID NO: 112 - DNA, 18 bp, Homo sapiens
  source 1..18: mol_type = other DNA
gtattgggtataaaggct

SEQ ID NO: 113 - DNA, 18 bp, Homo sapiens
  source 1..18: mol_type = other DNA
cataggtattgggtataa

SEQ ID NO: 114 - DNA, 18 bp, Homo sapiens
  source 1..18: mol_type = other DNA
ggcgtcataggtattggg

SEQ ID NO: 115 - DNA, 18 bp, Homo sapiens
  source 1..18: mol_type = other DNA
gcatgggcgtcataggta

SEQ ID NO: 116 - DNA, 18 bp, Homo sapiens
  source 1..18: mol_type = other DNA
cactggcatgggcgtcat

SEQ ID NO: 117 - DNA, 18 bp, Homo sapiens
  source 1..18: mol_type = other DNA
tgattcactggcatgggc

SEQ ID NO: 118 - DNA, 18 bp, Homo sapiens
  source 1..18: mol_type = other DNA
tggcttgattcactggca

SEQ ID NO: 119 - DNA, 18 bp, Homo sapiens
  source 1..18: mol_type = other DNA
tgtcttggcttgattcac

SEQ ID NO: 120 - DNA, 18 bp, Homo sapiens
  source 1..18: mol_type = other DNA
ctatatgtcttggcttga

SEQ ID NO: 121 - DNA, 18 bp, Homo sapiens
  source 1..18: mol_type = other DNA
ctgctctatatgtcttgg

SEQ ID NO: 122 - DNA, 18 bp, Homo sapiens
  source 1..18: mol_type = other DNA
tttacctgctctatatgt

SEQ ID NO: 123 - DNA, 18 bp, Homo sapiens
  source 1..18: mol_type = other DNA
acctttacctgctctata

SEQ ID NO: 124 - DNA, 18 bp, Homo sapiens
  source 1..18: mol_type = other DNA
ctcacctttacctgctct

SEQ ID NO: 125 - DNA, 18 bp, Homo sapiens
  source 1..18: mol_type = other DNA
attctcacctttacctgc

SEQ ID NO: 126 - DNA, 18 bp, Homo sapiens
  source 1..18: mol_type = other DNA
attattctcacctttacc

SEQ ID NO: 127 - DNA, 18 bp, Homo sapiens
  source 1..18: mol_type = other DNA
aggattattctcaccttt

SEQ ID NO: 128 - DNA, 18 bp, Homo sapiens
  source 1..18: mol_type = other DNA
ggcaggattattctcacc

SEQ ID NO: 129 - DNA, 18 bp, Homo sapiens
  source 1..18: mol_type = other DNA
acaggcaggattattctc

SEQ ID NO: 130 - DNA, 18 bp, Homo sapiens
  source 1..18: mol_type = other DNA
aacacaggcaggattatt

SEQ ID NO: 131 - DNA, 21 bp, Homo sapiens
  source 1..21: mol_type = other DNA
tttcctttaaatacctagtgt

SEQ ID NO: 132 - DNA, 22 bp, Homo sapiens
  source 1..22: mol_type = other DNA
atttcctttaaatacctagtgt

SEQ ID NO: 133 - DNA, 23 bp, Homo sapiens
  source 1..23: mol_type = other DNA
gatttcctttaaatacctagtgt

SEQ ID NO: 134 - DNA, 24 bp, Homo sapiens
  source 1..24: mol_type = other DNA
tgatttcctttaaatacctagtgt

SEQ ID NO: 135 - DNA, 25 bp, Homo sapiens
  source 1..25: mol_type = other DNA
atgatttcctttaaatacctagtgt

SEQ ID NO: 136 - DNA, 26 bp, Homo sapiens
  source 1..26: mol_type = other DNA
catgatttcctttaaatacctagtgt

SEQ ID NO: 137 - DNA, 27 bp, Homo sapiens
  source 1..27: mol_type = other DNA
tcatgatttcctttaaatacctagtgt